(19) United States
(12) Patent Application Publication
(10) Pub. No.: US 2023/0000997 A1
(43) Pub. Date: Jan. 5, 2023

(54) PROCESSES OF PREPARING POLYGLUTAMATED ANTIFOLATES AND USES OF THEIR COMPOSITIONS

(71) Applicant: L.E.A.F. HOLDINGS GROUP LLC, Gulph Mills, PA (US)

(72) Inventors: Clet NIYIKIZA, Gulph Mills, PA (US); Victor Mandla MOYO, Ringoes, NJ (US); Bolin GENG, Gulph Mills, PA (US)

(21) Appl. No.: 17/632,956

(22) PCT Filed: Aug. 6, 2020
(86) PCT No.: PCT/US2020/045132

§ 371 Date: Feb. 4, 2022

Related U.S. Application Data
(60) Provisional application No. 62/883,311, filed on Aug. 6, 2019.

(51) Int. Cl. A61K 47/64; A61K 31/519; A61K 9/127

(52) U.S. Cl. CPC A61K 47/645; A61K 31/519; A61K 9/1271

(57) ABSTRACT

Provided herein are methods of preparing polyglutamated compounds, such as polyglutamated antifolates, and/or pharmaceutical compositions such as liposomal compositions comprising the same, Also provided herein are substantially pure polyglutamated compounds, or a pharmaceutically acceptable salt thereof, or a pharmaceutical composition such as liposomal composition comprising the same. The present disclosure further provides methods of using the polyglutamated compounds and compositions to treat diseases including hyperproliferative diseases such as cancer, disorders of the immune system such as rheumatoid arthritis, and infectious diseases such as HIV, malaria, and schistomiasis.

### BACKGROUND

The present disclosure generally relates to methods of preparing polyglutamated compounds, in particular, polyglutamated antifolates, or pharmaceutically acceptable salts thereof, pharmaceutical compositions such as liposomal compositions comprising the polyglutamated compounds, or pharmaceutically acceptable salts and methods of using the compounds and compositions to treat diseases including hyperproliferative diseases such as cancer, disorders of the immune system such as rheumatoid arthritis, and infectious diseases such as HIV, malaria, and schistomiasis.

Folate is an essential cofactor that mediates the transfer of one-carbon units involved in nucleotide biosynthesis and DNA repair, the remethylation of homocysteine (Hcy), and the methylation of DNA, proteins, and lipids. The only circulating forms of folates in the blood are monoglutamates and folate monoglutamates are the only form of folate that is transported across the cell membrane—likewise, the monoglutamate form of polyglutamatable antifolates are transported across the cell membrane. Once taken up into cells, intracellular folate is converted to polyglutamates by the enzyme folylpoly-gamma-glutamate synthetase (FPGS).

Antifolate is transported into cells by the reduced folate carrier (RFC) system and folate receptors (FRs) α and β and by Proton Coupled Folate Transporter (PCFT) that is generally most active in a lower pH environment. RFC is the main transporter of antifolates at physiologic pH and is ubiquitously expressed in both normal and diseased cells. Consequently, Antifolate treatment often suffers from the dose-limiting toxicity that is a major obstacle in cancer chemotherapy. Once inside the cell, antifolates are polyglutamated by FPGS, which may add up to 6 glutamyl groups in an L-gamma carboxyl group linkage to the antifolate. The L-gamma polyglutamation of antifolates by FPGS serves at least two main therapeutic purposes: (1) it greatly enhances Antifolate affinity and inhibitory activity for DHFR; and (2) it facilitates the accumulation of polyglutamated antifolate, which unlike antifolate (monoglutamate), is not easily transported out of cells by cell efflux pumps.

While targeting folate metabolism and nucleotide biosynthesis is a well-established therapeutic strategy for cancer, for antifolates, clinical efficacy is limited by a lack of tumor selectivity and the presence of de novo and acquired drug resistance. Antifolates often act during DNA and RNA synthesis, and consequently have a greater toxic effect on rapidly dividing cells such as malignant and myeloid cells. Myelosuppression is typically the dose-limiting toxicity of antifolate therapy and has limited the clinical applications of antifolates.

Resistance to antifolate therapy is typically associated with one or more of, (a) increased cell efflux pump activity, (b) decreased transport of antifolates into cells (c) increased DHFR activity, (d) decreased folylpoly-gamma-glutamate synthetase (FPGS) activity, and (e) increased gamma-glutamyl hydrolase (GGH) activity, which cleaves gamma polyglutamate chains attached to folates and antifolates.

The challenge to the longstanding (>30 years) observation that higher-level polyglutamates of various antifolates have much greater potency compared to lower-level glutamates, has been that the scientific community has relied on the intracellular FPGS mediated mechanisms to convert the lower-level glutamates to their higher-level forms. The present disclosure provides the chemical synthesis, larger scale process and the means to deliver higher-level polyglutamate forms of antifolates directly into the cell, without having to rely on the cells machinery to achieve this goal.

### BRIEF SUMMARY

In various embodiments, the present disclosure is based in part on the advantageous synthetic methods described herein, which allow large-scale synthesis of polyglutamated compounds, in particular, polyglutamated Antifolates such as gamma-polyglutammated Antifolates, and/or alpha-polyglutammated Antifolates, e.g., in a substantially pure form.

In some embodiments, the present disclosure also provides pharmaceutical compositions such as liposomal compositions comprising the polyglutamated Antifolates such as the substantially pure polyglutamated Antifolates and methods of using the compositions. The provided polyglutamated Antifolates such as substantially pure polyglutamated Antifolates such as gamma polyglutamated Antifolate compositions and/or alpha-polyglutammated Antifolate compositions, can be used for example for overcoming the pharmacological challenges associated with the dose limiting toxicities and with treatment resistance associated with antifolate therapy. In some embodiments, the provided methods deliver to cancer cells a gamma or alpha polyglutamated form of the antifolate while (1) minimizing/reducing exposure to normal tissue cells, (2) optimizing/improving the cytotoxic effect of antifolate-based agents on cancer cells and (3) minimizing/reducing the impact of the efflux pumps, and other resistance mechanisms that limit the therapeutic efficacy of antifolates.

Some embodiments of the present disclosure are directed to a method of preparing a polyglutamated drug, in particular, a polyglutamated antifolate, or a pharmaceutically acceptable salt thereof. In some embodiments, the method comprises reacting a protected polyglutamate of Formula I, or a salt thereof, with an antifolate having a formula of Z—COOH, or an activated form thereof, under an amide forming condition to form a compound of Formula II, or a salt thereof, wherein each glutamate unit can independently be in a D-form or an L-form, Pg¹ at each occurrence is independently a carboxylic acid protecting group, and n can be an integer of 0-20, wherein Z is the residue of the antifolate.

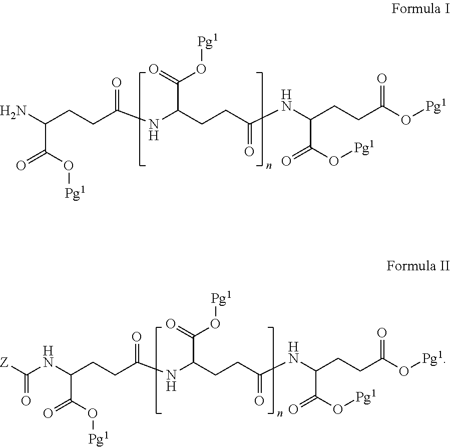

In some specific embodiments, Z is a residue of an antifolate, e.g., selected from methotrexate (MTX), pemetrexed (PMX), lometrexol (LTX), AG2034, raltitrexed (RTX), pralatrexate, GW1843, aminopterin, LY309887 and LY222306. In some embodiments, Z is a residue of pemetrexed. In some embodiments, n is 2-6, such as 2, 3, 4, or 5. In some embodiments, all glutamate units in Formula I or Formula II are in L-form. In some embodiments, all glutamate units in Formula I or Formula II are in D-form. In some embodiments, the reacting comprises reacting the compound of Formula I with the antifolate in the presence of an amide coupling reagent selected from chloroisobutyrate, DCC, DIC, PyBOP, PyAOP, EDCI, HATU, HBTU, TBTU, and T3P. In some embodiments, the protected polyglutamate of Formula I, or a salt thereof can be synthesized by the methods described herein, e.g., in a substantially pure form. In some embodiments, the method further comprises deprotecting the compound of Formula II or a salt thereof to provide a compound of Formula III, or a salt thereof:

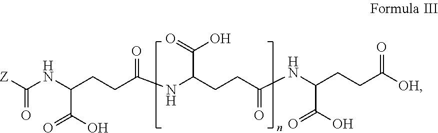

wherein each glutamate unit can independently be in a D-form or an L-form, Z and n are defined herein. In some embodiments, the method further comprises converting the compound of Formula III, or a salt thereof, into an alkali salt (e.g., a sodium salt) of Formula IV:

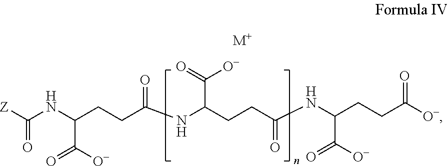

wherein each glutamate unit can independently be in a D-form or an L-form, Z and n are defined herein, M⁺ is an alkali counterion, such as Li⁺, Na⁺, or K⁺.

In some embodiments, the method comprises reacting a protected polyglutamate of Formula I-Alpha, or a salt thereof, with an antifolate having a formula of Z—COOH, or an activated form thereof, under an amide forming condition to form a compound of Formula II-Alpha, or a salt thereof, wherein each glutamate unit can independently be in a D-form or an L-form, Pg¹ at each occurrence is independently a carboxylic acid protecting group, and n can be an integer of 0-20, wherein Z is the residue of the antifolate.

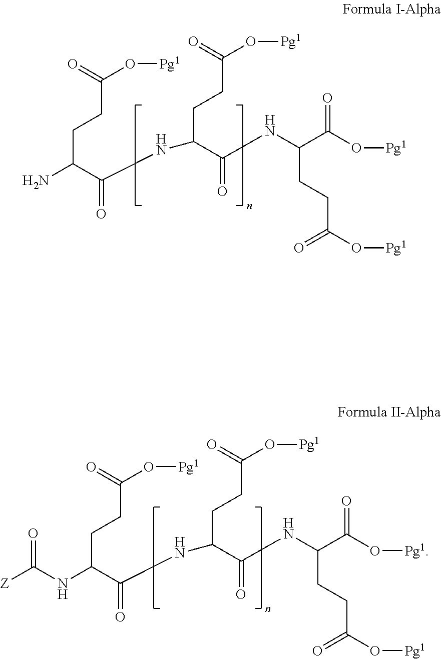

In some specific embodiments, Z is a residue of an antifolate, e.g., selected from methotrexate (MTX), pemetrexed (PMX), lometrexol (LTX), AG2034, raltitrexed (RTX), pralatrexate, GW1843, aminopterin, LY309887 and LY222306. In some embodiments, Z is a residue of pemetrexed. In some embodiments, n is 2-6, such as 2, 3, 4, or 5. In some embodiments, all glutamate units in Formula I-Alpha or Formula II-Alpha are in L-form. In some embodiments, all glutamate units in Formula I-Alpha or Formula II-Alpha are in D-form. In some embodiments, the reacting comprises reacting the compound of Formula I-Alpha with the antifolate in the presence of an amide coupling reagent selected from chloroisobutyrate, DCC, DIC, PyBOP, PyAOP, EDCI, HATU, HBTU, TBTU, and T3P. In some embodiments, the protected polyglutamate of Formula I-Alpha, or a salt thereof can be synthesized by the methods described herein, e.g., in a substantially pure form. In some embodiments, the method further comprises deprotecting the compound of Formula II-Alpha or a salt thereof to provide a compound of Formula III-Alpha, or a salt thereof:

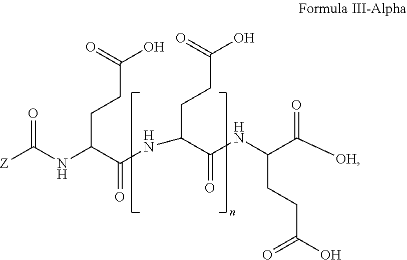

wherein each glutamate unit can independently be in a D-form or an L-form, Z and n are defined herein. In some embodiments, the method further comprises converting the compound of Formula III-Alpha, or a salt thereof, into an alkali salt (e.g., a sodium salt) of Formula IV-Alpha:

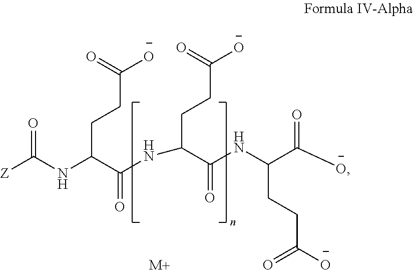

wherein each glutamate unit can independently be in a D-form or an L-form, Z and n are defined herein, M⁺ is an alkali counterion, such as Li⁺, Na⁺, or K⁺.

The synthetic methods herein can provide high purity synthetic intermediates or products that can be used in a pharmaceutical composition. For example, in some embodiments, the present disclosure provides a substantially pure compound of Formula III (e.g., Formula III-1-L, III-1-D as described herein), or a pharmaceutically acceptable salt thereof (e.g., HCl salt or sodium salt). In some embodiments, the present disclosure provides a substantially pure compound of Formula IV (e.g., Formula IV-1-L, IV-1-D as described herein). In some embodiments, Z in Formula III or IV is a residue of an antifolate, e.g., selected from methotrexate (MTX), pemetrexed (PMX), lometrexol (LTX), AG2034, raltitrexed (RTX), pralatrexate, GW1843, aminopterin, LY309887 and LY222306. In some embodiments, Z is a residue of pemetrexed. In some embodiments, n in Formula III or IV is 2-6, such as 2, 3, 4, or 5. In some embodiments, all glutamate units in Formula III or Formula IV are in L-form. In some embodiments, all glutamate units in Formula III or Formula IV are in D-form. In some embodiments, the substantially pure compound of Formula III (e.g., Formula III-1-L, III-1-D as described herein), or a pharmaceutically acceptable salt thereof (e.g., HCl salt or sodium salt), has a purity by HPLC of at least 90% and/or by weight of at least 90%. In some embodiments, the substantially pure compound of Formula IV (e.g., Formula IV-1-L, IV-1-D as described herein), or a pharmaceutically acceptable salt thereof, has a purity by HPLC of at least 90% and/or by weight of at least 90%.

In some embodiments, the present disclosure provides a substantially pure compound of Formula III-Alpha (e.g., Formula III-1-L-Alpha, III-1-D-Alpha as described herein), or a pharmaceutically acceptable salt thereof (e.g., HCl salt or sodium salt). In some embodiments, the present disclosure provides a substantially pure compound of Formula IV-Alpha (e.g., Formula IV-1-L-Alpha, IV-1-D-Alpha as described herein). In some embodiments, Z in Formula III-Alpha or IV-Alpha is a residue of an antifolate, e.g., selected from methotrexate (MTX), pemetrexed (PMX), lometrexol (LTX), AG2034, raltitrexed (RTX), pralatrexate, GW1843, aminopterin, LY309887 and LY222306. In some embodiments, Z is a residue of pemetrexed. In some embodiments, n in Formula III-Alpha or IV-Alpha is 2-6, such as 2, 3, 4, or 5. In some embodiments, all glutamate units in Formula III-Alpha or Formula IV-Alpha are in L-form. In some embodiments, all glutamate units in Formula III-Alpha or Formula IV-Alpha are in D-form. In some embodiments, the substantially pure compound of Formula III-Alpha (e.g., Formula III-1-L-Alpha, III-1-D-Alpha as described herein), or a pharmaceutically acceptable salt thereof (e.g., HCl salt or sodium salt), has a purity by HPLC of at least 90% and/or by weight of at least 90%. In some embodiments, the substantially pure compound of Formula IV-Alpha (e.g., Formula IV-1-L-Alpha, IV-1-D-Alpha as described herein), or a pharmaceutically acceptable salt thereof, has a purity by HPLC of at least 90% and/or by weight of at least 90%.

Some embodiments of the present disclosure are also directed to pharmaceutical compositions comprising compounds of Formula III or IV for example the substantially pure compounds of Formula III or IV as defined herein, e.g., a substantially pure compound of Formula III-1-L, III-1-D, or a pharmaceutically acceptable salt thereof, or Formula IV-1-L or IV-1-D, as described herein. In some embodiments, the pharmaceutical composition can be an aqueous solution or suspension. In some embodiments, the pharmaceutical composition can be a liposomal composition (e.g., described herein), which can be optionally pegylated. In some embodiments, the liposomal composition has a drug load of at least 10%. In some embodiments, the liposomal composition comprises a targeting moiety attached to one or both of a PEG (as applicable) and the exterior of the liposome, and wherein the targeting moiety has a specific affinity for a surface antigen on a target cell of interest.

Some embodiments of the present disclosure are also directed to pharmaceutical compositions comprising compounds of Formula III-Alpha or IV-Alpha for example the substantially pure compounds of Formula III-Alpha or IV-Alpha as defined herein, e.g., a substantially pure compound of Formula III-1-L-Alpha, III-1-D-Alpha, or a pharmaceutically acceptable salt thereof, or Formula IV-1-L-Alpha or IV-1-D-Alpha, as described herein. In some embodiments, the pharmaceutical composition can be an aqueous solution or suspension. In some embodiments, the pharmaceutical composition can be a liposomal composition (e.g., described herein), which can be optionally pegylated. In some embodiments, the liposomal composition has a drug load of at least 10%. In some embodiments, the liposomal composition comprises a targeting moiety attached to one or both of a PEG (as applicable) and the exterior of the liposome, and wherein the targeting moiety has a specific affinity for a surface antigen on a target cell of interest.

Some embodiments of the present disclosure are also directed to methods of treatment of diseases, such as proliferative diseases, diseases of an immune system, infectious diseases, etc., e.g., using a substantially pure polyglutamated Antifolate such as gamma polyglutamated Antifolate compositions and/or alpha polyglutamated Antifolate compositions described herein, or a pharmaceutical composition such as liposomal composition comprising the substantially pure polyglutamated Antifolate. In some embodiments, the substantially pure polyglutamated Antifolate comprises a substantially pure compound of Formula III-1-L, III-1-D, or a pharmaceutically acceptable salt thereof, or Formula IV-1-L or IV-1-D, as described herein. In some embodiments, the substantially pure polyglutamated Antifolate comprises a substantially pure compound of Formula III-1-L-Alpha, III-1-D-Alpha, or a pharmaceutically acceptable salt thereof, or Formula IV-1-L-Alpha or IV-1-D-Alpha, as described herein.

In some embodiments, the present disclosure provides a method for treating cancer that comprises administering an effective amount of for example the substantially pure polyglutamated Antifolate or pharmaceutical composition comprising the substantially pure polyglutamated Antifolate to a subject having or at risk of having cancer. In some embodiments, the cancer is selected from the group consisting of: lung cancer, pancreatic, breast cancer, ovarian cancer, lung cancer, prostate cancer, head and neck cancer, gastric cancer, gastrointestinal cancer, colon cancer, esophageal cancer, cervical cancer, kidney cancer, biliary duct cancer, gallbladder cancer, and a hematologic malignancy. In some embodiments, the cancer cell expresses on its surface the folate receptor bound by the targeting moiety of a liposomal composition.

In some embodiments, the present disclosure provides a method for treating a disorder of the immune system comprising administering an effective amount of for example the substantially pure polyglutamated Antifolate or pharmaceutical composition comprising the substantially pure polyglutamated Antifolate to a subject having or at risk of having a disorder of the immune system.

In some embodiments, the present disclosure provides a method for treating an infectious disease comprising administering an effective amount of for example the substantially pure polyglutamated Antifolate or pharmaceutical composition comprising the substantially pure polyglutamated Antifolate to a subject having or at risk of having a disorder of an infectious disease.

In some embodiments, the present disclosure provides a method for delivering polyglutamated antifolate to a tumor expressing a folate receptor on its surface, the method comprising administering for example the substantially pure polyglutamated Antifolate or pharmaceutical composition comprising the substantially pure polyglutamated Antifolate to a subject having the tumor in an amount to deliver a therapeutically effective dose of the polyglutamated antifolate to the tumor.

Some embodiments of the present disclosure are also directed to a method of preparing a liposomal polyglutamated antifolate composition. In some embodiments, the method comprises: forming a mixture comprising liposomal components and a polyglutamated antifolate in solution; homogenizing the mixture to form liposomes in the solution; and processing the mixture to form liposomes containing the polyglutamated antifolate. In some embodiments, the method comprises forming a mixture comprising: liposomal components and polyglutamated antifolate in a solution; homogenizing the mixture to form liposomes in the solution; processing the mixture to form liposomes entrapping and/or encapsulating polyglutamated antifolate; and providing the targeting moiety on a surface of the liposomes, for example, providing the targeting moiety having the specific affinity for at least one of folate receptor alpha (FR-a), folate receptor beta (FR-β) and folate receptor delta (FR-δ). In some embodiments, the polyglutamated antifolate is the substantially pure polyglutamated Antifolate as defined herein, for example, a substantially pure γPANTIFOL of Formula III-1-L, III-1-D, or a pharmaceutically acceptable salt thereof, or Formula IV-1-L or IV-1-D, a substantially pure aPANTIFOL of Formula III-1-L-Alpha, III-1-D-Alpha, or a pharmaceutically acceptable salt thereof, or Formula IV-1-L-Alpha or IV-1-D-Alpha.

It is to be understood that both the foregoing summary and the following detailed description are exemplary and explanatory only and are not restrictive of the invention herein.

### BRIEF DESCRIPTION OF THE DRAWINGS/FIGURES

FIG. 1 shows an HPLC trace of Compound J with a purity of 98.65%.

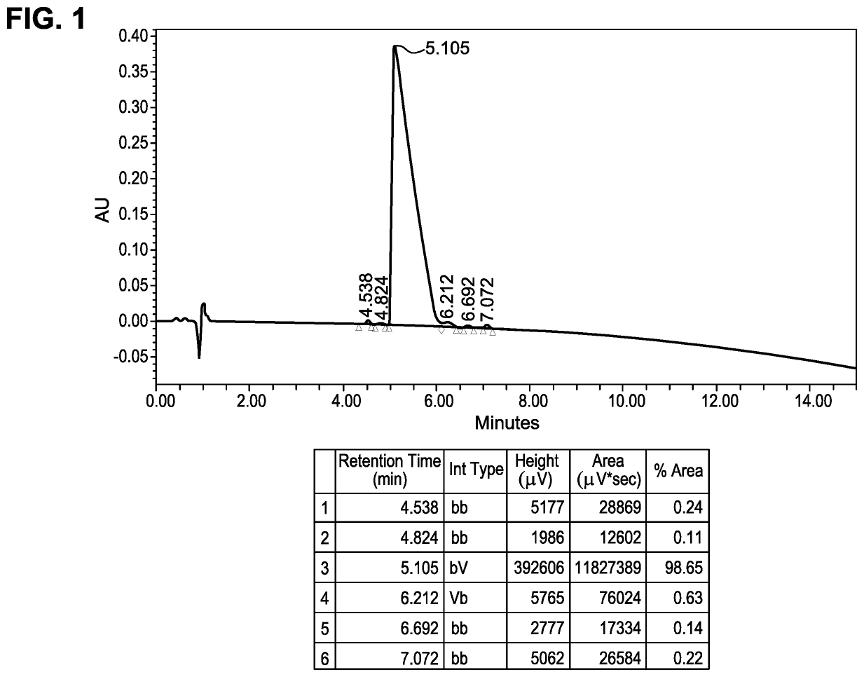

FIG. 2 shows an HPLC trace of Compound K with a purity of 99.17%.

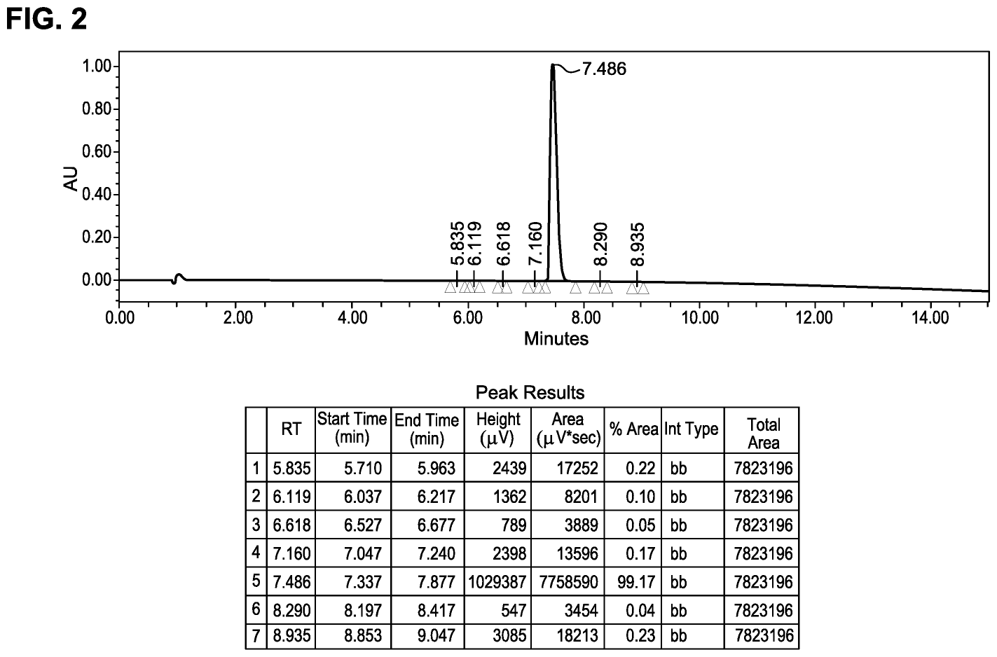

FIG. 3 shows an HPLC trace of Compound L with a purity of 98.03%.

FIG. 4 shows an HPLC trace of Compound 100 with a purity of 98.35%.

### DETAILED DESCRIPTION

The present disclosure generally relates to methods of preparing polyglutamated compounds, such as polyglutamated antifolates, and/or pharmaceutical compositions such as liposomal compositions comprising the same. In some embodiments, a substantially pure polyglutamated compound, or a pharmaceutically acceptable salt thereof, or a pharmaceutical composition such as liposomal composition comprising the same is also provided. In some embodiments, the present disclosure further provides methods of using the polyglutamated compounds and compositions to treat diseases including hyperproliferative diseases such as cancer, disorders of the immune system such as rheumatoid arthritis, and infectious diseases such as HIV, malaria, and schistomiasis.

The present disclosure is based in part on the advantageous synthetic methods described herein. As discussed herein, the synthetic methods described herein (1) can be readily adapted for large-scale synthesis, e.g., kilogram-scale synthesis; (2) can have a high yield, with no or minimized racemization during the synthesis, and simple procedures for purification, such as through crystallization; and (3) can provide high purity intermediates and/or products, including compounds of Formulae I, II, III, and IV and salts thereof related to gamma polyglutamated Antifolates and compounds of Formulae I-Alpha, II-Alpha, III-Alpha, and IV-Alpha and salts thereof related to alpha polyglutamated Antifolates. These high purity intermediates and/or products are also novel compositions of the present disclosure.

### Definitions

Unless defined otherwise, all technical and scientific terms used herein have the same meaning as commonly understood by one of ordinary skill in the art to which the disclosure pertains.

It is understood that wherever embodiments, are described herein with the language “comprising” otherwise analogous embodiments, described in terms of “containing” “consisting of” and/or “consisting essentially of” are also provided. However, when used in the claims as transitional phrases, each should be interpreted separately and in the appropriate legal and factual context (e.g., in claims, the transitional phrase “comprising” is considered more of an open-ended phrase while “consisting of” is more exclusive and “consisting essentially of” achieves a middle ground).

As used herein, the singular form “a”, “an”, and “the”, includes plural references unless it is expressly stated or is unambiguously clear from the context that such is not intended.

As used herein, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone). For example, in embodiments herein, the compounds of the present disclosure may be described as having a purity by HPLC of at least 90% and/or by weight of at least 90%. In such embodiments, it should be understood that the respective compound can have a purity by HPLC of at least 90%, have a purity by weight of at least 90%, or have a purity of at least 90% by both HPLC and by weight.

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

For the chemical structures herein, it is meant to be understood that proper valences are maintained for all moieties and combinations thereof.

It is also meant to be understood that a specific embodiment of a variable moiety herein can be the same or different as another specific embodiment having the same identifier.

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The disclosure additionally encompasses compounds described herein as individual isomers substantially free of other isomers, and alternatively, as mixtures of various isomers including racemic mixtures.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” or “compound(s) of the present invention” refers to any of the compounds described herein according to Formula I, Formula II, Formula III, Formula IV, Formula I-Alpha, Formula II-Alpha, Formula III-Alpha, Formula IV-Alpha, Formula II-Cyclic, Formula III-Cyclic, Formula II-Cyclic-Alpha, Formula III-Cyclic-Alpha, or any of the subformulae thereof, or synthetic precursors thereto, isotopically labeled compound(s) thereof (such as a deuterated analog wherein one of the hydrogen atoms is substituted with a deuterium atom with an abundance above its natural abundance), possible stereoisomers thereof (including diastereoisomers, enantiomers, and racemic mixtures), tautomers thereof, conformational isomers thereof, and/or salts such as pharmaceutically acceptable salts thereof (e.g., acid addition salt such as HCl salt or base addition salt such as Na salt). Hydrates and solvates of the compounds of the present disclosure are considered compositions of the present disclosure, wherein the compound(s) is in association with water or solvent, respectively.

Compounds of the present disclosure characterized by having a Formula III or Formula IV or Formula III-Alpha or Formula IV-Alpha or Formula III-Cyclic or Formula III-Cyclic-Alpha can be used in and/or for a pharmaceutical composition. Compounds of the present disclosure characterized by having a Formula I or Formula II or Formula I-Alpha or Formula II-Alpha are typically synthetic intermediates and not used for preparing a pharmaceutical composition directly.

The term “Gamma polyglutamated antifolate(s) of the present disclosure”, “Gamma polyglutamated Antifolate(s) of the present disclosure”, “γPANTIFOL of the present disclosure”, “γPANTIFOL described (or disclosed or defined) herein” and iterations thereof are used herein to refer to compounds of the present disclosure characterized by having a Formula III or Formula IV or Formula III-Cyclic defined herein, wherein the group Z in Formula III or Formula IV is a residue of an antifolate. A substantially pure “γPANTIFOL of the present disclosure” refers to a compound of the present disclosure characterized by having a Formula III or Formula IV or Formula III-Cyclic, wherein the group Z in Formula III or Formula IV is a residue of an antifolate, which is substantially pure, as defined herein. In embodiments described herein, unless otherwise obvious from context, the term “γPANTIFOL,” whether or not followed by the term “of the present disclosure” or “described (or disclosed or defined) herein” should be understood as referring to the “γPANTIFOL of the present disclosure.”

The term “Alpha polyglutamated antifolate(s) of the present disclosure”, “Alpha polyglutamated Antifolate(s) of the present disclosure”, “αPANTIFOL of the present disclosure”, “αPANTIFOL described (or disclosed or defined) herein” and iterations thereof are used herein to refer to compounds of the present disclosure characterized by having a Formula III-Alpha or Formula IV-Alpha or Formula III-Cyclic-Alpha defined herein, wherein the group Z in Formula III-Alpha or Formula IV-Alpha is a residue of an antifolate. A substantially pure “αPANTIFOL of the present disclosure” refers to a compound of the present disclosure characterized by having a Formula III-Alpha or Formula IV-Alpha or Formula III-Cyclic-Alpha, wherein the group Z in Formula III-Alpha or Formula IV-Alpha is a residue of an antifolate, which is substantially pure, as defined herein. In embodiments described herein, unless otherwise obvious from context, the term “αPANTIFOL,” whether or not followed by the term “of the present disclosure” or “described (or disclosed or defined) herein” should be understood as referring to the “αPANTIFOL of the present disclosure.”

The term “Polyglutamated antifolate(s) of the present disclosure”, “Polyglutamated Antifolate(s) of the present disclosure”, “PANTIFOL of the present disclosure”, “PANTIFOL described (or disclosed or defined) herein” and iterations thereof are used herein to refer to the αPANTIFOL of the present disclosure and/or γPANTIFOL of the present disclosure, as defined herein. A substantially pure “PANTIFOL of the present disclosure” refers to a substantially pure αPANTIFOL of the present disclosure or a substantially pure γPANTIFOL of the present disclosure, as defined herein. In embodiments described herein, unless otherwise obvious from context, the term “PANTIFOL,” whether or not followed by the term “of the present disclosure” or “described (or disclosed or defined) herein” should be understood as referring to the “PANTIFOL of the present disclosure.” In any of the embodiments described herein, unless otherwise obvious from context, the “PANTIFOL” can be an αPANTIFOL of the present disclosure as defined herein. In any of the embodiments described herein, unless otherwise obvious from context, the term “PANTIFOL” can also be a γPANTIFOL of the present disclosure as defined herein.

Compositions such as Liposomal compositions comprising the PANTIFOL described herein should also be understood as directed to either or both αPANTIFOL and γPANTIFOL. For example, in some embodiments, LP-PANTIFOL can be Lp-γPANTIFOL such as, PLp-γPANTIFOL, NTLp-γPANTIFOL, NTPLp-γPANTIFOL, TLp-γPANTIFOL or TPLp-γPANTIFOL. In some embodiments, LP-PANTIFOL can be Lp-αPANTIFOL such as, PLp-αPANTIFOL, NTLp-αPANTIFOL, NTPLp-αPANTIFOL, TLp-αPANTIFOL or TPLp-αPANTIFOL.

In any of the embodiments described herein, unless otherwise specified or obviously contrary from context, the γPANTIFOL or γPANTIFOL of the present disclosure can be a substantially pure γPANTIFOL of the present disclosure (e.g., Formula III-1-L, III-1-D, or a pharmaceutically acceptable salt thereof (e.g., HCl salt or sodium salt), or Formula IV-1-L or IV-1-D). In any of the embodiments described herein, unless otherwise specified or obviously contrary from context, the αPANTIFOL or αPANTIFOL of the present disclosure can be a substantially pure αPANTIFOL of the present disclosure (e.g., Formula III-1-L-Alpha, III-1-D-Alpha, or a pharmaceutically acceptable salt thereof (e.g., HCl salt or sodium salt), or Formula IV-1-L-Alpha or IV-1-D-Alpha). In any of the embodiments described herein, unless otherwise specified or obviously contrary from context, the PANTIFOL or PANTIFOL of the present disclosure can be a substantially pure αPANTIFOL of the present disclosure (e.g., Formula III-1-L-Alpha, III-1-D-Alpha, or a pharmaceutically acceptable salt thereof (e.g., HCl salt or sodium salt), or Formula IV-1-L-Alpha or IV-1-D-Alpha), a substantially pure γPANTIFOL of the present disclosure (e.g., Formula III-1-L, III-1-D, or a pharmaceutically acceptable salt thereof (e.g., HCl salt or sodium salt), or Formula IV-1-L or IV-1-D), or a combination thereof.

In any of the embodiments described herein, unless otherwise specified or obviously contrary from context, the γPANTIFOL or γPANTIFOL of the present disclosure can be an alkali salt of Formula IV-L or IV-D (e.g., IV-1-L or IV-1-D), such as in a substantially pure form. In any of the embodiments described herein, unless otherwise specified or obviously contrary from context, the αPANTIFOL or αPANTIFOL of the present disclosure can be an alkali salt of Formula IV-L-Alpha or IV-D-Alpha (e.g., IV-1-L-Alpha or IV-1-D-Alpha), such as in a substantially pure form.

In any of the embodiments described herein, unless otherwise specified or obviously contrary from context, the γPANTIFOL or γPANTIFOL of the present disclosure can be a compound of Formula III-L or III-D (e.g., III-1-L or III-1-D), or a pharmaceutically acceptable salt thereof, such as in a substantially pure form. In any of the embodiments described herein, unless otherwise specified or obviously contrary from context, the αPANTIFOL or αPANTIFOL of the present disclosure can be a compound of Formula III-L-Alpha or III-D-Alpha (e.g., III-1-L-Alpha or III-1-D-Alpha), or a pharmaceutically acceptable salt thereof, such as in a substantially pure form.

In any of the embodiments described herein, unless otherwise specified or obviously contrary from context, the γPANTIFOL or γPANTIFOL of the present disclosure can be a pharmaceutically acceptable acid addition salt, such as an HCl salt of Formula III-L or III-D (e.g., III-1-L or III-1-D), such as in a substantially pure form. In any of the embodiments described herein, unless otherwise specified or obviously contrary from context, the αPANTIFOL or αPANTIFOL of the present disclosure can be a pharmaceutically acceptable acid addition salt, such as an HCl salt of Formula III-L-Alpha or III-D-Alpha (e.g., III-1-L-Alpha or III-1-D-Alpha), such as in a substantially pure form.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention. For example, in some embodiments, the PANTIFOL of the present disclosure (e.g., Formula III-1-L, III-1-D, III-1-L-Alpha, III-1-D-Alpha, IV-1-L, IV-1-D, IV-1-L-Alpha, or IV-1-D-Alpha) can be isotope labeled, e.g., with ¹³C and/or ¹⁵N for use as a reference compound. It should be understood that these isotope-labeled or -enriched form and formulation containg such forms can also be used as the therapeutic or diagnostic agents.

As used herein, the phrase “administration” of a compound, “administering” a compound, or other variants thereof means providing the compound or a prodrug of the compound to the individual in need of treatment.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable.

The term “leaving group” is given its ordinary meaning in the art of synthetic organic chemistry and refers to an atom or a group capable of being displaced by a nucleophile. See, for example, Smith, March Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates.

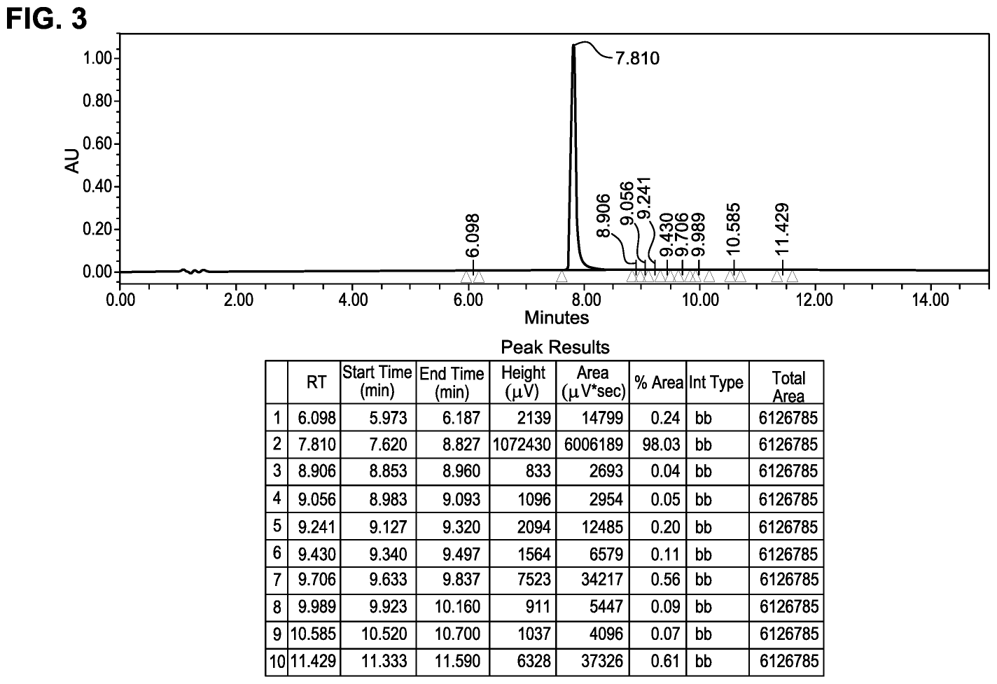

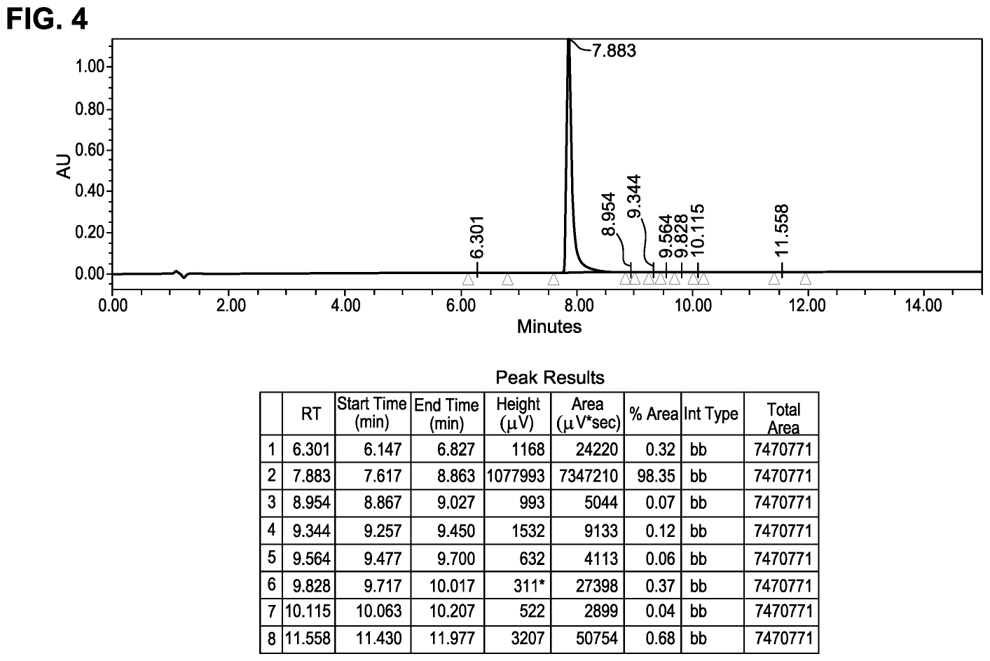

The terms “purity” and “impurities” are used according to their respective art accepted meaning. The terms “purity by HPLC”, “HPLC purity,” and iterations thereof are used to refer to the purity of the respective compound as measured using an HPLC method, e.g., the HPLC method described in the Examples section, expressed as HPLC area percentage. In any of the embodiments described herein, unless otherwise specified or contrary from context, the HPLC purity of a γPANTIFOL of the present disclosure can be measured in accordance with the HPLC Method 2 described in the Examples section, and expressed as the area percentage of the peak representing the compound in an HPLC trace using 210 nm as the detection method. FIGS. 3 and 4 show exemplary HPLC traces and purity determinations using HPLC Method 2. In some embodiments, other HPLC methods such as those using a different column, different gradients, etc. can also be used for measuring the purity of a compound of the present disclosure. Although weight percentage purity of a test sample can also be established by HPLC methods, as used herein, unless specifically referenced as purity by weight, the purity terms such as purity by HPLC or HPLC purity, or analogous terms should not be understood as referring to purity by weight. However, in embodiments herein, the substantially pure compound of the present disclosure can be substantially pure as measured by HPLC purity, by weight, or both. In some embodiments, the term “substantially pure” refers to purity by HPLC, such as an HPLC purity of at least 90%, e.g., at least 90%, at least 95%, at least 98%, or at least 99%. Unless otherwise specified or obvious from context, the HPLC purity herein does not indicate enantiomeric purity.

In some embodiments, the compounds of the present disclosure are described herein as being substantially free of an impurity or impurities. In such embodiments, unless otherwise specified or obvious from context, the percentage described refers to an amount of impurity or impurities as measured by HPLC, expressed as area percentage of the peak(s) representing the impurity or impurities. For example, in some embodiments, it is said that the compound of Formula III (e.g., Formula III-L or III-D) is substantially free (e.g., less than 10%, less than 5%, less than 2%, less than 1%, less than 0.5%, or less than 0.1%) of another compound of Formula III wherein n is not 4. In such embodiments, the “10%” etc. refers to the area percentage(s) of the peak(s) representing the compound(s) of Formula III wherein n is not 4. For the avoidance of doubt, when two or more impurities are present, substantially free of an impurity or impurities should be understood such that none of the individual impurity is present in an amount greater than the specified percentage, such as the “10%” above. In some embodiments, the total amount of the impurities is less than the specified percentage. Other analogous embodiments should be interpreted similarly.

When the compounds of the present disclosure are described herein as being substantially free of a specific enantiomer or diastereomer(s), the percentage described refers to an amount of the specific enantiomer or diastereomer(s), which can be measured by for example by a chiral HPLC, expressed as area percentage of the peak(s) representing the specific enantiomer or diastereomer(s).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

Unless indicated otherwise, the terms “Antifolate” and “ANTIFOL” are used interchangeably to include a salt, acid and and/or free base form of an antifolate (e.g., Antifolate disodium). Compositions containing a Antifolate salt may further contain any of a variety of cations, such as Na+, Mg2+, K+, NH4+, and/or Ca2+. In particular embodiments, the salts are pharmaceutically acceptable salts. Antifolate contains one L-gamma glutamyl group, and is therefore considered to be monoglutamated for the purpose of this disclosure.

The Antifolate can be any known or future derived folate or antifolate that is polyglutamated. In some embodiments, the Antifolate is selected from LV (etoposide), L-leucovorin (L-5-formyltetrahydrofolate); 5-CH3-THF, 5-methyltetrahydrofolate; FA, folic acid; PteGlu, pteroyl glutamate (FA); MTX, methotrexate; 2-dMTX, 2-desamino-MTX; 2-CH3-MTX, 2-desamino-2-methyl-MTX; AMT, aminopterin; 2-dAMT, 2-desamino-AMT; 2-CH3-AMT, 2-desamino-2-methyl-AMT; 10-EdAM, 10-ethyl-10-deazaaminopterin; PT523, N alpha-(4-amino-4-deoxypteroyl)-N delta-(hemiphthaloyl)-L-ornithine; DDATHF (lometrexol), 5,10-dideaza-5,6,7,8,-tetrahydrofolic acid; 5-d(i)H4PteGlu, 5-deaza-5,6,7,8-tetrahydroisofolic acid; N9-CH3-5-d(i)H4PteGlu, N9-methyl-5-deaza-5,6,7,8-tetrahydro-isofolic acid; 5-dPteHCysA, N alpha-(5-deazapteroyl)-L-homocysteic acid; 5-dPteAPBA, N alpha-(5-deazapteroyl)-DL-2-amino-4-phosphonobutanoic acid; 5-dPteOrn, N alpha-(5-deazapteroyl)-L-ornithine; 5-dH4PteHCysA, N alpha-(5-deaza-5,6,7,8-tetrahydropteroyl)-L-homocysteic acid; 5-dH4PteAPBA, N alpha-(5-deaza-5,6,7,8-tetrahydropteroyl)-DL-2-amino-4-phosphobutanoic acid; 5-dH4PteOro, N alpha-(5-deaza-5,6,7,8-tetrahydropteroyl)-L-ornithine; CB3717, N10-propargyl-5,8-dideazafolic acid; ICI-198,583, 2-desamino-2-methyl-N10-propargyl-5,8-dideazafolic acid; 4-H-ICI-198,583, 4-deoxy-ICI-198,583: 4-OCH3-ICI-198,583, 4-methoxy-ICI-198,583 Glu-to-Val-ICI-198,583; valine-ICI-198;583; Glu-to-Sub-ICI-198,583, 2-amino-suberate-ICI-198,583; 198,583, 7-methyl-ICI-198,583; ZD1694, N-[5(N-(3,4-dihydro-2-methyl-4-oxoquinazolin-6-yl-methyl)amino)2-thienyl)]-L-glutamic acid; 2-NH2-ZD1694, 2-amino-ZD1694; BW1843U89, (S)-2[5-(((1,2-dihydro-3-methyl-1-oxobenzo(f)quinazolin-9-yl)methyl)amino-)-1-oxo-2-isoindolinyl]-glutaric acid; LY231514, N-(4-(2-(2-amino-4,7-dihydro-4-oxo-3H-pyrrolo[2,3-D]pyrimidin-5-yl)ethyl)-benzoyl]-L-glutamic acid; IAHQ, 5,8-dideazaisofolic acid; 2-dIAHQ, 2-desamino-IAHQ; 2-CH3-dIAHQ, 2-desamino-2-methyl-IAHQ; 5-d(i)PteGlu, 5-deazaaiso-folic acid; N9-CH3-5-d(i)PteGlu, N9-methyl-5-deazaisofolic acid; N9-CHO-5-d(i)PteGlu, N9-formyl-5-deazaisofolic acid; AG337, 3,4-dihydro-2-amino-6-methly-4-oxo-5-(4-pyridylthio) quanazoline; and AG377, 2,4-diamino-6[N-(4-(phenysulfonyl)benzyl) ethyl) amino]quinazoline; or a stereoisomer thereof.

In some embodiments, the Antifolate is a member selected from Aminopterin, methotrexate, raltitrexed (also referred to as TOMUDEX®, ZD1694 (RTX)), plevitrexed (also referred to as BGC 9331; ZD9331), pemetrexed (also referred to as ALIMTA, LY231514), lometrexol (LMX) (5,10-dideazatetrahydrofolic acid), a cyclopenta[g]quinazoline with a dipeptide ligand, CB3717, CB300945 (also referred to as BGC945) or a stereoisomer thereof such as, 6-R,S-BGC 945 (ONX-0801), CB300638 (also referred to as BGC638), and BW1843U89.

The terms “polyglutamated-Antifolate”, “polyglutamated-ANTIFOL”, “ANTIFOL-PG”, “PANTIFOL” and iterations thereof, are used interchangeably herein to refer to a Antifolate composition that comprises at least one glutamyl group in addition to the glutamyl group in the Antifolate (i.e., ANTIFOL-PGn, wherein n≥1). Reference to the number of glutamyl groups in a γPANTIFOL (ANTIFOL-PG) herein takes into account the glutamyl group in the Antifolate. For example, a ANTIFOL-PG composition containing 5 glutamyl residues in addition to the glutamyl group of ANTIFOL is referred to herein as hexaglutamated Antifolate or Antifolate hexaglutamate. Polyglutamate chains comprise an N-terminal glutamyl group and one or more C-terminal glutamyl groups. The N-terminal glutamyl group of a polyglutamate chain is not linked to another glutamyl group via its amine group, but is linked to one or more glutamyl group via its carboxylic acid group. In some embodiments, the N-terminal glutamyl group of a polyglutamated-Antifolate is the glutamyl group of Antifolate. The C-terminal glutamyl group or groups of a polyglutamate chain are linked to another glutamyl group via their amine group, but are not linked to another glutamyl group via their carboxylic acid group.

The terms “alpha glutamyl group”, “alpha glutamate”, “alpha linkage”, and iterations thereof, as they relate to the linkage of a glutamyl group, refers to a glutamyl group that contains an alpha carboxyl group linkage. In some embodiments, none of the glutamyl groups of the provided polyglutamated Antifolates contain an alpha linkage.

The terms “gamma glutamyl group”, “gamma glutamate”, and “gamma linkage”, as they relate to the linkage of a glutamyl group, refers to a glutamyl group that contains a gamma carboxyl group linkage. In some embodiments, the gamma linkage is an amide bond between the gamma carboxyl group of one glutamyl group and a second glutamyl group. The gamma linkage can be between a glutamyl group and the glutamyl group in the Antifolate, or between the glutamyl group and a second glutamyl group that is not present in Antifolate, such as a glutamyl group within a polyglutamate chain attached to Antifolate. In some embodiments, the gamma linkage refers to the amide bond of the glutamyl group of the Antifolate. Reference to gamma linkages are inclusive of gamma linkage of the glutamyl group of the Antifolate unless it is expressly stated or is unambiguously clear from the context that such is not intended. In some embodiments, the gamma glutamyl group is in the L-form. In some embodiments, the gamma glutamyl group is in the D-form. As discussed herein, during antifolate therapy, antifolates enter the cell and are polyglutamated by the enzyme folylpoly-gamma-glutamate synthetase (FPGS), which adds L glutamyl groups serially to the gamma carboxyl group of the glutamate within the L-glutamyl group in the antifolate. Consequently, D-gamma polyglutamated antifolate compositions are not formed naturally within cells during antifolate therapy.

The terms “gamma polyglutamated Antifolate”, “γ-polyglutamated Antifolate”, “γPANTIFOL”, “gamma polyglutamated-Antifolate”, “polyglutamated-gamma-ANTIFOL”, “γANTIFOL-PG”, and iterations thereof, are used interchangeably herein to refer to a Antifolate composition that comprises at least one gamma glutamyl group having a gamma carboxyl group linkage in addition to the gamma glutamyl group in the Antifolate (e.g., ANTIFOL-PGn, wherein n≥1 γ glutamyl group). Reference to the number of glutamyl groups in a γPANTIFOL (γANTIFOL-PG) herein takes into account the γ-glutamyl group in the Antifolate. For example, a γANTIFOL-PG composition containing 5 γ-glutamyl groups in addition to the glutamyl group in the Antifolate may be referred to herein as gamma hexaglutamated Antifolate or gamma Antifolate hexaglutamate. For example, in some embodiments, a gamma tetraglutamate, pentaglutamate, or hexaglutamate Antifolate can be a compound of Formula III (e.g., III-L or III-D) or a pharmaceutically acceptable salt thereof or an alkali salt of Formula IV (e.g., IV-L or IV-D), wherein n is 2, 3, or 4, respectively.

The terms “alpha polyglutamated Antifolate”, “α-polyglutamated Antifolate”, “αPANTIFOL”, “alpha polyglutamated-Antifolate”, “polyglutamated-alpha-ANTIFOL”, “αANTIFOL-PG”, and iterations thereof, are used interchangeably herein to refer to a Antifolate composition that comprises at least one alpha glutamyl group having a alpha carboxyl group linkage in addition to the glutamyl group in the Antifolate (e.g., ANTIFOL-PGn, wherein n≥1 α glutamyl group). Reference to the number of glutamyl groups in a αPANTIFOL (αANTIFOL-PG) herein takes into account the glutamyl group in the Antifolate. For example, a αANTIFOL-PG composition containing 5 α-glutamyl groups in addition to the glutamyl group in the Antifolate may be referred to herein as alpha hexaglutamated Antifolate or alpha Antifolate hexaglutamate. For example, in some embodiments, an alpha tetraglutamate, pentaglutamate, or hexaglutamate Antifolate can be a compound of Formula III-Alpha (e.g., III-L-Alpha or III-D-Alpha) or a pharmaceutically acceptable salt thereof or an alkali salt of Formula IV-Alpha (e.g., IV-L-Alpha or IV-D-Alpha), wherein n is 2, 3, or 4, respectively.

As use herein, the term “isolated” refers to a composition which is in a form not found in nature. Isolated gamma polyglutamated compositions include those which have been purified to a degree that they are no longer in a form in which they are found in nature. In some embodiments, a gamma polyglutamated Antifolate which is isolated is substantially pure. Isolated compositions will be free or substantially free of material with which they are naturally associated such as other cellular components such as proteins and nucleic acids with which they may potentially be found in nature, or the environment in which they are prepared (e.g., cell culture). The gamma polyglutamated compositions may be formulated with diluents or adjuvants and still for practical purposes be isolated—for example, the gamma polyglutamated compositions will normally be mixed with pharmaceutically acceptable carriers or diluents when used in diagnosis or therapy. In some embodiments, the isolated gamma polyglutamated compositions (e.g., gamma polyglutamates and delivery vehicles such as liposomes containing the gamma polyglutamate contain less than 1% or less than 0.1% undesired DNA or protein content. In some embodiments, the gamma polyglutamate compositions (e.g., gamma polyglutamate and delivery vehicles such as liposomes containing the gamma polyglutamate) are “isolated.”

The term “targeting moiety” is used herein to refer to a molecule that provides an enhanced affinity for a selected target, e.g., a cell, cell type, tissue, organ, region of the body, or a compartment, e.g., a cellular, tissue or organ compartment. The targeting moiety can comprise a wide variety of entities. Targeting moieties can include naturally occurring molecules, or recombinant or synthetic molecules. In some embodiments, the targeting moiety is an antibody, antigen-binding antibody fragment, bispecific antibody or other antibody-based molecule or compound. In some embodiments, the targeting moiety is an aptamer, avimer, a receptor-binding ligand, a nucleic acid, a biotin-avidin binding pair, a peptide, protein, carbohydrate, lipid, vitamin, toxin, a component of a microorganism, a hormone, a receptor ligand or any derivative thereof. Other targeting moieties are known in the art and are encompassed by the disclosure.

The terms “specific affinity” or “specifically binds” mean that a targeting moiety such as an antibody or antigen binding antibody fragment, reacts or associates more frequently, more rapidly, with greater duration, with greater affinity, or with some combination of the above to the epitope, protein, or target molecule than with alternative substances, including proteins unrelated to the target epitope. Because of the sequence identity between homologous proteins in different species, specific affinity can, in several embodiments, include a binding agent that recognizes a protein or target in more than one species. Likewise, because of homology within certain regions of polypeptide sequences of different proteins, the term “specific affinity” or “specifically binds” can include a binding agent that recognizes more than one protein or target. It is understood that, in certain embodiments, a targeting moiety that specifically binds a first target may or may not specifically bind a second target. As such, “specific affinity” does not necessarily require (although it can include) exclusive binding, e.g., binding to a single target. Thus, a targeting moiety may, in certain embodiments, specifically bind more than one target. In certain embodiments, multiple targets may be bound by the same targeting moiety.

The term “epitope” refers to that portion of an antigen capable of being recognized and specifically bound by a targeting moiety (i.e., binding moiety) such as an antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

Expressions like “binding affinity for a target”, “binding to a target” and analogous expressions known in the art refer to a property of a targeting moiety which may be directly measured through the determination of the affinity constants, e.g., the amount of targeting moiety that associates and dissociates at a given antigen concentration. Different methods can be used to characterize the molecular interaction, such as, but not limited to, competition analysis, equilibrium analysis and microcalorimetric analysis, and real-time interaction analysis based on surface plasmon resonance interaction (for example using a BIACORE® instrument). These methods are well-known to the skilled person and are described, for example, in Neri et al., Tibtech 14:465-470 (1996), and Jansson et al., J. Biol. Chem. 272:8189-8197 (1997).

The term “delivery vehicle” refers generally to any compositions that acts to assist, promote or facilitate entry of polyglutamated Antifolate into a cell. Such delivery vehicles are known in the art and include, but are not limited to, liposomes, lipospheres, polymers (e.g., polymer-conjugates), peptides, proteins such as antibodies (e g, immunoconjugates, such as Antibody Drug Conjugates (ADCs) and antigen binding antibody fragments and derivatives thereof), cellular components, cyclic oligosaccharides (e.g., cyclodextrins), micelles, microparticles (e.g., microspheres), nanoparticles (e.g., lipid nanoparticles, biodegradable nanoparticles, and core-shell nanoparticles), hydrogels, lipoprotein particles, viral sequences, viral material, or lipid or liposome formulations, and combinations thereof. The delivery vehicle can be linked directly or indirectly to a targeting moiety. In some examples, the targeting moiety is selected from among a macromolecule, a protein, a peptide, a monoclonal antibody or a fatty acid lipid.

As used herein an “effective amount” refers to a dosage of an agent sufficient to provide a medically desirable result. The effective amount will vary with the desired outcome, the particular condition being treated or prevented, the age and physical condition of the subject being treated, the severity of the condition, the duration of the treatment, the nature of the concurrent or combination therapy (if any), the specific route of administration and like factors within the knowledge and expertise of the health practitioner. An “effective amount” can be determined empirically and in a routine manner, in relation to the stated purpose. In the case of cancer, the effective amount of an agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, duration of progression free survival (PFS), the response rates (RR), duration of response, and/or quality of life.

The terms “hyperproliferative disorder”, “proliferative disease”, and “proliferative disorder”, are used interchangeably herein to pertain to an unwanted or uncontrolled cellular proliferation of excessive or abnormal cells which is undesired, such as, neoplastic or hyperplastic growth, whether in vitro or in vivo. In some embodiments, the proliferative disease is cancer or tumor disease (including benign or cancerous) and/or any metastases, wherever the cancer, tumor and/or the metastasis is located. In some embodiments, the proliferative disease is a benign or malignant tumor. In some embodiments, the proliferative disease is a non-cancerous disease. In some embodiments, the proliferative disease is a hyperproliferative condition such as hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty.

“Cancer,” “tumor,” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (metastasize) as well as any of a number of characteristic structural and/or molecular features. “Tumor,” as used herein refers to all neoplastic cell growth and proliferation, whether malignant or benign, and all pre-cancerous and cancerous cells and tissues. A “cancerous tumor”, or “malignant cell” is understood as a cell having specific structural properties, lacking differentiation and being capable of invasion and metastasis. A cancer that can be treated using a PANTIFOL composition provided herein includes without limitation, a non-hematologic malignancy including such as for example, lung cancer, pancreatic cancer, breast cancer, ovarian cancer, prostate cancer, head and neck cancer, gastric cancer, gastrointestinal cancer, colorectal cancer, esophageal cancer, cervical cancer, liver cancer, kidney cancer, biliary duct cancer, gallbladder cancer, bladder cancer, sarcoma (e.g., osteosarcoma), brain cancer, central nervous system cancer, and melanoma; and a hematologic malignancy such as for example, a leukemia, a lymphoma, and other B cell malignancies, myeloma and other plasma cell dysplasias or dyscrasias. In some embodiments, the cancer is selected from: breast cancer, advanced head and neck cancer, lung cancer, stomach cancer, osteosarcoma, Non-Hodgkin's lymphoma (NHL), acute lymphoblastic leukemia (ALL), mycosis fungoides (cutaneous T-cell lymphoma) choriocarcinoma, chorioadenoma, nonleukemic meningeal cancer, soft tissue sarcoma (desmoid tumors, aggressive fibromatosis), bladder cancer, and central nervous system (CNS) cancer. Other types of cancer and tumors that may be treated using a PANTIFOL composition are described herein or otherwise known in the art. The term “metastasis” refers to spread or dissemination of a tumor, cancer or neoplasia to other sites, locations, regions or organ or tissue systems within the subject, in which the sites, locations regions or organ or tissue systems are distinct from the primary tumor, cancer or neoplasia. The terms “cancer,” “cancerous,” “cell proliferative disorder,” “proliferative disorder,” and “tumor” are not mutually exclusive as referred to herein.

Terms such as “treating,” or “treatment,” or “to treat” refer to both (a) therapeutic measures that cure, slow down, lessen symptoms of, and/or halt progression of a diagnosed pathologic condition or disorder and (b) prophylactic or preventative measures that prevent and/or slow the development of a targeted disease or condition. Thus, subjects in need of treatment include those already with the cancer, disorder or disease; those at risk of having the cancer or condition; and those in whom the infection or condition is to be prevented. Subjects are identified as “having or at risk of having” cancer, an infectious disease, a disorder of the immune system, a hyperproliferative disease, or another disease or disorder referred to herein using well-known medical and diagnostic techniques. In certain embodiments, a subject is successfully “treated” according to the methods provided herein if the subject shows, e.g., total, partial, or transient amelioration or elimination of a symptom associated with the disease or condition (e.g., cancer, inflammation, and rheumatoid arthritis). In specific embodiments, the terms treating,” or “treatment,” or “to treat” refer to the amelioration of at least one measurable physical parameter of a proliferative disorder, such as growth of a tumor, not necessarily discernible by the patient. In other embodiments, the terms treating,” or “treatment,” or “to treat” refer to the inhibition of the progression of a proliferative disorder, either physically by, e.g., stabilization of a discernible symptom, physiologically by, e.g., stabilization of a physical parameter, or both. In other embodiments, the terms treating,” or “treatment,” or “to treat” refer to the reduction or stabilization of tumor size, tumor cell proliferation or survival, or cancerous cell count. Treatment can be with a γ-PANTIFOL composition, alone or in combination with an additional therapeutic agent. Treatment can also be with a αPANTIFOL composition, alone or in combination with an additional therapeutic agent.

“Subject” and “patient,” and “animal” are used interchangeably and refer to mammals such as human patients and non-human primates, as well as experimental animals such as rabbits, rats, and mice, and other animals. Animals include all vertebrates, e.g., mammals and non-mammals, such as chickens, amphibians, and reptiles. “Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and other members of the class Mammalia known in the art. In a particular embodiment, the patient is a human.

“Treatment of a proliferative disorder” is used herein to include maintaining or decreasing tumor size, inducing tumor regression (either partial or complete), inhibiting tumor growth, and/or increasing the life span of a subject having the proliferative disorder. In one embodiment, the proliferative disorder is a solid tumor. Such tumors include, for example, lung cancer, pancreatic cancer, breast cancer, ovarian cancer, prostate cancer, head and neck cancer, gastric cancer, gastrointestinal cancer, colorectal cancer, esophageal cancer, cervical cancer, liver cancer, kidney cancer, biliary duct cancer, gallbladder cancer, bladder cancer, sarcoma (e.g., osteosarcoma), brain cancer, central nervous system cancer, and melanoma. In one embodiment, the proliferative disorder is a hematologic malignancy. Such hematologic malignancies include for example, a leukemia, a lymphoma and other B cell malignancies, myeloma and other plasma cell dysplasias or dyscrasias.

The term “autoimmune disease” as used herein is defined as a disorder that results from an autoimmune response. An autoimmune disease is the result of an inappropriate and excessive response to a self-antigen. Examples of autoimmune diseases include but are not limited to, Addison's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

The terms “inflammation” and “inflammatory disease” are used interchangeably and refer to a disease or disorder characterized or caused by inflammation. “Inflammation” refers to a local response to cellular injury that is marked by capillary dilatation, leukocytic infiltration, redness, heat, and pain that serves as a mechanism initiating the elimination of noxious agents and of damaged tissue. The site of inflammation includes the lungs, the pleura, a tendon, a lymph node or gland, the uvula, the vagina, the brain, the spinal cord, nasal and pharyngeal mucous membranes, a muscle, the skin, bone or bony tissue, a joint, the urinary bladder, the retina, the cervix of the uterus, the canthus, the intestinal tract, the vertebrae, the rectum, the anus, a bursa, a follicle, and the like. Such inflammatory diseases include, but are not limited to, inflammatory bowel disease, rheumatoid diseases (e.g., rheumatoid arthritis), other arthritic diseases (e.g., acute arthritis, acute gouty arthritis, bacterial arthritis, chronic inflammatory arthritis, degenerative arthritis (osteoarthritis), infectious arthritis, juvenile arthritis, mycotic arthritis, neuropathic arthritis, polyarthritis, proliferative arthritis, psoriatic arthritis, venereal arthritis, viral arthritis), fibrositis, pelvic inflammatory disease, acne, psoriasis, actinomycosis, dysentery, biliary cirrhosis, Lyme disease, heat rash, Stevens-Johnson syndrome, mumps, pemphigus vulgaris, and blastomycosis. Inflammatory bowel diseases are chronic inflammatory diseases of the gastrointestinal tract which include, without limitation, Crohn's disease, ulcerative colitis, and indeterminate colitis. Rheumatoid arthritis is a chronic inflammatory disease primarily of the joints, usually polyarticular, marked by inflammatory changes in the synovial membranes and articular structures and by muscle atrophy and rarefaction of the bones.

The term “therapeutic agent” is used herein to refer to an agent or a derivative or prodrug thereof, that can interact with a hyperproliferative cell such as a cancer cell or an immune cell, thereby reducing the proliferative status of the cell and/or killing the cell. Examples of therapeutic agents include, but are not limited to, chemotherapeutic agents, cytotoxic agents, platinum-based agents (e.g., cisplatin, carboplatin, oxaliplatin), taxanes (e.g., TAXOL®), etoposide, alkylating agents (e.g., cyclophosphamide, ifosamide), metabolic antagonists (e.g., an Antifolate (ANTIFOL), 5-fluorouracil gemcitabine, or derivatives thereof), antitumor antibiotics (e.g., mitomycin, doxorubicin), plant-derived antitumor agents (e.g., vincristine, vindesine, Taxol). Such agents may further include, but are not limited to, the anticancer agents trimetrexate, temozolomide, raltitrexed, S-(4-Nitrobenzyl)-6-thioinosine (NBMPR), 6-benzyguanidine (6-BG), bis-chloronitrosourea (BCNU) and CAMPTOTHECIN™, or a therapeutic derivative of any thereof. Additional examples of therapeutic agents that may be suitable for use in accordance with the disclosed methods include, without limitation, anti-restenosis, pro- or anti-proliferative, anti-inflammatory, anti-neoplastic, antimitotic, anti-platelet, anticoagulant, antifibrin, antithrombin, cytostatic, antibiotic and other anti-infective agents, anti-enzymatic, anti-metabolic, angiogenic, cytoprotective, angiotensin converting enzyme (ACE) inhibiting, angiotensin II receptor antagonizing and/or cardioprotective agents. “Therapeutic agents” also refer to salts, acids, and free base forms of the above agents.

As used herein, the term “chemotherapeutic agent” when used in relation to cancer therapy, refers to any agent that results in the death of cancer cells or inhibits the growth or spread of cancer cells. Examples of such chemotherapeutic agents include alkylating agents, antibiotics, antimetabolitic agents, plant-derived agents, and hormones. In some embodiments, the chemotherapeutic agent is cisplatin. In some embodiments, the chemotherapeutic agent is carboplatin. In some embodiments, the chemotherapeutic agent is oxaliplatin. In other embodiments, the chemotherapeutic agent is gemcitabine. In other embodiments, the chemotherapeutic agent is doxorubicin.

The term “antimetabolite” is used herein to refer to an antineoplastic drug that inhibits the utilization of a metabolite or a prodrug thereof. Examples of antimetabolites include Antifolate, pemetrexed, 5-fluorouracil, 5-fluorouracil prodrugs such as capecitabine, 5-fluorodeoxyuridine monophosphate, cytarabine, cytarabine prodrugs such as nelarabine, 5-azacytidine, gemcitabine, mercaptopurine, thioguanine, azathioprine, adenosine, pentostatin, erythrohydroxynonyladenine, and cladribine. Anti-metabolites useful for practicing the disclosed methods include nucleoside analogs, including a purine or pyrimidine analogs. In some embodiments, the polyglutamated Antifolate compositions are used in combination with an antimetabolite selection from fluoropyrimidine 5-fluorouracil, 5-fluoro-2′-deoxycytidine, cytarabine, gemcitabine, troxacitabine, decitabine, Azacytidine, pseudoisocytidine, Zebularine, Ancitabine, Fazarabine, 6-azacytidine, capecitabine, N4-octadecyl-cytarabine, elaidic acid cytarabine, fludarabine, cladribine, clofarabine, nelarabine, forodesine, and pentostatin, or a derivative thereof. In one example, the nucleoside analog is a substrate for a nucleoside deaminase that is adenosine deaminase or cytidine deaminase. In some examples, the nucleoside analog is selected from among fludarabine, cytarabine, gemcitabine, decitabine and azacytidine or derivatives thereof. In certain embodiments, the antimetabolite is 5-fluorouracil.

As used herein, a “taxane” is an anti-cancer agent that interferes with or disrupts microtubule stability, formation and/or function. Taxane agents include paclitaxel and docetaxel as well as derivatives thereof, wherein the derivatives function against microtubules by the same mode of action as the taxane from which they are derived. In certain embodiments, the taxane is paclitaxel or docetaxel, or a pharmaceutically acceptable salt, acid, or derivative of paclitaxel or docetaxel. In certain embodiments, the taxane is paclitaxel (TAXOL®), docetaxel (TAXOTERE®), albumin-bound paclitaxel (nab-paclitaxel; ABRAXANE®), DHA-paclitaxel, or PG-paclitaxel.

The term “pharmaceutically-acceptable carrier” or “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. Pharmaceutically-acceptable carriers can include for example, one or more compatible solid or liquid filler, diluents or encapsulating substances which are suitable for administration to a human or other subject.

### Method of Synthesis

Certain embodiments of the present disclosure are directed to a synthetic method of preparing a polyglutamated compound, such as a gamma polyglutamated compound, an alpha polyglutamated compound, or a pharmaceutically acceptable salt thereof.

### γPANTIFOL

Typically, the synthetic method for γPANTIFOL herein can include an amide coupling reaction of a polyglutamate of Formula I, or a salt thereof, with an antifolate having a formula of Z—COOH, or an activated form thereof, to form a polyglutamated compound of Formula II, or a salt thereof:

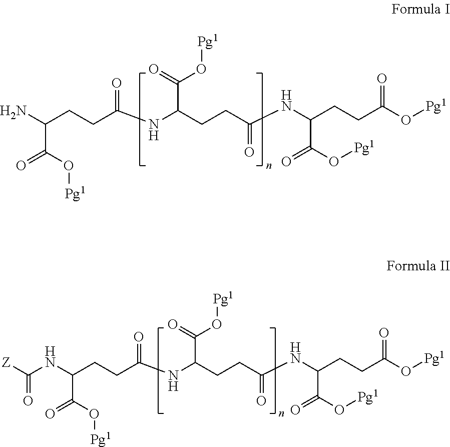

wherein each glutamate unit can independently be in a D-form or an L-form, Pg¹ at each occurrence is independently a carboxylic acid protecting group, and n can be an integer of 0-20, wherein Z is the residue of an Antifolate.

As used herein, Z—COOH should be understood as not including the monoglutamyl group which typically exists in an Antifolate. For example, the antifolate pemetrexed (in acid form) with a D or L mono-glutamate unit is known to have a structure of

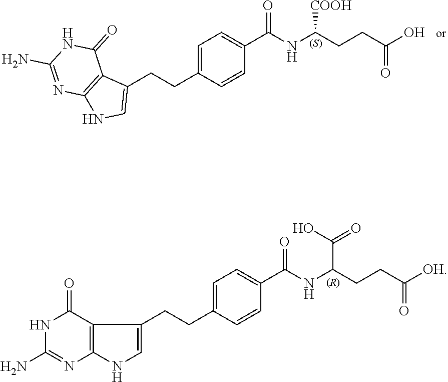

In some specific embodiments, Z can be said to be a residue of pemetrexed. In such embodiments, Z is a residue having the following formula:

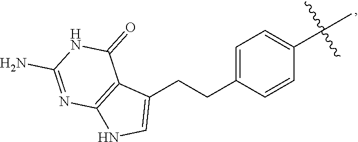 [SMILES: *c1ccc(CCc2c[nH]c3nc(N)[nH]c(=O)c23)cc1]

without the D- or L-glutamate unit. Residues of other Antifolates should be understood similarly.

As used herein, an activated form of a carboxylic acid can include any of those forms wherein the —OH of the carboxylic acid group is activated into a leaving group. Typical activated forms of a carboxylic acid include the corresponding acyl halides, anhydrides, N-linked acyl-heteroaryl (e.g., acyl imidazole (e.g., activation through carbonyl diimidazole), acyl pyridyl, etc.), activated esters, activated forms (1) by various carbodiimide derivatives such as N,N′-dicyclohexylcarbodiimide (DCC), diisopropylcarbodiimide (DIC), 1-Ethyl-3-(3-dimethylaminopropyl)carbodiimide (EDCI), (2) by phosophonic anhydride, such as propanephosphonic acid anhydride (T3P), (3) by uroniums such as 1-[Bis(dimethylamino)methylene]-1H-1,2,3-triazolo[4,5-b]pyridinium 3-oxid hexafluorophosphate (HATU), 2-(1H-Benzotriazole-1-yl)-1,1,3,3-tetramethylaminium tetrafluoroborate (TBTU), 2-(1H-benzotriazol-1-yl)-1,1,3,3-tetramethyluronium hexafluorophosphate (HBTU), or (4) by phosphoniums such as benzotriazol-1-yl-oxytripyrrolidinophosphonium hexafluorophosphate (PyBOP), 7-Azabenzotriazol-1-yloxy)tripyrrolidinophosphonium hexafluorophosphate (PyAOP), etc.

Non-limiting activated forms of a carboxylic acid include a group having a formula of —C(O)—O-G, wherein G is an optionally substituted alkyl, an optionally substituted alkenyl, an optionally substituted alkynyl, an optionally substituted aryl (e.g., optionally substituted phenyl, such as 4-nitrophenyl, 2-nitrophenyl, etc.), optionally substituted heteroaryl (e.g., benzotriazole, residue of 1-hydroxy-benzotriazole (HOBt), residue of 1-hydroxy-7-aza-benzotriazole (HOAt), etc.), optionally substituted heterocyclyl (e.g., N-succinimide), or G has a formula of —C(O)-G^{A}, wherein G^{A} can be optionally substituted alkyl (such as isobutyl), an optionally substituted alkenyl, an optionally substituted alkynyl, an optionally substituted aryl (e.g., optionally substituted phenyl), optionally substituted heteroaryl, optionally substituted carbocyclyl, or optionally substituted heterocyclyl.

The activated form of a carboxylic acid does not need to be isolated for the amide coupling reaction herein. For example, in some embodiments, the polyglutamate of Formula I, or a salt thereof, can react with a carboxylic acid of Z—COOH, in the presence of an amide coupling agent (e.g., chloroisobutyrate, DCC, DIC, PyBOP, PyAOP, EDCI, HATU, HBTU, TBTU, or T3P), which activates the carboxylic acid in situ. However, in some embodiments, an isolated activated form of a carboxylic acid can also be used for the synthetic methods herein.

Suitable conditions for the amide couplings between the polyglutamate of Formula I, or a salt thereof, with the carboxylic acid of Z—COOH, or an activated form thereof, are generally known in the art. Various amide coupling agents can be used for the synthetic methods herein. Non-limiting useful amide coupling agents include chloroisobutyrate, DCC, DIC, PyBOP, PyAOP, EDCI, HATU, HBTU, TBTU, or T3P. Typically, when a carbodiimide coupling agent is used, such as DCC, DIC, EDCI, etc., the amide coupling reaction is also carried out in the presence of a benzotriazole, such as 1-hydroxy-benzotriazole (HOBt), 1-hydroxy-7-aza-benzotriazole (HOAt), etc. In some embodiments, a base is also added for the amide coupling. Suitable bases include inorganic bases such as carbonates (e.g., Na₂CO₃, NaHCO₃) and organic bases such as amine bases (e.g., diisopropylethyl amine, triethyl amine, N-methylmorpholine, pyridine) etc. The amide coupling reaction herein is typically carried out under conditions such that no or minimized racemization of chiral center(s) occurs. Exemplary amide coupling reaction conditions are shown in the Examples section.

In some embodiments, the synthetic method herein further comprises deprotecting the Pg1 groups of Formula II, or a salt thereof, to form the free carboxylic acid compound of Formula III, or a salt thereof:

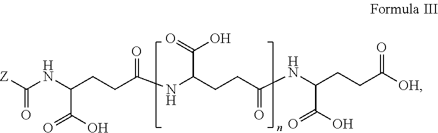

wherein Z and n are defined herein.

In some embodiments, each of the Pg¹ groups of Formula II can be deprotected under acidic conditions. For example, in some embodiments, each of the Pg¹ groups of Formula II is a tert-butyl group. In some embodiments, the deprotecting of the compound of Formula II can be effected with an acid, such as trifluoroacetic acid (TFA), HCl, etc.

In some embodiments, the synthetic method herein further comprises converting the free carboxylic acid compound of Formula III, or a salt thereof, into an alkali salt of Formula IV:

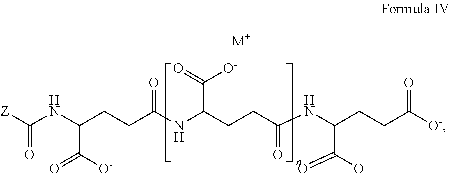

wherein M⁺ is an alkali counterion, such as Li⁺, Na⁺, or K⁺. The conversion can be typically carried out by contacting the compound of Formula III or a salt thereof with a suitable alkali base, such as NaOH, etc. In some embodiments, the alkali salt of Formula IV can be further isolated, purified, and/or crystallized by any suitable method, e.g., described herein. While the molar equivalent of M⁺ in Formula IV is not specified, Formula IV should not be understood as limited to having one molar equivalent of M⁺. To be clear, M⁺ in Formula IV typically can balance the negative charges of the carboxylic acid groups in Formula IV, with one mole of M⁺ per one mole of the negative charged carboxylic acid group in Formula IV. For example, in some embodiments, n is 4, and M⁺ is Na⁺, the alkali salt of Formula IV can be a hepta-sodium salt, i.e., 7 Na⁺ to counter balance the negative charges of the carboxylic acids so that Formula IV is neutral overall. In some embodiments, the alkali cation M⁺ can also be combined with one or more other cations (e.g., pharmaceutically acceptable cations) to counter balance the negative charges of the carboxylic acid groups so that Formula IV is overall neutral.

The synthetic methods described herein have various advantages. For example, the synthetic methods described herein (1) can be readily adapted for large-scale synthesis, e.g., kilogram-scale synthesis; (2) can have a high yield, with no or minimized racemization during the synthesis, and simple procedures for purification, such as through crystallization; and (3) can provide high purity intermediates and/or products, including compounds of Formulae I, II, III, and IV and salts thereof. (4) can reduce the requirements of manufacturing equipments due to a smaller number of repeating steps are used. These high purity intermediates and/or products are also novel compositions of the present disclosure.

While many of the embodiments described herein are directed to drugs (e.g., Antifolates) that are polyglutamated through a carboxylic acid group via an amide formation, the present disclosure is not limited to this mode of polyglutamation. For example, the present disclosure also contemplates polyglutamation of drug molecules (e.g., Antifolates) that have other function groups such that an amide bond, a carbon-nitrogen single bond, an ester bond, a carbamate, an urea, a sulfonamide, a sulfamate, a sulfamide, etc. can be formed through an NH₂ or COOH group of the polyglutamate of Formula I or a protected/deprotected derivative thereof.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4th ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wis., USA), Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

Formula I and Preparation

To prepare a high purity polyglutamated antifolate herein, the synthesis typically uses a substantially pure polyglutamate of Formula I or a salt thereof. For example, for the synthetic method, the compound of Formula I (e.g., Formula I-L or I-D) or a salt thereof (e.g., a pharmaceutically acceptable salt) can typically have a purity of at least 90% (e.g., at least 90%, at least 95%, at least 98%, at least 99%) by HPLC and/or by weight. Methods for preparing such substantially pure polyglutamate of Formula I are described herein.

In some embodiments, the substantially pure polyglutamate of Formula I are also in a stereoisomerically pure or substantially pure form. In some embodiments, the polyglutamate of Formula I can exist predominantly in one enantiomeric form, which can be free or substantially free (e.g., containing less than 5%, less than 2%, less than 1%, less than 0.5%, or less than 0.1%) of the other enantiomeric form. In some embodiments, the polyglutamate of Formula I can also exist predominantly as one diastereomer, which can be free or substantially free (e.g., containing less than 5%, less than 2%, less than 1%, less than 0.5%, or less than 0.1%) of any other diastereomer(s). The amount of enantiomer and/or diastereomer(s) can be readily determined by those skilled in the art, for example, using HPLC (e.g., a chiral HPLC).

In some embodiments, each of the glutamate units in Formula I is in an L-form, and the compound of Formula I is a compound of Formula I-L:

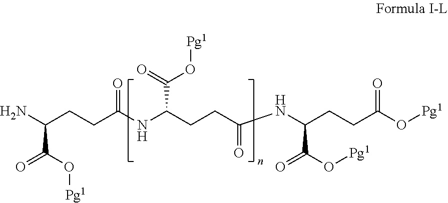

wherein Pg¹ and n are defined herein. In some embodiments, the polyglutamate of Formula I-L can be stereoisomerically pure or substantially pure. For example, in some embodiments, the polyglutamate of Formula I-L can be free or substantially free (e.g., containing less than 5%, less than 2%, less than 1%, less than 0.5%, or less than 0.1%) of the other enantiomeric form. In some embodiments, the polyglutamate of Formula I-L can also be free or substantially free (e.g., containing less than 5%, less than 2%, less than 1%, less than 0.5%, or less than 0.1%) of any other diastereomer(s). However, in some embodiments, the polyglutamate of Formula I-L can also exist in a racemic mixture or in a stereoisomeric mixture.

In some embodiments, each of the glutamate units in Formula I is in a D-form, and the compound of Formula I is a compound of Formula I-D:

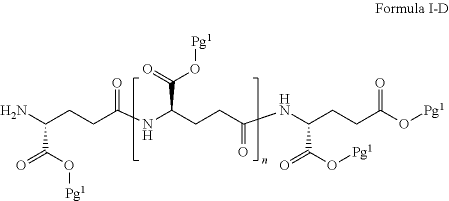

wherein Pg¹ and n are defined herein. In some embodiments, the polyglutamate of Formula I-D can also be stereoisomerically pure or substantially pure. For example, in some embodiments, the polyglutamate of Formula I-D can be free or substantially free (e.g., containing less than 5%, less than 2%, less than 1%, less than 0.5%, or less than 0.1%) of the other enantiomeric form. In some embodiments, the polyglutamate of Formula I-D can also be free or substantially free (e.g., containing less than 5%, less than 2%, less than 1%, less than 0.5%, or less than 0.1%) of any other diastereomers. However, in some embodiments, the polyglutamate of Formula I-D can also exist in a racemic mixture or in a stereoisomeric mixture.

Various carboxylic acid protecting groups are suitable for use as Pg¹ in Formula I (e.g., Formula I-L or I-D). Carboxylic acid protecting groups (or alternatively referred to herein as carboxyl protecting group) are generally known in the art, for example, as described in “Protective Groups in Organic Synthesis”, 4th ed. P. G. M. Wuts; T. W. Greene, John Wiley, 2007, and references cited therein. In some embodiments, Pg¹ in Formula I (e.g., Formula I-L or I-D) at each occurrence can be a carboxyl protecting group that can be removed under acidic conditions, such as a tertiary alkyl group, such as tert-butyl. In some embodiments, Pg¹ in Formula I (e.g., Formula I-L or I-D) at each occurrence can be a carboxyl protecting group that can be removed under basic conditions, such as methyl, ethyl, benzyl, etc. In some embodiments, Pg¹ in Formula I (e.g., Formula I-L or I-D) at each occurrence can be a carboxyl protecting group that can be removed through a nucleophilic attack, such as methyl, ethyl, benzyl. In some embodiments, Pg¹ in Formula I (e.g., Formula I-L or I-D) at each occurrence can be a carboxyl protecting group that can be removed through a photoreaction, i.e., the protecting group is a photoreleasable protecting group. Photoreleasable protecting groups are known in the art, for example, as described in Klan et al. “Photoremovable Protecting Groups in Chemistry and Biology: Reaction Mechanisms and Efficacy,” Chem. Rev. 113:119-191 (2013). In some embodiments, Pg¹ in Formula I (e.g., Formula I-L or I-D) at each occurrence can be a carboxyl protecting group that can be removed under hydrogenation conditions, such as benzyl.

Typically, all of the Pg¹ in Formula I (e.g., Formula I-L or I-D) are the same protecting group. However, in some embodiments, the Pg¹ groups in Formula I (e.g., Formula I-L or I-D) can also be different and can be deprotected under different conditions. For example, in some embodiments, the Pg¹ group for the C-terminal carboxylic acid group (either alpha-carboxylic acid group or gamma-carboxylic acid group) can be different from and/or orthogonal to the Pg¹ group(s) for the remaining carboxylic acid groups. In such embodiments, the Pg¹ group for the C-terminal carboxylic acid group (either alpha-carboxylic acid group or gamma-carboxylic acid group) can be selectively deprotected in the presence of the other Pg¹ group(s), and vice versa, which allows further functionalization of the C-terminal carboxylic acid group.

The polyglutamate of Formula I (e.g., Formula I-L or I-D) described herein can typically comprise 2-20 glutamate units, for example, 2-20, 2-15, 2-10, 2-6, 2-5, or more than 5. In some embodiments, the polyglutamate of Formula I (e.g., Formula I-L or I-D) can refer to a specific oligomer, with n being a specific integer. For example, in some embodiments, n in Formula I (e.g., Formula I-L or I-D) can be 0, 1, 2, 3, 4, 5, 6, 7, 8, 9, 10, 11, 12, 13, 14, 15, 16, 17, or 18. In some embodiments, the polyglutamate of Formula I (e.g., Formula I-L or I-D) can be a hexaglutamate (n is 4), which can be substantially free of a polyglutamate of Formula I wherein n is not 4. In some embodiments, the polyglutamate of Formula I can also refer to a mixture of polyglutamates which have different number of glutamate units. For example, in some embodiments, the polyglutamate of Formula I can comprise a mixture of polyglutamate of Formula I wherein n is 0-18, 0-13, 2-6, 0-8, 0-3, etc.

Compounds of Formula I (e.g., Formula I-L or I-D) are typically prepared from deprotection of a compound of Formula I-P, or a salt thereof:

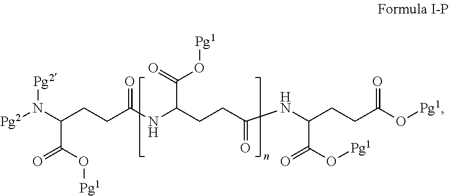

wherein Pg² and Pg^{2′} are independently hydrogen or a nitrogen protecting group, provided that at least one of Pg² and Pg^{2′} is a nitrogen protecting group; or Pg² and Pg^{2′} together with the nitrogen atom they are attached to form a cyclic protected amino group. As used herein, the term “amine protecting group” and “nitrogen protecting group” are used interchangeably. Nitrogen protecting groups are generally known in the art, for example, as described in “Protective Groups in Organic Synthesis”, 4^{th} ed. P. G. M. Wuts; T. W. Greene, John Wiley, 2007, and references cited therein. Non-limiting examples of suitable nitrogen protecting groups include carbobenzyloxy (Cbz) (removable by hydrogenolysis), p-methoxybenzyl carbonyl (Moz or MeOZ) (removable by hydrogenolysis), tert-butyloxycarbonyl (Boc) (removable by acids, such as HCl or trifluoroacetic acid, or by heating), 9-fluorenylmethyloxycarbonyl (FMOC) (removable by base, such as piperidine), acetyl (Ac) (removable by treatment with a base), benzoyl (Bz) (removable by treatment with a base, most often with aqueous or gaseous ammonia or methylamine), benzyl (Bn) (removable by hydrogenolysis), a carbamate (removable by acid and mild heating), p-methoxybenzyl (PMB) (removable by hydrogenolysis), 3,4-dimethoxybenzyl (DMPM) (removable by hydrogenolysis), p-methoxyphenyl (PMP) (removable by ammonium cerium(IV) nitrate), a succinimide (a cyclic imide) (removable by treatment with a base), tosyl (Ts) (removable by concentrated acid and strong reducing agents), and other sulfonamides (Nosyl and Nps) (removable by samarium iodide, tributyltin hydride, etc.). In some embodiments, neither of Pg² and Pg^{2′} is Fmoc.

Typically, the Pg² and Pg^{2′} are selected such that the deprotection can be carried out in high efficiency, such that the deprotected product, i.e., compound of Formula I or salts thereof, can be used directly for coupling with Z—COOH or an activated form thereof. For example, in some embodiments, one of Pg² and Pg^{2′} in Formula I-P is hydrogen, and the other of Pg² and Pg^{2′} is a nitrogen protecting group capable of being deprotected via hydrogenation, e.g., Pg² is benzyloxycarbonyl (Cbz). In such embodiments, the deprotection can be carried out in high efficiency and typically, the deprotected product can be used directly without further purification.

In any of the embodiments described herein, the Pg¹ groups and the amine protecting group(s) of Formula I-P can be orthogonal. For example, in some embodiments, the amine protecting group(s) of Formula I-P can be protecting groups removable under hydrogenation conditions but are stable under acidic conditions (e.g., TFA), whereas the Pg¹ groups are stable under hydrogenation conditions but are removable under acidic conditions (e.g., TFA). Alternatively, in some embodiments, the amine protecting group(s) of Formula I-P can be protecting groups that are stable under hydrogenation conditions but are removable under acidic conditions (e.g., TFA), whereas the Pg¹ groups are removable under hydrogenation conditions but are stable under acidic conditions (e.g., TFA). In some embodiments, one of Pg² and Pg^{2′} in Formula I-P is hydrogen, and the other of Pg² and Pg^{2′} is a nitrogen protecting group capable of being deprotected via hydrogenation, e.g., Pg² is benzyloxycarbonyl. Various conditions for hydrogenation are suitable. Typically, such hydrogenation can be carried out in the presence of a heterogenous catalyst, such as Pd/C, under H₂ gas, in a solvent such as an alcoholic solvent (e.g., methanol, ethanol, etc.). In some embodiments, all of the Pg¹ groups are acid deprotectable protecting groups such as tert-butyl.

For preparing a high purity compound of Formula I or salt thereof, the compound of Formula I-P (e.g., Formula I-P-L or I-P-D) or a salt thereof (e.g., a pharmaceutically acceptable salt) used is typically also substantially pure, for example, has a purity of at least 90% (e.g., at least 90%, at least 95%, at least 98%, at least 99%) by HPLC and/or by weight. In some embodiments, the compound of Formula I-P can also exist predominantly in one enantiomeric form, which can be free or substantially free (e.g., containing less than 5%, less than 2%, less than 1%, less than 0.5%, or less than 0.1%) of the other enantiomeric form. In some embodiments, the compound of Formula I-P can also exist predominantly as one diastereomer, which can be free or substantially free (e.g., containing less than 5%, less than 2%, less than 1%, less than 0.5%, or less than 0.1%) of any other diastereomer(s).

In some embodiments, each of the glutamate units in the compound of Formula I-P is in an L-form, and the compound of Formula I-P is a compound of Formula I-P-L:

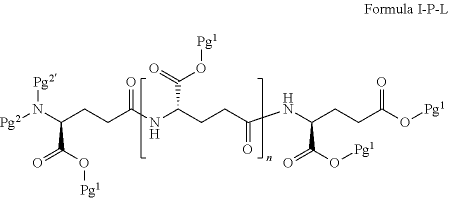

wherein Pg¹, Pg², Pg^{2′}, and n are defined herein. In some embodiments, the compound of Formula I-P-L can be stereoisomerically pure or substantially pure. For example, in some embodiments, the compound of Formula I-P-L can be free or substantially free (e.g., containing less than 5%, less than 2%, less than 1%, less than 0.5%, or less than 0.1%) of the other enantiomeric form. In some embodiments, the compound of Formula I-P-L can also be free or substantially free (e.g., containing less than 5%, less than 2%, less than 1%, less than 0.5%, or less than 0.1%) of any other diastereomer(s). However, in some embodiments, the compound of Formula I-P-L can also exist in a racemic mixture or in a stereoisomeric mixture.

In some embodiments, each of the glutamate units in Formula I-P is in a D-form, and the compound of Formula I-P is a compound of Formula I-P-D:

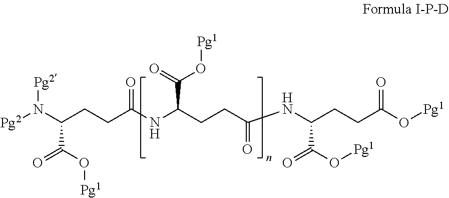

wherein Pg¹, Pg², Pg^{2′}, and n are defined herein. In some embodiments, the compound of Formula I-P-D can also be stereoisomerically pure or substantially pure. For example, in some embodiments, the compound of Formula I-P-D can be free or substantially free (e.g., containing less than 5%, less than 2%, less than 1%, less than 0.5%, or less than 0.1%) of the other enantiomeric form. In some embodiments, the compound of Formula I-P-D can also be free or substantially free (e.g., containing less than 5%, less than 2%, less than 1%, less than 0.5%, or less than 0.1%) of any other diastereomers. However, in some embodiments, the compound of Formula I-P-D can also exist in a racemic mixture or in a stereoisomeric mixture.

Compounds of Formula I-P (e.g., Formula I-P-L or I-P-D) are typically prepared from protected glutamate or protected polyglutamate via amide coupling reactions. For example, in some embodiments, the method of preparing a compound of Formula I-P comprises:

reacting an acid of Formula S-1, or an activated form thereof, with a protected polyglutamate of Formula S-2, or a salt thereof, under an amide forming condition to form a compound of Formula S-3, or a salt thereof:

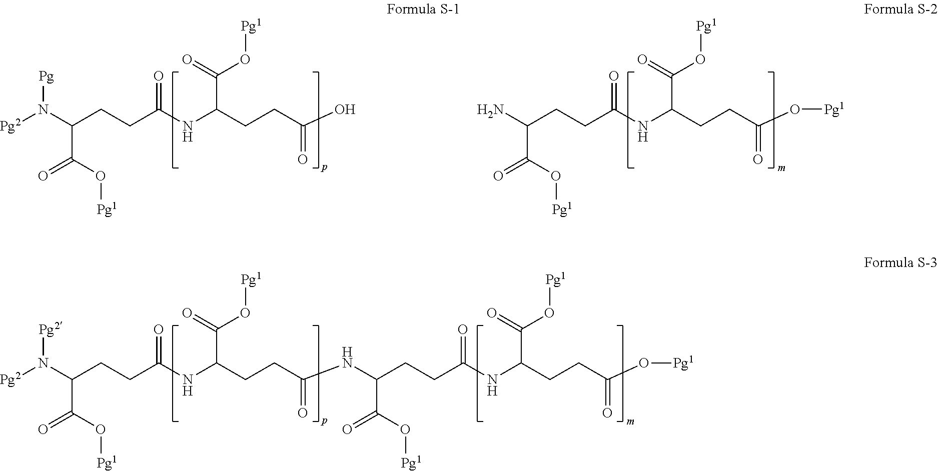

wherein Pg¹, Pg² and Pg^{2′} are defined herein, wherein each glutamate unit is independently in an L-form or D-form (e.g., all glutamate units are in L-form or all glutamate units are in D-form), wherein m+p=n, and n is defined herein. In some embodiments, m is 0-19, for example, 2-6 (e.g., 3 or 4). In some embodiments, p is 0-19. Typically, p is 0. However, in some embodiments, p is not 0. Those skilled in the art would understand that when m+p=n, Formula S-3 is the same as Formula I-P. In some embodiments, Formula S-3 can be deprotected to provide the compound of Formula I. Compounds of Formula S-2 can be prepared similarly.

In some embodiments, p in Formula S-1 is 0 and the glutamate units of Formula I-P are introduced one by one consecutively. For example, in some embodiments, the method of preparing a compound of Formula I-P comprises:
1) reacting an acid of Formula S-1-A, or an activated form thereof, with an amine of Formula S-2-A, or a salt thereof, under amide forming conditions to provide the dimer compound of Formula S-3-A:

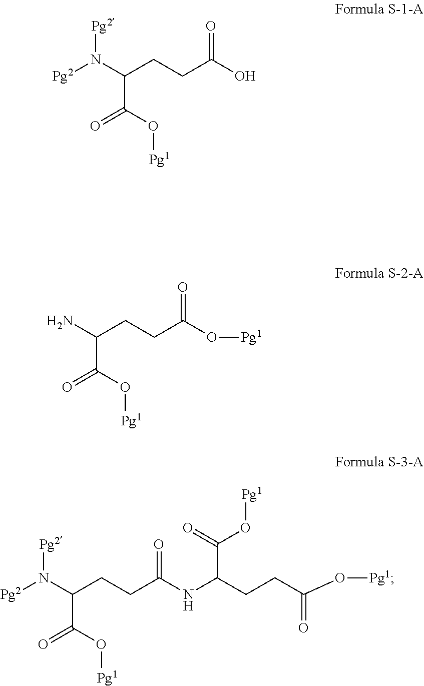

2) deprotecting the amine protecting group(s) of the compound of Formula S-3-A to form a compound of S-2-B, or a salt thereof;

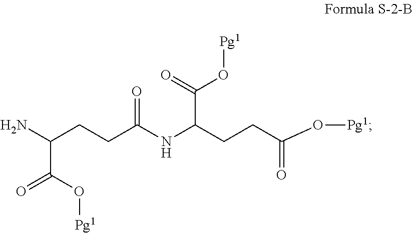

3) reacting the compound of Formula S-2-B or a salt thereof with the acid of Formula S-1-A, or an activated form thereof, under amide forming conditions to elongate the chain by one glutamate unit to provide the trimer compound of Formula S-3-B:

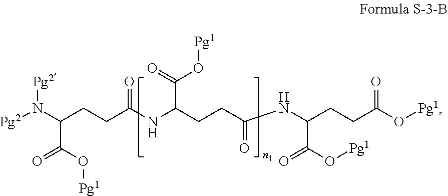

wherein n₁ is 1; and optionally
4) repeating the sequence of deprotecting the amine protecting group(s) and reacting the deprotected compound with the acid of Formula S-1-A, or an activated form thereof, under amide forming conditions to elongate the chain until the desired number of glutamate unit is reached to form the compound of Formula I-P:

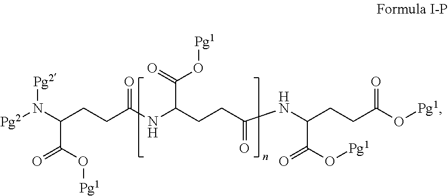

wherein Pg¹, Pg², Pg^{2′} and n are defined herein, wherein each glutamate unit is independently in an L-form or D-form (e.g., all glutamate units are in L-form or all glutamate units are in D-form). An example of preparing a compound of Formula I-P (n is 4) is provided in the Examples section.

In some embodiments, the synthetic method herein is for preparing a compound of Formula I-P-L. In such embodiments, the corresponding starting materials and/or intermediates used for the methods typically have each of the glutamate units in an L-form. For example, in some embodiments, each glutamate unit in each of Formula S-1, S-2, S-3, S-1-A, S-2-A, S-3-A, S-2-B, and S-3-B can be in the L-form. For example, in some embodiments, compounds of Formulae S-1, S-2, and S-3 can have a Formula S-1-L, S-2-L, or S-3-L, wherein the variables are defined herein, respectively:

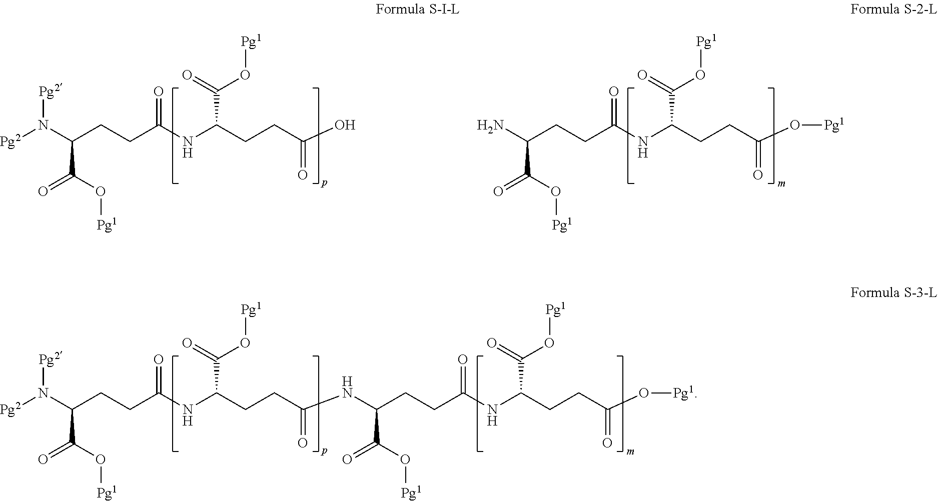

In some embodiments, the compound of Formula S-1-L, S-2-L, or S-3-L can be free or substantially free (e.g., containing less than 5%, less than 2%, less than 1%, less than 0.5%, or less than 0.1%) of the other enantiomeric form, respectively. In some embodiments, the compound of Formula S-1-L, S-2-L, or S-3-L can also be free or substantially free (e.g., containing less than 5%, less than 2%, less than 1%, less than 0.5%, or less than 0.1%) of any other diastereomers, respectively. However, in some embodiments, the compound of Formula S-1-L, S-2-L, or S-3-L can also exist in a racemic mixture or in a stereoisomeric mixture, respectively.

In some embodiments, the method of preparing a compound of Formula I-P is for preparing a compound of Formula I-P-D. In such embodiments, the corresponding starting materials and/or intermediates used for the methods have each of the glutamate units in a D-form. For example, in some embodiments, each glutamate unit in each of Formula S-1, S-2, S-3, S-1-A, S-2-A, S-3-A, S-2-B, and S-3-B can be in the D-form.

In some embodiments, compounds of Formula I (e.g., Formula I-L or I-D) can also be prepared using solid phase chemistry. For example, an initial glutamyl residue can be bonded to a Wang resin (or other suitable resins or solid supports) and additional glutamyl residues are added serially via solid phase peptide synthesis using F-moc chemistry. After the final glutamyl residue is added, the Antifolate precursor (e.g., pemetrexed precursor) is coupled to the peptide and the molecule is cleaved from the resin. In some embodiments, compounds of Formula I (e.g., Formula I-L or I-D) are not prepared using solid phase chemistry.

### Formula II and Preparation

The compound of Formula II is a polyglutamated antifolate, with Z in Formula II being a residue of a suitable antifolate. Non-limiting suitable antifolates include any of those described in WO 2018/031967, WO 2018/031968, WO 2018/031979, WO 2018/031980, WO 2019/094648, PCT/US2019/016989, and PCT/US2019/017004, the content of each of which is herein incorporated by reference in its entirety. Some exemplary antifolates are described herein. While embodiments of the present disclosure are directed to polyglutamated antifolates, the compound of Formula I, or a salt thereof, can form an amide with any other drug with a carboxylic acid group or an activated form thereof, to form a compound of Formula II, or a salt thereof, wherein Z in Formula II represents a residue of such drug.

The conversion of the compound of Formula I or a salt thereof into the corresponding compound of Formula II or a salt thereof can be typically carried out with no or minimized racemization of chiral centers. In some embodiments, the polyglutamates of Formula II are prepared in a stereoisomerically pure or substantially pure form in a large scale. For example, in some embodiments, the present disclosure provides the polyglutamates of Formula II in a stereoisomerically pure or substantially pure form in a batch size over 10 grams (such as a batch size of about 100 gram or more, about 1 kg or more, about 5 kg or more, about 10 kg or more, etc.). In some embodiments, the polyglutamate of Formula II can exist predominantly in one enantiomeric form, which can be free or substantially free (e.g., containing less than 5%, less than 2%, less than 1%, less than 0.5%, or less than 0.1%) of the other enantiomeric form. In some embodiments, the polyglutamate of Formula II can also exist predominantly as one diastereomer, which can be free or substantially free (e.g., containing less than 5%, less than 2%, less than 1%, less than 0.5%, or less than 0.1%) of any other diastereomer(s).

In some embodiments, each of the glutamate units in Formula II is in an L-form, and the compound of Formula II is a compound of Formula II-L:

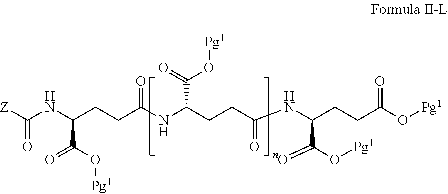

wherein Pg¹, Z, and n are defined herein. In some embodiments, the polyglutamate of Formula II-L can be stereoisomerically pure or substantially pure. For example, in some embodiments, the polyglutamate of Formula II-L can be free or substantially free (e.g., containing less than 5%, less than 2%, less than 1%, less than 0.5%, or less than 0.1%) of the other enantiomeric form. In some embodiments, the polyglutamate of Formula II-L can also be free or substantially free (e.g., containing less than 5%, less than 2%, less than 1%, less than 0.5%, or less than 0.1%) of any other diastereomer(s). However, in some embodiments, the polyglutamate of Formula II-L can also exist in a racemic mixture or in a stereoisomeric mixture.

In some embodiments, each of the glutamate units in Formula II is in a D-form, and the compound of Formula II is a compound of Formula II-D:

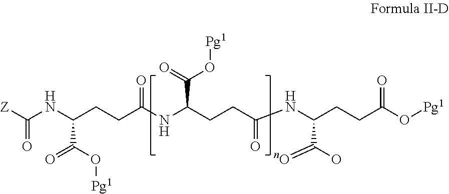

wherein Pg¹, Z, and n are defined herein. In some embodiments, the polyglutamate of Formula II-D can be stereoisomerically pure or substantially pure. For example, in some embodiments, the polyglutamate of Formula II-D can be free or substantially free (e.g., containing less than 5%, less than 2%, less than 1%, less than 0.5%, or less than 0.1%) of the other enantiomeric form. In some embodiments, the polyglutamate of Formula II-D can also be free or substantially free (e.g., containing less than 5%, less than 2%, less than 1%, less than 0.5%, or less than 0.1%) of any other diastereomer(s). However, in some embodiments, the polyglutamate of Formula II-D can also exist in a racemic mixture or in a stereoisomeric mixture.

In some specific embodiments, Z in Formula II (e.g., Formula II-L or II-D) can be a residue of an antifolate selected from methotrexate (MTX), pemetrexed (PMX), lometrexol (LTX), AG2034, raltitrexed (RTX), pralatrexate, GW1843, aminopterin, LY309887 and LY222306.

In some specific embodiments, Z in Formula II (e.g., Formula II-L or II-D) can be a residue of pemetrexed having the following formula:

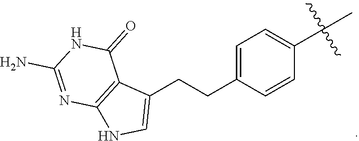 [SMILES: *c1ccc(CCc2c[nH]c3nc(N)[nH]c(=O)c23)cc1]

In some specific embodiments, Z in Formula II (e.g., Formula II-L or II-D) can be a residue having the following formula:

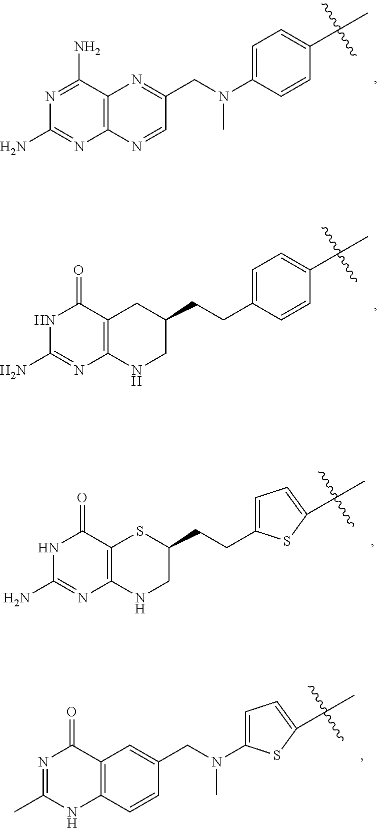 [SMILES: *c1ccc(N(C)Cc2cnc3nc(N)nc(N)c3n2)cc1 ; *c1ccc(CC[C@H]2CNc3nc(N)[nH]c(=O)c3C2)cc1 ; *c1ccc(CC[C@H]2CNc3nc(N)[nH]c(=O)c3S2)s1 ; *c1ccc(N(C)Cc2ccc3[nH]c(C)nc(=O)c3c2)s1]

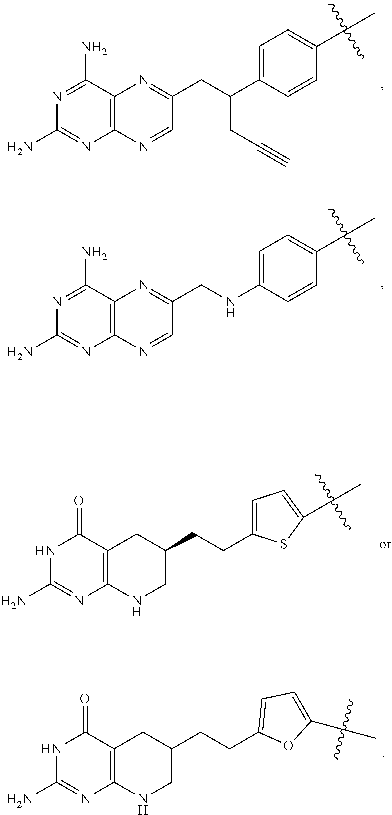 [SMILES: *c1ccc(C(CC#C)Cc2cnc3nc(N)nc(N)c3n2)cc1 ; *c1ccc(NCc2cnc3nc(N)nc(N)c3n2)cc1 ; *c1ccc(CC[C@H]2CNc3nc(N)[nH]c(=O)c3C2)s1 ; *c1ccc(CCC2CNc3nc(N)[nH]c(=O)c3C2)o1]

In some embodiments, Z can be a residue having the following formula:

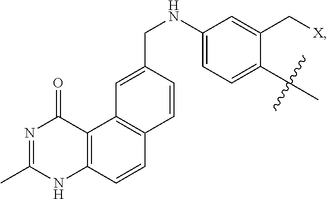

wherein X is a leaving group. In some embodiments, Z—COOH, upon reaction with the compound Formula I under an amide forming condition, can form an intermediate, which can be further converted into a cyclic structure:

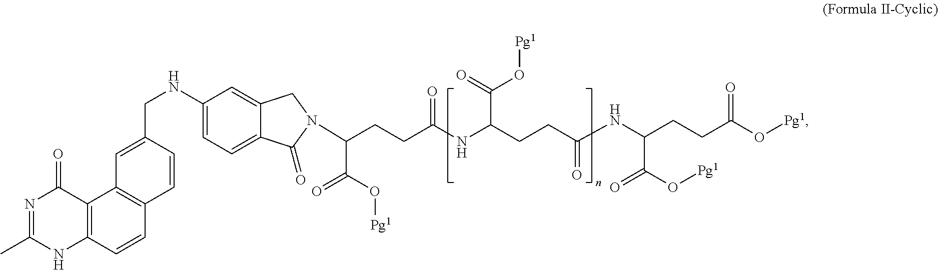

wherein each glutamate unit is independently in an L-form or D-form (e.g., all glutamate units are in L-form or all glutamate units are in D-form), Pg¹ and n (e.g., n can be an integer of 0-20, such as 2, 3, or 4) are defined herein, which can be further converted into the compound of the following formula or a pharmaceutically acceptable salt thereof:

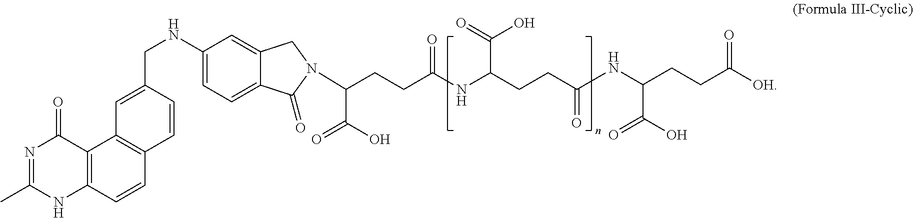

The Pg¹ and n for Formula II can be any of those described herein as suitable for the polyglutamate of Formula I. For example, in some specific embodiments, n in Formula II (e.g., Formula II-L or II-D) can be an integer of 0-20, for example, 2-20, 2-15, 2-10, 2-6, 2-5, or more than 5. In some specific embodiments, Pg¹ in Formula II (e.g., Formula II-L or II-D) can be an acid labile carboxylic acid protecting group, such as tert-butyl.

The compound of Formula II (e.g., Formula II-L or II-D) or a salt thereof (e.g., a pharmaceutically acceptable salt) is typically substantially pure, for example, has a purity of at least 90% (e.g., at least 90%, at least 95%, at least 98%, at least 99%) by HPLC and/or by weight. In some embodiments, the compound of Formula II can also be in a solid form, such as a crystalline form, an amorphous form, or a mixture thereof. For example, in some embodiments, the compound of Formula II can be purified through crystallization, such as using a suitable solvent system. Examples of such crystallization are shown in the Examples section.

In some embodiments, the compound of Formula II (e.g., Formula II-L or II-D) can be a substantially pure specific oligomer, for example, a substantially pure tetraglutamate (n is 2), a substantially pure pentaglutamate (n is 3), a substantially pure hexaglutamate (n is 4), a substantially pure heptaglutamate (n is 5), etc. In some specific embodiments, the compound of Formula II (e.g., Formula II-L or II-D) can be a substantially pure hexaglutamate, wherein n in Formula II can be 4. For example, in such embodiments, the compound of Formula II (e.g., Formula II-L or II-D) can be substantially free (e.g., less than 10%, less than 5%, less than 2%, less than 1%, less than 0.5%, or less than 0.1%) of another compound of Formula II wherein n is not 4. Compounds of Formula II with the recited purity profile can be prepared by controlling the purity of the corresponding polyglutamate of Formula I used for the amide coupling reaction with Z—COOH or an activated form thereof. Exemplary procedures are described in the Examples section.

### Formula III and Preparation

The compound of Formula II, or a salt thereof, can be deprotected to form a compound of Formula III, or a salt thereof. In some embodiments, the compound of Formula III or a salt thereof can be substantially pure. In some embodiments, the compound of Formula III can be in an acid addition salt, such as a TFA salt. The acid addition salt of Formula III can be substantially pure, which can be used by itself in a pharmaceutical composition. In some embodiments, the acid addition salt of Formula III can also be used as an intermediate to prepare a high purity salt of Formula III, such as an alkali salt of Formula IV. When used as an intermediate, the acid addition salt of Formula III does not have to be a pharmaceutically acceptable salt.

In some embodiments, the compound of Formula III is present in a form of a pharmaceutically acceptable salt, e.g., a sodium salt, which includes monosodium, disodium, trisodium, etc., with the number of sodium up to the number of negatively charged carboxylic acid groups in Formula III. For example, when n is 4, there are a total of 7 carboxylic acid groups in Formula III (not considering any potential carboxylic acid group in Z group), and the salt can be a monosalt, disalt, trisalt, and up to hepta-salt, such as hepta-sodium salt. In some embodiments, the compound of Formula III can be in a form of a pharmaceutically acceptable acid addition salt, such as an HCl salt. In some embodiments, the acid addition salt such as HCl salt or the base addition salt such as a sodium salt can be used for controlling osmolarity, such as maintaining appropriate osmolarity in liposomal encapsulation.

As will be understood by those skilled in the art, when the PANTIFOL of the present disclosure is formulated, further processed, or administered, the actual ionization state of the PANTIFOL will depend on the pH of the medium encompassing the PANTIFOL. For example, when a compound of Formula III or its pharmaceutically acceptable salt(s) is formulated, further processed, or administered, the actual ionization state of the compound of Formula III will depend on the pH of the medium encompassing the compound of Formula III. Taking a hexglutamated Antifolate (n is 4) of the present disclosure as an example, the seven carboxylic acid groups can be partially ionized when the medium pH is about 6.5 to 7.0, and can be fully ionized at a higher pH such as greater than 10. Thus, when formulated, for example, as a liposomal composition herein, the compound of Formula III or its pharmaceutically acceptable salt(s) can become partially ionized or fully ionized depending on the pH of the formulation medium, regardless of whether the free form or a salt form of the compound of Formula III (e.g., a HCl salt of Formula III or an alkali salt of Formula IV) is used as the starting drug substance for the formulation. The compositions of the present disclosure such as the liposomal compositions herein should not be understood as to be limited to any particular ionization state of the compound of Formula III. In some embodiments, the ionization state of the compound of Formula III in a composition, for example, in a liposomal composition, can also be controlled by adjusting the medium pH. In some embodiments, the ionization state of the compound of Formula III in a composition can be monitored by measuring the osmolarity of the composition.

The phrase “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts have been described in S. M. Berge et al. J. Pharmaceutical Sciences, 1977, 66:1-19.

Compounds of Formula III can contain both a basic and an acidic functionality, and can be converted to a pharmaceutically acceptable salt, when desired, by using a suitable acid or base.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Examples of acids which can be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid. In some embodiments, the pharmaceutically acceptable salt of compounds of Formula III is an acid addition salt such as HCl salt.

Basic addition salts can be prepared by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like. In some embodiments, the pharmaceutically acceptable salt of compounds of Formula III is a base addition salt such as an alkali salt, an alkaline earth metal salt, etc. as described herein.

In some embodiments, each of the Pg¹ groups of Formula II is an acid labile protecting group, which can be deprotected under acidic conditions. In some embodiments, the Pg¹ groups of Formula II are the same acid labile protecting group. For example, in some embodiments, each of the Pg¹ groups can be a tert-butyl group. In some embodiments, the deprotecting of the compound of Formula II can be effected with an acid, such as trifluoroacetic acid (TFA), HCl, etc.

The conversion of a compound of Formula II or a salt thereof into the corresponding compound of Formula III or a salt thereof can be typically carried out with no or minimized racemization of chiral centers. In some embodiments, the polyglutamates of Formula III are prepared in a stereoisomerically pure or substantially pure form in large scales. For example, in some embodiments, the present disclosure provides the polyglutamates of Formula III in a stereoisomerically pure or substantially pure form in a batch size over 10 grams (such as a batch size of about 100 gram or more, about 1 kg or more, about 5 kg or more, about 10 kg or more, etc.). In some embodiments, the polyglutamate of Formula III can exist predominantly in one enantiomeric form, which can be free or substantially free (e.g., containing less than 5%, less than 2%, less than 1%, less than 0.5%, or less than 0.1%) of the other enantiomeric form. In some embodiments, the polyglutamate of Formula III can also exist predominantly as one diastereomer, which can be free or substantially free (e.g., containing less than 5%, less than 2%, less than 1%, less than 0.5%, or less than 0.1%) of any other diastereomer(s).

In some embodiments, each of the glutamate units in Formula III is in an L-form, and the compound of Formula III is a compound of Formula III-L:

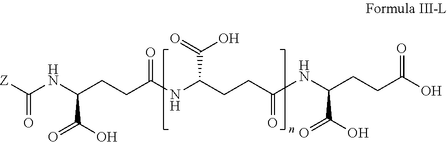

wherein Z and n are defined herein. In some embodiments, the polyglutamate of Formula III-L can be stereoisomerically pure or substantially pure. For example, in some embodiments, the polyglutamate of Formula III-L can be free or substantially free (e.g., containing less than 5%, less than 2%, less than 1%, less than 0.5%, or less than 0.1%) of the other enantiomeric form. In some embodiments, the polyglutamate of Formula III-L can also be free or substantially free (e.g., containing less than 5%, less than 2%, less than 1%, less than 0.5%, or less than 0.1%) of any other diastereomer(s). However, in some embodiments, the polyglutamate of Formula III-L can also exist in a racemic mixture or in a stereoisomeric mixture.

In some embodiments, each of the glutamate units in Formula III is in a D-form, and the compound of Formula III is a compound of Formula III-D:

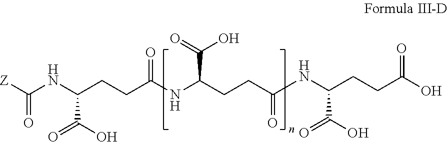

wherein Z and n are defined herein. In some embodiments, the polyglutamate of Formula III-D can be stereoisomerically pure or substantially pure. For example, in some embodiments, the polyglutamate of Formula III-D can be free or substantially free (e.g., containing less than 5%, less than 2%, less than 1%, less than 0.5%, or less than 0.1%) of the other enantiomeric form. In some embodiments, the polyglutamate of Formula III-D can also be free or substantially free (e.g., containing less than 5%, less than 2%, less than 1%, less than 0.5%, or less than 0.1%) of any other diastereomer(s). However, in some embodiments, the polyglutamate of Formula III-D can also exist in a racemic mixture or in a stereoisomeric mixture.

In some specific embodiments, Z in Formula III (e.g., Formula III-L or III-D) can be a residue of an antifolate selected from methotrexate (MTX), pemetrexed (PMX), lometrexol (LTX), AG2034, raltitrexed (RTX), pralatrexate, GW1843, aminopterin, LY309887 and LY222306.

In some specific embodiments, Z in Formula III (e.g., Formula III-L or III-D) can be a residue of pemetrexed having the following formula:

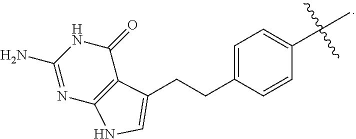 [SMILES: *c1ccc(CCc2c[nH]c3nc(N)[nH]c(=O)c23)cc1]

In some specific embodiments, Z in Formula III (e.g., Formula III-L or III-D) can be a residue having the following formula:

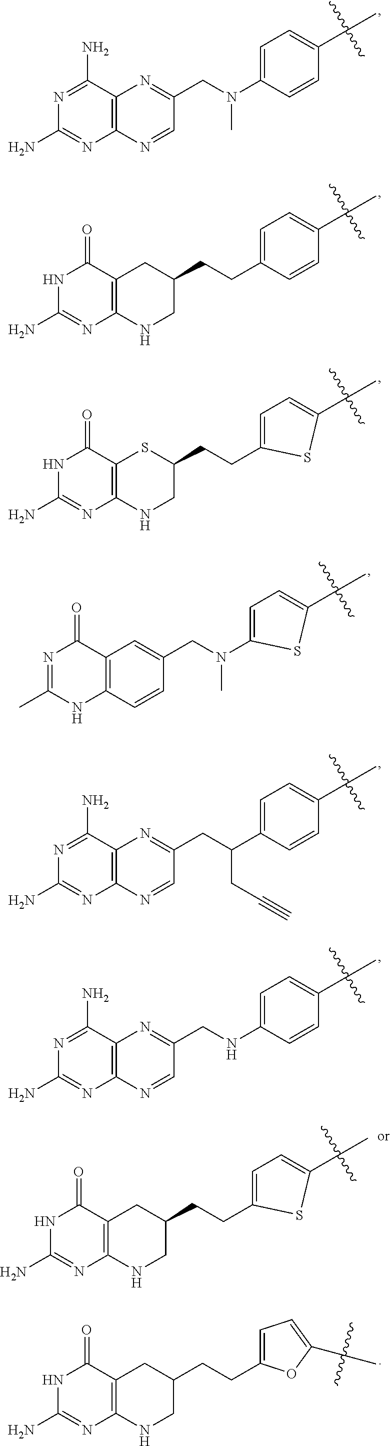

In some specific embodiments, n in Formula III (e.g., Formula III-L or III-D) can be an integer of 0-20, for example, 2-20, 2-15, 2-10, 2-6, 2-5, or more than 5.

The compound of Formula III (e.g., Formula III-L or III-D) or a salt thereof (e.g., a pharmaceutically acceptable salt) is typically substantially pure, for example, has a purity of at least 90% (e.g., at least 90%, at least 95%, at least 98%, at least 99%) by HPLC and/or by weight. The term substantially pure, when referring to the compound of Formula III or a salt thereof can refer to a substantially pure mixture of oligomers (e.g., n is 2-5), which means that it is substantially free of impurities that are not the specified mixture of oligomers. In some embodiments, the term substantially pure, when referring to the compound of Formula III or a salt thereof can also refer to a substantially pure specific oligomer (e.g., n is 2, 3, 4, or 5), which means that it is substantially free of impurities that are not the specific oligomer.

In some embodiments, the compound of Formula III (e.g., Formula III-L or III-D) can be a substantially pure specific oligomer, e.g., with a purity of at least 90% (e.g., at least 90%, at least 95%, at least 98%, at least 99%) by HPLC and/or by weight. For example, the compound of Formula III (e.g., Formula III-L or III-D) can be a substantially pure tetraglutamate (n is 2), a substantially pure pentaglutamate (n is 3), a substantially pure hexaglutamate (n is 4), a substantially pure heptaglutamate (n is 5), etc. In some specific embodiments, the compound of Formula III (e.g., Formula III-L or III-D) can be a substantially pure hexaglutamate, wherein n in Formula III is 4. For example, in such embodiments, the compound of Formula III (e.g., Formula III-L or III-D) can be substantially free (e.g., less than 10%, less than 5%, less than 2%, less than 1%, less than 0.5%, or less than 0.1%) of another compound of Formula III wherein n is not 4.

Compounds of Formula III with the recited purity profile can be prepared by controlling the purity of the corresponding polyglutamate of Formula I used for the amide coupling reaction with Z—COOH or an activated form thereof and/or the protected polyglutamated antifolate of Formula II. Exemplary procedures are described in the Examples section.

In some embodiments, compounds of Formula III (e.g., Formula III-L or III-D) can also be prepared using solid phase chemistry. For example, an initial glutamyl residue can be bonded to a Wang resin (or other suitable resins or solid supports) and additional glutamyl residues are added serially via solid phase peptide synthesis using F-moc chemistry. After the final glutamyl residue is added the Antifolate precursor (e.g., pemetrexed precursor) is coupled to the peptide and the molecule is cleaved from the resin. In some embodiments, compounds of Formula III (e.g., Formula I-L or I-D) are not prepared using solid phase chemistry.

As shown in the Examples section, two pemetrexed gamma polyglutamate (in L form or D form) were synthesized on solid phase resin and compouds were fully characterized by LC-MS, NMR with a HPLC purity of 96%, 98%, exact mass: 1072.3621; Calculated (M+H): 1073.3699. Found (M+H): 1073.3687.

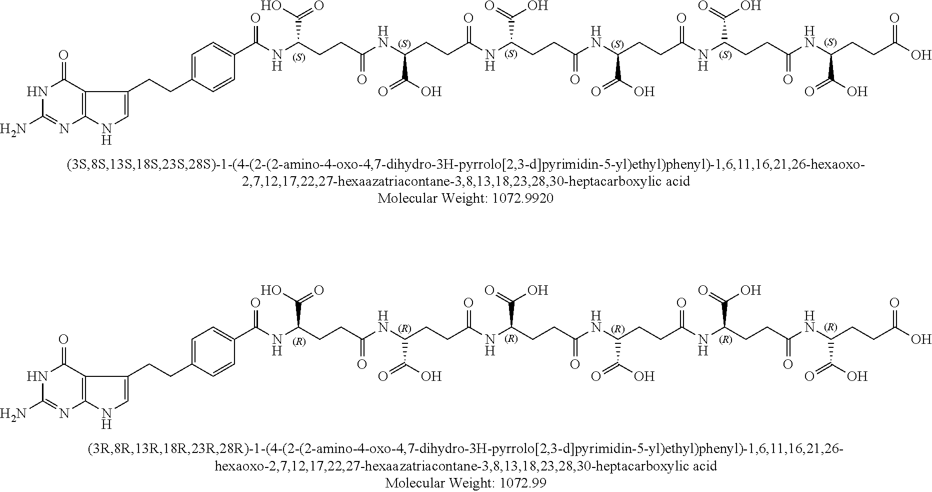

### Formula IV and Preparation

In some embodiments, the present disclosure also provides an alkali salt of Formula IV (e.g., described herein). Without wishing to be bound by theories, it is believed that the use of alkali salt can be beneficial in various ways. The alkali salt is typically more water soluble than the corresponding free acid form or an acid addition salt or other salts. As shown in the Examples, a representative alkali salt of Formula IV is highly water soluble. Thus, in some embodiments, the alkali salt of Formula IV can be more suitable for preparing a pharmaceutical composition where a good aqueous solubility is beneficial, such as preparing an aqueous solution formulation, or preparing a liposomal composition described herein. Also, with a further processing step, the alkali salt can be prepared in a higher purity than the free acid form or the acid addition salt. For example, in some embodiments, the alkali salt can be prepared from a substantially pure acid addition salt of Formula III, and the alkali salt resulted can be further purified, such as through crystallization, to form a solid form of the alkali salt, which is typically substantially pure. This process can greatly enhance large-scale manufacturing and can lead to a high purity active pharmaceutical ingredient useful for preparing various pharmaceutical compositions, e.g., as described herein.

In some embodiments, the alkali salt of Formula IV is in a solid form. For example, in some embodiments, the alkali salt of Formula IV can be an anhydrous form, a hydrate, a solvate, or a mixture thereof. In some embodiments, the alkali salt of Formula IV is a solvate, such as an ethanol solvate.

The alkali salt can be prepared by converting a compound of Formula III, or a salt thereof, e.g., a substantially pure acid addition salt of Formula III, into the alkali salt of Formula IV by treating with a suitable base, such as NaOH. In some embodiments, a substantially pure compound of Formula III or an acid addition salt thereof in a solid form can be used for preparation of the salt of Formula IV. In some embodiments, the alkali salt of Formula IV can be prepared by a method comprising: adding a substantially pure compound of Formula III or a salt thereof to an aqueous alkali base solution, e.g., NaOH solution, to form the alkali salt in water; and adding the alkali salt in water to a solvent (e.g., ethanol) to precipitate the alkali salt. In some embodiments, the precipitated alkali salt can be further dissolved in water, and the aqueous solution can be added to a solvent (e.g., ethanol) to precipitate the alkali salt. In some embodiments, the process of dissolving and precipitating in a solvent can be repeated to achieve a desired purity. In some embodiments, the solvent for precipitating is an alcoholic solvent, such as a C₁₋₄ alcohol (e.g., ethanol). In some embodiments, the substantially pure compound of Formula III or an acid addition salt thereof (e.g., a TFA salt) in a solid form can be first dissolved, partially dissolved, suspended, or otherwise mixed in a suitable solvent, which can be for example, water, a C₁₋₄ alcohol (e.g., ethanol), or a mixture thereof, and the suitable base (e.g., NaOH) can be added concurrently or sequentially in any order to the solvent, which can convert the acid addition salt of Formula III into an alkali salt of Formula IV.

In some embodiments, the presend disclosure also provide a method of isolating, purifying, and/or crystallizing the alkali salt of Formula IV to provide a substantially pure salt of Formula IV. In some embodiments, the crystallizing can comprise dissolving the alkali salt of Formula IV in water, and then adding the aqueous solution into a solvent to precipitate the alkali salt. In some embodiments, the solvent is an alcoholic solvent, such as a C₁₋₄ alcohol (e.g., methanol, ethanol, isopropanol, etc.). In some embodiments, the solvent is ethanol. Other isolation, purification, and crystallization techniques are known in the art and can be used for the methods herein. Typically, the precipitated alkali salt of Formula IV is substantially pure. In some embodiments, the substantially pure salt of Formula IV is a hydrate or a solvate. In some embodiments, the substantially pure salt of Formula IV is in a crystalline form, an amorphous form, or a mixture thereof.

Typically, the conversion of the compound of Formula III, or a salt thereof, into the alkali salt of Formula IV can be carried out with no or minimized racemization of chiral centers. In some embodiments, the alkali salt of Formula IV are prepared in a stereoisomerically pure or substantially pure form in a large scale. For example, in some embodiments, the present disclosure provides the alkali salt of Formula IV in a stereoisomerically pure or substantially pure form in a batch size over 10 grams (such as a batch size of about 100 gram or more, about 1 kg or more, about 5 kg or more, about 10 kg or more, etc.). In some embodiments, the alkali salt of Formula IV can exist predominantly in one enantiomeric form, which can be free or substantially free (e.g., containing less than 5%, less than 2%, less than 1%, less than 0.5%, or less than 0.1%) of the other enantiomeric form. In some embodiments, the alkali salt of Formula IV can also exist predominantly as one diastereomer, which can be free or substantially free (e.g., containing less than 5%, less than 2%, less than 1%, less than 0.5%, or less than 0.1%) of any other diastereomer(s).

In some embodiments, each of the glutamate units in Formula IV is in an L-form, and the compound of Formula IV is a compound of Formula IV-L:

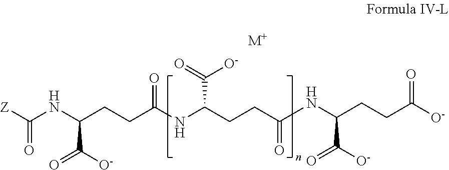

wherein Z, M⁺, and n are defined herein. In some embodiments, the alkali salt of Formula IV-L can be stereoisomerically pure or substantially pure. For example, in some embodiments, the alkali salt of Formula IV-L can be free or substantially free (e.g., containing less than 5%, less than 2%, less than 1%, less than 0.5%, or less than 0.1%) of the other enantiomeric form. In some embodiments, the alkali salt of Formula IV-L can also be free or substantially free (e.g., containing less than 5%, less than 2%, less than 1%, less than 0.5%, or less than 0.1%) of any other diastereomer(s). However, in some embodiments, the alkali salt of Formula IV-L can also exist in a racemic mixture or in a stereoisomeric mixture.

In some embodiments, each of the glutamate units in Formula IV is in a D-form, and the compound of Formula IV is a compound of Formula IV-D:

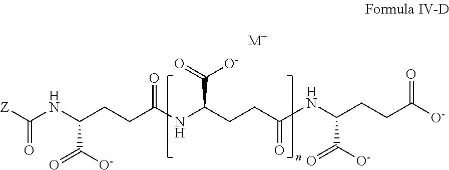

wherein Z, M⁺, and n are defined herein. In some embodiments, the alkali salt of Formula IV-D can be stereoisomerically pure or substantially pure. For example, in some embodiments, the alkali salt of Formula IV-D can be free or substantially free (e.g., containing less than 5%, less than 2%, less than 1%, less than 0.5%, or less than 0.1%) of the other enantiomeric form. In some embodiments, the alkali salt of Formula IV-D can also be free or substantially free (e.g., containing less than 5%, less than 2%, less than 1%, less than 0.5%, or less than 0.1%) of any other diastereomer(s). However, in some embodiments, the alkali salt of Formula IV-D can also exist in a racemic mixture or in a stereoisomeric mixture.

In some specific embodiments, Z in Formula IV (e.g., Formula IV-L or IV-D) can be a residue of an antifolate selected from methotrexate (MTX), pemetrexed (PMX), lometrexol (LTX), AG2034, raltitrexed (RTX), pralatrexate, GW1843, aminopterin, LY309887 and LY222306.

In some specific embodiments, Z in Formula IV (e.g., Formula IV-L or IV-D) can be a residue of pemetrexed having the following formula:

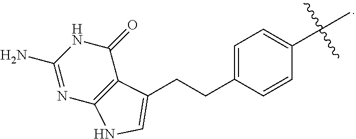 [SMILES: *c1ccc(CCc2c[nH]c3nc(N)[nH]c(=O)c23)cc1]

In some specific embodiments, Z in Formula IV (e.g., Formula IV-L or IV-D) can be a residue having the following formula:

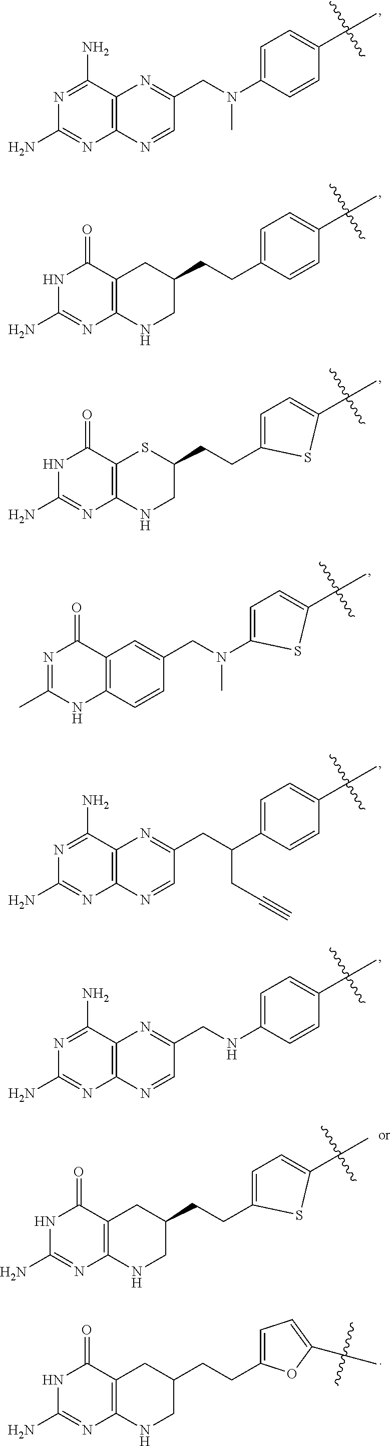

In some specific embodiments, n in Formula IV (e.g., Formula IV-L or IV-D) can be an integer of 0-20, for example, 2-20, 2-15, 2-10, 2-6, 2-5, or more than 5. In some embodiments, M⁺ is Na⁺. In some embodiments, n is 4, M⁺ is Na⁺, and the alkali salt of Formula IV is a hepta-sodium salt.

The compound of Formula IV (e.g., Formula IV-L or IV-D) is typically substantially pure, for example, has a purity of at least 90% (e.g., at least 90%, at least 95%, at least 98%, at least 99%) by HPLC and/or by weight. The term substantially pure, when referring to the compound of Formula IV can refer to a substantially pure mixture of oligomers (e.g., n is 2-5), which means that it is substantially free of impurities that are not the specified mixture of oligomers. In some embodiments, the term substantially pure, when referring to the compound of Formula IV can also refer to a substantially pure specific oligomer (e.g., n is 2, 3, 4, or 5), which means that it is substantially free of impurities that are not the specific oligomer.

In some embodiments, the compound of Formula IV (e.g., Formula IV-L or IV-D) can be a substantially pure specific oligomer, e.g., with a purity of at least 90% (e.g., at least 90%, at least 95%, at least 98%, at least 99%) by HPLC and/or by weight. For example, the compound of Formula IV (e.g., Formula IV-L or IV-D) can be a substantially pure tetraglutamate (n is 2), a substantially pure pentaglutamate (n is 3), a substantially pure hexaglutamate (n is 4), a substantially pure heptaglutamate (n is 5), etc. In some specific embodiments, the compound of Formula IV (e.g., Formula IV-L or IV-D) can be a substantially pure hexaglutamate, wherein n in Formula IV can be 4. For example, in such embodiments, the compound of Formula IV (e.g., Formula IV-L or IV-D) can be substantially free (e.g., less than 10%, less than 5%, less than 2%, less than 1%, less than 0.5%, or less than 0.1%) of another compound of Formula IV wherein n is not 4.

Compounds of Formula IV with the recited purity profile can be prepared by controlling the purity of the corresponding polyglutamate of Formula I used for the amide coupling reaction with Z—COOH or an activated form thereof, the protected polyglutamate of Formula II, and/or the compound of Formula III or salts thereof. Exemplary procedures are described in the Examples section.

### Exemplary Specific Compounds

In some embodiments, the present disclosure also provides exemplary specific compounds of Formula III-1, or a pharmaceutically acceptable salt thereof:

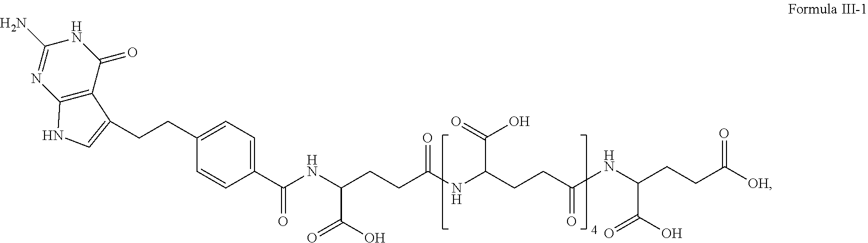

wherein each glutamate unit is independently in an L-form or D-form. In some embodiments, the compound of Formula III-1 can be in a form of a pharmaceutically acceptable acid addition salt, such as an HCl salt. In some embodiments, the compound of Formula III-1 can be in a form of a pharmaceutically acceptable base addition salt, such as a sodium salt, e.g., monosodium, disodium, trisodium, tetrasodium, pentasodium, hexasodium, or hepta-sodium salt. In some embodiments, the present disclosure also provide exemplary specific compounds Formula III-1-L, Formula III-1-D, a mixture thereof, or a pharmaceutically acceptable salt thereof:

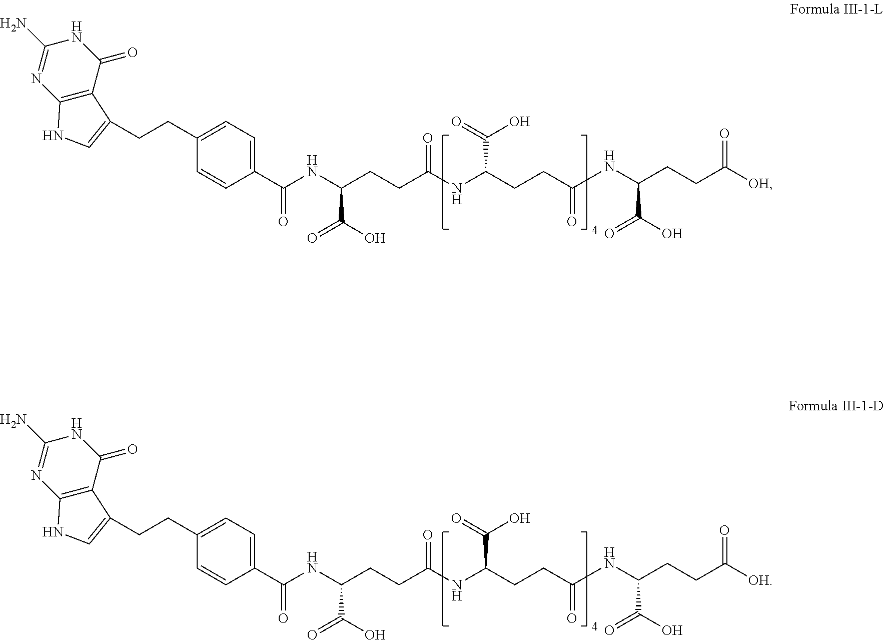

In some embodiments, the compound of Formula III-1-L, or a pharmaceutically acceptable salt thereof (e.g., described herein) can be substantially pure, for example, it can be substantially free (e.g., less than 5%, less than 2%, less than 1%, less than 0.5%, or less than 0.1%) of a compound of Formula III-2, or a pharmaceutically acceptable salt thereof:

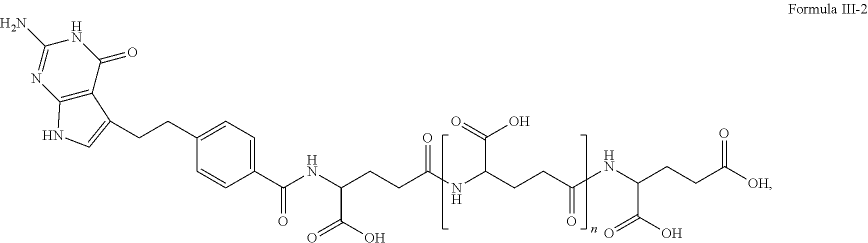

wherein n in Formula III-2 is an integer that is not 4, or n is 4 and at least one of the glutamate units is not in an L-form. In some embodiments, the compound of Formula III-1-L, or a pharmaceutically acceptable salt thereof can be characterized as having a purity by HPLC of at least 90% and/or by weight of at least 90%, e.g., a purity by HPLC of at least 90%, at least 95%, at least 98%, or at least 99%. In some embodiments, the compound of Formula III-1-L, or a pharmaceutically acceptable salt thereof is in a solid form, such as a crystalline form, an amorphous form, or a mixture thereof. In some embodiments, the compound of Formula III-1-L, or a pharmaceutically acceptable salt thereof can be a hydrate or a solvate, which can be in a crystalline form, an amorphous form, or a mixture thereof. In some embodiments, a pharmaceutical batch of the substantially pure compound of Formula III-1-L, or a pharmaceutically acceptable salt thereof is provided. In some embodiments, the pharmaceutical batch is at least 10 grams, such as a batch size of about 100 gram or more, about 1 kg or more, about 5 kg or more, about 10 kg or more, etc. In some embodiments, the pharmaceutical batch is in a solid form, such as a crystalline form, an amorphous form, or a mixture thereof. In some embodiments, the compound of Formula III-1-L can be in a form of a pharmaceutically acceptable acid addition salt, such as an HCl salt. In some embodiments, the compound of Formula III-1-L can be in a form of a pharmaceutically acceptable base addition salt, such as a sodium salt, e.g., monosodium, disodium, trisodium, tetrasodium, pentasodium, hexasodium, or hepta-sodium salt.

In some embodiments, the compound of Formula III-1-D, or a pharmaceutically acceptable salt thereof (e.g., described herein) can be substantially pure, for example, it can be substantially free (e.g., less than 5%, less than 2%, less than 1%, less than 0.5%, or less than 0.1%) of a compound of Formula III-2, or a pharmaceutically acceptable salt thereof:

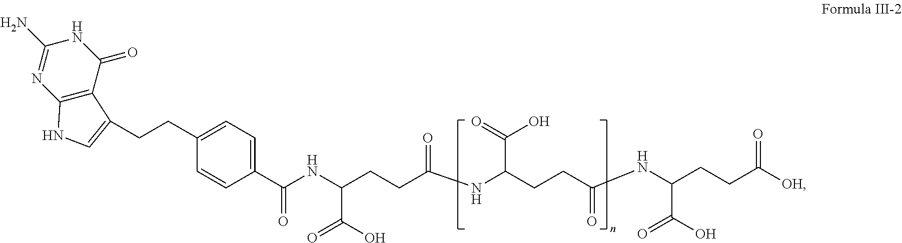

wherein n in Formula III-2 is an integer that is not 4, or n is 4 and at least one of the glutamate units is not in a D-form. In some embodiments, the compound of Formula III-1-D, or a pharmaceutically acceptable salt thereof can be characterized as having a purity by HPLC of at least 90% and/or by weight of at least 90%, e.g., a purity by HPLC of at least 90%, at least 95%, at least 98%, or at least 99%. In some embodiments, the compound of Formula III-1-D, or a pharmaceutically acceptable salt thereof is in a solid form, such as a crystalline form, an amorphous form, or a mixture thereof. In some embodiments, the compound of Formula III-1-D, or a pharmaceutically acceptable salt thereof can be a hydrate or a solvate, which can be in a crystalline form, an amorphous form, or a mixture thereof. In some embodiments, a pharmaceutical batch of the substantially pure compound of Formula III-1-D, or a pharmaceutically acceptable salt thereof is provided. In some embodiments, the pharmaceutical batch is at least 10 grams, such as a batch size of about 100 gram or more, about 1 kg or more, about 5 kg or more, about 10 kg or more, etc. In some embodiments, the pharmaceutical batch is in a solid form, such as a crystalline form, an amorphous form, or a mixture thereof. In some embodiments, the compound of Formula III-1-D can be in a form of a pharmaceutically acceptable acid addition salt, such as an HCl salt. In some embodiments, the compound of Formula III-1-D can be in a form of a pharmaceutically acceptable base addition salt, such as a sodium salt, e.g., monosodium, disodium, trisodium, tetrasodium, pentasodium, hexasodium, or hepta-sodium salt.

In some embodiments, the present disclosure also provides a hepta-sodium salt of Formula IV-1:

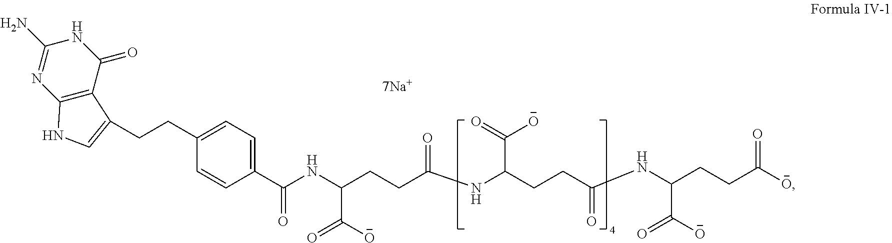

wherein each glutamate unit is independently in an L-form or D-form.

In some embodiments, the present disclosure also provides Formula IV-1-L, Formula IV-1-D, or a mixture thereof:

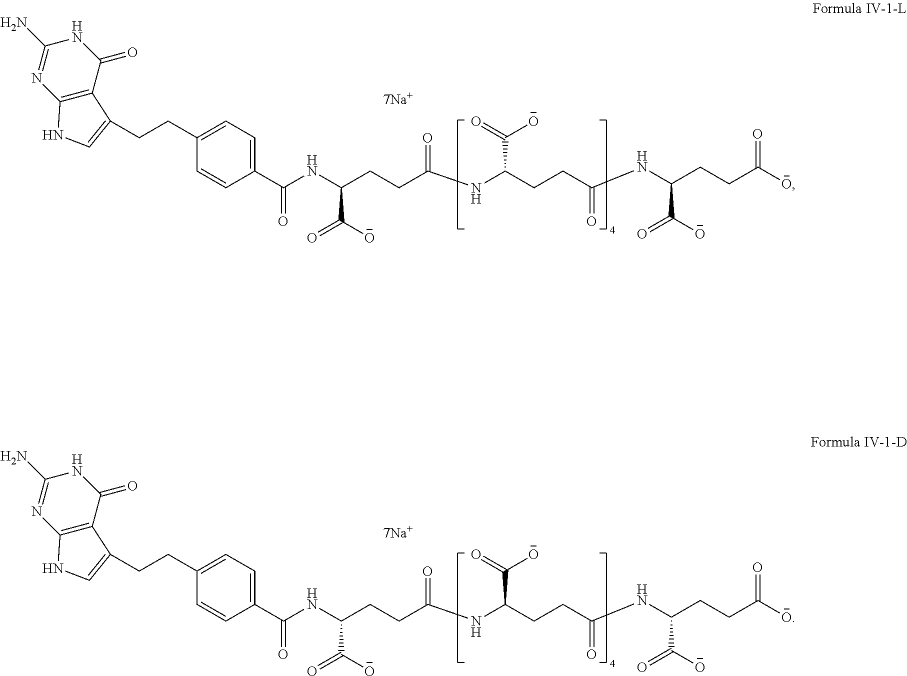

In some embodiments, the compound of Formula IV-1-L can be substantially pure, for example, it can be characterized as having a purity by HPLC of at least 90% and/or by weight of at least 90%, e.g., a purity by HPLC of at least 90%, at least 95%, at least 98%, or at least 99%. In some embodiments, the compound of Formula IV-1-L can be free or substantially free (e.g., containing less than 5%, less than 2%, less than 1%, less than 0.5%, or less than 0.1%) of the other enantiomeric form. In some embodiments, the compound of Formula IV-1-L can also be free or substantially free (e.g., containing less than 5%, less than 2%, less than 1%, less than 0.5%, or less than 0.1%) of any other diastereomer(s). In some embodiments, the compound of Formula IV-1-L can also be free or substantially free (e.g., containing less than 5%, less than 2%, less than 1%, less than 0.5%, or less than 0.1%) of a non-sodium salt of Formula III-1-L. In some embodiments, the compound of Formula IV-1-L is in a solid form, such as a crystalline form, an amorphous form, or a mixture thereof. In some embodiments, the compound of Formula IV-1-L can be a hydrate or a solvate, which can be in a crystalline form, an amorphous form, or a mixture thereof. In some embodiments, a pharmaceutical batch of the substantially pure compound of Formula IV-1-L is provided. In some embodiments, the pharmaceutical batch is at least 10 grams, such as a batch size of about 100 gram or more, about 1 kg or more, about 5 kg or more, about 10 kg or more, etc. In some embodiments, the pharmaceutical batch is in a solid form, such as a crystalline form, an amorphous form, or a mixture thereof.

In some embodiments, the compound of Formula IV-1-D can be substantially pure, for example, it can be characterized as having a purity by HPLC of at least 90% and/or by weight of at least 90%, e.g., a purity by HPLC of at least 90%, at least 95%, at least 98%, or at least 99%. In some embodiments, the compound of Formula IV-1-D can be free or substantially free (e.g., containing less than 5%, less than 2%, less than 1%, less than 0.5%, or less than 0.1%) of the other enantiomeric form. In some embodiments, the compound of Formula IV-1-D can also be free or substantially free (e.g., containing less than 5%, less than 2%, less than 1%, less than 0.5%, or less than 0.1%) of any other diastereomer(s). In some embodiments, the compound of Formula IV-1-D can also be free or substantially free (e.g., containing less than 5%, less than 2%, less than 1%, less than 0.5%, or less than 0.1%) of a non-sodium salt of Formula III-1-D. In some embodiments, the compound of Formula IV-1-D is in a solid form, such as a crystalline form, an amorphous form, or a mixture thereof. In some embodiments, the compound of Formula IV-1-D can be a hydrate or a solvate, which can be in a crystalline form, an amorphous form, or a mixture thereof. In some embodiments, a pharmaceutical batch of the substantially pure compound of Formula IV-1-D is provided. In some embodiments, the pharmaceutical batch is at least 10 grams, such as a batch size of about 100 gram or more, about 1 kg or more, about 5 kg or more, about 10 kg or more, etc. In some embodiments, the pharmaceutical batch is in a solid form, such as a crystalline form, an amorphous form, or a mixture thereof.

In some embodiments, the present disclosure also provides specific synthetic intermediates and products of Compound A, Compound B, Compound C, Compound D, Compound E, Compound F, Compound G, Compound H, Compound I, Compound J, Compound K, Compound L, Compound 100, and Compound 110, as shown in the Examples section. In some embodiments, each of the compounds A-L and 100, and 110, is substantially pure, e.g., with a HPLC purity and/or purity by weight greater than 90% (e.g., greater than 95%, greater than 98%, or greater than 99%).

In some embodiments, the present disclosure also provides a synthetic method of Compound III-1-L comprising a process substantially according to the scheme shown below:

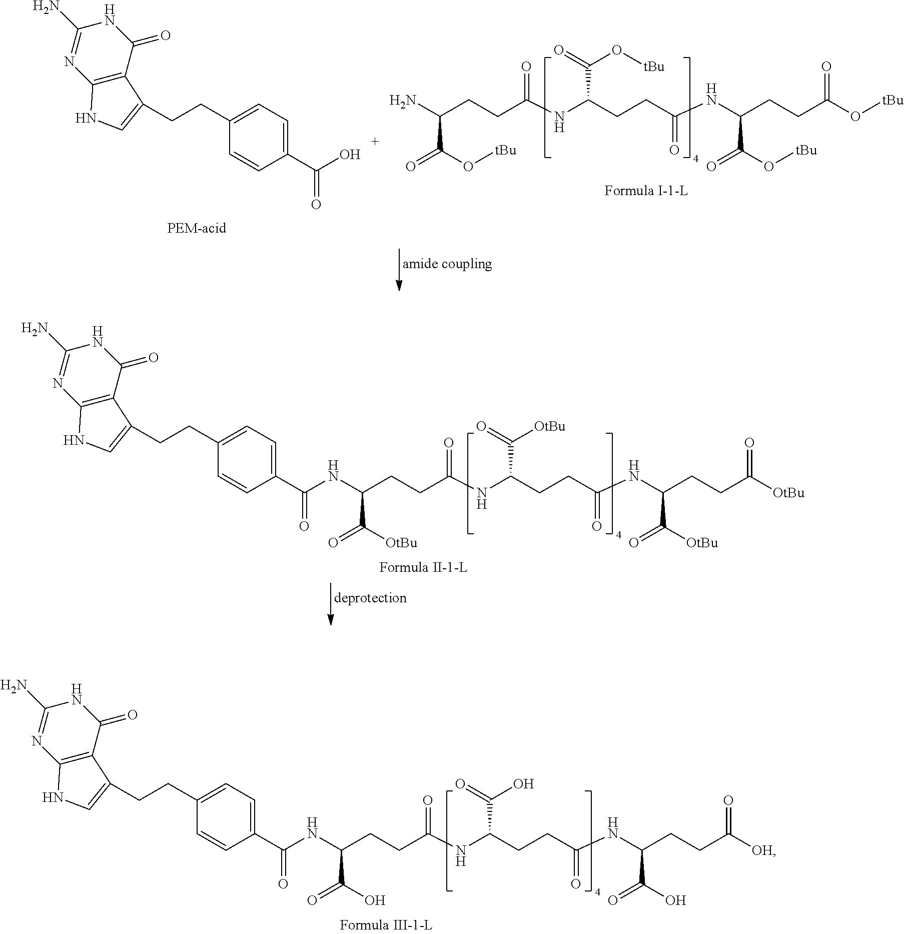

wherein PEM-Acid or an activated form thereof is coupled with a compound of Formula I-1-L to provide a protected polyglutamate of Formula II-1-L, which can be followed by a deprotection step to provide the compound of Formula III-1-L. In some embodiments, the method further comprises converting the compound of Formula III-1-L or a salt thereof into the alkali salt of Formula IV-1-L. In some embodiments, each of the compounds or salts of Formula I-1-L, II-1-L, III-1-L and IV-1-L can be substantially pure, e.g., a HPLC purity and/or purity by weight of greater than 90% (e.g., greater than 95%, greater than 98%, or greater than 99%).

In some embodiments, the present disclosure also provides a synthetic method of preparing an alkali salt of Compound IV-1-L from a compound of Formula III-1-L or a salt thereof, e.g., a substantially pure compound of Formula III-1-L or a salt thereof. In some embodiments, the compound of Formula III-1-L, e.g., substantially pure compound of Formula III-1-L, or an acid addition salt thereof in a solid form can be used for preparation of the salt of Formula IV-1-L. In some embodiments, the alkali salt of Formula IV-1-L can be prepared by a method comprising: adding a substantially pure compound of Formula III-1-L or a salt thereof to an aqueous alkali base solution, e.g., NaOH solution, to form the alkali salt in water; and adding the alkali salt in water to a solvent (e.g., ethanol) to precipitate the alkali salt. In some embodiments, the precipitated alkali salt can be further dissolved in water, and the aqueous solution can be added to a solvent (e.g., ethanol) to precipitate the alkali salt. In some embodiments, the process of dissolving and precipitating in a solvent can be repeated to achieve a desired purity. In some embodiments, the solvent for precipitating is an alcoholic solvent, such as a C₁₋₄ alcohol (e.g., ethanol). In some embodiments, the substantially pure compound of Formula III-1-L or an acid addition salt thereof (e.g., a TFA salt) in a solid form can be first dissolved, partially dissolved, suspended, or otherwise mixed in a suitable solvent, which can be for example, water, a C₁₋₄ alcohol (e.g., ethanol), or a mixture thereof, and the suitable base (e.g., NaOH) can be added concurrently or sequentially in any order to the solvent, which can convert the acid addition salt of Formula III-1-L into an alkali salt of Formula IV-1-L.

In some embodiments, the present disclosure also provides a method of isolating, purifying, and/or crystallizing the alkali salt of Formula IV-1-L to provide a substantially pure salt of Formula IV-1-L. In some embodiments, the crystallizing can comprise dissolving the alkali salt of Formula IV-1-L into water, and then adding the aqueous solution into a solvent to precipitate the alkali salt. In some embodiments, the solvent is an alcoholic solvent, such as a C₁₋₄ alcohol (e.g., methanol, ethanol, isopropanol, etc.). In some embodiments, the solvent is ethanol. Other isolation, purification, and crystallization techniques are known in the art and can be used for the methods herein. Typically, the precipitated alkali salt of Formula IV-1-L is substantially pure, e.g., a HPLC purity and/or purity by weight of greater than 90% (e.g., greater than 95%, greater than 98%, or greater than 99%). In some embodiments, the substantially pure salt of Formula IV-1-L is a hydrate or a solvate. In some embodiments, the substantially pure salt of Formula IV-1-L is in a crystalline form, an amorphous form, or a mixture thereof.

In some embodiments, the present disclosure also provides a synthetic method of Compound III-1-D comprising a process substantially according to the scheme shown below:

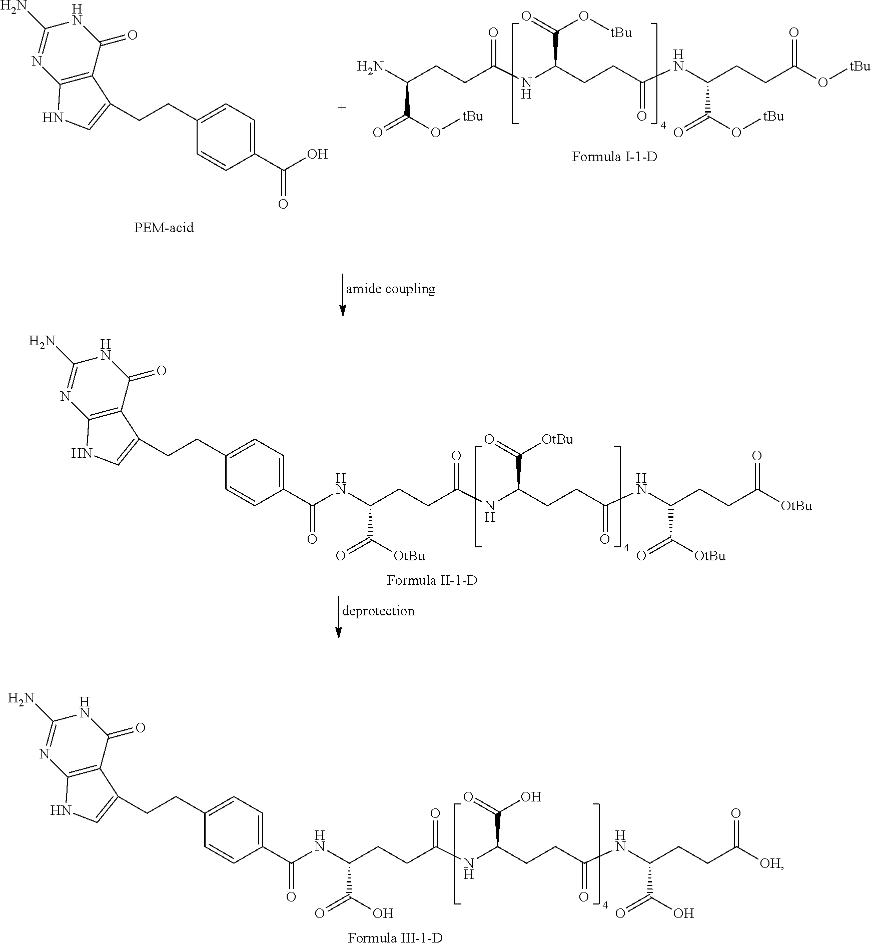

wherein PEM-Acid or an activated form thereof is coupled with a compound of Formula I-1-D to provide a protected polyglutamate of Formula II-1-D, which can be followed by a deprotection step to provide the compound of Formula III-1-D. In some embodiments, the method further comprises converting the compound of Formula III-1-D or a salt thereof into the alkali salt of Formula IV-1-D. In some embodiments, each of the compounds or salts of Formula I-1-D, II-1-D, III-1-D and IV-1-D can be substantially pure, e.g., a HPLC purity and/or purity by weight of greater than 90% (e.g., greater than 95%, greater than 98%, or greater than 99%).

In some embodiments, the present disclosure also provides a synthetic method of preparing an alkali salt of Compound IV-1-D from a compound of Formula III-1-D or a salt thereof, e.g., a substantially pure compound of Formula III-1-D or a salt thereof. In some embodiments, the compound of Formula III-1-D, e.g., substantially pure compound of Formula III-1-D, or an acid addition salt thereof in a solid form can be used for preparation of the salt of Formula IV-1-D. In some embodiments, the alkali salt of Formula IV-1-D can be prepared by a method comprising: adding a substantially pure compound of Formula III-1-D or a salt thereof to an aqueous alkali base solution, e.g., NaOH solution, to form the alkali salt in water; and adding the alkali salt in water to a solvent (e.g., ethanol) to precipitate the alkali salt. In some embodiments, the precipitated alkali salt can be further dissolved in water, and the aqueous solution can be added to a solvent (e.g., ethanol) to precipitate the alkali salt. In some embodiments, the process of dissolving and precipitating in a solvent can be repeated to achieve a desired purity. In some embodiments, the solvent for precipitating is an alcoholic solvent, such as a C1-4 alcohol (e.g., ethanol). In some embodiments, the substantially pure compound of Formula III-1-D or an acid addition salt thereof (e.g., a TFA salt) in a solid form can be first dissolved, partially dissolved, suspended, or otherwise mixed in a suitable solvent, which can be for example, water, a C₁₋₄ alcohol (e.g., ethanol), or a mixture thereof, and the suitable base (e.g., NaOH) can be added concurrently or sequentially in any order to the solvent, which can convert the acid addition salt of Formula III-1-D into an alkali salt of Formula IV-1-D.

In some embodiments, the present disclosure also provides a method of isolating, purifying, and/or crystallizing the alkali salt of Formula IV-1-D to provide a substantially pure salt of Formula IV-1-D. In some embodiments, the crystallizing can comprise dissolving the alkali salt of Formula IV-1-D into water, and then adding the aqueous solution into a solvent to precipitate the alkali salt. In some embodiments, the solvent is an alcoholic solvent, such as a C₁₋₄ alcohol (e.g., methanol, ethanol, isopropanol, etc.). In some embodiments, the solvent is ethanol. Other isolation, purification, and crystallization techniques are known in the art and can be used for the methods herein. Typically, the precipitated alkali salt of Formula IV-1-D is substantially pure, e.g., a HPLC purity and/or purity by weight of greater than 90% (e.g., greater than 95%, greater than 98%, or greater than 99%). In some embodiments, the substantially pure salt of Formula IV-1-D is a hydrate or a solvate. In some embodiments, the substantially pure salt of Formula IV-1-D is in a crystalline form, an amorphous form, or a mixture thereof.

As described herein, the substantially pure compounds herein can exist in solid forms, such as a substantially pure γPANTIFOL of the present disclosure (e.g., Formula III-1-L, III-1-D, or a pharmaceutically acceptable salt thereof, or Formula IV-1-L or IV-1-D). In some embodiments, a pharmaceutical composition in a non-solid form can be prepared from dissolving, suspending, or otherwise mixing a solid form of the substantially pure compounds herein with other ingredients.

In some embodiments, a composition comprising the substantially pure compound or salt herein and one or more other ingredients can be understood as a composition obtained from directly or indirectly mixing the substantially pure compound or salt herein with the one or more other ingredients, such as water, pharmaceutically acceptable excipients, etc.

### αPANTIFOL

Although many of the embodiments described herein are directed to gamma-polyglutamated drugs, the novel synthetic methods, pharmaceutical compositions, and methods of treatment are not so limited. For example, the present disclosure also contemplates alpha-polyglutamated Antifolates. In some embodiments, the alpha-polyglutamated Antifolates can be prepared by reacting compounds of Formula I-Alpha with Z—COOH or an activated form thereof, to form alpha-polyglutamated drugs (Formula II-Alpha) or salts thereof under an amide forming condition. Compounds of Formula II-Alpha or salts thereof can then be deprotected to provide compounds of Formula III-Alpha, or a pharmaceutically acceptable salt thereof. Compounds of Formula IV-Alpha can be typically prepared from compounds of Formula III-Alpha or a salt thereof with a suitable alkali base such as NaOH. Compounds of Formula I-Alpha can be obtained through various methods, such as by reacting Formula S-1-Alpha with S-2-Alpha to provide the alpha-linked polyglutamate Formula S-3-Alpha, which can then be deprotected to provide the compound of Formula I-Alpha or salts thereof. Suitable amide coupling conditions and variables including Pg¹, Pg², Pg^{2′}, m, n, p, M⁺, and Z can be any of those described herein in the context of describing the gamma-polyglutamated drugs. Further, for any of the embodiments herein wherein gamma-polyglutamated Antifolate of the present disclosure is recited, alternative embodiments are also provided with the gamma-polyglutamated Antifolate replaced with a corresponding alpha-polyglutammated Antifolate. For example, for an embodiment directed to a pharmaceutical composition such as a liposomal composition comprising a substantially pure compound of Formula III or a pharmaceutically acceptable salt thereof, an alternative embodiment is also provided for a pharmaceutical composition comprising a substantially pure compound of Formula III-Alpha or a pharmaceutically acceptable salt thereof. In some embodiments of Formula III-Alpha, n can be 4; Z can be a residue of pemetrexed having the following formula:

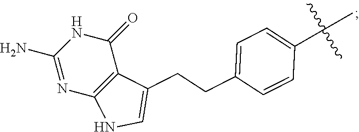 [SMILES: *c1ccc(CCc2c[nH]c3nc(N)[nH]c(=O)c23)cc1]

all glutamate units can be in L-form or all glutamate units can be in D-form; and/or the compound of Formula III-Alpha can be in a substantially pure form (e.g., at least 90% by HPLC and/or by weight). In some embodiments, the compounds of Formula III-Alpha are in the form of a sodium salt having a Formula IV-Alpha, such as a hepta-sodium salt when n is 4.

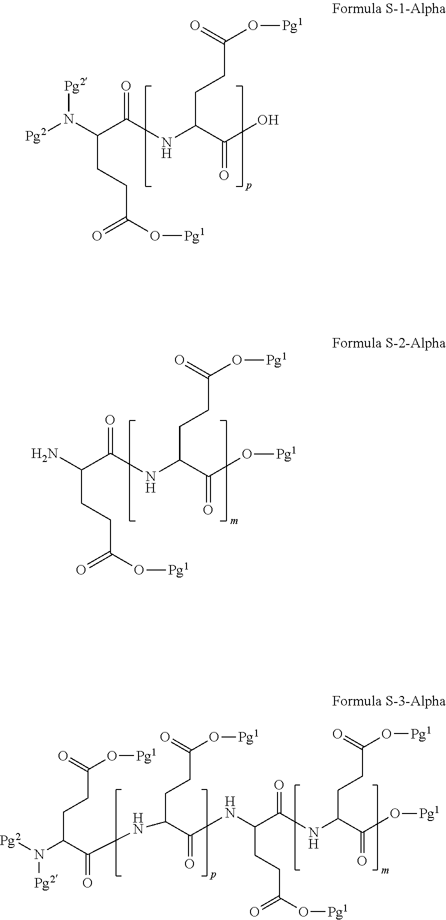

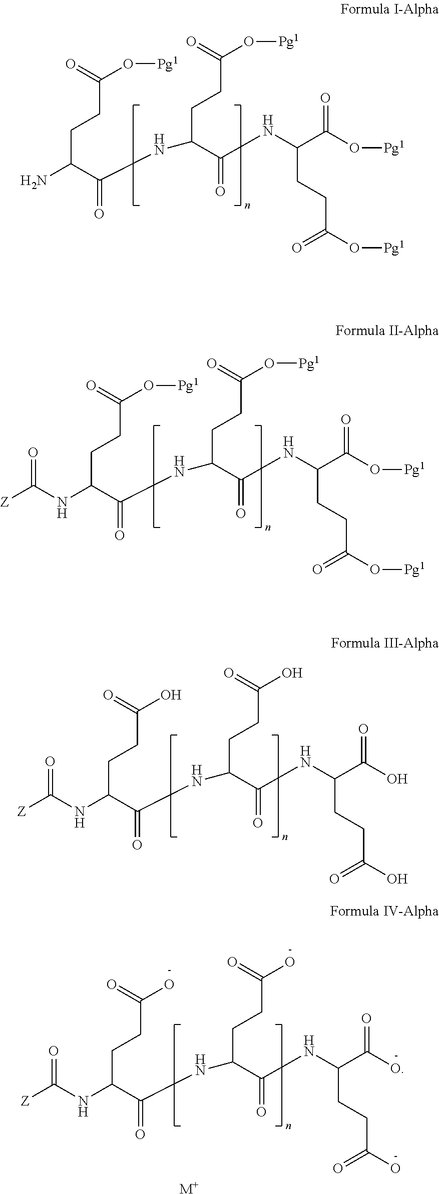

Typically, the synthetic method for αPANTIFOL herein can include an amide coupling reaction of a polyglutamate of Formula I-Alpha, or a salt thereof, with an antifolate having a formula of Z—COOH, or an activated form thereof, to form a polyglutamated compound of Formula II-Alpha, or a salt thereof:

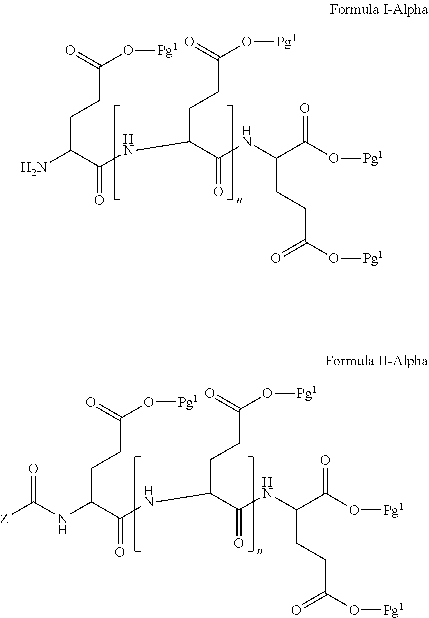

wherein each glutamate unit can independently be in a D-form or an L-form, Pg¹ at each occurrence is independently a carboxylic acid protecting group, and n can be an integer of 0-20, wherein Z is the residue of an Antifolate.

As with the synthesis of the γPANTIFOL here in, the activated form of a carboxylic acid does not need to be isolated for the amide coupling reaction herein. For example, in some embodiments, the polyglutamate of Formula I-Alpha, or a salt thereof, can react with a carboxylic acid of Z—COOH, in the presence of an amide coupling agent (e.g., chloroisobutyrate, DCC, DIC, PyBOP, PyAOP, EDCI, HATU, HBTU, TBTU, or T3P), which activates the carboxylic acid in situ. However, in some embodiments, an isolated activated form of a carboxylic acid can also be used for the synthetic methods herein.

Suitable conditions for the amide couplings between the polyglutamate of Formula I-Alpha, or a salt thereof, with the carboxylic acid of Z—COOH, or an activated form thereof, are generally known in the art. Various amide coupling agents can be used for the synthetic methods herein. Non-limiting useful amide coupling agents include chloroisobutyrate, DCC, DIC, PyBOP, PyAOP, EDCI, HATU, HBTU, TBTU, or T3P. Typically, when a carbodiimide coupling agent is used, such as DCC, DIC, EDCI, etc., the amide coupling reaction is also carried out in the presence of a benzotriazole, such as 1-hydroxy-benzotriazole (HOBt), 1-hydroxy-7-aza-benzotriazole (HOAt), etc. In some embodiments, a base is also added for the amide coupling. Suitable bases include inorganic bases such as carbonates (e.g., Na₂CO₃, NaHCO₃) and organic bases such as amine bases (e.g., diisopropylethyl amine, triethyl amine, N-methylmorpholine, pyridine) etc. The amide coupling reaction herein is typically carried out under conditions such that no or minimized racemization of chiral center(s) occurs. Exemplary amide coupling reaction conditions are shown in the Examples section.

In some embodiments, the synthetic method herein further comprises deprotecting the Pg¹ groups of Formula II-Alpha, or a salt thereof, to form the free carboxylic acid compound of Formula III-Alpha, or a salt thereof:

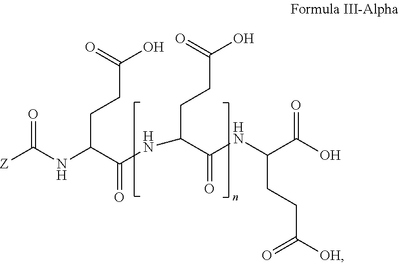

wherein Z and n are defined herein.

In some embodiments, each of the Pg¹ groups of Formula II-Alpha can be deprotected under acidic conditions. For example, in some embodiments, each of the Pg¹ groups of Formula II-Alpha is a tert-butyl group. In some embodiments, the deprotecting of the compound of Formula II-Alpha can be effected with an acid, such as trifluoroacetic acid (TFA), HCl, etc.

In some embodiments, the synthetic method herein further comprises converting the free carboxylic acid compound of Formula III-Alpha, or a salt thereof, into an alkali salt of Formula IV-Alpha:

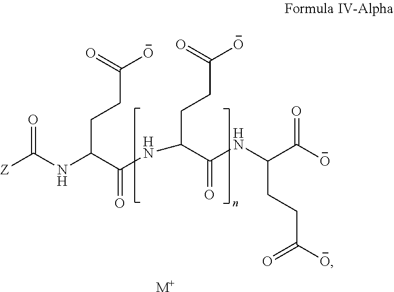

wherein M⁺ is an alkali counterion, such as Li⁺, Na⁺, or K⁺. The conversion can be typically carried out by contacting the compound of Formula III-Alpha or a salt thereof with a suitable alkali base, such as NaOH, etc. In some embodiments, the alkali salt of Formula IV-Alpha can be further isolated, purified, and/or crystallized by any suitable method, e.g., described herein. While the molar equivalent of M⁺ in Formula IV-Alpha is not specified, Formula IV-Alpha should not be understood as limited to having one molar equivalent of M⁺. To be clear, M⁺ in Formula IV-Alpha typically can balance the negative charges of the carboxylic acid groups in Formula IV-Alpha, with one mole of M⁺ per one mole of the negative charged carboxylic acid group in Formula IV-Alpha. For example, in some embodiments, n is 4, and M⁺ is Na⁺, the alkali salt of Formula IV-Alpha can be a hepta-sodium salt, i.e., 7 Na⁺ to counter balance the negative charges of the carboxylic acids so that Formula IV-Alpha is neutral overall. In some embodiments, the alkali cation M⁺ can also be combined with one or more other cations (e.g., pharmaceutically acceptable cations) to counter balance the negative charges of the carboxylic acid groups so that Formula IV-Alpha is overall neutral.

The synthetic methods described herein have various advantages. For example, the synthetic methods described herein (1) can be readily adapted for large-scale synthesis, e.g., kilogram-scale synthesis; (2) can have a high yield, with no or minimized racemization during the synthesis, and simple procedures for purification, such as through crystallization; and (3) can provide high purity intermediates and/or products, including compounds of Formulae I-Alpha, II-Alpha, III-Alpha, and IV-Alpha and salts thereof. (4) can reduce the requirements of manufacturing equipments due to a smaller number of repeating steps are used. These high purity intermediates and/or products are also novel compositions of the present disclosure.

Formula I-Alpha and Preparation

To prepare a high purity alpha polyglutamated antifolate herein, the synthesis typically uses a substantially pure polyglutamate of Formula I-Alpha or a salt thereof. For example, for the synthetic method, the compound of Formula I-Alpha (e.g., Formula I-L-Alpha or I-D-Alpha) or a salt thereof (e.g., a pharmaceutically acceptable salt) can typically have a purity of at least 90% (e.g., at least 90%, at least 95%, at least 98%, at least 99%) by HPLC and/or by weight.

In some embodiments, the substantially pure polyglutamate of Formula I-Alpha are also in a stereoisomerically pure or substantially pure form. In some embodiments, the polyglutamate of Formula I-Alpha can exist predominantly in one enantiomeric form, which can be free or substantially free (e.g., containing less than 5%, less than 2%, less than 1%, less than 0.5%, or less than 0.1%) of the other enantiomeric form. In some embodiments, the polyglutamate of Formula I-Alpha can also exist predominantly as one diastereomer, which can be free or substantially free (e.g., containing less than 5%, less than 2%, less than 1%, less than 0.5%, or less than 0.1%) of any other diastereomer(s).

In some embodiments, each of the glutamate units in Formula I-Alpha is in an L-form, and the compound of Formula I-Alpha is a compound of Formula I-L-Alpha:

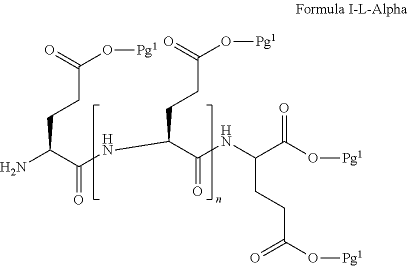

wherein Pg¹ and n are defined herein. In some embodiments, the polyglutamate of Formula I-L-Alpha can be stereoisomerically pure or substantially pure. For example, in some embodiments, the polyglutamate of Formula I-L-Alpha can be free or substantially free (e.g., containing less than 5%, less than 2%, less than 1%, less than 0.5%, or less than 0.1%) of the other enantiomeric form. In some embodiments, the polyglutamate of Formula I-L-Alpha can also be free or substantially free (e.g., containing less than 5%, less than 2%, less than 1%, less than 0.5%, or less than 0.1%) of any other diastereomer(s). However, in some embodiments, the polyglutamate of Formula I-L-Alpha can also exist in a racemic mixture or in a stereoisomeric mixture.

In some embodiments, each of the glutamate units in Formula I-Alpha is in a D-form, and the compound of Formula I-Alpha is a compound of Formula I-D-Alpha:

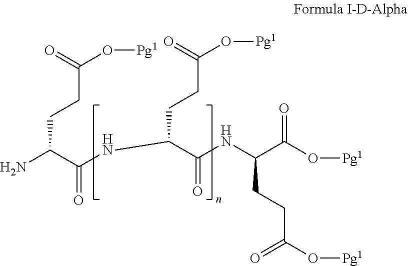

wherein Pg¹ and n are defined herein. In some embodiments, the polyglutamate of Formula I-D-Alpha can also be stereoisomerically pure or substantially pure. For example, in some embodiments, the polyglutamate of Formula I-D-Alpha can be free or substantially free (e.g., containing less than 5%, less than 2%, less than 1%, less than 0.5%, or less than 0.1%) of the other enantiomeric form. In some embodiments, the polyglutamate of Formula I-D-Alpha can also be free or substantially free (e.g., containing less than 5%, less than 2%, less than 1%, less than 0.5%, or less than 0.1%) of any other diastereomers. However, in some embodiments, the polyglutamate of Formula I-D-Alpha can also exist in a racemic mixture or in a stereoisomeric mixture.

Various carboxylic acid protecting groups are suitable for use as Pg¹ in Formula I-Alpha (e.g., Formula I-L-Alpha or I-D-Alpha). Carboxylic acid protecting groups (or alternatively referred to herein as carboxyl protecting group) are generally known in the art, for example, as described in “Protective Groups in Organic Synthesis”, 4th ed. P. G. M. Wuts; T. W. Greene, John Wiley, 2007, and references cited therein. In some embodiments, Pg¹ in Formula I-Alpha (e.g., Formula I-L-Alpha or I-D-Alpha) at each occurrence can be a carboxyl protecting group that can be removed under acidic conditions, such as a tertiary alkyl group, such as tert-butyl. In some embodiments, Pg1 in Formula I-Alpha (e.g., Formula I-L-Alpha or I-D-Alpha) at each occurrence can be a carboxyl protecting group that can be removed under basic conditions, such as methyl, ethyl, benzyl, etc. In some embodiments, Pg¹ in Formula I-Alpha (e.g., Formula I-L-Alpha or I-D-Alpha) at each occurrence can be a carboxyl protecting group that can be removed through a nucleophilic attack, such as methyl, ethyl, benzyl. In some embodiments, Pg¹ in Formula I-Alpha (e.g., Formula I-L-Alpha or I-D-Alpha) at each occurrence can be a carboxyl protecting group that can be removed through a photoreaction, i.e., the protecting group is a photoreleasable protecting group. Photoreleasable protecting groups are known in the art, for example, as described in Klan et al. “Photoremovable Protecting Groups in Chemistry and Biology: Reaction Mechanisms and Efficacy,” Chem. Rev. 113:119-191 (2013). In some embodiments, Pg¹ in Formula I-Alpha (e.g., Formula I-L-Alpha or I-D-Alpha) at each occurrence can be a carboxyl protecting group that can be removed under hydrogenation conditions, such as benzyl.

Typically, all of the Pg¹ in Formula I-Alpha (e.g., Formula I-L-Alpha or I-D-Alpha) are the same protecting group. However, in some embodiments, the Pg¹ groups in Formula I-Alpha (e.g., Formula I-L-Alpha or I-D-Alpha) can also be different and can be deprotected under different conditions. For example, in some embodiments, the Pg¹ group for the C-terminal carboxylic acid group (either alpha-carboxylic acid group or gamma-carboxylic acid group) can be different from and/or orthogonal to the Pg¹ group(s) for the remaining carboxylic acid groups. In such embodiments, the Pg¹ group for the C-terminal carboxylic acid group (either alpha-carboxylic acid group or gamma-carboxylic acid group) can be selectively deprotected in the presence of the other Pg¹ group(s), and vice versa, which allows further functionalization of the C-terminal carboxylic acid group.

The polyglutamate of Formula I-Alpha (e.g., Formula I-L-Alpha or I-D-Alpha) described herein can typically comprise 2-20 glutamate units, for example, 2-20, 2-15, 2-10, 2-6, 2-5, or more than 5. In some embodiments, the polyglutamate of Formula I-Alpha (e.g., Formula I-L-Alpha or I-D-Alpha) can refer to a specific oligomer, with n being a specific integer. For example, in some embodiments, n in Formula I-Alpha (e.g., Formula I-L-Alpha or I-D-Alpha) can be 0, 1, 2, 3, 4, 5, 6, 7, 8, 9, 10, 11, 12, 13, 14, 15, 16, 17, or 18. In some embodiments, the polyglutamate of Formula I-Alpha (e.g., Formula I-L-Alpha or I-D-Alpha) can be a hexaglutamate (n is 4), which can be substantially free of a polyglutamate of Formula I-Alpha wherein n is not 4. In some embodiments, the polyglutamate of Formula I-Alpha can also refer to a mixture of polyglutamates which have different number of glutamate units. For example, in some embodiments, the polyglutamate of Formula I-Alpha can comprise a mixture of polyglutamate of Formula I-Alpha wherein n is 0-18, 0-13, 2-6, 0-8, 0-3, etc.

Compounds of Formula I-Alpha (e.g., Formula I-L-Alpha or I-D-Alpha) are typically prepared from deprotection of a compound of Formula I-P-Alpha, or a salt thereof:

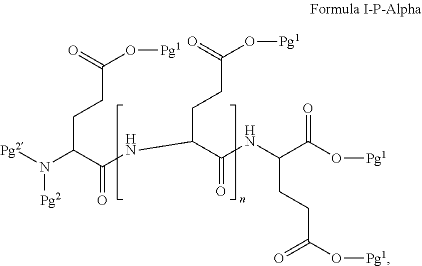

wherein Pg² and Pg^{2′} are independently hydrogen or a nitrogen protecting group, provided that at least one of Pg² and Pg^{2′} is a nitrogen protecting group; or Pg² and Pg^{2′} together with the nitrogen atom they are attached to form a cyclic protected amino group. Nitrogen protecting groups are generally known in the art, for example, as described in “Protective Groups in Organic Synthesis”, 4^{th} ed. P. G. M. Wuts; T. W. Greene, John Wiley, 2007, and references cited therein. Non-limiting examples of suitable nitrogen protecting groups include carbobenzyloxy (Cbz) (removable by hydrogenolysis), p-methoxybenzyl carbonyl (Moz or MeOZ) (removable by hydrogenolysis), tert-butyloxycarbonyl (Boc) (removable by acids, such as HCl or trifluoroacetic acid, or by heating), 9-fluorenylmethyloxycarbonyl (FMOC) (removable by base, such as piperidine), acetyl (Ac) (removable by treatment with a base), benzoyl (Bz) (removable by treatment with a base, most often with aqueous or gaseous ammonia or methylamine), benzyl (Bn) (removable by hydrogenolysis), a carbamate (removable by acid and mild heating), p-methoxybenzyl (PMB) (removable by hydrogenolysis), 3,4-dimethoxybenzyl (DMPM) (removable by hydrogenolysis), p-methoxyphenyl (PMP) (removable by ammonium cerium(IV) nitrate), a succinimide (a cyclic imide) (removable by treatment with a base), tosyl (Ts) (removable by concentrated acid and strong reducing agents), and other sulfonamides (Nosyl and Nps) (removable by samarium iodide, tributyltin hydride, etc.). In some embodiments, neither of Pg² and Pg^{2′} is Fmoc.

Typically, the Pg² and Pg^{2′} are selected such that the deprotection can be carried out in high efficiency, such that the deprotected product, i.e., compound of Formula I-Alpha or salts thereof, can be used directly for coupling with Z—COOH or an activated form thereof. For example, in some embodiments, one of Pg² and Pg^{2′} in Formula I-P-Alpha is hydrogen, and the other of Pg² and Pg^{2′} is a nitrogen protecting group capable of being deprotected via hydrogenation, e.g., Pg² is benzyloxycarbonyl (Cbz). In such embodiments, the deprotection can be carried out in high efficiency and typically, the deprotected product can be used directly without further purification.

In any of the embodiments described herein, the Pg¹ groups and the amine protecting group(s) of Formula I-P-Alpha can be orthogonal. For example, in some embodiments, the amine protecting group(s) of Formula I-P-Alpha can be protecting groups removable under hydrogenation conditions but are stable under acidic conditions (e.g., TFA), whereas the Pg¹ groups are stable under hydrogenation conditions but are removable under acidic conditions (e.g., TFA). Alternatively, in some embodiments, the amine protecting group(s) of Formula I-P-Alpha can be protecting groups that are stable under hydrogenation conditions but are removable under acidic conditions (e.g., TFA), whereas the Pg¹ groups are removable under hydrogenation conditions but are stable under acidic conditions (e.g., TFA). In some embodiments, one of Pg² and Pg^{2′} in Formula I-P-Alpha is hydrogen, and the other of Pg² and Pg^{2′} is a nitrogen protecting group capable of being deprotected via hydrogenation, e.g., Pg² is benzyloxycarbonyl. Various conditions for hydrogenation are suitable. Typically, such hydrogenation can be carried out in the presence of a heterogenous catalyst, such as Pd/C, under H₂ gas, in a solvent such as an alcoholic solvent (e.g., methanol, ethanol, etc.). In some embodiments, all of the Pg¹ groups are acid deprotectable protecting groups such as tert-butyl.

For preparing a high purity compound of Formula I-Alpha or salt thereof, the compound of Formula I-P-Alpha (e.g., Formula I-P-L-Alpha or I-P-D-Alpha) or a salt thereof (e.g., a pharmaceutically acceptable salt) used is typically also substantially pure, for example, has a purity of at least 90% (e.g., at least 90%, at least 95%, at least 98%, at least 99%) by HPLC and/or by weight. In some embodiments, the compound of Formula I-P-Alpha can also exist predominantly in one enantiomeric form, which can be free or substantially free (e.g., containing less than 5%, less than 2%, less than 1%, less than 0.5%, or less than 0.1%) of the other enantiomeric form. In some embodiments, the compound of Formula I-P-Alpha can also exist predominantly as one diastereomer, which can be free or substantially free (e.g., containing less than 5%, less than 2%, less than 1%, less than 0.5%, or less than 0.1%) of any other diastereomer(s).

In some embodiments, each of the glutamate units in the compound of Formula I-P-Alpha is in an L-form, and the compound of Formula I-P-Alpha is a compound of Formula I-P-L-Alpha:

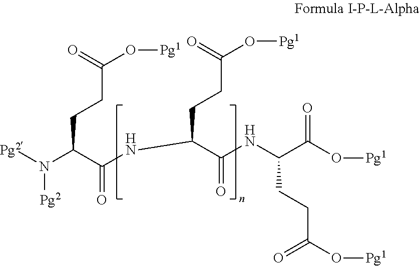

wherein Pg¹, Pg², Pg^{2′}, and n are defined herein. In some embodiments, the compound of Formula I-P-L-Alpha can be stereoisomerically pure or substantially pure. For example, in some embodiments, the compound of Formula I-P-L-Alpha can be free or substantially free (e.g., containing less than 5%, less than 2%, less than 1%, less than 0.5%, or less than 0.1%) of the other enantiomeric form. In some embodiments, the compound of Formula I-P-L-Alpha can also be free or substantially free (e.g., containing less than 5%, less than 2%, less than 1%, less than 0.5%, or less than 0.1%) of any other diastereomer(s). However, in some embodiments, the compound of Formula I-P-L-Alpha can also exist in a racemic mixture or in a stereoisomeric mixture.

In some embodiments, each of the glutamate units in Formula I-P-Alpha is in a D-form, and the compound of Formula I-P-Alpha is a compound of Formula I-P-D-Alpha:

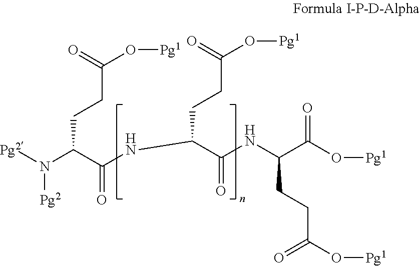

wherein Pg¹, Pg², Pg^{2′}, and n are defined herein. In some embodiments, the compound of Formula I-P-D-Alpha can also be stereoisomerically pure or substantially pure. For example, in some embodiments, the compound of Formula I-P-D-Alpha can be free or substantially free (e.g., containing less than 5%, less than 2%, less than 1%, less than 0.5%, or less than 0.1%) of the other enantiomeric form. In some embodiments, the compound of Formula I-P-D-Alpha can also be free or substantially free (e.g., containing less than 5%, less than 2%, less than 1%, less than 0.5%, or less than 0.1%) of any other diastereomers. However, in some embodiments, the compound of Formula I-P-D-Alpha can also exist in a racemic mixture or in a stereoisomeric mixture.

Compounds of Formula I-P-Alpha (e.g., Formula I-P-L-Alpha or I-P-D-Alpha) are typically prepared from protected glutamate or protected polyglutamate via amide coupling reactions. For example, in some embodiments, the method of preparing a compound of Formula I-P-Alpha comprises:
a) reacting an acid of Formula S-1-Alpha, or an activated form thereof, with a protected polyglutamate of Formula S-2-Alpha, or a salt thereof, under an amide forming condition to form a compound of Formula S-3-Alpha, or a salt thereof:

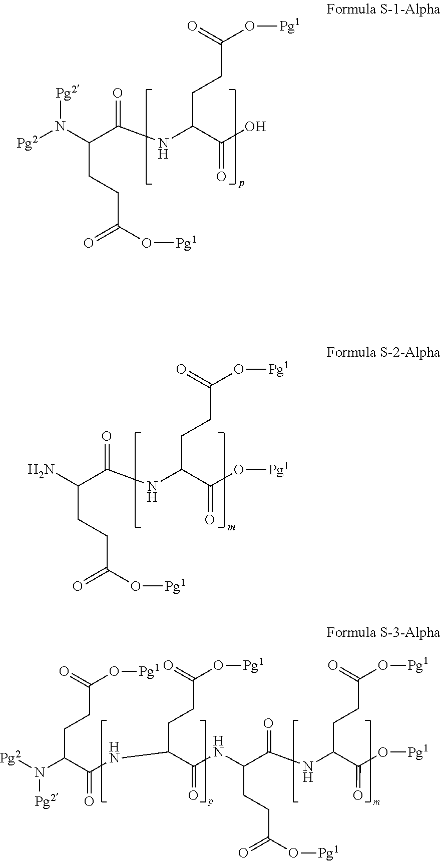

wherein Pg¹, Pg² and Pg^{2′} are defined herein, wherein each glutamate unit is independently in an L-form or D-form (e.g., all glutamate units are in L-form or all glutamate units are in D-form), wherein m+p=n, and n is defined herein. In some embodiments, m is 0-19, for example, 2-6 (e.g., 3 or 4). In some embodiments, p is 0-19. Typically, p is 0. However, in some embodiments, p is not 0. Those skilled in the art would understand that when m+p=n, Formula S-3-Alpha is the same as Formula I-P-Alpha. In some embodiments, Formula S-3-Alpha can be deprotected to provide the compound of Formula I-Alpha. Compounds of Formula S-2-Alpha can be prepared similarly.

In some embodiments, p in Formula S-1-Alpha is 0 and the glutamate units of Formula I-P-Alpha are introduced one by one consecutively. For example, in some embodiments, the method of preparing a compound of Formula I-P-Alpha comprises:
1) reacting an acid of Formula S-1-A-Alpha, or an activated form thereof, with an amine of Formula S-2-A-Alpha, or a salt thereof, under amide forming conditions to provide the dimer compound of Formula S-3-A-Alpha:

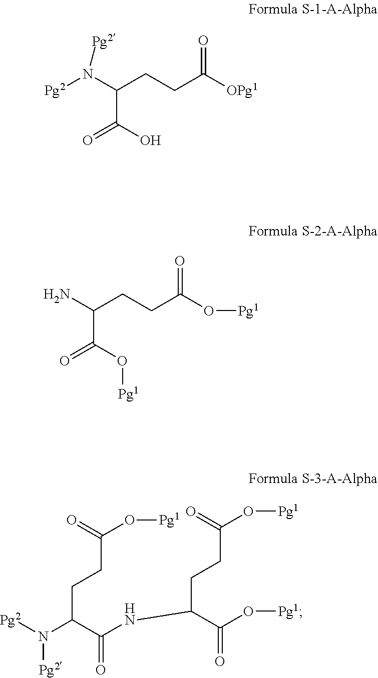

2) deprotecting the amine protecting group(s) of the compound of Formula S-3-A-Alpha to form a compound of S-2-B-Alpha, or a salt thereof;

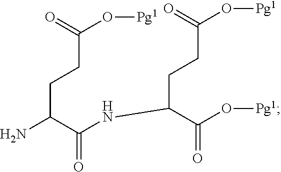

3) reacting the compound of Formula S-2-B-Alpha or a salt thereof with the acid of Formula S-1-A-Alpha, or an activated form thereof, under amide forming conditions to elongate the chain by one glutamate unit to provide the trimer compound of Formula S-3-B-Alpha:

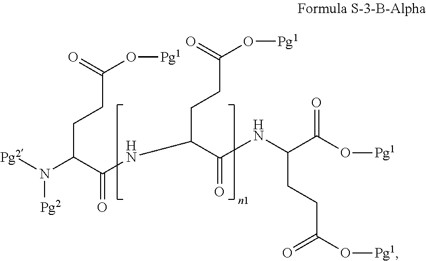

wherein n₁ is 1; and optionally
4) repeating the sequence of deprotecting the amine protecting group(s) and reacting the deprotected compound with the acid of Formula S-1-A-Alpha, or an activated form thereof, under amide forming conditions to elongate the chain until the desired number of glutamate unit is reached to form the compound of Formula I-P-Alpha:

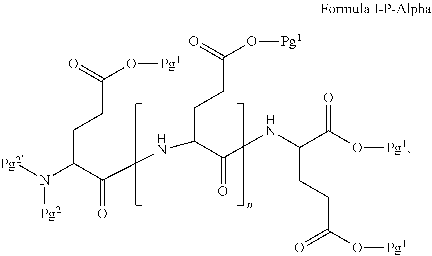

wherein Pg¹, Pg², Pg^{2′} and n are defined herein, wherein each glutamate unit is independently in an L-form or D-form (e.g., all glutamate units are in L-form or all glutamate units are in D-form). An example of preparing a compound of Formula I-P-Alpha (n is 4) is provided in the Examples section.

In some embodiments, the synthetic method herein is for preparing a compound of Formula I-P-L-Alpha. In such embodiments, the corresponding starting materials and/or intermediates used for the methods typically have each of the glutamate units in an L-form. For example, in some embodiments, each glutamate unit in each of Formula S-1-Alpha, S-2-Alpha, S-3-Alpha, S-1-A-Alpha, S-2-A-Alpha, S-3-A-Alpha, S-2-B-Alpha, and S-3-B-Alpha can be in the L-form. For example, in some embodiments, compounds of Formulae S-1-Alpha, S-2-Alpha, and S-3-Alpha can have a Formula S-1-L-Alpha, S-2-L-Alpha, or S-3-L-Alpha, wherein the variables are defined herein, respectively:

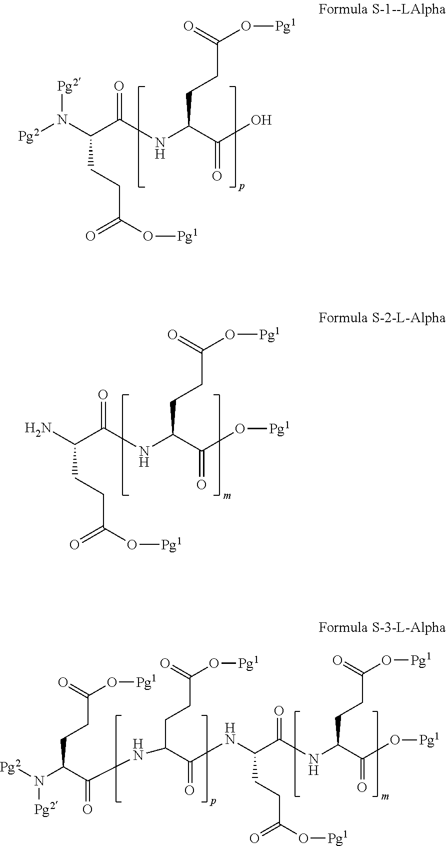

In some embodiments, the compound of Formula S-1-L-Alpha, S-2-L-Alpha, or S-3-L-Alpha can be free or substantially free (e.g., containing less than 5%, less than 2%, less than 1%, less than 0.5%, or less than 0.1%) of the other enantiomeric form, respectively. In some embodiments, the compound of Formula S-1-L-Alpha, S-2-L-Alpha, or S-3-L-Alpha can also be free or substantially free (e.g., containing less than 5%, less than 2%, less than 1%, less than 0.5%, or less than 0.1%) of any other diastereomers, respectively. However, in some embodiments, the compound of Formula S-1-L-Alpha, S-2-L-Alpha, or S-3-L-Alpha can also exist in a racemic mixture or in a stereoisomeric mixture, respectively.

In some embodiments, the method of preparing a compound of Formula I-P-Alpha is for preparing a compound of Formula I-P-D-Alpha. In such embodiments, the corresponding starting materials and/or intermediates used for the methods have each of the glutamate units in a D-form. For example, in some embodiments, each glutamate unit in each of Formula S-1-Alpha, S-2-Alpha, S-3-Alpha, S-1-A-Alpha, S-2-A-Alpha, S-3-A-Alpha, S-2-B-Alpha, and S-3-B-Alpha can be in the D-form.

In some embodiments, compounds of Formula I-Alpha (e.g., Formula I-L-Alpha or I-D-Alpha) can also be prepared using solid phase chemistry. For example, an initial glutamyl residue can be bonded to a Wang resin (or other suitable resins or solid supports) and additional glutamyl residues are added serially via solid phase peptide synthesis using F-moc chemistry. After the final glutamyl residue is added the Antifolate precursor (e.g., pemetrexed precursor) is coupled to the peptide and the molecule is cleaved from the resin. In some embodiments, compounds of Formula I-Alpha (e.g., Formula I-L-Alpha or I-D-Alpha) are not prepared using solid phase chemistry.

### Formula II-Alpha and Preparation

The compound of Formula II-Alpha is a polyglutamated antifolate, with Z in Formula II-Alpha being a residue of a suitable antifolate. Non-limiting suitable antifolates include any of those described in WO 2018/031967, WO 2018/031968, WO 2018/031979, WO 2018/031980, WO 2019/094648, PCT/US2019/016989, and PCT/US2019/017004, the content of each of which is herein incorporated by reference in its entirety. Some exemplary antifolates are described herein. While embodiments of the present disclosure are directed to polyglutamated antifolates, the compound of Formula I, or a salt thereof, can form an amide with any other drug with a carboxylic acid group or an activated form thereof, to form a compound of Formula II-Alpha, or a salt thereof, wherein Z in Formula II-Alpha represents a residue of such drug.

The conversion of the compound of Formula I-Alpha or a salt thereof into the corresponding compound of Formula II-Alpha or a salt thereof can be typically carried out with no or minimized racemization of chiral centers. In some embodiments, the polyglutamates of Formula II-Alpha are prepared in a stereoisomerically pure or substantially pure form in a large scale. For example, in some embodiments, the present disclosure provides the polyglutamates of Formula II-Alpha in a stereoisomerically pure or substantially pure form in a batch size over 10 grams (such as a batch size of about 100 gram or more, about 1 kg or more, about 5 kg or more, about 10 kg or more, etc.). In some embodiments, the polyglutamate of Formula II-Alpha can exist predominantly in one enantiomeric form, which can be free or substantially free (e.g., containing less than 5%, less than 2%, less than 1%, less than 0.5%, or less than 0.1%) of the other enantiomeric form. In some embodiments, the polyglutamate of Formula II-Alpha can also exist predominantly as one diastereomer, which can be free or substantially free (e.g., containing less than 5%, less than 2%, less than 1%, less than 0.5%, or less than 0.1%) of any other diastereomer(s).

In some embodiments, each of the glutamate units in Formula II-Alpha is in an L-form, and the compound of Formula II-Alpha is a compound of Formula II-L-Alpha:

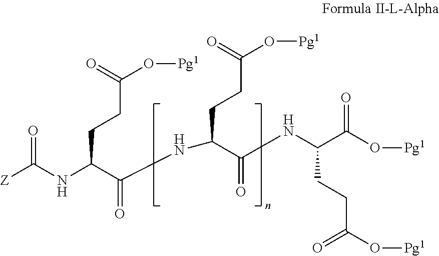

wherein Pg¹, Z, and n are defined herein. In some embodiments, the polyglutamate of Formula II-L-Alpha can be stereoisomerically pure or substantially pure. For example, in some embodiments, the polyglutamate of Formula II-L-Alpha can be free or substantially free (e.g., containing less than 5%, less than 2%, less than 1%, less than 0.5%, or less than 0.1%) of the other enantiomeric form. In some embodiments, the polyglutamate of Formula II-L-Alpha can also be free or substantially free (e.g., containing less than 5%, less than 2%, less than 1%, less than 0.5%, or less than 0.1%) of any other diastereomer(s). However, in some embodiments, the polyglutamate of Formula II-L-Alpha can also exist in a racemic mixture or in a stereoisomeric mixture.

In some embodiments, each of the glutamate units in Formula II-Alpha is in a D-form, and the compound of Formula II-Alpha is a compound of Formula II-D-Alpha:

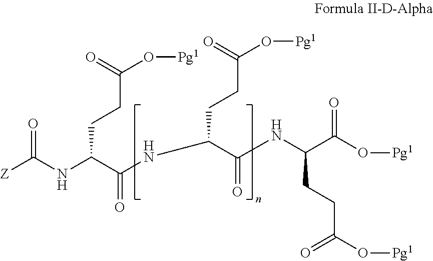

wherein Pg¹, Z, and n are defined herein. In some embodiments, the polyglutamate of Formula II-D-Alpha can be stereoisomerically pure or substantially pure. For example, in some embodiments, the polyglutamate of Formula II-D-Alpha can be free or substantially free (e.g., containing less than 5%, less than 2%, less than 1%, less than 0.5%, or less than 0.1%) of the other enantiomeric form. In some embodiments, the polyglutamate of Formula II-D-Alpha can also be free or substantially free (e.g., containing less than 5%, less than 2%, less than 1%, less than 0.5%, or less than 0.1%) of any other diastereomer(s). However, in some embodiments, the polyglutamate of Formula II-D-Alpha can also exist in a racemic mixture or in a stereoisomeric mixture.

In some specific embodiments, Z in Formula II-Alpha (e.g., Formula II-L-Alpha or II-D-Alpha) can be a residue of an antifolate selected from methotrexate (MTX), pemetrexed (PMX), lometrexol (LTX), AG2034, raltitrexed (RTX), pralatrexate, GW1843, aminopterin, LY309887 and LY222306.

In some specific embodiments, Z in Formula II-Alpha (e.g., Formula II-L-Alpha or II-D-Alpha) can be a residue of pemetrexed having the following formula:

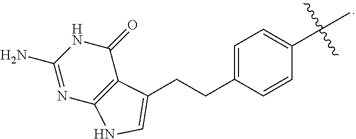 [SMILES: *c1ccc(CCc2c[nH]c3nc(N)[nH]c(=O)c23)cc1]

In some specific embodiments, Z in Formula II-Alpha (e.g., Formula II-L-Alpha or II-D-Alpha) can be a residue having the following formula:

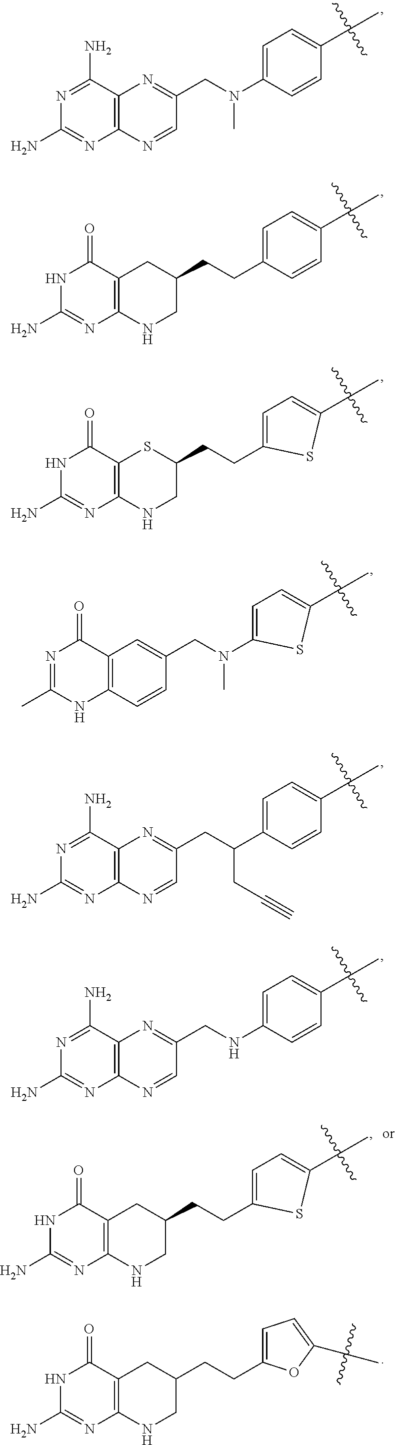

In some embodiments, Z can be a residue having the following formula:

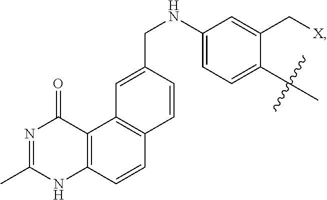

wherein X is a leaving group. In some embodiments, Z—COOH, upon reaction with the compound Formula I-Alpha under an amide forming condition, can form an intermediate, which can be further converted into a cyclic structure:

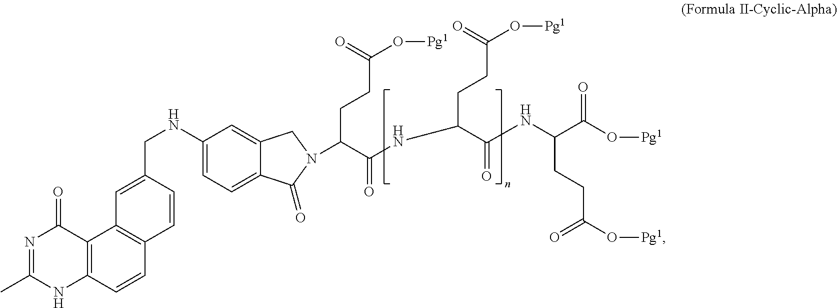

wherein each glutamate unit is independently in an L-form or D-form (e.g., all glutamate units are in L-form or all glutamate units are in D-form), Pg¹ and n (e.g., n can be 0-20, such as 2, 3, or 4) are defined herein, which can be further converted into the compound of the following formula or a pharmaceutically acceptable salt thereof:

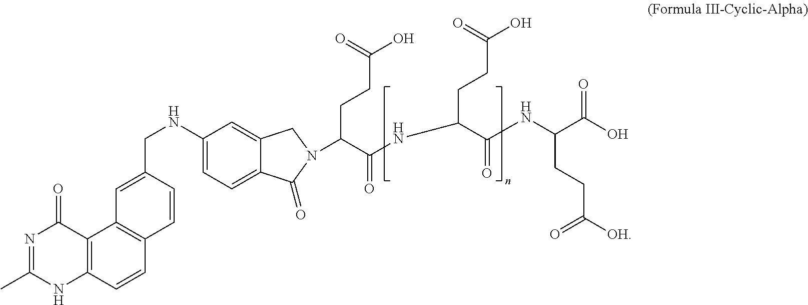

The Pg¹ and n for Formula II-Alpha can be any of those described herein as suitable for the polyglutamate of Formula I-Alpha. For example, in some specific embodiments, n in Formula II-Alpha (e.g., Formula II-L-Alpha or II-D-Alpha) can be an integer of 0-20, for example, 2-20, 2-15, 2-10, 2-6, 2-5, or more than 5. In some specific embodiments, Pg¹ in Formula II-Alpha (e.g., Formula II-L-Alpha or II-D-Alpha) can be an acid labile carboxylic acid protecting group, such as tert-butyl.

The compound of Formula II-Alpha (e.g., Formula II-L-Alpha or II-D-Alpha) or a salt thereof (e.g., a pharmaceutically acceptable salt) is typically substantially pure, for example, has a purity of at least 90% (e.g., at least 90%, at least 95%, at least 98%, at least 99%) by HPLC and/or by weight. In some embodiments, the compound of Formula II-Alpha can also be in a solid form, such as a crystalline form, an amorphous form, or a mixture thereof. For example, in some embodiments, the compound of Formula II-Alpha can be purified through crystallization, such as using a suitable solvent system.

In some embodiments, the compound of Formula II-Alpha (e.g., Formula II-L-Alpha or II-D-Alpha) can be a substantially pure specific oligomer, for example, a substantially pure tetraglutamate (n is 2), a substantially pure pentaglutamate (n is 3), a substantially pure hexaglutamate (n is 4), a substantially pure heptaglutamate (n is 5), etc. In some specific embodiments, the compound of Formula II-Alpha (e.g., Formula II-L-Alpha or II-D-Alpha) can be a substantially pure hexaglutamate, wherein n in Formula II-Alpha can be 4. For example, in such embodiments, the compound of Formula II-Alpha (e.g., Formula II-L-Alpha or II-D-Alpha) can be substantially free (e.g., less than 10%, less than 5%, less than 2%, less than 1%, less than 0.5%, or less than 0.1%) of another compound of Formula II-Alpha wherein n is not 4. Compounds of Formula II-Alpha with the recited purity profile can be prepared by controlling the purity of the corresponding polyglutamate of Formula I-Alpha used for the amide coupling reaction with Z—COOH or an activated form thereof. Exemplary procedures are described in the Examples section.

### Formula III-Alpha and Preparation

The compound of Formula II-Alpha, or a salt thereof, can be deprotected to form a compound of Formula III-Alpha, or a salt thereof. In some embodiments, the compound of Formula III-Alpha or a salt thereof can be substantially pure. In some embodiments, the compound of Formula III-Alpha can be in an acid addition salt, such as a TFA salt. The acid addition salt of Formula III-Alpha can be substantially pure, which can be used by itself in a pharmaceutical composition. In some embodiments, the acid addition salt of Formula III-Alpha can also be used as an intermediate to prepare a high purity salt of Formula III-Alpha, such as an alkali salt of Formula IV-Alpha. When used as an intermediate, the acid addition salt of Formula III-Alpha does not have to be a pharmaceutically acceptable salt.

In some embodiments, the compound of Formula III-Alpha is present in a form of a pharmaceutically acceptable salt, e.g., a sodium salt, which includes monosodium, disodium, trisodium, etc., with the number of sodium up to the number of negatively charged carboxylic acid groups in Formula III-Alpha. For example, when n is 4, there are a total of 7 carboxylic acid groups in Formula III-Alpha (not considering any potential carboxylic acid group in Z group), and the salt can be a monosalt, disalt, trisalt, and up to hepta-salt, such as hepta-sodium salt. In some embodiments, the compound of Formula III-Alpha can be in a form of a pharmaceutically acceptable acid addition salt, such as an HCl salt. In some embodiments, the acid addition salt such as HCl salt or the base addition salt such as a sodium salt can be used for controlling osmolarity, such as maintaining appropriate osmolarity in liposomal encapsulation.

As will be understood by those skilled in the art, when the compound of Formula III-Alpha or its pharmaceutically acceptable salt(s) is formulated, further processed, or administered, the actual ionization state of the compound of Formula III-Alpha will depend on the pH of the medium encompassing the compound of Formula III-Alpha. Taking a hexglutamated Antifolate (n is 4) of the present disclosure as an example, the seven carboxylic acid group can be partially ionized when the medium pH is about 6.5 to 7.0, and can be fully ionized at a higher pH such as greater than 10. When formulated, for example, as a liposomal composition herein, the compound of Formula III-Alpha or its pharmaceutically acceptable salt(s) can become partially ionized or fully ionized depending on the pH of the formulation medium, regardless of whether the free form or a salt form of the compound of Formula III-Alpha (e.g., a HCl salt of Formula III-Alpha or an alkali salt of Formula IV-Alpha) is used as the starting drug substance for the formulation. The compositions of the present disclosure such as the liposomal compositions herein should not be understood as to be limited to any particular ionization state of the compound of Formula III-Alpha. In some embodiments, the ionization state of the compound of Formula III-Alpha in a composition, for example, in a liposomal composition, can also be controlled by adjusting the medium pH. In some embodiments, the ionization state of the compound of Formula III-Alpha in a composition can be monitored by measuring the osmolarity of the composition.

Compounds of Formula III-Alpha can contain both a basic and an acidic functionality, and can be converted to a pharmaceutically acceptable salt, when desired, by using a suitable acid or base.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Examples of acids which can be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid. In some embodiments, the pharmaceutically acceptable salt of compounds of Formula III-Alpha is an acid addition salt such as HCl salt.

Basic addition salts can be prepared by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like. In some embodiments, the pharmaceutically acceptable salt of compounds of Formula III-Alpha is a base addition salt such as an alkali salt, an alkaline earth metal salt, etc. as described herein.

In some embodiments, each of the Pg¹ groups of Formula II-Alpha is an acid labile protecting group, which can be deprotected under acidic conditions. In some embodiments, the Pg¹ groups of Formula II-Alpha are the same acid labile protecting group. For example, in some embodiments, each of the Pg¹ groups can be a tert-butyl group. In some embodiments, the deprotecting of the compound of Formula II-Alpha can be effected with an acid, such as trifluoroacetic acid (TFA), HCl, etc.

The conversion of a compound of Formula II-Alpha or a salt thereof into the corresponding compound of Formula III-Alpha or a salt thereof can be typically carried out with no or minimized racemization of chiral centers. In some embodiments, the polyglutamates of Formula III-Alpha are prepared in a stereoisomerically pure or substantially pure form in large scales. For example, in some embodiments, the present disclosure provides the polyglutamates of Formula III-Alpha in a stereoisomerically pure or substantially pure form in a batch size over 10 grams (such as a batch size of about 100 gram or more, about 1 kg or more, about 5 kg or more, about 10 kg or more, etc.). In some embodiments, the polyglutamate of Formula III-Alpha can exist predominantly in one enantiomeric form, which can be free or substantially free (e.g., containing less than 5%, less than 2%, less than 1%, less than 0.5%, or less than 0.1%) of the other enantiomeric form. In some embodiments, the polyglutamate of Formula III-Alpha can also exist predominantly as one diastereomer, which can be free or substantially free (e.g., containing less than 5%, less than 2%, less than 1%, less than 0.5%, or less than 0.1%) of any other diastereomer(s).

In some embodiments, each of the glutamate units in Formula III-Alpha is in an L-form, and the compound of Formula III-Alpha is a compound of Formula III-L-Alpha:

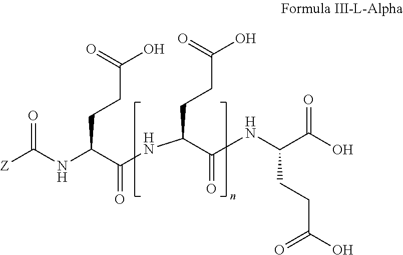

wherein Z and n are defined herein. In some embodiments, the polyglutamate of Formula III-L-Alpha can be stereoisomerically pure or substantially pure. For example, in some embodiments, the polyglutamate of Formula III-L-Alpha can be free or substantially free (e.g., containing less than 5%, less than 2%, less than 1%, less than 0.5%, or less than 0.1%) of the other enantiomeric form. In some embodiments, the polyglutamate of Formula III-L-Alpha can also be free or substantially free (e.g., containing less than 5%, less than 2%, less than 1%, less than 0.5%, or less than 0.1%) of any other diastereomer(s). However, in some embodiments, the polyglutamate of Formula III-L-Alpha can also exist in a racemic mixture or in a stereoisomeric mixture.

In some embodiments, each of the glutamate units in Formula III-Alpha is in a D-form, and the compound of Formula III-Alpha is a compound of Formula III-D-Alpha:

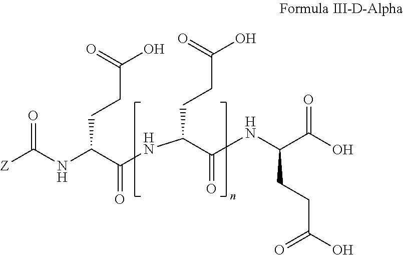

wherein Z and n are defined herein. In some embodiments, the polyglutamate of Formula III-D-Alpha can be stereoisomerically pure or substantially pure. For example, in some embodiments, the polyglutamate of Formula III-D-Alpha can be free or substantially free (e.g., containing less than 5%, less than 2%, less than 1%, less than 0.5%, or less than 0.1%) of the other enantiomeric form. In some embodiments, the polyglutamate of Formula III-D-Alpha can also be free or substantially free (e.g., containing less than 5%, less than 2%, less than 1%, less than 0.5%, or less than 0.1%) of any other diastereomer(s). However, in some embodiments, the polyglutamate of Formula III-D-Alpha can also exist in a racemic mixture or in a stereoisomeric mixture.

In some specific embodiments, Z in Formula III-Alpha (e.g., Formula III-L-Alpha or III-D-Alpha) can be a residue of an antifolate selected from methotrexate (MTX), pemetrexed (PMX), lometrexol (LTX), AG2034, raltitrexed (RTX), pralatrexate, GW1843, aminopterin, LY309887 and LY222306.

In some specific embodiments, Z in Formula III-Alpha (e.g., Formula III-L-Alpha or III-D-Alpha) can be a residue of pemetrexed having the following formula:

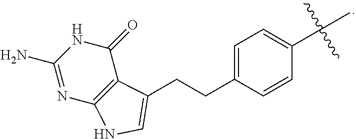 [SMILES: *c1ccc(CCc2c[nH]c3nc(N)[nH]c(=O)c23)cc1]

In some specific embodiments, Z in Formula III-Alpha (e.g., Formula III-L-Alpha or III-D-Alpha) can be a residue having the following formula:

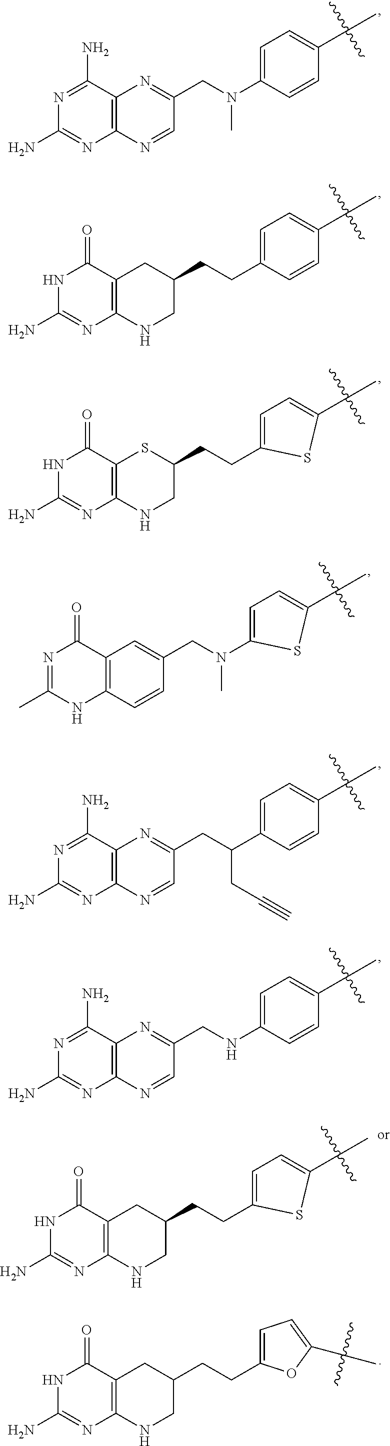

In some specific embodiments, n in Formula III-Alpha (e.g., Formula III-L-Alpha or III-D-Alpha) can be an integer of 0-20, for example, 2-20, 2-15, 2-10, 2-6, 2-5, or more than 5.

The compound of Formula III-Alpha (e.g., Formula III-L-Alpha or III-D-Alpha) or a salt thereof (e.g., a pharmaceutically acceptable salt) is typically substantially pure, for example, has a purity of at least 90% (e.g., at least 90%, at least 95%, at least 98%, at least 99%) by HPLC and/or by weight. The term substantially pure, when referring to the compound of Formula III-Alpha or a salt thereof can refer to a substantially pure mixture of oligomers (e.g., n is 2-5), which means that it is substantially free of impurities that are not the specified mixture of oligomers. In some embodiments, the term substantially pure, when referring to the compound of Formula III-Alpha or a salt thereof can also refer to a substantially pure specific oligomer (e.g., n is 2, 3, 4, or 5), which means that it is substantially free of impurities that are not the specific oligomer.

In some embodiments, the compound of Formula III-Alpha (e.g., Formula III-L-Alpha or III-D-Alpha) can be a substantially pure specific oligomer, e.g., with a purity of at least 90% (e.g., at least 90%, at least 95%, at least 98%, at least 99%) by HPLC and/or by weight. For example, the compound of Formula III-Alpha (e.g., Formula III-L-Alpha or III-D-Alpha) can be a substantially pure tetraglutamate (n is 2), a substantially pure pentaglutamate (n is 3), a substantially pure hexaglutamate (n is 4), a substantially pure heptaglutamate (n is 5), etc. In some specific embodiments, the compound of Formula III-Alpha (e.g., Formula III-L-Alpha or III-D-Alpha) can be a substantially pure hexaglutamate, wherein n in Formula III-Alpha is 4. For example, in such embodiments, the compound of Formula III-Alpha (e.g., Formula III-L-Alpha or III-D-Alpha) can be substantially free (e.g., less than 10%, less than 5%, less than 2%, less than 1%, less than 0.5%, or less than 0.1%) of another compound of Formula III-Alpha wherein n is not 4.

Compounds of Formula III-Alpha with the recited purity profile can be prepared by controlling the purity of the corresponding polyglutamate of Formula I-Alpha used for the amide coupling reaction with Z—COOH or an activated form thereof and/or the protected polyglutamated antifolate of Formula II-Alpha.

In some embodiments, compounds of Formula III-Alpha (e.g., Formula III-L-Alpha or III-D-Alpha) can also be prepared using solid phase chemistry. For example, an initial glutamyl residue can be bonded to a Wang resin (or other suitable resins or solid supports) and additional glutamyl residues are added serially via solid phase peptide synthesis using F-moc chemistry. After the final glutamyl residue is added the Antifolate precursor (e.g., pemetrexed precursor) is coupled to the peptide and the molecule is cleaved from the resin. In some embodiments, compounds of Formula III-Alpha (e.g., Formula III-L-Alpha or III-D-Alpha) are not prepared using solid phase chemistry.

### Formula IV-Alpha and Preparation

In some embodiments, the present disclosure also provides an alkali salt of Formula IV-Alpha (e.g., described herein). Without wishing to be bound by theories, it is believed that the use of alkali salt can be beneficial in various ways. The alkali salt is typically more water soluble than the corresponding free acid form or an acid addition salt or other salts. Thus, in some embodiments, the alkali salt of Formula IV-Alpha can be more suitable for preparing a pharmaceutical composition where good aqueous solubility is beneficial, such as preparing an aqueous solution formulation, or preparing a liposomal composition described herein. Also, with a further processing step, the alkali salt can be prepared in a higher purity than the free acid form or the acid addition salt. For example, in some embodiments, the alkali salt can be prepared from a substantially pure acid addition salt of Formula III-Alpha, and the alkali salt resulted can be further purified, such as through crystallization, to form a solid form of the alkali salt, which is typically substantially pure. This process can greatly enhance large-scale manufacturing and can lead to a high purity active pharmaceutical ingredient useful for preparing various pharmaceutical compositions, e.g., as described herein.

In some embodiments, the alkali salt of Formula IV-Alpha is in a solid form. For example, in some embodiments, the alkali salt of Formula IV-Alpha can be an anhydrous form, a hydrate, a solvate, or a mixture thereof. In some embodiments, the alkali salt of Formula IV-Alpha is a solvate, such as an ethanol solvate.

The alkali salt can be prepared by converting a compound of Formula III-Alpha, or a salt thereof, e.g., a substantially pure acid addition salt of Formula III-Alpha, into the alkali salt of Formula IV-Alpha by treating with a suitable base, such as NaOH. In some embodiments, a substantially pure compound of Formula III-Alpha or an acid addition salt thereof in a solid form can be used for preparation of the salt of Formula IV-Alpha. In some embodiments, the alkali salt of Formula IV-Alpha can be prepared by a method comprising: adding a substantially pure compound of Formula III-Alpha or a salt thereof to an aqueous alkali base solution, e.g., NaOH solution, to form the alkali salt in water; and adding the alkali salt in water to a solvent (e.g., ethanol) to precipitate the alkali salt. In some embodiments, the precipitated alkali salt can be further dissolved in water, and the aqueous solution can be added to a solvent (e.g., ethanol) to precipitate the alkali salt. In some embodiments, the process of dissolving and precipitating in a solvent can be repeated to achieve a desired purity. In some embodiments, the solvent for precipitating is an alcoholic solvent, such as a C₁₋₄ alcohol (e.g., ethanol). In some embodiments, the substantially pure compound of Formula III-Alpha or an acid addition salt thereof (e.g., a TFA salt) in a solid form can be first dissolved, partially dissolved, suspended, or otherwise mixed in a suitable solvent, which can be for example, water, a C₁₋₄ alcohol (e.g., ethanol), or a mixture thereof, and the suitable base (e.g., NaOH) can be added concurrently or sequentially in any order to the solvent, which can convert the acid addition salt of Formula III-Alpha into an alkali salt of Formula IV-Alpha.

In some embodiments, the presend disclosure also provide a method of isolating, purifying, and/or crystallizing the alkali salt of Formula IV-Alpha to provide a substantially pure salt of Formula IV-Alpha. In some embodiments, the crystallizing can comprise dissolving the alkali salt of Formula IV-Alpha in water, and then adding the aqueous solution into a solvent to precipitate the alkali salt. In some embodiments, the solvent is an alcoholic solvent, such as a C₁₋₄ alcohol (e.g., methanol, ethanol, isopropanol, etc.). In some embodiments, the solvent is ethanol. Other isolation, purification, and crystallization techniques are known in the art and can be used for the methods herein. Typically, the precipitated alkali salt of Formula IV-Alpha is substantially pure. In some embodiments, the substantially pure salt of Formula IV-Alpha is a hydrate or a solvate. In some embodiments, the substantially pure salt of Formula IV-Alpha is in a crystalline form, an amorphous form, or a mixture thereof.

Typically, the conversion of the compound of Formula III-Alpha, or a salt thereof, into the alkali salt of Formula IV-Alpha can be carried out with no or minimized racemization of chiral centers. In some embodiments, the alkali salt of Formula IV-Alpha are prepared in a stereoisomerically pure or substantially pure form in a large scale. For example, in some embodiments, the present disclosure provides the alkali salt of Formula IV-Alpha in a stereoisomerically pure or substantially pure form in a batch size over 10 grams (such as a batch size of about 100 gram or more, about 1 kg or more, about 5 kg or more, about 10 kg or more, etc.). In some embodiments, the alkali salt of Formula IV-Alpha can exist predominantly in one enantiomeric form, which can be free or substantially free (e.g., containing less than 5%, less than 2%, less than 1%, less than 0.5%, or less than 0.1%) of the other enantiomeric form. In some embodiments, the alkali salt of Formula IV-Alpha can also exist predominantly as one diastereomer, which can be free or substantially free (e.g., containing less than 5%, less than 2%, less than 1%, less than 0.5%, or less than 0.1%) of any other diastereomer(s).

In some embodiments, each of the glutamate units in Formula IV-Alpha is in an L-form, and the compound of Formula IV-Alpha is a compound of Formula IV-L-Alpha:

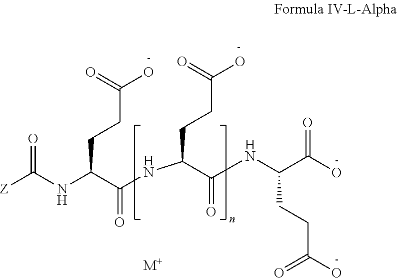

wherein Z, M⁺, and n are defined herein. In some embodiments, the alkali salt of Formula IV-L-Alpha can be stereoisomerically pure or substantially pure. For example, in some embodiments, the alkali salt of Formula IV-L-Alpha can be free or substantially free (e.g., containing less than 5%, less than 2%, less than 1%, less than 0.5%, or less than 0.1%) of the other enantiomeric form. In some embodiments, the alkali salt of Formula IV-L-Alpha can also be free or substantially free (e.g., containing less than 5%, less than 2%, less than 1%, less than 0.5%, or less than 0.1%) of any other diastereomer(s). However, in some embodiments, the alkali salt of Formula IV-L-Alpha can also exist in a racemic mixture or in a stereoisomeric mixture.

In some embodiments, each of the glutamate units in Formula IV-Alpha is in a D-form, and the compound of Formula IV-Alpha is a compound of Formula IV-D-Alpha:

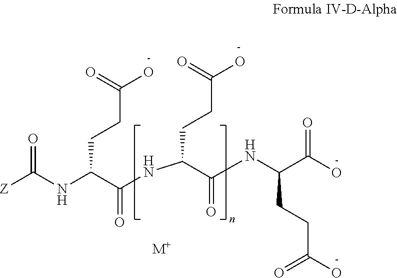

wherein Z, M⁺, and n are defined herein. In some embodiments, the alkali salt of Formula IV-D-Alpha can be stereoisomerically pure or substantially pure. For example, in some embodiments, the alkali salt of Formula IV-D-Alpha can be free or substantially free (e.g., containing less than 5%, less than 2%, less than 1%, less than 0.5%, or less than 0.1%) of the other enantiomeric form. In some embodiments, the alkali salt of Formula IV-D-Alpha can also be free or substantially free (e.g., containing less than 5%, less than 2%, less than 1%, less than 0.5%, or less than 0.1%) of any other diastereomer(s). However, in some embodiments, the alkali salt of Formula IV-D-Alpha can also exist in a racemic mixture or in a stereoisomeric mixture.

In some specific embodiments, Z in Formula IV-Alpha (e.g., Formula IV-L-Alpha or IV-D-Alpha) can be a residue of an antifolate selected from methotrexate (MTX), pemetrexed (PMX), lometrexol (LTX), AG2034, raltitrexed (RTX), pralatrexate, GW1843, aminopterin, LY309887 and LY222306.

In some specific embodiments, Z in Formula IV-Alpha (e.g., Formula IV-L-Alpha or IV-D-Alpha) can be a residue of pemetrexed having the following formula:

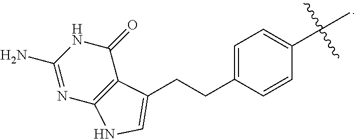 [SMILES: *c1ccc(CCc2c[nH]c3nc(N)[nH]c(=O)c23)cc1]

In some specific embodiments, Z in Formula IV-Alpha (e.g., Formula IV-L-Alpha or IV-D-Alpha) can be a residue having the following formula:

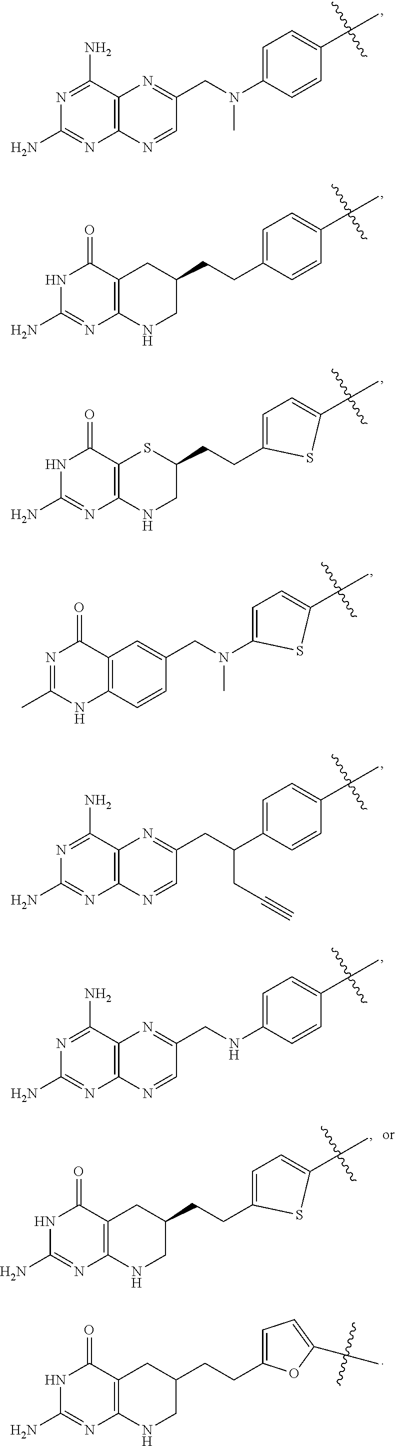

In some specific embodiments, n in Formula IV-Alpha (e.g., Formula IV-L-Alpha or IV-D-Alpha) can be an integer of 0-20, for example, 2-20, 2-15, 2-10, 2-6, 2-5, or more than 5. In some embodiments, M⁺ is Na⁺. In some embodiments, n is 4, M⁺ is Na⁺, and the alkali salt of Formula IV is a hepta-sodium salt.

The compound of Formula IV-Alpha (e.g., Formula IV-L-Alpha or IV-D-Alpha) is typically substantially pure, for example, has a purity of at least 90% (e.g., at least 90%, at least 95%, at least 98%, at least 99%) by HPLC and/or by weight. The term substantially pure, when referring to the compound of Formula IV-Alpha can refer to a substantially pure mixture of oligomers (e.g., n is 2-5), which means that it is substantially free of impurities that are not the specified mixture of oligomers. In some embodiments, the term substantially pure, when referring to the compound of Formula IV-Alpha can also refer to a substantially pure specific oligomer (e.g., n is 2, 3, 4, or 5), which means that it is substantially free of impurities that are not the specific oligomer.

In some embodiments, the compound of Formula IV-Alpha (e.g., Formula IV-L-Alpha or IV-D-Alpha) can be a substantially pure specific oligomer, e.g., with a purity of at least 90% (e.g., at least 90%, at least 95%, at least 98%, at least 99%) by HPLC and/or by weight. For example, the compound of Formula IV-Alpha (e.g., Formula IV-L-Alpha or IV-D-Alpha) can be a substantially pure tetraglutamate (n is 2), a substantially pure pentaglutamate (n is 3), a substantially pure hexaglutamate (n is 4), a substantially pure heptaglutamate (n is 5), etc. In some specific embodiments, the compound of Formula IV-Alpha (e.g., Formula IV-L-Alpha or IV-D-Alpha) can be a substantially pure hexaglutamate, wherein n in Formula IV-Alpha can be 4. For example, in such embodiments, the compound of Formula IV-Alpha (e.g., Formula IV-L-Alpha or IV-D-Alpha) can be substantially free (e.g., less than 10%, less than 5%, less than 2%, less than 1%, less than 0.5%, or less than 0.1%) of another compound of Formula IV-Alpha wherein n is not 4.

Compounds of Formula IV-Alpha with the recited purity profile can be prepared by controlling the purity of the corresponding polyglutamate of Formula I-Alpha used for the amide coupling reaction with Z—COOH or an activated form thereof, the protected polyglutamate of Formula II-Alpha, and/or the compound of Formula III-Alpha or salts thereof. Exemplary procedures are described in the Examples section.

### Exemplary Specific Alpha-Polyglutamated Compounds

In some embodiments, the present disclosure also provides exemplary specific compounds of Formula III-1-Alpha, or a pharmaceutically acceptable salt thereof:

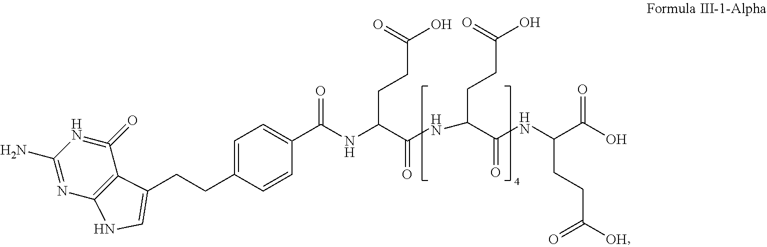

wherein each glutamate unit is independently in an L-form or D-form. In some embodiments, the compound of Formula III-1-Alpha can be in a form of a pharmaceutically acceptable acid addition salt, such as an HCl salt. In some embodiments, the compound of Formula III-1-Alpha can be in a form of a pharmaceutically acceptable base addition salt, such as a sodium salt, e.g., monosodium, disodium, trisodium, tetrasodium, pentasodium, hexasodium, or hepta-sodium salt. In some embodiments, the present disclosure also provides exemplary specific compounds Formula III-1-L-Alpha, Formula III-1-D-Alpha, a mixture thereof, or a pharmaceutically acceptable salt thereof:

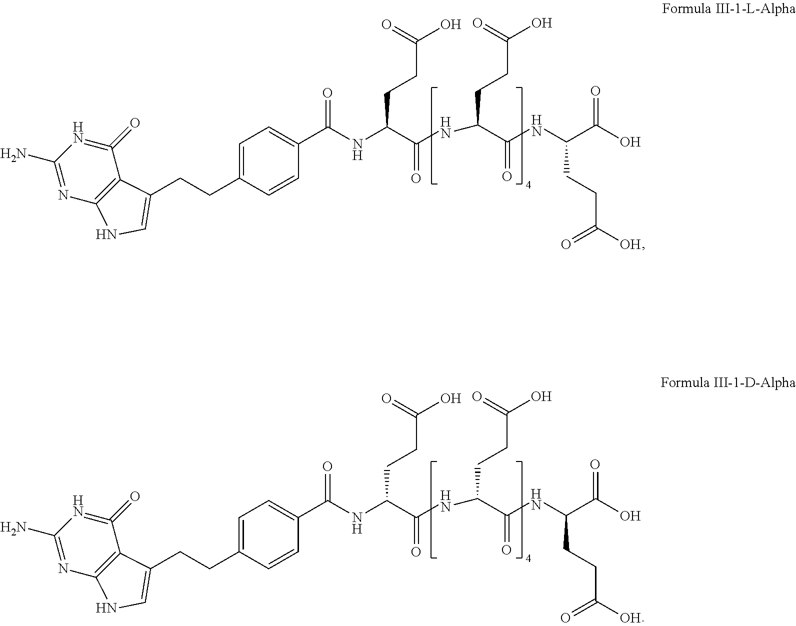

In some embodiments, the compound of Formula III-1-L-Alpha, or a pharmaceutically acceptable salt thereof (e.g., described herein) can be substantially pure, for example, it can be substantially free (e.g., less than 5%, less than 2%, less than 1%, less than 0.5%, or less than 0.1%) of a compound of Formula III-2-Alpha, or a pharmaceutically acceptable salt thereof:

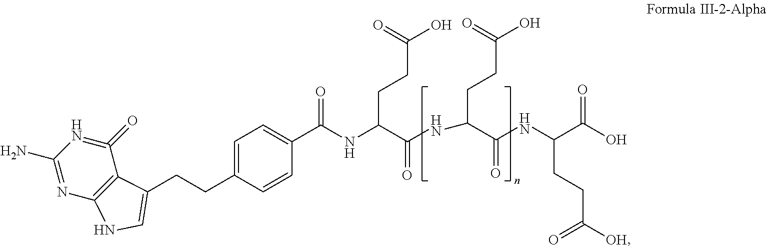

wherein n in Formula III-2-Alpha is an integer that is not 4, or n is 4 and at least one of the glutamate units is not in an L-form. In some embodiments, the compound of Formula III-1-L-Alpha, or a pharmaceutically acceptable salt thereof can be characterized as having a purity by HPLC of at least 90% and/or by weight of at least 90%, e.g., a purity by HPLC of at least 90%, at least 95%, at least 98%, or at least 99%. In some embodiments, the compound of Formula III-1-L-Alpha, or a pharmaceutically acceptable salt thereof is in a solid form, such as a crystalline form, an amorphous form, or a mixture thereof. In some embodiments, the compound of Formula III-1-L-Alpha, or a pharmaceutically acceptable salt thereof can be a hydrate or a solvate, which can be in a crystalline form, an amorphous form, or a mixture thereof. In some embodiments, a pharmaceutical batch of the substantially pure compound of Formula III-1-L-Alpha, or a pharmaceutically acceptable salt thereof is provided. In some embodiments, the pharmaceutical batch is at least 10 grams, such as a batch size of about 100 gram or more, about 1 kg or more, about 5 kg or more, about 10 kg or more, etc. In some embodiments, the pharmaceutical batch is in a solid form, such as a crystalline form, an amorphous form, or a mixture thereof. In some embodiments, the compound of Formula III-1-L-Alpha can be in a form of a pharmaceutically acceptable acid addition salt, such as an HCl salt. In some embodiments, the compound of Formula III-1-L-Alpha can be in a form of a pharmaceutically acceptable base addition salt, such as a sodium salt, e.g., monosodium, disodium, trisodium, tetrasodium, pentasodium, hexasodium, or hepta-sodium salt.

In some embodiments, the compound of Formula III-1-D-Alpha, or a pharmaceutically acceptable salt thereof (e.g., described herein) can be substantially pure, for example, it can be substantially free (e.g., less than 5%, less than 2%, less than 1%, less than 0.5%, or less than 0.1%) of a compound of Formula III-2-Alpha, or a pharmaceutically acceptable salt thereof:

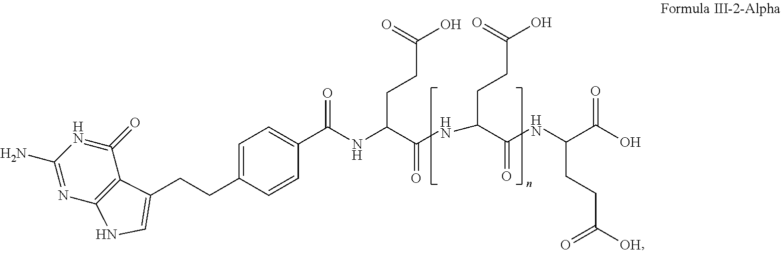

wherein n in Formula III-2-Alpha is an integer that is not 4, or n is 4 and at least one of the glutamate units is not in a D-form. In some embodiments, the compound of Formula III-1-D-Alpha, or a pharmaceutically acceptable salt thereof can be characterized as having a purity by HPLC of at least 90% and/or by weight of at least 90%, e.g., a purity by HPLC of at least 90%, at least 95%, at least 98%, or at least 99%. In some embodiments, the compound of Formula III-1-D-Alpha, or a pharmaceutically acceptable salt thereof is in a solid form, such as a crystalline form, an amorphous form, or a mixture thereof. In some embodiments, the compound of Formula III-1-D-Alpha, or a pharmaceutically acceptable salt thereof can be a hydrate or a solvate, which can be in a crystalline form, an amorphous form, or a mixture thereof. In some embodiments, a pharmaceutical batch of the substantially pure compound of Formula III-1-D-Alpha, or a pharmaceutically acceptable salt thereof is provided. In some embodiments, the pharmaceutical batch is at least 10 grams, such as a batch size of about 100 gram or more, about 1 kg or more, about 5 kg or more, about 10 kg or more, etc. In some embodiments, the pharmaceutical batch is in a solid form, such as a crystalline form, an amorphous form, or a mixture thereof. In some embodiments, the compound of Formula III-1-D-Alpha can be in a form of a pharmaceutically acceptable acid addition salt, such as an HCl salt. In some embodiments, the compound of Formula III-1-D-Alpha can be in a form of a pharmaceutically acceptable base addition salt, such as a sodium salt, e.g., monosodium, disodium, trisodium, tetrasodium, pentasodium, hexasodium, or hepta-sodium salt.

In some embodiments, the present disclosure also provides a hepta-sodium salt of Formula IV-1-Alpha:

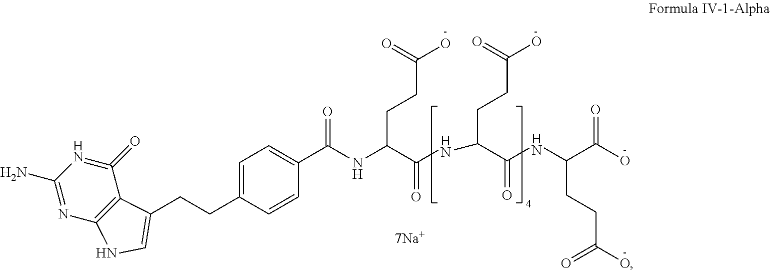

wherein each glutamate unit is independently in an L-form or D-form.

In some embodiments, the present disclosure also provides Formula IV-1-L-Alpha, Formula IV-1-D-Alpha, or a mixture thereof:

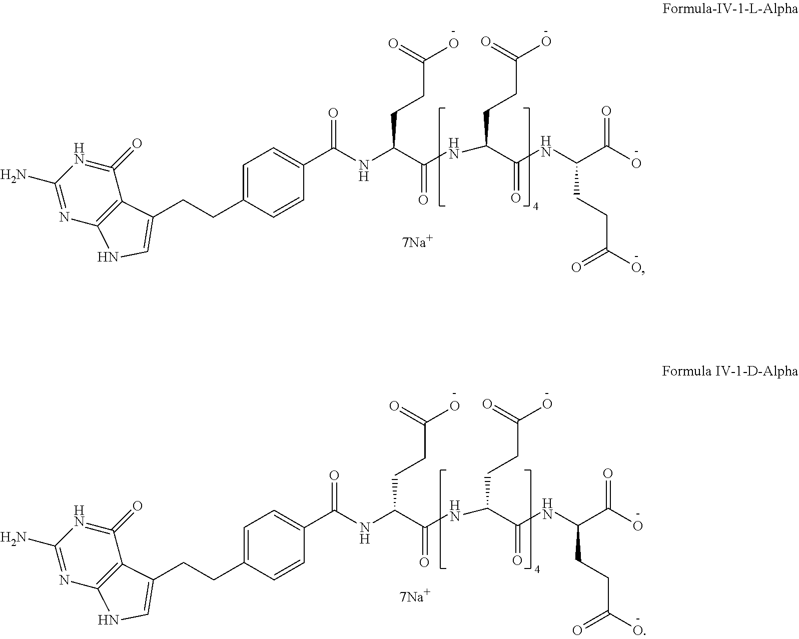

In some embodiments, the compound of Formula IV-1-L-Alpha can be substantially pure, for example, it can be characterized as having a purity by HPLC of at least 90% and/or by weight of at least 90%, e.g., a purity by HPLC of at least 90%, at least 95%, at least 98%, or at least 99%. In some embodiments, the compound of Formula IV-1-L-Alpha can be free or substantially free (e.g., containing less than 5%, less than 2%, less than 1%, less than 0.5%, or less than 0.1%) of the other enantiomeric form. In some embodiments, the compound of Formula IV-1-L-Alpha can also be free or substantially free (e.g., containing less than 5%, less than 2%, less than 1%, less than 0.5%, or less than 0.1%) of any other diastereomer(s). In some embodiments, the compound of Formula IV-1-L-Alpha can also be free or substantially free (e.g., containing less than 5%, less than 2%, less than 1%, less than 0.5%, or less than 0.1%) of a non-sodium salt of Formula III-1-L-Alpha. In some embodiments, the compound of Formula IV-1-L-Alpha is in a solid form, such as a crystalline form, an amorphous form, or a mixture thereof. In some embodiments, the compound of Formula IV-1-L-Alpha can be a hydrate or a solvate, which can be in a crystalline form, an amorphous form, or a mixture thereof. In some embodiments, a pharmaceutical batch of the substantially pure compound of Formula IV-1-L-Alpha is provided. In some embodiments, the pharmaceutical batch is at least 10 grams, such as a batch size of about 100 gram or more, about 1 kg or more, about 5 kg or more, about 10 kg or more, etc. In some embodiments, the pharmaceutical batch is in a solid form, such as a crystalline form, an amorphous form, or a mixture thereof.

In some embodiments, the compound of Formula IV-1-D-Alpha can be substantially pure, for example, it can be characterized as having a purity by HPLC of at least 90% and/or by weight of at least 90%, e.g., a purity by HPLC of at least 90%, at least 95%, at least 98%, or at least 99%. In some embodiments, the compound of Formula IV-1-D-Alpha can be free or substantially free (e.g., containing less than 5%, less than 2%, less than 1%, less than 0.5%, or less than 0.1%) of the other enantiomeric form. In some embodiments, the compound of Formula IV-1-D-Alpha can also be free or substantially free (e.g., containing less than 5%, less than 2%, less than 1%, less than 0.5%, or less than 0.1%) of any other diastereomer(s). In some embodiments, the compound of Formula IV-1-D-Alpha can also be free or substantially free (e.g., containing less than 5%, less than 2%, less than 1%, less than 0.5%, or less than 0.1%) of a non-sodium salt of Formula III-1-D-Alpha. In some embodiments, the compound of Formula IV-1-D-Alpha is in a solid form, such as a crystalline form, an amorphous form, or a mixture thereof. In some embodiments, the compound of Formula IV-1-D-Alpha can be a hydrate or a solvate, which can be in a crystalline form, an amorphous form, or a mixture thereof. In some embodiments, a pharmaceutical batch of the substantially pure compound of Formula IV-1-D-Alpha is provided. In some embodiments, the pharmaceutical batch is at least 10 grams, such as a batch size of about 100 gram or more, about 1 kg or more, about 5 kg or more, about 10 kg or more, etc. In some embodiments, the pharmaceutical batch is in a solid form, such as a crystalline form, an amorphous form, or a mixture thereof.

In some embodiments, the present disclosure also provides specific synthetic intermediates, such as compounds 1, 2, 3, 4, 5, and 6, and products of Compound 200, 210, 220, 230 as shown in the Examples section. In some embodiments, each of the intermediates and compounds 200, 210, 220 and 230 is substantially pure, e.g., with a HPLC purity and/or purity by weight greater than 90% (e.g., greater than 95%, greater than 98%, or greater than 99%).

In some embodiments, the present disclosure also provides a synthetic method of Compound III-1-L-Alpha comprising a process substantially according to the scheme shown below:

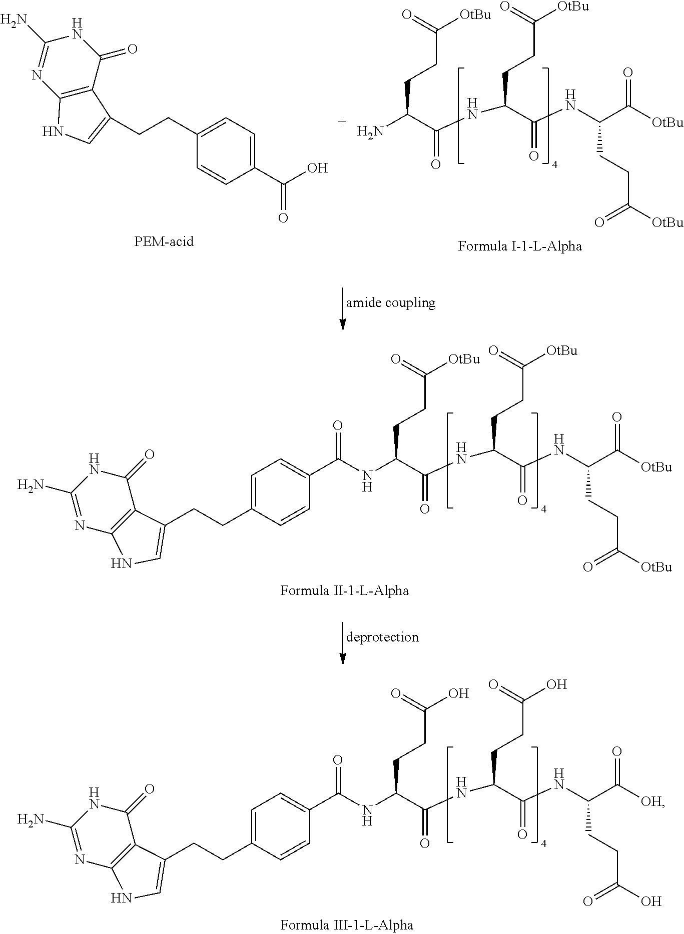

wherein PEM-Acid or an activated form thereof is coupled with a compound of Formula I-1-L-Alpha to provide a protected polyglutamate of Formula II-1-L-Alpha, which can be followed by a deprotection step to provide the compound of Formula III-1-L-Alpha. In some embodiments, the method further comprises converting the compound of Formula III-1-L-Alpha or a salt thereof into the alkali salt of Formula IV-1-L-Alpha. In some embodiments, each of the compounds or salts of Formula I-1-L-Alpha, II-1-L-Alpha, III-1-L-Alpha and IV-1-L-Alpha can be substantially pure, e.g., a HPLC purity and/or purity by weight of greater than 90% (e.g., greater than 95%, greater than 98%, or greater than 99%).

In some embodiments, the present disclosure also provides a synthetic method of preparing an alkali salt of Compound IV-1-L-Alpha from a compound of Formula III-1-L-Alpha or a salt thereof, e.g., a substantially pure compound of Formula III-1-L-Alpha or a salt thereof. In some embodiments, the compound of Formula III-1-L-Alpha, e.g., substantially pure compound of Formula III-1-L-Alpha, or an acid addition salt thereof in a solid form can be used for preparation of the salt of Formula IV-1-L-Alpha. In some embodiments, the alkali salt of Formula IV-1-L-Alpha can be prepared by a method comprising: adding a substantially pure compound of Formula III-1-L-Alpha or a salt thereof to an aqueous alkali base solution, e.g., NaOH solution, to form the alkali salt in water; and adding the alkali salt in water to a solvent (e.g., ethanol) to precipitate the alkali salt. In some embodiments, the precipitated alkali salt can be further dissolved in water, and the aqueous solution can be added to a solvent (e.g., ethanol) to precipitate the alkali salt. In some embodiments, the process of dissolving and precipitating in a solvent can be repeated to achieve a desired purity. In some embodiments, the solvent for precipitating is an alcoholic solvent, such as a C₁₋₄ alcohol (e.g., ethanol). In some embodiments, the substantially pure compound of Formula III-1-L-Alpha or an acid addition salt thereof (e.g., a TFA salt) in a solid form can be first dissolved, partially dissolved, suspended, or otherwise mixed in a suitable solvent, which can be for example, water, a C₁₋₄ alcohol (e.g., ethanol), or a mixture thereof, and the suitable base (e.g., NaOH) can be added concurrently or sequentially in any order to the solvent, which can convert the acid addition salt of Formula III-1-L-Alpha into an alkali salt of Formula IV-1-L-Alpha.

In some embodiments, the present disclosure also provides a method of isolating, purifying, and/or crystallizing the alkali salt of Formula IV-1-L-Alpha to provide a substantially pure salt of Formula IV-1-L-Alpha. In some embodiments, the crystallizing can comprise dissolving the alkali salt of Formula IV-1-L-Alpha into water, and then adding the aqueous solution into a solvent to precipitate the alkali salt. In some embodiments, the solvent is an alcoholic solvent, such as a C₁₋₄ alcohol (e.g., methanol, ethanol, isopropanol, etc.). In some embodiments, the solvent is ethanol. Other isolation, purification, and crystallization techniques are known in the art and can be used for the methods herein. Typically, the precipitated alkali salt of Formula IV-1-L-Alpha is substantially pure, e.g., a HPLC purity and/or purity by weight of greater than 90% (e.g., greater than 95%, greater than 98%, or greater than 99%). In some embodiments, the substantially pure salt of Formula IV-1-L-Alpha is a hydrate or a solvate. In some embodiments, the substantially pure salt of Formula IV-1-L-Alpha is in a crystalline form, an amorphous form, or a mixture thereof.

In some embodiments, the present disclosure also provides a synthetic method of Compound III-1-D-Alpha comprising a process substantially according to the scheme shown below:

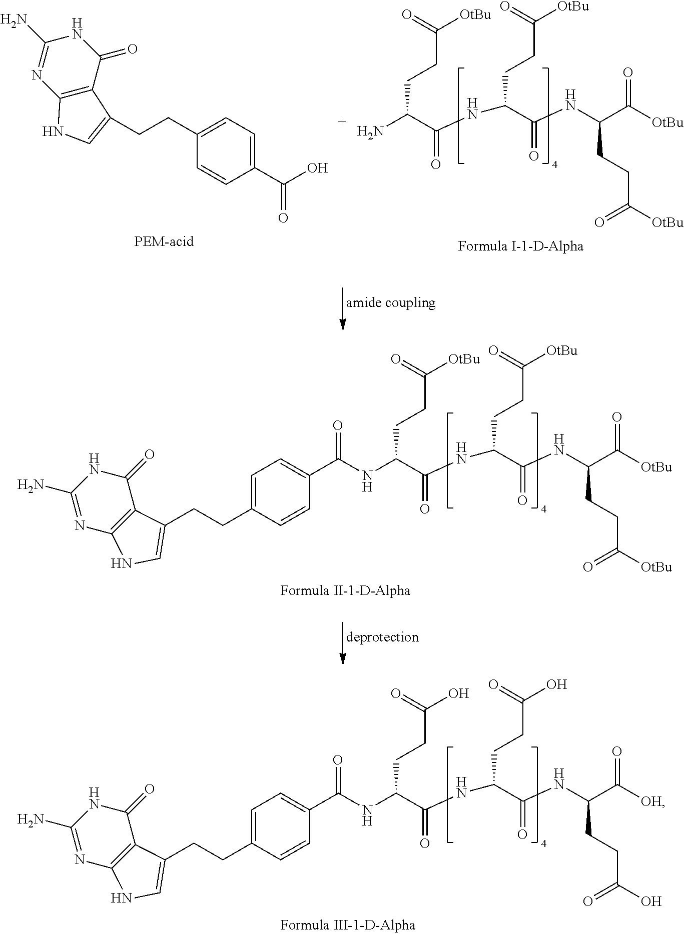

wherein PEM-Acid or an activated form thereof is coupled with a compound of Formula I-1-D-Alpha to provide a protected polyglutamate of Formula II-1-D-Alpha, which can be followed by a deprotection step to provide the compound of Formula III-1-D-Alpha. In some embodiments, the method further comprises converting the compound of Formula III-1-D-Alpha or a salt thereof into the alkali salt of Formula IV-1-D-Alpha. In some embodiments, each of the compounds or salts of Formula I-1-D-Alpha, II-1-D-Alpha, III-1-D-Alpha and IV-1-D-Alpha can be substantially pure, e.g., a HPLC purity and/or purity by weight of greater than 90% (e.g., greater than 95%, greater than 98%, or greater than 99%).

In some embodiments, the present disclosure also provides a synthetic method of preparing an alkali salt of Compound IV-1-D-Alpha from a compound of Formula III-1-D-Alpha or a salt thereof, e.g., a substantially pure compound of Formula III-1-D-Alpha or a salt thereof. In some embodiments, the compound of Formula III-1-D-Alpha, e.g., substantially pure compound of Formula III-1-D-Alpha, or an acid addition salt thereof in a solid form can be used for preparation of the salt of Formula IV-1-D-Alpha. In some embodiments, the alkali salt of Formula IV-1-D-Alpha can be prepared by a method comprising: adding a substantially pure compound of Formula III-1-D-Alpha or a salt thereof to an aqueous alkali base solution, e.g., NaOH solution, to form the alkali salt in water; and adding the alkali salt in water to a solvent (e.g., ethanol) to precipitate the alkali salt. In some embodiments, the precipitated alkali salt can be further dissolved in water, and the aqueous solution can be added to a solvent (e.g., ethanol) to precipitate the alkali salt. In some embodiments, the process of dissolving and precipitating in a solvent can be repeated to achieve a desired purity. In some embodiments, the solvent for precipitating is an alcoholic solvent, such as a C₁₋₄ alcohol (e.g., ethanol). In some embodiments, the substantially pure compound of Formula III-1-D-Alpha or an acid addition salt thereof (e.g., a TFA salt) in a solid form can be first dissolved, partially dissolved, suspended, or otherwise mixed in a suitable solvent, which can be for example, water, a C₁₋₄ alcohol (e.g., ethanol), or a mixture thereof, and the suitable base (e.g., NaOH) can be added concurrently or sequentially in any order to the solvent, which can convert the acid addition salt of Formula III-1-D-Alpha into an alkali salt of Formula IV-1-D-Alpha.

In some embodiments, the present disclosure also provides a method of isolating, purifying, and/or crystallizing the alkali salt of Formula IV-1-D-Alpha to provide a substantially pure salt of Formula IV-1-D-Alpha. In some embodiments, the crystallizing can comprise dissolving the alkali salt of Formula IV-1-D-Alpha into water, and then adding the aqueous solution into a solvent to precipitate the alkali salt. In some embodiments, the solvent is an alcoholic solvent, such as a C₁₋₄ alcohol (e.g., methanol, ethanol, isopropanol, etc.). In some embodiments, the solvent is ethanol. Other isolation, purification, and crystallization techniques are known in the art and can be used for the methods herein. Typically, the precipitated alkali salt of Formula IV-1-D-Alpha is substantially pure, e.g., a HPLC purity and/or purity by weight of greater than 90% (e.g., greater than 95%, greater than 98%, or greater than 99%). In some embodiments, the substantially pure salt of Formula IV-1-D-Alpha is a hydrate or a solvate. In some embodiments, the substantially pure salt of Formula IV-1-D-Alpha is in a crystalline form, an amorphous form, or a mixture thereof.

As described herein, the substantially pure compounds herein can exist in solid forms, such as a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha). In some embodiments, a pharmaceutical composition in a non-solid form can be prepared from dissolving, suspending, or otherwise mixing a solid form of the substantially pure compounds herein with other ingredients.

### Compositions and Methods of Using

As explained in detail in WO 2018/031967, WO 2018/031968, WO 2018/031979, WO 2018/031980, WO 2019/094648, PCT/US2019/016989, and PCT/US2019/017004, the content of each of which is herein incorporated by reference in its entirety, PANTIFOL complexes and compositions are useful for treating or preventing various diseases, including but are not limited to hyperproliferative diseases such as cancer, disorders of the immune system including inflammation and autoimmune disease such as rheumatoid arthritis, and infectious diseases such as HIV, malaria, and schistomiasis.

Accordingly, in various embodiments, the present disclosure also provides complexes, pharmaceutical compositions (e.g., liposomal compositions), and methods of treatments directed to one or more PANTIFOL such as αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

### Polyglutamated Antifolate Complexes

In some embodiments, the PANTIFOL such as αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof, can also form complexes with other compositions including therapeutic agents, for example, cytotoxic compounds such as platinum-based compounds, as described in PCT/US2019/017004. Accordingly, in some embodiments, the disclosure also provides a PANTIFOL/complex comprising the PANTIFOL (e.g., αPANTIFOL and/or γPANTIFOL) of the present disclosure (e.g., described herein) and a therapeutic agent or a salt or acid thereof. In some embodiments, the therapeutic agent is a cytotoxic compound such as a chemotherapeutic agent. In further embodiments, the PANTIFOL/complex contains a platinum-based drug such as platinum-based chemotherapeutic agent (e.g., carboplatin and cisplatin). In other embodiments, the PANTIFOL/complex contains a taxane-based chemotherapeutic agent (e.g., carboplatin and cisplatin). In other embodiments, the PANTIFOL/complex contains a cyclodextrin. In further embodiments, the PANTIFOL/complex is encapsulated in a liposome.

In additional embodiments, the molar ratio of PANTIFOL/therapeutic agent in the complex is in the range 1-10:1. In some embodiments, the molar ratio of PANTIFOL/therapeutic agent in the complex is 1:1, 2:1, 3:1, 4:1, 5:1, 6:1, 7:1, or 10:1. In some embodiments, the PANTIFOL/therapeutic agent complex is encapsulated in a liposome (e.g., as described herein or otherwise known in the art). In some embodiments, the molar ratio of PANTIFOL/therapeutic agent in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of PANTIFOL/therapeutic agent in the complex is: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In some embodiments, the PANTIFOL/therapeutic agent complex is encapsulated in a liposome (e.g., as described herein or otherwise known in the art).

In an alternative embodiment, the PANTIFOL complex comprises αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof, and cyclodextrin. In some embodiments, the molar ratio of PANTIFOL (e.g., PANTIFOL salt)/cyclodextrin in the complex is in the range 1-20:1, or any range therein between. In some embodiments, the molar ratio of PANTIFOL/cyclodextrin in the complex is in the range 1-10:1, or any range therein between. In further embodiments, the molar ratio of PANTIFOL/cyclodextrin in the complex is in the range 2-8:1, or any range therein between. In some embodiments, the molar ratio of PANTIFOL/cyclodextrin in the complex is: 1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the molar ratio of PANTIFOL/cyclodextrin in the complex is: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In other embodiments, the molar ratio of PANTIFOL/cyclodextrin in the complex is in the range 1:1-20, 1:1-10, or 1:2-8, or any range therein between. In some embodiments, the molar ratio of PANTIFOL/cyclodextrin in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/cyclodextrin in the complex is: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the PANTIFOL/cyclodextrin complex is encapsulated in a liposome (e.g., as described herein or otherwise known in the art).

In some embodiments, the disclosure provides a composition comprising a PANTIFOL/platinum-based chemotherapeutic agent complex. In some embodiments, the complex comprises a PANTIFOL of the present disclosure, such as αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the platinum-based chemotherapeutic agent is selected from: cisplatin, carboplatin, and oxaliplatin, or a salt or acid thereof. In other embodiments, the PANTIFOL/platinum-based chemotherapeutic agent complex comprises an analog of a cisplatin, carboplatin, oxaliplatin, or a salt or acid thereof. In some embodiments, the molar ratio of PANTIFOL/platinum-based agent in the complex is in the range 1-20:1, or any range therein between. In some embodiments, the molar ratio of PANTIFOL/platinum-based agent in the complex is in the range 1-10:1, or any range therein between. In further embodiments, the molar ratio of PANTIFOL/platinum-based agent in the complex is in the range 2-8:1, or any range therein between. In some embodiments, the molar ratio of PANTIFOL/platinum-based agent in the complex is 11:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the molar ratio of PANTIFOL/platinum-based agent in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In other embodiments, the molar ratio of PANTIFOL/platinum-based chemotherapeutic agent in the complex is in the range 1:1-20, 1:1-10, or 1:2-8, or any range therein between. In some embodiments, the molar ratio of PANTIFOL/platinum-based agent in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/platinum-based agent in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In additional embodiments, the PANTIFOL/platinum-based agent complex is encapsulated in a liposome.

In additional embodiments, the PANTIFOL/platinum-based chemotherapeutic agent complex comprises an analog of a cisplatin, carboplatin, oxaliplatin, or a salt or acid thereof. In some embodiments, the complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the molar ratio of PANTIFOL/platinum-based analog in the complex is in the range 1-20:1, or any range therein between. In some embodiments, the molar ratio of PANTIFOL/platinum-based analog in the complex is in the range 1-10:1, or any range therein between. In further embodiments, the molar ratio of PANTIFOL/platinum-based agent in the complex is in the range 2-8:1, or any range therein between. In some embodiments, the molar ratio of PANTIFOL/platinum-based analog in the complex is 11:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the molar ratio of PANTIFOL/platinum-based analog in the complex is 11:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In some embodiments, the molar ratio of PANTIFOL/platinum-based agent in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/platinum-based agent in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In additional embodiments, the PANTIFOL/platinum-based analog complex is encapsulated in a liposome.

In further embodiments, the disclosure provides a complex containing PANTIFOL and cisplatin or a salt or acid thereof. In some embodiments, the complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the molar ratio of PANTIFOL/cisplatin (or cisplatin salt or acid) in the complex is in the range 1-20:1, or any range therein between. In some embodiments, the molar ratio of PANTIFOL/cisplatin (or cisplatin salt or acid) in the complex is in the range 1-10:1, or any range therein between. In further embodiments, the molar ratio of PANTIFOL/cisplatin (or cisplatin salt or acid) in the complex is in the range 2-8:1, or any range therein between. In some embodiments, the molar ratio of PANTIFOL/cisplatin (or cisplatin salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/cisplatin (or cisplatin salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In some embodiments, the molar ratio of PANTIFOL/cisplatin (or cisplatin salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/cisplatin (or cisplatin salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In additional embodiments, the PANTIFOL/cisplatin (or cisplatin salt or acid) complex is encapsulated in a liposome.

In another embodiment, the disclosure provides a complex containing PANTIFOL and carboplatin or a salt or acid thereof. In some embodiments, the complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the molar ratio of PANTIFOL/carboplatin (or carboplatin salt or acid) in the complex is in the range 1-20:1, or any range therein between. In further embodiments, the molar ratio of PANTIFOL/carboplatin (or carboplatin salt or acid) in the complex is in the range 1-10:1, or any range therein between. In further embodiments, the molar ratio of PANTIFOL/carboplatin (or carboplatin salt or acid) in the complex is in the range 2-8:1, or any range therein between. In some embodiments, the molar ratio of PANTIFOL/carboplatin (or carboplatin salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the molar ratio of PANTIFOL/carboplatin (or carboplatin salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In some embodiments, the molar ratio of PANTIFOL/carboplatin in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/carboplatin in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In additional embodiments, the PANTIFOL/carboplatin (or carboplatin salt or acid) complex is encapsulated in a liposome.

In another embodiment, the disclosure provides a complex containing PANTIFOL and oxaliplatin, or a salt or acid thereof. In some embodiments, the complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the molar ratio of PANTIFOL/oxaliplatin (or oxaliplatin salt or acid) in the complex is in the range 1-20:1, or any range therein between. In further embodiments, the molar ratio of PANTIFOL/oxaliplatin (or oxaliplatin salt or acid) in the complex is in the range 1-10:1, or any range therein between. In further embodiments, the molar ratio of PANTIFOL/oxaliplatin (or oxaliplatin salt or acid) in the complex is in the range 2-8:1, or any range therein between. In some embodiments, the molar ratio of PANTIFOL/oxaliplatin (or oxaliplatin salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/oxaliplatin (or oxaliplatin salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In some embodiments, the molar ratio of PANTIFOL/oxaliplatin (or oxaliplatin salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/oxaliplatin (or oxaliplatin salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In additional embodiments, the PANTIFOL/oxaliplatin (or oxaliplatin salt or acid) complex is encapsulated in a liposome.

In additional embodiments, the disclosure provides a complex comprising PANTIFOL and a platinum-based chemotherapeutic agent (platinum) selected from: nedaplatin, heptaplatin, lobaplatin, stratoplatin, paraplatin, platinol, cycloplatin, dexormaplatin, spiroplatin, picoplatin, triplatin, tetraplatin, iproplatin, ormaplatin, zeniplatin, platinum-triamine, traplatin, enloplatin, JM216, NK121, CI973, DWA 2114R, NDDP, and dedaplatin, or a salt or acid thereof. In other embodiments, the PANTIFOL/platinum-based chemotherapeutic agent complex comprises an analog of nedaplatin, heptaplatin, lobaplatin, stratoplatin, paraplatin, platinol, cycloplatin, dexormaplatin, spiroplatin, picoplatin, triplatin, tetraplatin, iproplatin, ormaplatin, zeniplatin, platinum-triamine, traplatin, enloplatin, JM216, NK121, CI973, DWA 2114R, NDDP, or dedaplatin, or a salt or acid thereof. In some embodiments, the molar ratio of PANTIFOL/platinum-based chemotherapeutic agent (“platinum”) (or platinum-based chemotherapeutic agent salt or acid) in the complex is in the range 1-20:1, or any range therein between. In some embodiments, the complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In further embodiments, the molar ratio of PANTIFOL/platinum (or platinum salt or acid) in the complex is in the range 1-10:1, or any range therein between. In further embodiments, the molar ratio of PANTIFOL/platinum (or platinum salt or acid) in the complex is in the range 2-8:1, or any range therein between. In some embodiments, the molar ratio of PANTIFOL/platinum (or platinum salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the molar ratio of PANTIFOL/platinum (or platinum salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In some embodiments, the molar ratio of PANTIFOL/platinum (or platinum salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/platinum (or platinum salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In additional embodiments, the PANTIFOL/platinum (or salt or acid or analog thereof) complex is encapsulated in a liposome.

In some embodiments, the disclosure provides a composition comprising a PANTIFOL/taxane-based chemotherapeutic agent (taxane) complex. In some embodiments, the complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the taxane-based chemotherapeutic agent is selected from: paclitaxel (PTX), docetaxel (DTX), larotaxel (LTX), and cabazitaxel (CTX), or a salt or acid thereof. In some embodiments, the molar ratio of PANTIFOL/taxane (or taxane salt or acid) in the complex in the complex is in the range 1-20:1, or any range therein between. In further embodiments, the molar ratio of PANTIFOL/taxane (or taxane salt or acid) in the complex is in the range 1-10:1, or any range therein between. In further embodiments, the molar ratio of PANTIFOL/taxane (or taxane salt or acid) in the complex is in the range 2-8:1, or any range therein between. In some embodiments, the molar ratio of PANTIFOL/taxane (or taxane salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the molar ratio of PANTIFOL/taxane (or taxane salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In some embodiments, the molar ratio of PANTIFOL/taxane (or taxane salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/taxane (or taxane salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In additional embodiments, the PANTIFOL/taxane (or taxane salt or acid) complex is encapsulated in a liposome.

In additional embodiments, the disclosure provides a complex comprising PANTIFOL and paclitaxel (PTX), or a salt or acid thereof. In other embodiments, the PANTIFOL/paclitaxel (or paclitaxel salt or acid) chemotherapeutic agent complex comprises an analog of paclitaxel (PTX), or a salt or acid thereof. In some embodiments, the complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the molar ratio of PANTIFOL/paclitaxel (or paclitaxel salt or acid) in the complex is in the range 1-20:1, or any range therein between. In further embodiments, the molar ratio of PANTIFOL/paclitaxel (or paclitaxel salt or acid) in the complex is in the range 1-10:1, or any range therein between. In further embodiments, the molar ratio of PANTIFOL/paclitaxel (or paclitaxel salt or acid) in the complex is in the range 2-8:1, or any range therein between. In some embodiments, the molar ratio of PANTIFOL/paclitaxel (or paclitaxel salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the molar ratio of PANTIFOL/paclitaxel (or paclitaxel salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In some embodiments, the molar ratio of PANTIFOL/paclitaxel (or paclitaxel salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/paclitaxel (or paclitaxel salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In additional embodiments, the PANTIFOL/paclitaxel (or paclitaxel salt or acid) complex is encapsulated in a liposome.

In additional embodiments, the disclosure provides a complex comprising PANTIFOL and docetaxel (DTX), or a salt or acid thereof. In other embodiments, the PANTIFOL/docetaxel complex comprises an analog of docetaxel (DTX), or a salt or acid thereof. In some embodiments, the complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the molar ratio of PANTIFOL/docetaxel (or docetaxel salt or acid) in the complex is in the range 1-20:1, or any range therein between. In some embodiments, the molar ratio of PANTIFOL/docetaxel (or docetaxel salt or acid) in the complex is in the range 1-10:1, or any range therein between. In further embodiments, the molar ratio of PANTIFOL/docetaxel (or docetaxel salt or acid) in the complex is in the range 2-8:1, or any range therein between. In some embodiments, the molar ratio of PANTIFOL/docetaxel (or docetaxel salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the molar ratio of PANTIFOL/docetaxel (or docetaxel salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In some embodiments, the molar ratio of PANTIFOL/docetaxel (or docetaxel salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/docetaxel (or docetaxel salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In additional embodiments, the PANTIFOL/docetaxel (or docetaxel salt or acid) complex is encapsulated in a liposome.

In additional embodiments, the disclosure provides a complex comprising PANTIFOL and larotaxel (LTX), or a salt or acid thereof. In some embodiments, the complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the molar ratio of PANTIFOL/larotaxel (or larotaxel salt or acid) in the complex is in the range 1-20:1, or any range therein between. In further embodiments, the molar ratio of PANTIFOL/larotaxel (or larotaxel salt or acid) in the complex is in the range 1-10:1, or any range therein between. In further embodiments, the molar ratio of PANTIFOL/larotaxel (or larotaxel salt or acid) in the complex is in the range 2-8:1, or any range therein between. In some embodiments, the molar ratio of PANTIFOL/larotaxel (or larotaxel salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the molar ratio of PANTIFOL/larotaxel (or larotaxel salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In some embodiments, the molar ratio of PANTIFOL/larotaxel (or larotaxel salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/larotaxel (or larotaxel salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In additional embodiments, the PANTIFOL/larotaxel (or larotaxel salt or acid) complex is encapsulated in a liposome.

In additional embodiments, the disclosure provides a complex comprising PANTIFOL and cabazitaxel (CTX), or a salt or acid thereof. In some embodiments, the complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the molar ratio of PANTIFOL/cabazitaxel (or cabazitaxel salt or acid) in the complex is in the range 1-20:1, or any range therein between. In further embodiments, the molar ratio of PANTIFOL/cabazitaxel (or cabazitaxel salt or acid) in the complex is in the range 1-10:1, or any range therein between. In further embodiments, the molar ratio of PANTIFOL/cabazitaxel (or cabazitaxel salt or acid) in the complex is in the range 2-8:1, or any range therein between. In some embodiments, the molar ratio of PANTIFOL/cabazitaxel (or cabazitaxel salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the molar ratio of PANTIFOL/cabazitaxel (or cabazitaxel salt or acid) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In some embodiments, the molar ratio of PANTIFOL/cabazitaxel (or cabazitaxel salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/cabazitaxel (or cabazitaxel salt or acid) in the complex is: 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In additional embodiments, the PANTIFOL/cabazitaxel (or cabazitaxel salt or acid) complex is encapsulated in a liposome.

In additional embodiments, the disclosure provides a complex comprising PANTIFOL and another anti-metabolite, or a salt or acid thereof. In some embodiments, the complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. An anti-metabolite is a chemical with a structure that is similar to a metabolite required for normal biochemical reactions, yet different enough to interfere with one or more normal functions of cells, such as cell division. In some embodiments, the disclosure provides a complex comprising PANTIFOL (e.g., αPANTIFOL and/or γPANTIFOL) and Antifolate (ANTIFOL), or a salt or acid thereof. In some embodiments, the disclosure provides a complex comprising PANTIFOL (e.g., αPANTIFOL and/or γPANTIFOL) and an anti-metabolite selected from, gemcitabine, fluorouracil, capecitabine, an antifolate (e.g., Antifolate, raltitrexed), tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salt or acids, acids, or derivatives of any of these. In some embodiments, the molar ratio of PANTIFOL/anti-metabolite (or anti-metabolite salt or acid, or prodrug) in the complex is in the range 1-20:1, or any range therein between. In further embodiments, the molar ratio of PANTIFOL/anti-metabolite (or anti-metabolite salt or acid, or prodrug) in the complex is in the range 1-10:1, or any range therein between. In further embodiments, the molar ratio of PANTIFOL/anti-metabolite (or anti-metabolite salt or acid, or prodrug) in the complex is in the range 2-8:1, or any range therein between. In some embodiments, the molar ratio of PANTIFOL/anti-metabolite (or anti-metabolite salt or acid, or prodrug) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the molar ratio of PANTIFOL/anti-metabolite (or anti-metabolite salt or acid, or prodrug) in the complex is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In some embodiments, the molar ratio of PANTIFOL/anti-metabolite (or anti-metabolite salt or acid, or prodrug) in the complex is 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the molar ratio of PANTIFOL/anti-metabolite (or anti-metabolite salt or acid, or prodrug) in the complex is 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In additional embodiments, the PANTIFOL/anti-metabolite (or anti-metabolite salt or acid, or prodrug) complex is encapsulated in a liposome.

In additional embodiments, the disclosure provides a complex of PANTIFOL (e.g., a αPANTIFOL and/or γPANTIFOL disclosed herein) and a cyclodextrin. Cyclodextrins (CDs) are groups of cyclic oligosaccharides which have been shown to improve physicochemical properties of many drugs through formation of complexes. CDs are cyclic oligosaccharides composed of several D-glucose units linked by α-(1,4) bonds. This cyclic configuration provides a hydrophobic internal cavity and gives the CDs a truncated cone shape. Many hydroxyl groups are situated on the edges of the ring which make the CDs both lipophilic and soluble in water. As a result, CDs are able to form complexes with a wide variety of hydrophobic agents, and thus change the physical-chemical properties of these complexed agents. In some embodiments, the complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

The terms “cyclodextrin” or “CD” unless otherwise specified herein, refer generally to a parent or derivatized cyclic oligosaccharide containing a variable number of (α-1,4)-linked D-glucopyranoside units that is able to form a complex with a Antifolate-PG. Each cyclodextrin glucopyranoside subunit has secondary hydroxyl groups at the 2 and 3 positions and a primary hydroxyl group at the 6-position. The terms “parent”, “underivatized”, or “inert”, cyclodextrin refer to a cyclodextrin containing D-glucopyranoside units having the basic formula C6H12O6 and a glucose structure without any additional chemical substitutions (e.g., α-cyclodextrin consisting of 6 D-glucopyranoside units, a β-cyclodextrin consisting of 7 D-glucopyranoside units, and a γ-cyclodextrin cyclodextrin consisting of 8 D-glucopyranoside units). The physical and chemical properties of a parent cyclodextrin can be modified by derivatizing the hydroxyl groups with other functional groups. Any substance located within the cyclodextrin internal phase is said to be “complexed” with the cyclodextrin, or to have formed a complex (inclusion complex) with the cyclodextrin.

As used herein, there are no particular limitations on the cyclodextrin component of the PANTIFOL/cyclodextrin complexes so long as the cyclodextrins can form complexes with the PANTIFOL. In particular embodiments, the cyclodextrins have been derivatized to bear ionizable (e.g., weakly basic and/or weakly acidic) functional groups to facilitate complex formation with PANTIFOL and/or liposome encapsulation.

Modifications of the hydroxyl groups of cyclodextrins, such as those facing away from the cyclodextrin interior phase, with ionizable chemical groups is known to facilitate the loading of cyclodextrins and therapeutic agents complexed with the cyclodextrins. In some embodiments, the cyclodextrin of the PANTIFOL/cyclodextrin complex has at least 2, 3, 4, 5, 6, 6, 7, 8, 9, or 10, hydroxyl group substituted with an ionizable chemical group. The term “charged cyclodextrin” refers to a cyclodextrin having one or more of its hydroxyl groups substituted with a charged moiety. Such a moiety can itself be a charged group or it can comprise an organic moiety (e.g., a C1-C6 alkyl or C1-C6 alkyl ether moiety) substituted with one or more charged moieties.

In some embodiments, the “ionizable” or “charged” moieties of a CD derivative are weakly ionizable. Weakly ionizable moieties are those that are either weakly basic or weakly acidic. Weakly basic functional groups (W) have a pKa of between about 6.0-9.0, 6.5-8.5, 7.0-8.0, 7.5-8.0, and any range in between inclusive according to CH3-W. Similarly, weakly acidic functional groups (X) have a log dissociation constant (pKa) of between about 3.0-7.0, 4.0-6.5, 4.5-6.5, 5.0-6.0, 5.0-5.5, and any range in between inclusive according to CH3-X. Representative anionic moieties include, without limitation, carboxylate, carboxymethyl, succinyl, sulfonyl, phosphate, sulfoalkyl ether, sulphate carbonate, thiocarbonate, dithiocarbonate, phosphate, phosphonate, sulfonate, nitrate, and borate groups. Representative cationic moieties include, without limitation, amino, guanidine, and quarternary ammonium groups.

In another embodiment, the derivatized cyclodextrin is a “polyanion” or “polycation.” A polyanion is a derivatized cyclodextrin having more than one negatively charged group resulting in net a negative ionic charge of more than two units. A polycation is a derivatized cyclodextrin having more than one positively charged group resulting in net positive ionic charger of more than two units.

In another embodiment, the derivatized cyclodextrin is a “chargeable amphiphile.” By “chargeable” is meant that the amphiphile has a pK in the range pH 4 to pH 8 or 8.5. A chargeable amphiphile may therefore be a weak acid or base. By “amphoteric” herein is meant a derivatized cyclodextrin having a ionizable groups of both anionic and cationic character wherein: (a) at least one, and optionally both, of the cation and anionic amphiphiles is chargeable, having at least one charged group with a pK between 4 and 8 to 8.5, (b) the cationic charge prevails at pH 4, and (c) the anionic charge prevails at pH 8 to 8.5.

In some embodiments, the “ionizable” or “charged” derivatized cyclodextrin as a whole, whether polyionic, amphiphilic, or otherwise, are weakly ionizable (i.e., have a pKai of between about 4.0-8.5, 4.5-8.0, 5.0-7.5, 5.5-7.0, 6.0-6.5, and any range in between inclusive).

Any one, some, or all hydroxyl groups of any one, some or all α-D-glucopyranoside units of a cyclodextrin can be modified to an ionizable chemical group as described herein. Since each cyclodextrin hydroxyl group differs in chemical reactivity, reaction with a modifying moiety can produce an amorphous mixture of positional and optical isomers. Alternatively, certain chemistry can allow for pre-modified α-D-glucopyranoside units to be reacted to form uniform products.

The aggregate substitution that occurs for cyclodextrin derivatives in a mixture is described by a term referred to as the degree of substitution. For example, a 6-ethylenediamino-β-cyclodextrin with a degree of substitution of seven would be composed of a distribution of isomers of 6-ethylenediamino-β-cyclodextrin in which the average number of ethylenediamino groups per 6-ethylenediamino-β-cyclodextrin molecule is seven. The degree of substitution for a cyclodextrin derivative mixture can routinely be determined using mass spectrometry or nuclear magnetic resonance spectroscopy.

In one embodiment, at least one hydroxyl moieties facing away from the cyclodextrin interior is substituted with an ionizable chemical group. For example, the C2, C3, C6, C2 and C3, C2 and C6, C3 and C6, and all three of C2-C3-C6 hydroxyls of at least one α-D-glucopyranoside unit are substituted with an ionizable chemical group. Any such combination of hydroxyls can similarly be combined with at least two, three, four, five, six, seven, eight, nine, ten, eleven, up to all of the alpha-D-glucopyranoside units in the modified cyclodextrin as well as in combination with any degree of substitution described herein. One such derivative is a sulfoalkyl ether cyclodextrin (SAE-CD). Sulfobutyl ether derivatives of beta cyclodextrin (SBE-β-CD) have been demonstrated to have significantly improved aqueous solubility compared to the parent cyclodextrin.

Additional cyclodextrin derivatives that may be complexed with therapeutic agents in the disclosed liposome compositions include sugammadex or Org-25969, in which the 6-hydroxy groups on γ-CD have been replaced by carboxythio acetate ether linkages, and hydroxybutenyl-β-CD. Alternative forms of cyclodextrin include: 2,6-Di-O-methyl-β-CD (DIMEB), 2-hydroxylpropyl-3-cyclodextrin (HP-β-CD), randomly methylated-β-cyclodextrin (RAMEB), sulfobutyl ether β-cyclodextrin (SBE-β-CD), and sulfobutylether-γ-cyclodextrin (SBEγCD), sulfobutylated beta-cyclodextrin sodium salt, (2-Hydroxypropyl)-alpha-cyclodextrin, (2-Hydroxypropyl)-beta-cyclodextrin, (2-Hydroxy-propyl)-γ-cyclodextrin, 2,6-di-O-methyl)-beta-cyclodextrin (DIMEB-50 Heptakis), 2,3,6-tri-O-methyl)-beta-cyclodextrin (TRIMEB Heptakis), methyl-beta-cyclodextrin, octakis (6-deoxy-6-iodo)-γ-cyclodexrin, and, octakis (6-deoxy-6-bromo)-gamma-cyclodexrin.

In some embodiments, the cyclodextrin(s) has a high solubility in water in order to facilitate entrapment of a larger amount of the cyclodextrin in the liposome internal phase. In some embodiments, the water solubility of the cyclodextrin is at least 10 mg/mL, 20 mg/mL, 30 mg/mL, 40 mg/mL, 50 mg/mL, 60 mg/mL, 70 mg/mL, 80 mg/mL, 90 mg/mL, 100 mg/mL or higher. In some embodiments, the water solubility of the cyclodextrin(s) is within a range of 10-150 mg/mL, 20-100 mg/mL 20-75 mg/mL, and any range in between inclusive.

In some embodiments, a large association constant between the cyclodextrin and the PANTIFOL and/or other therapeutic agent complexed with cyclodextrin is preferable and can be obtained by selecting the number of glucose units in the cyclodextrin based on the size of the therapeutic agent (see, for example, Albers et al., Crit. Rev. Therap. Drug Carrier Syst. 12:311-337 (1995); Stella et al., Toxicol. Pathol. 36:30-42 (2008). When the association constant depends on pH, the cyclodextrin can be selected such that the association constant becomes large at the pH of the liposome internal phase. As a result, the solubility (nominal solubility) of the therapeutic agent in the presence of cyclodextrin can be further improved. In some embodiments, the association constant of the cyclodextrin with the therapeutic agent is 100, 200, 300, 400, 500, 600, 700, 800, 900, 1,000, or higher. In some embodiments, the association constant of the cyclodextrin with the therapeutic agent is in the range 100-1, 200, 200-1,000, 300-750, and any range therein between.

In some embodiments, the cyclodextrin of the PANTIFOL/cyclodextrin complex and/or cyclodextrin/therapeutic agent complex is underivatized.

In some embodiments, the cyclodextrin of the PANTIFOL/cyclodextrin complex and/or cyclodextrin/therapeutic agent complex is derivatized. In further embodiments, the cyclodextrin derivative of the complex has the structure of Formula CD-1:

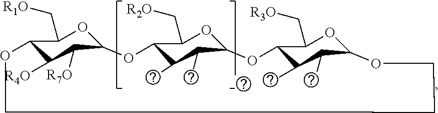

wherein: n is 4, 5, or 6;
wherein R₁, R₂, R₃, R₄, R₅, R₆, R₇, R₈, and R₉ are each, independently, —H, a straight chain or branched C₁-C₈-alkylene group, or an optionally substituted straight-chain or branched C₁-C₆ group, wherein at least one of R₁, R₂, R₃, R₄, R₅, R₆, R₇, R₈ and R₉ is a straight-chain or branched C₁-C₈-alkylene (e.g., C₁-C₈-(alkylene)-SO₃⁻ group);

In some embodiments, the cyclodextrin derivative of the PANTIFOL/cyclodextrin complex and/or cyclodextrin/therapeutic agent complex has the structure of Formula CD-2:

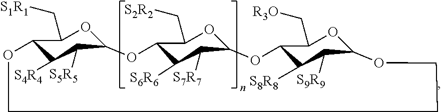

wherein: n is 4, 5, or 6;
wherein R1, R2, R3, R4, R5, R6, R7, R8, and R9 are each, independently, —O— or a —O—(C2-C6 alkylene)-SO3- group; wherein at least one of R1 and R2 is independently a —O—(C2-C6 alkylene)-SO3- group; and S1, S2, S3, S4, S5, S6, S7, S8, and S9 are each, independently, a pharmaceutically acceptable cation. In further embodiments, the pharmaceutically acceptable cation is selected from: an alkali metal such as Li+, Na+, or K+; an alkaline earth metal such as Ca+2, or Mg⁺² and ammonium ions and amine cations such as the cations of (C1-C6)-alkylamines, piperidine, pyrazine, (C1-C6)-alkanolamine and (C4-C8)-cycloalkanolamine. In some embodiments, at least one of R1 and R2 is independently a —O—(C2-C6 alkylene)-SO3- group that is a —O—(CH₂)ₘSO3- group, wherein m is 2 to 6, preferably 2 to 4, (e.g., —O-CH2CH2CH2SO3- or —O—CH2CH2CH2CH2SO3-); and S₁, S₂, S₃, S₄, S₅, S₆, S₇, S₈, and S₉ are each, independently, H or a pharmaceutically cation which includes for example, alkali metals (e.g., Li⁺, Na⁺, K⁺) alkaline earth metals (e.g., Ca⁺², Mg⁺²), ammonium ions and amine cations such as the cations of (C1-C6)-alkylamines, piperidine, pyrazine, (C₁-C₆)-alkanol-amine and (C₄-C₈)-cycloalkanolamine:

In some embodiments, a cyclodextrin derivative of the PANTIFOL/cyclodextrin complex and/or cyclodextrin/therapeutic agent complex is a cyclodextrin disclosed in U.S. Pat. Nos. 6,133,248, 5,874,418, 6,046,177, 5,376,645, 5,134,127, 7,034,013, 6,869,939; and Intl. Appl. Publ. No. WO 02005/117911, the contents each of which is herein incorporated by reference in its priority.

In some embodiments, the cyclodextrin derivative of the PANTIFOL/cyclodextrin complex and/or cyclodextrin/therapeutic agent complex is a sulfoalkyl ether cyclodextrin. In some embodiments, the cyclodextrin derivative of complex is a sulfobutyl ether-3-cyclodextrin such as CAPTISOL® (CyDex Pharma. Inc., Lenexa, Kans.). Methods for preparing sulfobutyl ether-3-cyclodextrin and other sulfoalkyl ether cyclodextrins are known in the art.

In some embodiments, the cyclodextrin derivative in of the PANTIFOL/cyclodextrin complex and/or cyclodextrin/therapeutic agent complex is a compound of Formula CD-3:

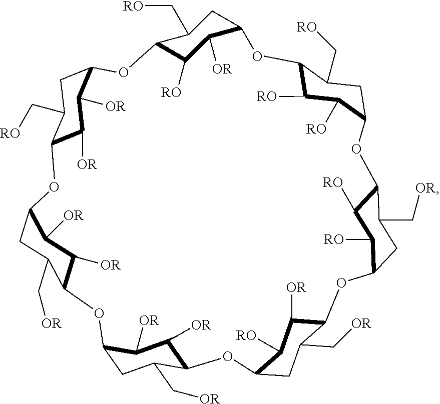

wherein R equals:

(a) (H)_{21-X} or (—(CH₂)₄—SO₃Na)x, and x=1.0-10.0, 1.0-5.0, 6.0-7.0, or 8.0-10.0;

(b) (H)_{21-X} or (—(CH₂CH(OH)CH₃)x, and x=1.0-10.0, 1.0-5.0, 6.0-7.0, or 8.0-10.0;

(c) (H)_{21-X} or (sulfoalkyl ethers)x, and x=1.0-10.0, 1.0-5.0, 6.0-7.0, or 8.0-10.0; or

(d) (H)_{21-X} or (—(CH₂)₄—SO₃Na)x, and x=1.0-10.0, 1.0-5.0, 6.0-7.0, or 8.0-10.0.

In additional embodiments, the PANTIFOL/cyclodextrin complex and/or cyclodextrin/therapeutic agent complex is encapsulated in a liposome (e.g., as described herein or otherwise known in the art).

### PANTIFOL Delivery Vehicles

In alternative embodiments, the disclosure provides PANTIFOL delivery systems and their use to deliver a payload of PANTIFOL (e.g., αPANTIFOL and/or γPANTIFOL) to a cell or cells in vitro or in vivo. In some embodiments, the PANTIFOL (e.g., αPANTIFOL and/or γPANTIFOL) of the present disclosure is complexed with or incorporated into a delivery vehicle. Such delivery vehicles are known in the art and include, but are not limited to, liposomes, lipospheres, polymers, peptides, proteins, antibodies (e.g., ADCs such as Antibody-PANTIFOL conjugates), cellular components, cyclic oligosaccharides (e.g., cyclodextrins), nanoparticles (e.g., lipid nanoparticles, biodegradable nanoparticles, and core-shell nanoparticles), lipoprotein particles, and combinations thereof. In particular embodiments, the delivery vehicle is a liposome. In other particular embodiments, the delivery vehicle is an antibody or an antigen binding antibody fragment. In some embodiments, the PANTIFOL delivery system comprises a PANTIFOL (e.g., αPANTIFOL and/or γPANTIFOL) of the present disclosure. Without wishing to be bound by theories, the polyglutamated Antifolates can become highly negatively charged under various physiological conditions, which render them less permeable to cells without a delivery vehicle, e.g., the liposomes described herein. The inventors have also tested representative PANTIFOLs (without any delivery vehicle) in a Caco-2 permeability assay and found the tested PANTIFOL essentially impermeable.

### Liposomes

In some embodiments, the disclosure provides liposomal compositions that comprise a liposome encapsulating (i.e., filled with) a PANTIFOL, such as αPANTIFOL and/or γPANTIFOL, alternatively referred to herein as Lp-PANTIFOL. In some embodiments, the disclosure provides liposomal compositions that comprise a liposome encapsulating (i.e., filled with) a gamma polyglutamated Antifolate (e.g., a γPANTIFOL disclosed herein), alternatively referred to herein as Lp-γPANTIFOL. In some embodiments, the disclosure provides liposomal compositions that comprise a liposome encapsulating (i.e., filled with) an alpha polyglutamated Antifolate (e.g., an αPANTIFOL disclosed herein), alternatively referred to herein as Lp-αPANTIFOL. In some embodiments, the liposomal composition comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the liposomal composition comprises a liposome that is anionic or neutral. In some embodiments, the liposomal composition comprises a liposome that is cationic. In some embodiments, the Lp-PANTIFOL composition is not pegylated. In some embodiments, the Lp-PANTIFOL composition is non-targeted (NTLp-PANTIFOL). In other embodiments, the Lp-PANTIFOL composition comprises a targeting moiety (TLp-PANTIFOL). In some embodiments, the liposomal composition comprises a liposome having a diameter in the range of 20 nm to 500 nm, or any range therein between. In some embodiments, the liposomal composition comprises a liposome having a diameter in the range of 20 nm to 400 nm, or any range therein between. In some embodiments, the liposomal composition comprises a liposome having a diameter in the range of 20 nm to 200 nm, or any range therein between. In further embodiments, the liposomal composition comprises a liposome having a diameter in the range of 20 nm to 150 nm, or any range therein between. In further embodiments, the liposomal composition comprises a liposome having a diameter in the range of 80 nm to 120 nm, or any range therein between. In additional embodiments, 30-70%, 30-60%, or 30-50% w/w polyglutamated Antifolate, or any range therein between, is encapsulated (entrapped) in the Lp-PANTIFOL during the process of preparing the liposomes. In some embodiments, the Lp-PANTIFOL composition comprises at least 1%, 5%, 10%, 15%, 20%, 25%, 30%, 35, 40%, 45%, 50%, 55%, 60%, 65%, 70%, 75%, or more than 75%, w/w of the polyglutamated Antifolate. In some embodiments, at least 1%, 5%, 10%, 15%, 20%, 25%, 30%, 35%, 40%, 45%, 50%, 55%, 60%, 65%, 70%, 75% or more than 75%, w/w, polyglutamated Antifolate, is encapsulated in the Lp-PANTIFOL during the process of preparing the liposomes.

In some embodiments, the provided liposomes further comprise an immunostimulatory agent, a detectable marker, or both disposed on its exterior. The immunostimulatory agent or detectable marker can be ionically bonded or covalently bonded to an exterior of the liposome, including, for example, optionally to a steric stabilizer component of the liposome.

The terms “immunostimulatory agents”, also known as “immunostimulants”, and “immunostimulators”, refer to substances that stimulate an immune (including a preexisting immune response) by inducing activation or increasing activity of any of the components of the immune system. These immunostimulatory agents can include one or more of a hapten, an adjuvant, a protein immunostimulating agent, a nucleic acid immunostimulating agent, and a chemical immunostimulating agent. Many adjuvants contain a substance designed to stimulate immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A; IFN-alpha, IFN-gamma, FLT3-ligand; and immunostimulatory antibodies (e.g., anti-CTLA-4, anti-CD28, anti-CD3). Cytokines, such as GM-CSF, interleukin-2, -7, -12, and -15, and other like growth factors, can also be used as adjuvants. In a preferred embodiment, the immunostimulant can be at least one selected from fluorescein, DNP, beta glucan, beta-1,3-glucan, beta-1,6-glucan. In an additional preferred embodiment, the immunostimulant is a Toll-like receptor (TLR) modulating agent. In further embodiments, the Toll-like receptor (TLR) modulating agent is one or more of: OXPAC, PGPC, an eritoran lipid (e.g., E5564), and a resolvin.

In some embodiments, the provided liposomes further comprise an agent, that increase uptake of liposomes into a cellular compartment of interest including the cytosol. In some embodiments, the agent provides the liposome contents with the ability to bypass lysosomes (e.g., chloroquine). In some embodiments, the agent improves the update of the liposome contents by mitochondria (e.g., sphingomyelin and a component of mitoport).

A detectable marker may, for example, include, at least, a radioisotope, a fluorescent compound, a bioluminescent compound, chemiluminescent compound, a metal chelator, an enzyme, a dye, an ink, a magnetic compound, a biocatalyst or a pigment that is detectable by any suitable means known in the art, e.g., magnetic resonance imaging (MRI), optical imaging, fluorescent/luminescent imaging, or nuclear imaging techniques.

In some embodiments, the immunostimulatory agent and/or detectable marker is attached to the exterior by co-incubating it with the liposome. For example, the immunostimulatory agent and/or detectable marker may be associated with the liposomal membrane by hydrophobic interactions or by an ionic bond such as an avidin/biotin bond or a metal chelation bond (e.g., Ni-NTA). Alternatively, the immunostimulatory agent or detectable marker may be covalently bonded to the exterior of the liposome such as, for example, by being covalently bonded to a liposomal component or to the steric stabilizer which is the PEG.

One example reagent is fluorescein isothiocyanate (FITC) which, based on our experiments, surprisingly serves as both an immunostimulant and a detectable marker.

In some embodiments, the liposomes further comprise an agent that increases the uptake of liposomes into a cellular compartment of interest including the cytosol.

In some embodiments, the liposomes comprise a mitochondrial-targeting agent. In some embodiments, the liposomes comprise triphenylphosphonium (TPP). Methods and mechanisms for surface functionalizing liposomes with TPP are known in the art (e.g., attaching TPP to the lipid anchor via a peg spacer group and modifying TPP with a stearyl group (stearyl triphenylphosphonium (STPP)). In some embodiments, the liposomes comprise high-density octa-arginine. In some embodiments, the liposomes comprise sphingomyelin and/or a sphingomyelin metabolite. Sphingomyelin metabolite used to formulate the liposomes of the present invention can include, for example ceramide, sphingosine or sphingosine 1-phosphate. In some embodiments, the liposomes comprise Rhodamine 123. In some embodiments, the liposomes comprise, a mitochondrion penetrating peptide. In some embodiments, the liposomes comprise, a mitochondrion penetrating agent selected from: a mitofusin peptide, a mitochondrial targeting signal peptide, and Antennapedia helix III homeodomain cell-penetrating peptide (ANT) (e.g., comprising RQIKIWFQNRRMKWKKRKKRRQRRR, RKKRRXRRRGC), or a mitochondrial penetrating fragment thereof. In some embodiments, the liposomes comprise, a mitochondria penetrating polynucleotide sequence selected from: RQIKIWFQNRRMKWKKRK KRRQRRR (SEQ ID NO:1), RKKRRXR RRGC where X is any natural or non-natural amino acid (SEQ ID NO:2), CCGCCAAGAAGCG (SEQ ID NO:3), GCGTGCACACGCGCGTA GACTTCCCCCGCAAGTCACTCGTTAGCCCGCCAAGAAGCGACCCCTCCGGGG CGAGCTGAGCGGCGTGGCGCGGGGGCGTCAT (SEQ ID NO:4), ACGTGCATACGCA CGTAGACATTCCCCGCTTCCCACTCCAAAGTCCGCCAAGAAGCGTATC CCGCTGAG CGGCGTGGCGCGGGGGCGTCATCCGTCAGCTC (SEQ ID NO:5), or ACTTCCCCCG CAAGTCACTCGTTAGCCCGCCAAGAAGCGACCCCTCCGGGGCGAGCTG (SEQ ID NO:6)), or a mitochondrial penetrating fragment thereof.

In some embodiments, liposomes in the provided liposome compositions comprise a mitochondria penetrating agent selected from the group: a guanidine-rich peptoid, tetraguanidinium, triguanidinium, diguanidinium, monoguanidinium, a guanidine-rich polycarbamate, a beta-oligoarginine, a proline-rich dendrimer, and a phosphonium salt (e.g., methyltriphenyl-phosphonium and/or tetraphenylphosphonium).

In some embodiments, liposomes in the provided liposome compositions comprise sphingomyelin and/or stearyl-octa-arginine. In some embodiments, the liposomes comprise sphingomyelin and/or stearyl-octa-arginine. In some embodiments, the liposomes comprise DOPE, sphingomyelin, stearyl-octa-arginine sphingomyelin and stearyl-octa-arginine. In some embodiments, the liposomes comprise DOPE, sphingomyelin, stearyl-octa-arginine sphingomyelin and stearyl-octa-arginine at a molar ratio of 9:2:1. In some embodiments, the liposomes comprise the MITO-Porter® system or a variant thereof.

In some embodiments, liposomes in the provided liposome compositions comprise an agent such as a cell penetrating agent that that facilitates delivery of the liposome across a cell membrane and provides the liposome with the ability to bypass the endocytic pathway and the harsh environment of lysosomes. Cell penetrating agents are known in the art and can routinely be used and adapted for manufacture and use of the provided liposome compositions. In some embodiments, the cell penetrating/lysosome bypassing agent is chloroquine. In some embodiments, the cell penetrating agent is a cell penetrating peptide. In some embodiments, liposomes in the provided liposome compositions comprise a cell penetrating agent selected from the group: RKKRRQRRR (SEQ ID NO:7), GRKKRRQRRRTPQ (SEQ ID NO:8), YGRK KRRQRRR (SEQ ID NO:9), AAVALLPAVLLALLA (SEQ ID NO:10), MGLGLHLLV LAAALQ (SEQ ID NO:11), GALFLGFLGAAGSTM (SEQ ID NO:12), AGYLLGKINLKA LAALAKKIL (SEQ ID NO:13), RVIRVWFQNKRCKDKK (SEQ ID NO:14), RQIKIWFQN RRMKWKK (SEQ ID NO:15), GLFEAIAGFIENGWEGMIDG (SEQ ID NO:16), GWTLNSA GYLLGKIN (SEQ ID NO:17), RSQSRSRYYRQRQRS (SEQ ID NO:18), LAIPEQEY (SEQ ID NO:19), LGIAEQEY (SEQ ID NO:20), LGIPAQEY (SEQ ID NO:21), LGIPEAEY (SEQ ID NO:22), LGIPEQAY (SEQ ID NO:23), LGIAEAEY (SEQ ID NO:24), LGIPEAAY (SEQ ID NO:25), LGIAEQAY (SEQ ID NO:26), LGIAEAAY (SEQ ID NO:27), LLIILRRRIRKQAHA HSK (SEQ ID NO:28), LKALAALAKKIL (SEQ ID NO:29), KLALKLALKALKAALKLA (SEQ ID NO:30), KETWWETWWTEWSQPKKKRKV (SEQ ID NO:31), DHQLNPAF (SEQ ID NO:32), DPKGDPKG (SEQ ID NO:33), VTVTVTVTVTGKGDPKPD (SEQ ID NO:34), RQIKIWFQNRRMKWKK (SEQ ID NO:35), GRKKRRQRRRPPQ (SEQ ID NO:36), GWTLNS AGYLLGKINLKALAALAKKIL (SEQ ID NO:37), GRKKRRQRRR (SEQ ID NO:38), RRRRRRR (SEQ ID NO:39), RRRRRRRR (SEQ ID NO:40), RRRRRRRRR (SEQ ID NO:41), RRRRRRRR RR (SEQ ID NO:42), RRRRRRRRRRR (SEQ ID NO:43), and YTIWMPENP RPGTPCDIFTNSRGKRASNGGG G(R)n wherein n=2-15 R in the L- and/or D-form (SEQ ID NO:44), or a cell permeating fragment thereof.

As discussed above, the liposomes may comprise a steric stabilizer that increase their longevity in circulation. For those embodiments, which incorporate a steric stabilizer, the steric stabilizer may be at least one member selected from polyethylene glycol (PEG), poly-L-lysine (PLL), monosialoganglioside (GM1), poly(vinyl pyrrolidone) (PVP), poly(acrylamide) (PAA), poly(2-methyl-2-oxazoline), poly(2-ethyl-2-oxazoline), phosphatidyl polyglycerol, poly[N-(2-hydroxypropyl) methacrylamide], amphiphilic poly-N-vinylpyrrolidones, L-amino-acid-based polymer, oligoglycerol, copolymer containing polyethylene glycol and polypropylene oxide, Poloxamer 188, and polyvinyl alcohol. In some embodiments, the steric stabilizer or the population of steric stabilizer is PEG. In one embodiment, the steric stabilizer is a PEG. In a further embodiment, the PEG has a number average molecular weight (Mn) of 200 to 5000 daltons. These PEG(s) can be of any structure such as linear, branched, star or comb structure and are commercially available.

In some embodiments, the disclosure provides liposomal compositions that comprise a pegylated liposome comprising a PANTIFOL (PLp-PANTIFOL). In some embodiments, the pegylated liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the liposomal composition comprises a pegylated liposome that is anionic or neutral. In some embodiments, the liposomal composition comprises a pegylated liposome that is cationic. In some embodiments, the PLp-PANTIFOL composition is non-targeted (NTPLp-PANTIFOL). In other embodiments, the PLp-PANTIFOL composition comprises a targeting moiety (TPLp-PANTIFOL). In additional embodiments, the liposomal composition comprises a pegylated liposome that comprises 30-70%, 30-60%, or 30-50% liposome entrapped polyglutamated Antifolate, or any range therein between. In some embodiments, the liposomal composition comprises a pegylated liposome that comprises at least 1%, 5%, 10%, 15%, 20%, 25%, 30%, 35%, 40%, 45%, 50%, 55%, 60%, 65%, 70%, or 75%, liposome entrapped polyglutamated Antifolate. In some embodiments, the liposomal composition comprises a pegylated liposome having a diameter in the range of 20 nm to 200 nm. In further embodiments, the liposomal composition comprises a pegylated liposome having a diameter in the range of 80 nm to 120 nm.

In some embodiments, greater than 70%, 80% or 90% of the polyglutamated Antifolate in a provided liposomal composition is pentaglutamated. In some embodiments, greater than 70%, 80% or 90% of the polyglutamated Antifolate in a provided composition is hexaglutamated. In some embodiments, greater than 70%, 80% or 90% of the polyglutamated Antifolate in the composition has 4-10, 4-6, or more than 5, γ-glutamyl groups. In some embodiments, greater than 70%, 80% or 90% of the polyglutamated Antifolate in the composition has 4-10, 4-6, or more than 5, α-glutamyl groups.

In some embodiments, greater than 30%, 40%, 50%, 60%, 70%, 80% or 90%, of the polyglutamated Antifolate in a provided liposomal composition is tetraglutamated. In some embodiments, greater than 30%, 40%, 50%, 60%, 70%, 80% or 90%, of the polyglutamated Antifolate in a provided liposomal composition is pentaglutamated. In some embodiments, greater than 30%, 40%, 50%, 60%, 70%, 80% or 90%, of the polyglutamated Antifolate in a provided liposomal composition is hexaglutamated.

In some embodiments, the polyglutamated Antifolate compositions (e.g., polyglutamates and delivery vehicles such as liposomes containing the polyglutamates) are in an aqueous solution. In some embodiments, the polyglutamated Antifolate composition is administered in a liposomal composition at between about 0.005 and about 5000 mg/M2 (meter of body surface area squared), or between about 0.1 and about 1000 mg/M2, or any range therein between. In some embodiments, the PANTIFOL composition is administered in a liposomal composition at about 1 mg/kg to about 500 mg/kg, 1 mg/kg to about 250 mg/kg, 1 mg/kg to about 200 mg/kg, 1 mg/kg to about 150 mg/kg, 1 mg/kg to about 100 mg/kg, 1 mg/kg to about 50 mg/kg, about 1 mg/kg to about 25 mg/kg, about 1 mg/kg to about 20 mg/kg, about 1 mg/kg to about 15 mg/kg, about 1 mg/kg to about 10 mg/kg, or about 1 mg/kg to about 5 mg/kg, or any range therein between.

### Liposome Composition

The lipids and other components of the liposomes contained in the liposomal compositions can be any lipid, lipid combination and ratio, or combination of lipids and other liposome components and their respective ratios known in the art. However, it will be understood by one skilled in the art that liposomal encapsulation of any particular drug, such as, and without limitation, the polyglutamated Antifolate discussed herein, may involve substantial routine experimentation to achieve a useful and functional liposomal formulation. In general, the provided liposomes may have any liposome structure, e.g., structures having an inner space sequestered from the outer medium by one or more lipid bilayers, or any microcapsule that has a semi-permeable membrane with a lipophilic central part where the membrane sequesters an interior. The lipid bilayer can be any arrangement of amphiphilic molecules characterized by a hydrophilic part (hydrophilic moiety) and a hydrophobic part (hydrophobic moiety). Usually amphiphilic molecules in a bilayer are arranged into two dimensional sheets in which hydrophobic moieties are oriented inward the sheet while hydrophilic moieties are oriented outward. Amphiphilic molecules forming the provided liposomes can be any known or later discovered amphiphilic molecules, e.g., lipids of synthetic or natural origin or biocompatible lipids. The liposomes can also be formed by amphiphilic polymers and surfactants, e.g., polymerosomes and niosomes. For the purpose of this disclosure, without limitation, these liposome-forming materials also are referred to as “lipids”.

The liposome composition formulations provided herein can be in liquid or dry form such as a dry powder or dry cake. The dry powder or dry cake may have undergone primary drying under, for example, lyophilization conditions or optionally, the dry cake or dry powder may have undergone both primary drying only or both primary drying and secondary drying. In the dry form, the powder or cake may, for example, have between 1% to 6% moisture, for example, such as between 2% to 5% moisture or between 2% to 4% moisture. One example method of drying is lyophilization (also called freeze-drying, or cyrodessication). Any of the compositions and methods of the disclosure may include liposomes, lyophilized liposomes or liposomes reconstituted from lyophilized liposomes. In some embodiments, the disclosed compositions and methods include one or more lyoprotectants or cryoprotectants. These protectants are typically polyhydroxy compounds such as sugars (mono-, di-, and polysaccharides), polyalcohols, and their derivatives, glycerol, or polyethyleneglycol, trehalose, maltose, sucrose, glucose, lactose, dextran, glycerol, or aminoglycosides. In further embodiments, the lyoprotectants or cryoprotectants comprise up to 10% or up to 20% of a solution outside the liposome, inside the liposome, or both outside and inside the liposome.

In some embodiments, the liposomes include a steric stabilizer that increases their longevity in circulation. One or more steric stabilizers such as a hydrophilic polymer (Polyethylene glycol (PEG)), a glycolipid (monosialoganglioside (GM1)) or others occupies the space immediately adjacent to the liposome surface and excludes other macromolecules from this space. Consequently, access and binding of blood plasma opsonins to the liposome surface are hindered, and thus interactions of macrophages with such liposomes, or any other clearing mechanism, are inhibited and longevity of the liposome in circulation is enhanced. In some embodiments, the steric stabilizer or the population of steric stabilizers is a PEG or a combination comprising PEG. In further embodiments, the steric stabilizer is a PEG or a combination comprising PEG with a number average molecular weight (Mn) of 200 to 5000 daltons. These PEG(s) can be of any structure such as linear, branched, star or comb structure and are commercially available.

In some embodiments, the liposomal composition comprises a liposome having a diameter in the range of 20 nm to 150 nm, or any range therein between. In some embodiments, the liposomal composition comprises a liposome that contains a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof, and has a diameter in the range of 20 nm to 150 nm. In further embodiments, the liposomal composition comprises a liposome having a diameter in the range of 30 nm to 150 nm, or any range therein between. In some embodiments, the liposomal composition comprises a liposome that contains a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof, and has a diameter in the range of 30 nm to 150 nm, or any range therein between. In further embodiments, the liposomal composition comprises a liposome having a diameter in the range of 80 nm to 120 nm, or any range therein between. In some embodiments, the liposomal composition comprises a liposome that contains a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof, and has a diameter in the range of 80 nm to 120 nm. In further embodiments, the liposomal composition comprises a liposome having a diameter in the range of 40 nm to 70 nm, or any range therein between. In some embodiments, liposomes comprise a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof, and have a diameter in the range of 40 nm-70 nm.

The properties of liposomes are influenced by the nature of lipids used to make the liposomes. A wide variety of lipids have been used to make liposomes. These include cationic, anionic and neutral lipids. In some embodiments, the liposomes comprising the polyglutamated Antifolate are anionic or neutral. In other embodiments, the provided liposomes are cationic. The determination of the charge (e.g., anionic, neutral or cationic) can routinely be determined by measuring the zeta potential of the liposome. The zeta potential of the liposome can be positive, zero or negative. In some embodiments, the zeta potential of the liposome is less than or equal to zero. In some embodiments, the zeta potential of the liposome is in a range of 0 to −150 mV. In another embodiment, the zeta potential of the liposome is in the range of −30 to −50 mV.

In some embodiments, cationic lipids are used to make cationic liposomes which are commonly used as gene transfection agents. The positive charge on cationic liposomes enables interaction with the negative charge on cell surfaces. Following binding of the cationic liposomes to the cell, the liposome is transported inside the cell through endocytosis.

In some preferred embodiments, a neutral to anionic liposome is used. In a preferred embodiment, an anionic liposome is used. Using a mixture of, for example, neutral lipids such as HSPC and anionic lipids such as PEG-DSPE results in the formation of anionic liposomes which are less likely to non-specifically bind to normal cells. Specific binding to tumor cells can be achieved by using a tumor targeting antibody such as, for example, a folate receptor antibody, including, for example, folate receptor alpha antibody, folate receptor beta antibody and/or folate receptor delta antibody.

As an example, at least one (or some) of the lipids is/are amphipathic lipids, defined as having a hydrophilic and a hydrophobic portion (typically a hydrophilic head and a hydrophobic tail). The hydrophobic portion typically orients into a hydrophobic phase (e.g., within the bilayer), while the hydrophilic portion typically orients toward the aqueous phase (e.g., outside the bilayer). The hydrophilic portion can comprise polar or charged groups such as carbohydrates, phosphate, carboxylic, sulfato, amino, sulfhydryl, nitro, hydroxy and other like groups. The hydrophobic portion can comprise apolar groups that include without limitation long chain saturated and unsaturated aliphatic hydrocarbon groups and groups substituted by one or more aromatic, cyclo-aliphatic or heterocyclic group(s). Examples of amphipathic compounds include, but are not limited to, phospholipids, aminolipids and sphingolipids.

Typically, for example, the lipids are phospholipids. Phospholipids include without limitation phosphatidylcholine, phosphatidylethanolamine, phosphatidylglycerol, phosphatidylinositol, phosphatidylserine, and the like. It is to be understood that other lipid membrane components, such as cholesterol, sphingomyelin, and cardiolipin, can be used.

The lipids comprising the liposomes provided herein can be anionic and neutral (including zwitterionic and polar) lipids including anionic and neutral phospholipids. Neutral lipids exist in an uncharged or neutral zwitterionic form at a selected pH. At physiological pH, such lipids include, for example, dioleoylphosphatidylglycerol (DOPG), diacylphosphatidylcholine, diacylphosphatidylethanolamine, ceramide, sphingomyelin, cephalin, cholesterol, cerebrosides and diacylglycerols. Examples of zwitterionic lipids include without limitation dioleoylphosphatidylcholine (DOPC), dimyristoylphos-phatidylcholine (DMPC), and dioleoylphosphatidylserine (DOPS). Anionic lipids are negatively charged at physiological pH. These lipids include without limitation phosphatidylglycerol, cardiolipin, diacylphosphatidylserine, diacylphosphatidic acid, N-dode-canoyl phosphatidylethanolamines, N-succinyl phosphatidylethanolamines, N-glutarylphosphatidylethanolamines, lysylphosphatidylglycerols, palmitoyloleyolphos-phatidylglycerol (POPG), and other anionic modifying groups joined to neutral lipids.

Collectively, anionic and neutral lipids are referred to herein as non-cationic lipids. Such lipids may contain phosphorus but they are not so limited. Examples of non-cationic lipids include lecithin, lysolecithin, phosphatidylethanolamine, lysophosphatidylethan-olamine, dioleoylphosphati-dylethanolamine (DOPE), dipalmitoyl phosphatidyl ethanol-amine (DPPE), dimyristoylphosphoethanolamine (DMPE), distearoyl-phosphatidy 1-ethan-olamine (DSPE), palmitoyloleoyl-phosphatidylethanolamine (POPE) palmitoyl-oleoylphosphatidylcholine (POPC), egg phosphatidylcholine (EPC), distearoylphosphat-idylcholine (DSPC), dioleoylphosphatidylcholine (DOPC), dipalmitoylphospha-tidylcholine (DPPC), dioleoylphosphatidylglycerol (DOPG), dipalmitoylphospha-tidylglycerol (DPPG), palmitoyloleyolphosphatidylglycerol (POPG), 16-0-monomethyl PE, 16-0-dimethyl PE, 18-1-trans PE, palmitoyloleoyl-phosphatidylethanolamine (POPE), 1-stearoyl-2-oleoylphosphatidyethanolamine (SOPE), phosphatidylserine, phosphatidyl-inositol, sphingomyelin, cephalin, cardiolipin, phosphatidic acid, cerebrosides, dicetyl-phosphate, and cholesterol.

The liposomes may be assembled using any liposomal assembly method using liposomal components (also referred to as liposome components) known in the art. Liposomal components include, for example, lipids such as DSPE, HSPC, cholesterol and derivatives of these components. Other suitable lipids are commercially available for example, by Avanti Polar Lipids, Inc. (Alabaster, Ala., USA). A partial listing of available negatively or neutrally charged lipids suitable for making anionic liposomes, can be, for example, at least one of the following: DLPC, DMPC, DPPC, DSPC, DOPC, DMPE, DPPE, DOPE, DMPA.Na, DPPA.Na, DOPA.Na, DMPG.Na, DPPG.Na, DOPG.Na, DMPS.Na, DPPS.Na, DOPS.Na, DOPE-Glutaryl.(Na)2, Tetramyristoyl Cardiolipin.(Na)2, DSPE-mPEG-2000.Na, DSPE-mPEG-5000.Na, and DSPE-Maleimide PEG-2000.Na.

In some embodiments, the PANTIFOL compositions provided herein are formulated in a liposome comprising a cationic lipid. In one embodiment, the cationic lipid is selected from, but not limited to, a cationic lipid described in Intl. Appl. Publ. Nos. WO2012/040184, WO2011/153120, WO2011/149733, WO2011/090965, WO2011/043913, WO2011/022460, WO2012/061259, WO2012/054365, WO2012/044638, WO2010/080724, WO2010/21865 and WO2008/103276, U.S. Pat. Nos. 7,893,302, 7,404,969 and 8,283,333 and US Appl. Publ. Nos. US20100036115 and US20120202871; each of which is herein incorporated by reference in their entirety. In another embodiment, the cationic lipid may be selected from, but not limited to, Formula A described in Intl. Appl. Publ. Nos. WO2012/040184, WO2011/153120, WO201/1149733, WO2011/090965, WO2011/043913, WO2011/022460, WO2012/061259, WO2012/054365 and WO2012/044638; each of which is herein incorporated by reference in their entirety. In yet another embodiment, the cationic lipid may be selected from, but not limited to, Formula CLI-CLXXIX of International Publication No. WO2008103276, Formula CLI-CLXXIX of U.S. Pat. No. 7,893,302, Formula CLI-CLXXXXII of U.S. Pat. No. 7,404,969 and Formula I-VI of US Patent Publication No. US20100036115; each of which is herein incorporated by reference in their entirety. As a non-limiting example, the cationic lipid may be selected from (20Z,23Z)—N,N-dimethylnonacosa-20,23-dien-10-amine, (17Z,20Z)—N,N-dimemyl-hexacosa-17,20-dien-9-amine, (1Z,19Z)—N5N-dimethylpenta cosa-16, 19-dien-8-amine, (13Z,16Z)—N,N-dimethyldocosa-13,16-dien-5-amine, (12Z, 15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyl-tricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexa-cosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyl-triaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methyl-nonacosa-20,23-dien-10-amine, 1-[(11Z,14Z)-1-nonylicosa-11,14-dien-1-yl] pyrrolidine, (20Z)—N,N-dimethyl-heptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethyl-nonacos-20-en-10-amine, (22Z)—N,N-dimethylhentriacont-22-en-10-amine, (16Z)—N,N-dimethylpenta-cos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclo-propyl] eptadecan-8-amine, 1-[(1S,2R)-2-hexylcyclo-propyl]-N,N-dimethyl nonadecan-10-amine, N,N-dimethyl-1-[(1S,2R)-2-octylcyclo-propyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcyclopropyl]methyl}cyclo-propyl]nonadecan-10-amine,N,N-dimethyl-1-[(1S,2R)-2-octylcyclopropyl]hexadecan-8-amine, N,N-dimethyl-[(1R,2S)-2-undecyl-cyclopropyl]tetradecan-5-amine, N,N-dimethyl-3-{17-[(1S, 2R)-2-octylcyclopropyl]heptyl} dodecan-1-amine, 1-[(1R,2S)-2-heptylcyclopropyl]-N,N-dimethyloctadecan-9-amine, 1-[(1S,2R)-2-decylcyclopropyl]-N,N-dimethyl-penta-decan-6-amine, N,N-dimethyl-1-[(1S,2R)-2-octylcyclopropyl] pentadecan-8-amine, R—N,N-dimethyl-1-[(9Z,12Z)-octadeca-9,12-dien-1-yloxy]-3-(octyloxy)propa-n-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy) methyl]ethyl}pyrrolidine, (2S)—N,N-dimethyl-1-[(9Z,12Z)-octadeca-9,12-dien-1-yloxy]-3-[(5Z-)-oct-5-en-1-yloxy] propan-2-amine, 1-{2-[(9Z,12Z)-octadeca-9,12-dien-1-yloxy]-1-[(octyloxy)methyl] ethyl} azetidine, (2S)-1-(hexyloxy)-N,N-dimethyl-3-[(9Z,12Z)-octadeca-9,12-dien-1-ylo-xy]propan-2-amine, (2S)-1-(heptyloxy)-N,N-dimethyl-3-[(9Z,12Z)-octadeca-9,12-dien-1-yloxy]pr-opan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy) propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)pro-pan-2-amine, (2S)-1-(hexyloxy)-3-[(11Z,14Z)-icosa-11,14-dien-1-yloxy]-N,N-dimethylprop-an-2-amine, 1-[(11Z,14Z)-icosa-11,14-dien-1-yloxy]-N,N-dimethyl 1-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z, 16Z)-docosa-13,16-dien-1-yloxy]-3-(hexyloxy)-N,N-dime-thyl-propan-2-amine, (2S)-1-[(13Z)-docos-13-en-1-yloxy]-3-(hexyloxy)-N,N-dimethyl propan-2-amine, 1-[(13Z)-docos-13-en-1-yloxy]-N,N-dimethyl-3-(octyloxy) propan-2-amine, 1-[(9Z)-hexadec-9-en-1-yloxy]-N,N-dimethyl-3-(octyloxy) propan-2-amine, (2R)—N,N-dimethyl-H(1-metoyloctyl)oxy]-3-[(9Z,12Z)-octa-deca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]-methyl}cyclopropyl] octyl} oxy) propan-2-amine, N,N-dimethyl-1-{[-(2-oclylcyclopropyl)octyl]oxy}-3-(octyloxy) propan-2-amine and (11E,20Z,23Z)—N,N-dimethylnonacosa-11,20,2-trien-10-amine or a pharmaceutically acceptable salt or acid or stereoisomer thereof.

In one embodiment, the lipid may be a cleavable lipid such as those described in in Intl. Publ. No. WO2012/170889, which is herein incorporated by reference in its entirety

The cationic lipid can routinely be synthesized using methods known in the art and/or as described in Intl. Publ. Nos. WO2012/040184, WO2011/153120, WO2011/149733, WO2011/090965, WO201/1043913, WO2011/022460, WO2012/061259, WO2012/054365, WO2012/044638, WO2010/080724 and WO2010/21865; each of which is herein incorporated by reference in its entirety.

Lipid derivatives can include, for example, at least, the bonding (preferably covalent bonding) of one or more steric stabilizers and/or functional groups to the liposomal component after which the steric stabilizers and/or functional groups should be considered part of the liposomal components. Functional groups comprises groups that can be used to attach a liposomal component to another moiety such as a protein. Such functional groups include, at least, maleimide. These steric stabilizers include at least one from polyethylene glycol (PEG); poly-L-lysine (PLL); monosialoganglioside (GM1); poly(vinyl pyrrolidone) (PVP); poly(acrylamide) (PAA); poly(2-methyl-2-oxazoline); poly(2-ethyl-2-oxazoline); phosphatidyl polyglycerol; poly[N-(2-hydroxy-propyl) methacrylamide]; amphiphilic poly-N-vinylpyrrolidones; L-amino-acid-based polymer; and polyvinyl alcohol.

In some embodiments, the PANTIFOL compositions are formulated in a lipid-polycation complex. The formation of the lipid-polycation complex may be accomplished using methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012/013326; herein incorporated by reference in its entirety. In another embodiment, the PANTIFOL is formulated in a lipid-polycation complex which further includes a neutral lipid such as, but not limited to, cholesterol or dioleoyl phosphatidylethanolamine (DOPE).

Since the components of a liposome can include any molecule(s) (e.g., chemical/reagent/protein) that is bound to it, in some embodiments, the components of the provided liposomes include, at least, a member selected from the group DSPE, DSPE-PEG, DSPE-maleimide, HSPC; HSPC-PEG; HSPC-maleimide; cholesterol; cholesterol-PEG; and cholesterol-maleimide. In some embodiments, the components of the provided liposomes include DSPE, DSPE-PEG, DSPE-maleimide, HSPC; HSPC-PEG; HSPC-maleimide; cholesterol; cholesterol-PEG; and cholesterol-maleimide. In a preferred embodiment, the liposomal components that make up the liposome comprises DSPE; DSPE-FITC; DSPE-maleimide; cholesterol; and HSPC.

In additional embodiments, the liposomes of the liposome compositions provided herein comprise oxidized phospholipids. In some embodiments, the liposomes comprise an oxidize phospholipid of a member selected from phosphatidylserines, phosphatidylinositols, phosphatidylethanolamines, phosphatidyl-cholines and 1-palmytoyl-2-arachidonoyl-sn-glycero-2-phosphate. In some embodiments, the phospholipids have unsaturated bonds. In some embodiments, the phospholipids are arachidonic acid containing phospholipids. In additional embodiments, the phospholipids are sn-2-oxygenated. In additional embodiments, the phospholipids are not fragmented.

In some embodiments, the liposomes of the disclosed liposome compositions comprise oxidized 1-palmitoyl-2-arachidonoyl-sn-glycero-3-phosphorylcholine (OxPAPC). The term “oxPAPC”, as used herein, refers to lipids generated by the oxidation of 1-palmitoyl-2-arachidonyl-sn-glycero-3-phosphorylcholine (PAPC), which results in a mixture of oxidized phospholipids containing either fragmented or full length oxygenated sn-2 residues. Well-characterized oxidatively fragmented species contain a five-carbon sn-2 residue bearing omega-aldehyde or omega-carboxyl groups. Oxidation of arachidonic acid residue also produces phospholipids containing esterified isoprostanes. In some embodiments, the oxPAPC includes HOdiA-PC, KOdiA-PC, HOOA-PC and KOOA-PC species, among other oxidized products present in oxPAPC. In further embodiments, the oxPAPCs are epoxyisoprostane-containing phospholipids. In further embodiments, the oxPAPC is 1-palmitoyl-2-(5,6-epoxyisoprostane E2)-sn-glycero-3-phosphocholine (5,6-PEIPC), 1-palmitoyl-2-(epoxy-cyclo-pentenone)-sn-glycero-3-phosphorylcholine (PECPC) and/or 1-palmitoyl-2-(epoxy-isoprostane E2)-sn-glycero-4-phosphocholine (PEIPC). In some embodiments, the phospholipids have unsaturated bonds. In some embodiments, the phospholipids are arachidonic acid containing phospholipids. In additional embodiments, the phospholipids are sn-2-oxygenated. In additional embodiments, the phospholipids are not fragmented.

In some embodiments, the liposomal polyglutamated Antifolate composition is pegylated (i.e., a pegylated liposomal gamma polyglutamated (e.g., pentaglutamated or hexaglutamated) antifolate (PLp-PANTIFOL or TPLp-PANTIFOL). In some embodiments, the PLp-PANTIFOL or TPLp-PANTIFOL is water soluble. That is, the PLp-PANTIFOL or TPLp-PANTIFOL is in the form an aqueous solution.

In some embodiments, the liposomes of the disclosed liposome compositions comprise a lipid selected from: 1-palmitoyl-2-glutaroyl-sn-glycero-3-phosphocholine (PGPC); 1-palmitoyl-2-(9′oxo-nonanoyl)-sn-glycero-3-phosphocholine; 1-palmitoyl-2-arachinodoyl-sn-glycero-3-phosphocholine; 1-palmitoyl-2-myristoyl-sn-glycero-3-phosphocholine; 1-palmitoyl-2-hexadecyl-sn-glycero-3-phosphocholine; 1-palmitoyl-2-azelaoyl-sn-glycero-3-phosphocholine; and 1-palmitoyl-2-acetoyl-sn-glycero-3-phospho-choline. In further embodiments, the liposome comprises PGPC.

In some embodiments, the pH of solutions comprising the liposome composition is from pH 2 to 8, or any range therein between. In some embodiments, the pH of solutions comprising the liposome composition is from pH 5 to 8 or from pH 2 to 6, or any range therein between. In some embodiments, the pH of solutions comprising the liposome composition is from pH 5 to 8, or any range therein between. In some embodiments, the pH of solutions comprising the liposome composition is from pH 6 to 7, or any range therein between. In some embodiments, the pH of solutions comprising the liposome composition is from 6 to 7.5, from 6.5 to 7.5, from 6.7 to 7.5, or from 6.3 to 7.0, or any range therein between.

In some embodiments, at least one component of the liposome lipid bilayer is functionalized (or reactive). As used herein, a functionalized component is a component that comprises a reactive group that can be used to crosslink reagents and moieties to the lipid. If the lipid is functionalized, any liposome that it forms is also functionalized. In some embodiments, the reactive group is one that will react with a crosslinker (or other moiety) to form crosslinks. The reactive group in the liposome lipid bilayer is located anywhere on the lipid that allows it to contact a crosslinker and be crosslinked to another moiety (e.g., a steric stabilizer or targeting moiety). In some embodiments, the reactive group is in the head group of the lipid, including for example a phospholipid. In some embodiments, the reactive group is a maleimide group. Maleimide groups can be crosslinked to each other in the presence of dithiol crosslinkers including but not limited to dithiolthrietol (DTT).

It is to be understood that the use of other functionalized lipids, other reactive groups, and other crosslinkers beyond those described above is further contemplated. In addition to the maleimide groups, other examples of contemplated reactive groups include but are not limited to other thiol reactive groups, amino groups such as primary and secondary amines, carboxyl groups, hydroxyl groups, aldehyde groups, alkyne groups, azide groups, carbonyls, halo acetyl (e.g., iodoacetyl) groups, imidoester groups, N-hydroxysuccinimide esters, sulfhydryl groups, and pyridyl disulfide groups.

Functionalized and non-functionalized lipids are available from a number of commercial sources including Avanti Polar Lipids (Alabaster, Ala.) and Lipoid LLC (Newark, N.J.).

### Liposome Interior Space

In further non-limiting embodiments, the provided liposomes enclose an interior space. In some embodiments, the interior space comprises, but is not limited to, an aqueous solution. In some embodiments, the interior space comprises a polyglutamated Antifolate as provided herein. In additional embodiments, the interior space of the liposome comprises a tonicity agent. In some embodiments. In some embodiments, the concentration (weight percent) of the tonicity agent is 0.1-20%, 1-20%, 0.5-15%, 1-15%, or 1-50%, or any range therein between. In some embodiments, the interior space of the liposome includes a sugar (e.g., trehalose, maltose, sucrose, lactose, mannose, mannitol, glycerol, dextrose, fructose, etc.). In further embodiments, the concentration (weight percent) of the sugar is 0.1-20%, 1-20%, 0.5-15%, 1%-15%, or 1-50%, or any range therein between. In some embodiments, the pH of the interior space of the liposome is from pH 2 to 8, or any range therein between. In some embodiments, the pH of solutions comprising the liposome composition is from pH 5 to 8, or any range therein between. In some embodiments, the pH of solutions comprising the liposome composition is from pH 6 to 7, or any range therein between. In some embodiments, the pH of solutions comprising the liposome composition is from 6 to 7.5, from 6.5 to 7.5, from 6.7 to 7.5, or from 6.3 to 7.0, or any range therein between. In some embodiments, the interior space comprises buffer. In further embodiments, the buffer a buffer selected from HEPES, citrate, or sodium phosphate (e.g., monobasic and/or dibasic sodium phosphate). In some embodiments, the buffer is HEPES. In some embodiments, the buffer is citrate. In some embodiments, the buffer is sodium phosphate (e.g., monobasic and/or dibasic sodium phosphate). In some embodiments, the buffer is at a concentration of 15 to 200 mM, or any range therein between. In further embodiments, the buffer is at a concentration of between 5 to 200 mM, 15-200, between 5 to 100 mM, between 15 to 100 mM, between 5 to 50 mM, between 15 to 50 mM, between 5 to 25 mM, between 5 to 20 mM, between 5 to 15 mM, or any range therein between. In some embodiments, the buffer is HEPES at a concentration of 15 to 200 mM, or any range therein between. In some embodiments, the buffer is citrate at a concentration of 15 to 200 mM, or any range therein between. In some embodiments, the buffer is sodium phosphate at a concentration of 15 to 200 mM, or any range therein between. In some embodiments, the interior space of the liposome comprises a total concentration of sodium acetate and calcium acetate of between 5 mM to 500 mM, or 50 mM to 500 mM, or any range therein between.

In some embodiments, the interior space of the liposome includes glutamine, glutamate, and/or polyglutamate (e.g., diglutamate, triglutamate, tetraglutamate, and/or pentaglutamate, containing one or more gamma glutamyl group linkages, or 1 or more alpha glutamyl linkages). In further embodiments, the concentration weight percent of the glutamine, glutamate, and/or polyglutamate is 0.1-20%, 1-20%, 0.5-15%, 1%-15%, 5-20%, or 1-50%, or any range therein between. In some embodiments the interior space of the liposome includes glutamine. In some embodiments the interior space of the liposome includes glutamate. In some embodiments the interior space of the liposome includes polyglutamate. In some embodiments, the concentration (weight percent) of glutamine, glutamate, and/or polyglutamate is 1-15%, or any range therein between. In an additional embodiment, the glutamine, glutamate, and/or polyglutamate is present at about 5% to 20% weight percent of the glutamine, glutamate, and/or polyglutamate or any combination of one or more lyoprotectants or cryoprotectants at a total concentration of 5% to 20%. In some embodiments, the interior space comprises buffer. In further embodiments, the buffer is HEPES buffer or citrate buffer. In further embodiments, the citrate buffer is at a concentration of between 5 to 200 mM. In some embodiments, the interior space has a pH of between 2.8 to 6. In some embodiments, the pH of solutions comprising the liposome composition is from 6 to 7.5, from 6.5 to 7.5, from 6.7 to 7.5, or from 6.3 to 7.0, or any range therein between. In some embodiments, the interior space comprises buffer. In some embodiments, the buffer is selected from HEPES, citrate, or sodium phosphate (e.g., monobasic and/or dibasic sodium phosphate). In some embodiments, the buffer is HEPES. In some embodiments, the buffer is citrate. In some embodiments, the buffer is sodium phosphate (e.g., monobasic and/or dibasic sodium phosphate). In some embodiments, the buffer is at a concentration of 15 to 200 mM, or any range therein between. In further embodiments, the buffer is at a concentration of between 5 to 200 mM, 15-200, between 5 to 100 mM, between 15 to 100 mM, between 5 to 50 mM, between 15 to 50 mM, between 5 to 25 mM, between 5 to 20 mM, between 5 to 15 mM, or any range therein between. In some embodiments, the buffer is HEPES at a concentration of 15 to 200 mM, or any range therein between. In some embodiments, the buffer is citrate at a concentration of 15 to 200 mM, or any range therein between. In some embodiments, the buffer is sodium phosphate at a concentration of 15 to 200 mM, or any range therein between. In additional embodiments, the interior space of the liposome comprises sodium acetate and/or calcium acetate. In some embodiments, the interior space of the liposome comprises a total concentration of sodium acetate and calcium acetate of between 5 mM to 500 mM, or 50 mM to 500 mM, or any range therein between. In some embodiments, the interior space of the liposome comprises a total concentration of sodium acetate and calcium acetate of between 50 mM to 500 mM.

In some embodiments, the interior space of the liposome includes glutamine. In further embodiments, the concentration weight percent of the glutamine is 0.1-20%, 1-20%, 0.5-15%, 1%-15%, 5-20%, or 1-50%, or any range therein between. In some embodiments, the concentration (weight percent) of glutamine is 1-15%, or any range therein between. In an additional embodiment, the glutamine is present at about 5% to 20% weight percent of glutamine or any combination of one or more lyoprotectants or cryoprotectants at a total concentration of 5% to 20%. In some embodiments, the interior space comprises buffer. In further embodiments, the buffer is HEPES buffer or citrate buffer. In further embodiments, the citrate buffer is at a concentration of between 5 to 200 mM. In some embodiments, the interior space has a pH of between 2.8 to 6. In some embodiments, the pH of solutions comprising the liposome composition is from 6 to 7.5, from 6.5 to 7.5, from 6.7 to 7.5, or from 6.3 to 7.0, or any range therein between. In some embodiments, the interior space comprises buffer. In some embodiments, the buffer is selected from HEPES, citrate, or sodium phosphate (e.g., monobasic and/or dibasic sodium phosphate). In some embodiments, the buffer is HEPES. In some embodiments, the buffer is citrate. In some embodiments, the buffer is sodium phosphate (e.g., monobasic and/or dibasic sodium phosphate). In some embodiments, the buffer is at a concentration of 15 to 200 mM, or any range therein between. In further embodiments, the buffer is at a concentration of between 5 to 200 mM, 15-200, between 5 to 100 mM, between 15 to 100 mM, between 5 to 50 mM, between 15 to 50 mM, between 5 to 25 mM, between 5 to 20 mM, between 5 to 15 mM, or any range therein between. In some embodiments, the buffer is HEPES at a concentration of 15 to 200 mM, or any range therein between. In some embodiments, the buffer is citrate at a concentration of 15 to 200 mM, or any range therein between. In some embodiments, the buffer is sodium phosphate at a concentration of 15 to 200 mM, or any range therein between. In additional embodiments, the interior space of the liposome comprises sodium acetate and/or calcium acetate. In some embodiments, the interior space of the liposome comprises a total concentration of sodium acetate and calcium acetate of between 5 mM to 500 mM, or 50 mM to 500 mM, or any range therein between. In some embodiments, the interior space of the liposome comprises a total concentration of sodium acetate and calcium acetate of between 50 mM to 500 mM.

In some embodiments, the interior space of the liposome includes trehalose. In further embodiments, the concentration weight percent of trehalose is 0.1-20%, 1-20%, 0.5-15%, 1%-15%, 5-20%, or 1-50%, or any range therein between. In further embodiments, the concentration (weight percent) of trehalose is 1-15%, or any range therein between. In an additional embodiment, the trehalose is present at about 5% to 20% weight percent of trehalose or any combination of one or more lyoprotectants or cryoprotectants at a total concentration of 5% to 20%. In some embodiments, the interior space comprises buffer. In further embodiments, the buffer is HEPES buffer or citrate buffer. In further embodiments, the citrate buffer is at a concentration of between 5 to 200 mM. In some embodiments, the interior space has a pH of between 2.8 to 6. In some embodiments, the pH of solutions comprising the liposome composition is from 6 to 7.5, from 6.5 to 7.5, from 6.7 to 7.5, or from 6.3 to 7.0, or any range therein between. In some embodiments, the interior space comprises buffer. In some embodiments, the buffer is selected from HEPES, citrate, or sodium phosphate (e.g., monobasic and/or dibasic sodium phosphate). In some embodiments, the buffer is HEPES. In some embodiments, the buffer is citrate. In some embodiments, the buffer is sodium phosphate (e.g., monobasic and/or dibasic sodium phosphate). In some embodiments, the buffer is at a concentration of 15 to 200 mM, or any range therein between. In further embodiments, the buffer is at a concentration of between 5 to 200 mM, 15-200, between 5 to 100 mM, between 15 to 100 mM, between 5 to 50 mM, between 15 to 50 mM, between 5 to 25 mM, between 5 to 20 mM, between 5 to 15 mM, or any range therein between. In some embodiments, the buffer is HEPES at a concentration of 15 to 200 mM, or any range therein between. In some embodiments, the buffer is citrate at a concentration of 15 to 200 mM, or any range therein between. In some embodiments, the buffer is sodium phosphate at a concentration of 15 to 200 mM, or any range therein between. In additional embodiments, the interior space of the liposome comprises sodium acetate and/or calcium acetate. In some embodiments, the interior space of the liposome comprises a total concentration of sodium acetate and calcium acetate of between 5 mM to 500 mM, or 50 mM to 500 mM, or any range therein between. In some embodiments, the interior space of the liposome comprises a total concentration of sodium acetate and calcium acetate of between 50 mM to 500 mM.

In some embodiments, the interior space of the liposome includes dextrose. In further embodiments, the concentration weight percent of dextrose is 0.1-20%, 1-20%, 0.5-15%, 1-15%, 5-20%, or 1-50%, or any range therein between. In yet further embodiments, the concentration (weight percent) of dextrose is 1-15%, or any range therein between. In an additional embodiment, the dextrose is present at about 5% to 20% weight percent of dextrose or any combination of one or more lyoprotectants or cryoprotectants at a total concentration of 5% to 20%. In some embodiments, the pH of solutions comprising the liposome composition is from 6 to 7.5, from 6.5 to 7.5, from 6.7 to 7.5, or from 6.3 to 7.0, or any range therein between. In some embodiments, the interior space comprises buffer. In some embodiments, the buffer is selected from HEPES, citrate, or sodium phosphate (e.g., monobasic and/or dibasic sodium phosphate). In some embodiments, the buffer is HEPES. In some embodiments, the buffer is citrate. In some embodiments, the buffer is sodium phosphate (e.g., monobasic and/or dibasic sodium phosphate). In some embodiments, the buffer is at a concentration of 15 to 200 mM, or any range therein between. In yet further embodiments, the buffer is at a concentration of between 5 to 200 mM, 15-200, between 5 to 100 mM, between 15 to 100 mM, between 5 to 50 mM, between 15 to 50 mM, between 5 to 25 mM, between 5 to 20 mM, between 5 to 15 mM, or any range therein between. In some embodiments, the buffer is HEPES at a concentration of 15 to 200 mM, or any range therein between. In some embodiments, the buffer is citrate at a concentration of 15 to 200 mM, or any range therein between. In some embodiments, the buffer is sodium phosphate at a concentration of 15 to 200 mM, or any range therein between. In additional embodiments, the interior space of the liposome comprises sodium acetate and/or calcium acetate. In some embodiments, the interior space of the liposome comprises a total concentration of sodium acetate and calcium acetate of between 5 mM to 500 mM, or 50 mM to 500 mM, or any range therein between.

In additional embodiments, the disclosure provides liposomal compositions that comprise a liposome encapsulating (i.e., filled with) a polyglutamated Antifolate (e.g., a γPANTIFOL or αPANTIFOL disclosed herein). In some embodiments, the liposomal composition comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the disclosure provides a liposomal composition comprising a targeted and pegylated liposome that comprises a polyglutamated Antifolate (TPLp-PANTIFOL). In some embodiments, the liposomal composition comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the targeted pegylated liposomal polyglutamated (e.g., pentaglutamated or hexaglutamated) Antifolate comprises a medium comprising a liposome including an interior space; an aqueous polyglutamated Antifolate disposed within the interior space; and a targeting moiety comprising a protein with specific affinity for at least one folate receptor, and wherein the targeting moiety disposed at the exterior of the liposome. In some embodiments, the medium is an aqueous solution. In some embodiments, the interior space, the exterior space (e.g., the medium), or both the interior space and the medium contains one or more lyoprotectants or cryoprotectants which are listed above. In some embodiments, the cryoprotectant is mannitol, trehalose, sorbitol, or sucrose.

In some embodiments, the liposome encapsulating polyglutamated Antifolate (i.e., Lp-PANTIFOL, including PLp-PANTIFOL, TPLp-PANTIFOL, TLp-PANTIFOL, and NTLp-PANTIFOL) has an interior space that contains less than 500,000 or less than 200,000 molecules of polyglutamated Antifolate (e.g., Formula III-1-L, III-1-D, or a pharmaceutically acceptable salt thereof, or Formula IV-1-L or IV-1-D, or Formula III-1-L-Alpha, III-1-D-Alpha, or a pharmaceutically acceptable salt thereof, or Formula IV-1-L-Alpha or IV-1-D-Alpha). In some embodiments, the liposome interior space contains between 10 to 100,000 molecules of polyglutamated Antifolate, or any range therein between. In some embodiments, the liposome interior space contains between 10,000 to 100,000 molecules of polyglutamated Antifolate, or any range therein between. In some embodiments, the liposome is not pegylated and has an interior space that contains less than 500,000 or less than 200,000 molecules of polyglutamated Antifolate. In some embodiments, the liposome is not pegylated and the interior space of the liposome contains between 10 to 100,000 molecules of polyglutamated Antifolate, or any range therein between. In further embodiments, the liposome is not pegylated and the interior space of the liposome contains between 10,000 to 100,000 molecules of polyglutamated Antifolate, or any range therein between. In some embodiments, the liposome comprises a targeting moiety, is not pegylated (TLp-PANTIFOL), and has an interior space that contains less than 500,000 or less than 200,000 molecules of polyglutamated Antifolate. In some embodiments, the liposome comprises a targeting moiety, is not pegylated, and the interior space of the liposome contains between 10 to 100,000 molecules of polyglutamated Antifolate, or any range therein between. In further embodiments, the liposome comprises a targeting moiety, is not pegylated, and the interior space of the liposome contains between 10,000 to 100,000 molecules of polyglutamated Antifolate, or any range therein between. In some embodiments, the liposome does not comprise a targeting moiety, is not pegylated, (NTLp-PANTIFOL) and has an interior space that contains less than 500,000 or less than 200,000 molecules of polyglutamated Antifolate. In some embodiments, the liposome does not comprise a targeting moiety, is not pegylated, and the interior space of the liposome contains between 10 to 100,000 molecules of polyglutamated Antifolate, or any range therein between. In further embodiments, the liposome does not comprise a targeting moiety, is not pegylated, and the interior space of the liposome contains between 10,000 to 100,000 molecules of polyglutamated Antifolate, or any range therein between.

In some embodiments, the liposome encapsulates polyglutamated Antifolate of the present disclosure containing 2-10 glutamyl groups (e.g., Lp-PANTIFOL, including PLp-PANTIFOL, TPLp-PANTIFOL, TLp-PANTIFOL, and NTLp-PANTIFOL) and has an interior space that contains less than 500,000 or less than 200,000 molecules of polyglutamated Antifolate containing 2-10 glutamyl groups. In some embodiments, the liposome interior space contains between 10 to 100,000 molecules of polyglutamated Antifolate containing 2-10 glutamyl groups, or any range therein between. In further embodiments, the liposome interior space contains between 10,000 to 100,000 molecules of polyglutamated Antifolate containing 2-10 glutamyl groups, or any range therein between. In some embodiments, the liposome is not pegylated and has an interior space that contains less than 500,000 or less than 200,000 molecules of polyglutamated Antifolate containing 2-10 glutamyl groups. In some embodiments, the liposome is not pegylated and the interior space of the liposome contains between 10 to 100,000 molecules of polyglutamated Antifolate containing 2-10 glutamyl groups, or any range therein between. In further embodiments, the liposome is not pegylated and the interior space of the liposome contains between 10,000 to 100,000 molecules of polyglutamated Antifolate containing 2-10 glutamyl groups, or any range therein between. In some embodiments, the liposome comprises a targeting moiety, is not pegylated (TLp-PANTIFOL) and has an interior space that contains less than 500,000 or less than 200,000 molecules of polyglutamated Antifolate containing 2-10 glutamyl groups. In some embodiments, the liposome comprises a targeting moiety, is not pegylated, and the interior space of the liposome contains between 10 to 100,000 molecules of polyglutamated Antifolate containing 2-10 glutamyl groups, or any range therein between. In further embodiments, the liposome comprises a targeting moiety, is not pegylated, and the interior space of the liposome contains between 10,000 to 100,000 molecules of polyglutamated Antifolate containing 2-10 glutamyl groups, or any range therein between. In some embodiments, the liposome is non-targeted and unpegylated (NTLp-PANTIFOL) and has an interior space that contains less than 500,000 or less than 200,000 molecules of polyglutamated Antifolate containing 2-10 glutamyl groups. In some embodiments, the liposome does not comprise a targeting moiety, is not pegylated, and the interior space of the liposome contains between 10 to 100,000 molecules of polyglutamated Antifolate containing 2-10 glutamyl groups, or any range therein between. In further embodiments, the liposome is non-targeted and unpegylated and the interior space of the liposome contains between 10,000 to 100,000 molecules of polyglutamated Antifolate containing 2-10 glutamyl groups, or any range therein between.

In some embodiments, the liposome encapsulates tetraglutamated Antifolate of the present disclosure (e.g., Lp-PANTIFOL, including PLp-PANTIFOL, TPLp-PANTIFOL, TLp-PANTIFOL, and NTLp-PANTIFOL) and has an interior space that contains less than 500,000 or less than 200,000 molecules of tetraglutamated Antifolate. In some embodiments, the liposome interior space contains between 10 to 100,000 molecules of tetraglutamated Antifolate, or any range therein between. In some embodiments, the liposome interior space contains between 10,000 to 100,000 molecules of tetraglutamated Antifolate, or any range therein between. In some embodiments, the liposome is not pegylated and has an interior space that contains less than 500,000 or less than 200,000 molecules of tetraglutamated Antifolate. In some embodiments, the liposome is not pegylated and the interior space of the liposome contains between 10 to 100,000 molecules of tetraglutamated Antifolate, or any range therein between. In further embodiments, the liposome is not pegylated and the interior space of the liposome contains between 10,000 to 100,000 molecules of tetraglutamated Antifolate, or any range therein between. In some embodiments, the liposome comprises a targeting moiety, is not pegylated (TLp-PANTIFOL) and has an interior space that contains less than 500,000 or less than 200,000 molecules of tetraglutamated Antifolate. In some embodiments, the liposome comprises a targeting moiety, is not pegylated, and the interior space of the liposome contains between 10 to 100,000 molecules of tetraglutamated Antifolate, or any range therein between. In further embodiments, the liposome comprises a targeting moiety, is not pegylated, and the interior space of the liposome contains between 10,000 to 100,000 molecules of tetraglutamated Antifolate, or any range therein between. In some embodiments, the liposome does not comprise a targeting moiety, is not pegylated, (NTLp-PANTIFOL) and has an interior space that contains less than 500,000 or less than 200,000 molecules of tetraglutamated Antifolate. In some embodiments, the liposome does not comprise a targeting moiety, is not pegylated, and the interior space of the liposome contains between 10 to 100,000 molecules of tetraglutamated Antifolate, or any range therein between. In further embodiments, the liposome does not comprise a targeting moiety, is not pegylated, and the interior space of the liposome contains between 10,000 to 100,000 molecules of tetraglutamated Antifolate, or any range therein between. In some embodiments, the tetraglutamated Antifolate is gamma tetraglutamated Antifolate. In some embodiments, the tetraglutamated Antifolate is alpha tetraglutamated Antifolate.

In some embodiments, the liposome encapsulates pentaglutamated Antifolate of the present disclosure (e.g., Lp-PANTIFOL, including PLp-PANTIFOL, TPLp-PANTIFOL, TLp-PANTIFOL, and NTLp-PANTIFOL) and has an interior space that contains less than 500,000 or less than 200,000 molecules of pentaglutamated Antifolate. In some embodiments, the liposome interior space contains between 10 to 100,000 molecules of pentaglutamated Antifolate, or any range therein between. In some embodiments, the liposome interior space contains between 10,000 to 100,000 molecules of pentaglutamated Antifolate, or any range therein between. In some embodiments, the liposome is not pegylated and has an interior space that contains less than 500,000 or less than 200,000 molecules of pentaglutamated Antifolate. In some embodiments, the liposome is not pegylated and the interior space of the liposome contains between 10 to 100,000 molecules of pentaglutamated Antifolate, or any range therein between. In further embodiments, the liposome is not pegylated and the interior space of the liposome contains between 10,000 to 100,000 molecules of pentaglutamated Antifolate, or any range therein between. In some embodiments, the liposome comprises a targeting moiety, is not pegylated (TLp-PANTIFOL) and has an interior space that contains less than 500,000 or less than 200,000 molecules of pentaglutamated Antifolate. In some embodiments, the liposome comprises a targeting moiety, is not pegylated, and the interior space of the liposome contains between 10 to 100,000 molecules of pentaglutamated Antifolate, or any range therein between. In further embodiments, the liposome comprises a targeting moiety, is not pegylated, and the interior space of the liposome contains between 10,000 to 100,000 molecules of pentaglutamated Antifolate, or any range therein between. In some embodiments, the liposome is non-targeted and unpegylated (NTLp-PANTIFOL) and has an interior space that contains less than 500,000 or less than 200,000 molecules of pentaglutamated Antifolate. In some embodiments, the liposome does not contain a targeting moiety and it not pegylated, and the interior space of the liposome contains between 10 to 100,000 molecules of pentaglutamated Antifolate, or any range therein between. In further embodiments, the liposome is non-targeted and unpegylated and the interior space of the liposome contains between 10,000 to 100,000 molecules of pentaglutamated Antifolate, or any range therein between. In some embodiments, the pentaglutamated Antifolate is gamma pentaglutamated Antifolate. In some embodiments, the pentaglutamated Antifolate is alpha pentaglutamated Antifolate.

In some embodiments, the liposome encapsulates hexaglutamated Antifolate of the present disclosure (i.e., Lp-PANTIFOL, including PLp-PANTIFOL, TPLp-PANTIFOL, TLp-PANTIFOL, and NTLp-PANTIFOL) and has an interior space that contains less than 500,000 or less than 200,000 molecules of hexaglutamated Antifolate. In some embodiments, the liposome interior space contains between 10 to 100,000 molecules of hexaglutamated Antifolate, or any range therein between. In further embodiments, the liposome interior space contains between 10,000 to 100,000 molecules of hexaglutamated Antifolate, or any range therein between. In some embodiments, the liposome is not pegylated and has an interior space that contains less than 500,000 or less than 200,000 molecules of hexaglutamated Antifolate. In some embodiments, the liposome is not pegylated and the interior space of the liposome contains between 10 to 100,000 molecules of hexaglutamated Antifolate, or any range therein between. In further embodiments, the liposome is not pegylated and the interior space of the liposome contains between 10,000 to 100,000 molecules of hexaglutamated Antifolate, or any range therein between. In some embodiments, the liposome comprises a targeting moiety, is not pegylated (TLp-PANTIFOL) and has an interior space that contains less than 500,000 or less than 200,000 molecules of hexaglutamated Antifolate. In some embodiments, the liposome comprises a targeting moiety, is not pegylated, and the interior space of the liposome contains between 10 to 100,000 molecules of hexaglutamated Antifolate, or any range therein between. In further embodiments, the liposome comprises a targeting moiety, is not pegylated, and the interior space of the liposome contains between 10,000 to 100,000 molecules of hexaglutamated Antifolate, or any range therein between. In some embodiments, the liposome is non-targeted and unpegylated (NTLp-PANTIFOL) and has an interior space that contains less than 500,000 or less than 200,000 molecules of hexaglutamated Antifolate. In some embodiments, the liposome does not comprise a targeting moiety, is not pegylated, and the interior space of the liposome contains between 10 to 100,000 molecules of hexaglutamated Antifolate, or any range therein between. In further embodiments, the liposome does not comprise a targeting moiety, is not pegylated, and the interior space of the liposome contains between 10,000 to 100,000 molecules of hexaglutamated Antifolate, or any range therein between. In some embodiments, the hexaglutamated Antifolate is gamma hexaglutamated Antifolate. In some embodiments, the hexaglutamated Antifolate is alpha hexaglutamated Antifolate.

In some embodiments, the disclosure provides a liposomal polyglutamated Antifolate composition wherein the liposome encapsulates polyglutamated Antifolate or a salt or acid thereof, and one or more aqueous pharmaceutically acceptable carriers. In some embodiments, the liposome interior space contains trehalose. In some embodiments, the liposome interior space contains 5% to 20% weight of trehalose. In some embodiments, the liposome interior space contains HBS at a concentration of between 1 to 200 mM and a pH of between 2 to 8. In some embodiments, liposome interior space has a pH 5-8, or any range therein between. In some embodiments, liposome interior space has a pH 6-7, or any range therein between. In some embodiments, the liposome interior space has a total concentration of sodium acetate and calcium acetate of between 50 mM to 500 mM, or any range therein between.

### Non-Polyglutamated Polyglutamatable Antifolates

In some embodiments, the liposome polyglutamated Antifolate (e.g., Lp-PANTIFOL, including PLp-PANTIFOL, TPLp-PANTIFOL, TLp-PANTIFOL, and NTLp-PANTIFOL) compositions comprise a PANTIFOL of the present disclosure and one or more non-polyglutamated, polyglutamatable antifolate compositions.

In some embodiments, the Lp-PANTIFOL (e.g., PLp-PANTIFOL, TPLp-PANTIFOL, TLp-PANTIFOL, and NTLp-PANTIFOL) comprises polyglutamated Antifolate e.g., a αPANTIFOL and/or γPANTIFOL of the present disclosure and the Antifolate (ANTIFOL). In some embodiments, the Lp-PANTIFOL (i.e., liposome polyglutamated Antifolate) comprises αPANTIFOL and/or gamma polyglutamated Antifolate of the present disclosure and a polyglutamatable antifolate selected from the group consisting of: methotrexate (MTX), pemetrexed (PMX), lometrexol (LMX), raltitrexed (RTX), pralatrexate, AG2034, GW1843, aminopterin, LY309887 and LY222306. In some embodiments, the Lp-PANTIFOL comprises αPANTIFOL and/or gamma polyglutamated Antifolate of the present disclosure and lometrexol. In some embodiments, the Lp-PANTIFOL comprises αPANTIFOL and/or gamma polyglutamated Antifolate of the present disclosure and pemetrexed. In some embodiments, the Lp-PANTIFOL comprises αPANTIFOL and/or gamma polyglutamated Antifolate of the present disclosure and leucovorin. In some embodiments, the Lp-PANTIFOL comprises αPANTIFOL and/or gamma polyglutamated Antifolate of the present disclosure and a triazine antifolate derivative (e.g., a sulphonyl fluoride triazine such as NSC 127755). In some embodiments, the Lp-PANTIFOL comprises αPANTIFOL and/or gamma polyglutamated Antifolate of the present disclosure and a serine hydroxymethyltransferase (SHMT2) inhibitor. In some embodiments, the SHMT2 inhibitor is an antifolate (e.g., a polyglutamatable or nonpolyglutamatable antifolate). In some embodiments, the SHMT2 inhibitor is an antifolate.

### Non-Polyglutamatable Antifolates

In some embodiments, the Lp-PANTIFOL (e.g., PLp-PANTIFOL, TPLp-PANTIFOL, TLp-PANTIFOL, and NTLp-PANTIFOL) comprises a αPANTIFOL and/or gamma polyglutamated Antifolate (e.g., a αPANTIFOL and/or γPANTIFOL of the present disclosure) and a so-called “non-polyglutamatable” antifolate. In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the liposome comprises a αPANTIFOL and/or gamma polyglutamated Antifolate of the present disclosure and a non-polyglutamatable antifolate that inhibits one or more enzymes in the folate cycle metabolic pathway. In further embodiments, the non-polyglutamatable antifolate inhibits one or more enzymes selected from: thymidylate synthase (TS), dihydrofolate reductase (DHFR), glycinamide ribonucleotide (GAR) transformylase, and aminoimidazole carboxamide ribonucleotide (AICAR) transformylase. In some embodiments, the liposome comprises a polyglutamated Antifolate of the present disclosure and a non-polyglutamatable antifolate that inhibits DHFR. In some embodiments, the liposome comprises a polyglutamated Antifolate of the present disclosure and a non-polyglutamatable antifolate that inhibits TS. In some embodiments, the liposome comprises a polyglutamated Antifolate of the present disclosure and a non-polyglutamatable antifolate that inhibits GAR or AICAR transformylase. In further embodiments, the non-polyglutamatable antifolate is selected from: trimetrexate (TMQ), piritrexim (BW301U), and talotrexin (PT523). In further embodiments, the non-polyglutamatable antifolate is selected from: nolatrexed (AG337), plevitrexed (ZD9331, BGC9331), and BGC 945 (ONX 0801), or a pharmaceutically acceptable salt thereof.

### Platinums

In some embodiments, the liposome comprises a polyglutamated Antifolate (e.g., Lp-PANTIFOL, such as e.g., PLp-PANTIFOL, TPLp-PANTIFOL, TLp-PANTIFOL, and NTLp-PANTIFOL) comprises a αPANTIFOL and/or gamma polyglutamated Antifolate (e.g., a αPANTIFOL and/or γPANTIFOL of the present disclosure) and a platinum-based chemotherapeutic agent or a salt or acid, thereof. In some embodiments, the polyglutamated Antifolate/platinum-based agent complex comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the Lp-PANTIFOL comprises a platinum-based chemotherapeutic agent selected from: cisplatin, carboplatin, and oxaliplatin, or a salt or acid thereof. In other embodiments, the Lp-PANTIFOL comprises an analog of a platinum-based chemotherapeutic agent selected from: cisplatin, carboplatin, or oxaliplatin, or a salt or acid thereof.

In some embodiments, the Lp-PANTIFOL comprises a αPANTIFOL and/or gamma polyglutamated Antifolate of the present disclosure and cisplatin or a salt or acid thereof. In some embodiments, the Lp-PANTIFOL comprises a αPANTIFOL and/or gamma polyglutamated Antifolate of the present disclosure and a cisplatin analog, or a salt or acid thereof.

In some embodiments, the Lp-PANTIFOL comprises a αPANTIFOL and/or gamma polyglutamated Antifolate of the present disclosure and carboplatin, or a salt or acid thereof. In some embodiments, the liposome comprises a αPANTIFOL and/or gamma polyglutamated Antifolate of the present disclosure and carboplatin analog, or a salt or acid thereof.

In some embodiments, the Lp-PANTIFOL comprises a αPANTIFOL and/or gamma polyglutamated Antifolate of the present disclosure and oxaliplatin, or a salt or acid thereof. In some embodiments, the liposome comprises a αPANTIFOL and/or gamma polyglutamated Antifolate of the present disclosure and an oxaliplatin analog, or a salt or acid thereof.

In some embodiments, the liposome comprises a αPANTIFOL and/or gamma polyglutamated Antifolate (e.g., a αPANTIFOL and/or γPANTIFOL of the present disclosure) and a platinum-based chemotherapeutic agent selected from: nedaplatin, heptaplatin, and lobaplatin, nedaplatin, heptaplatin, and lobaplatin or a salt or acid thereof. In some embodiments, the Lp-PANTIFOL comprises a αPANTIFOL and/or gamma polyglutamated Antifolate of the present disclosure and an analog of a platinum-based chemotherapeutic agent selected from: nedaplatin, heptaplatin, and lobaplatin, or a salt or acid thereof.

In some embodiments, the Lp-PANTIFOL comprises a αPANTIFOL and/or gamma polyglutamated Antifolate of the present disclosure and a platinum-based chemotherapeutic agent selected from: stratoplatin, paraplatin, platinol, cycloplatin, dexormaplatin, spiroplatin, picoplatin, triplatin, tetraplatin, iproplatin, ormaplatin, zeniplatin, platinum-triamine, traplatin, enloplatin, JM-216, 254-S, NK 121, CI-973, DWA 2114R, NDDP, and dedaplatin, or a salt or acid thereof. In some embodiments, the Lp-PANTIFOL comprises a αPANTIFOL and/or gamma polyglutamated Antifolate of the present disclosure and an analog of a platinum-based chemotherapeutic agent selected from: stratoplatin, paraplatin, platinol, cycloplatin, dexormaplatin, spiroplatin, picoplatin, triplatin, tetraplatin, iproplatin, ormaplatin, zeniplatin, platinum-triamine, traplatin, enloplatin, JM-216, 254-S, NK 121, CI-973, DWA 2114R, NDDP, and dedaplatin, or a salt or acid thereof.

In some embodiments, the liposome composition comprises liposomes that further contain one or more of an immunostimulatory agent, a detectable marker and a maleimide disposed on at least one of the PEG and the exterior of the liposome.

### Cyclodextrins

In additional embodiments, the liposome comprise a PANTIFOL (e.g., a γPANTIFOL and/or αPANTIFOL of the present disclosure) and a cyclodextrin (e.g., a cyclodextrin described herein). In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the PANTIFOL liposome is a targeted liposomal composition.

In some embodiments, the PANTIFOL liposome comprises a complex formed by a cyclodextrin and a therapeutic agent. In some embodiments, the therapeutic agent is a cytotoxic compound or a salt or acid thereof. In a further embodiment, the therapeutic agent is a chemotherapeutic agent or a salt or acid thereof. In another embodiment, the therapeutic agent is a platinum-based drug. In another embodiment, the therapeutic agent is a taxane-based drug. In further embodiments, the therapeutic agent of the cyclodextrin/therapeutic agent complex is selected from: gemcitabine, a gemcitabine-based therapeutic agent, doxorubicin, an antifolate, an antifolate-based chemotherapeutic, or a salt or acid, acid or free base form thereof. In some embodiments, the PANTIFOL liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the PANTIFOL liposome is a targeted liposomal composition. In additional embodiments, the molar ratio of cyclodextrin/therapeutic agent in the complex is in the range 1-10:1. In some embodiments, the molar ratio of PANTIFOL/therapeutic agent in the complex is 1:1, 2:1, 3:1, 4:1, 5:1, 6:1, 7:1, or 10:1. In some embodiments, the molar ratio of PANTIFOL/therapeutic agent in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of cyclodextrin/therapeutic agent in the complex is: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In additional embodiments, the cyclodextrin/platinum-based agent complex is encapsulated in a liposome (e.g., as described herein or otherwise known in the art).

In some embodiments, the PANTIFOL liposome comprises PANTIFOL and a cyclodextrin/platinum-based chemotherapeutic agent complex. In some embodiments, the platinum-based chemotherapeutic agent is selected from: cisplatin, carboplatin, and oxaliplatin, or a salt or acid thereof. In other embodiments, the cyclodextrin/platinum-based chemotherapeutic agent complex comprises an analog of a cisplatin, carboplatin, oxaliplatin, or a salt or acid thereof. In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the PANTIFOL liposome is a targeted liposomal composition. In some embodiments, the molar ratio of cyclodextrin/platinum-based agent in the complex is in the range 1-10:1. In some embodiments, the molar ratio of cyclodextrin/platinum-based agent in the complex is 1:1, 2:1, 3:1, 4:1, 5:1, 6:1, 7:1, or 10:1. In some embodiments, the molar ratio of cyclodextrin/platinum-based agent in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of cyclodextrin/platinum-based agent in the complex is: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In additional embodiments, the cyclodextrin/platinum-based agent complex is encapsulated in a liposome.

In some embodiments, the platinum-based chemotherapeutic agent is selected from: cisplatin, carboplatin, and oxaliplatin, or a salt or acid thereof. In other embodiments, the cyclodextrin/platinum-based chemotherapeutic agent complex comprises an analog of a cisplatin, carboplatin, oxaliplatin, or a salt or acid thereof. In some embodiments, the molar ratio of cyclodextrin/platinum-based agent in the complex is in the range 1-10:1. In some embodiments, the molar ratio of cyclodextrin/platinum-based agent in the complex is 1:1, 2:1, 3:1, 4:1, 5:1, 6:1, 7:1, or 10:1. In some embodiments, the molar ratio of cyclodextrin/platinum-based agent in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of cyclodextrin/platinum-based agent in the complex is: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1.

In further embodiments, the disclosure provides a complex containing cyclodextrin and cisplatin or a salt or acid thereof. In some embodiments, the molar ratio of cyclodextrin/cisplatin (or cisplatin salt or acid) in the complex is in the range 1-10:1. In some embodiments, the molar ratio of cyclodextrin/cisplatin (or cisplatin salt or acid) in the complex is 1:1, 2:1, 3:1, 4:1, 5:1, 6:1, 7:1, or 10:1. In some embodiments, the molar ratio of cyclodextrin/cisplatin (or cisplatin salt or acid) in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of cyclodextrin/cisplatin (or cisplatin salt or acid) in the complex is: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In additional embodiments, the cyclodextrin/cisplatin (or cisplatin salt or acid) complex is encapsulated in a liposome.

In another embodiment, the disclosure provides a complex containing cyclodextrin and carboplatin or a salt or acid thereof. In some embodiments, the molar ratio of cyclodextrin/carboplatin (or carboplatin salt or acid) in the complex is in the range 1-10:1. In some embodiments, the molar ratio of cyclodextrin/carboplatin (or carboplatin salt or acid) in the complex is 1:1, 2:1, 3:1, 4:1, 5:1, 6:1, 7:1, or 10:1. In some embodiments, the molar ratio of cyclodextrin/carboplatin (or carboplatin salt or acid) in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of cyclodextrin/carboplatin (or carboplatin salt or acid) in the complex is: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In additional embodiments, the cyclodextrin/carboplatin (or carboplatin salt or acid) complex is encapsulated in a liposome (e.g., as described herein or otherwise known in the art).

In another embodiment, the disclosure provides a complex containing cyclodextrin and oxaliplatin, or a salt or acid thereof. In some embodiments, the molar ratio of cyclodextrin/oxaliplatin (or oxaliplatin salt or acid) in the complex is in the range 1-10:1. In some embodiments, the molar ratio of cyclodextrin/oxaliplatin (or oxaliplatin salt or acid) in the complex is 1:1, 2:1, 3:1, 4:1, 5:1, 6:1, 7:1, or 10:1. In some embodiments, the molar ratio of cyclodextrin/oxaliplatin (or oxaliplatin salt or acid) in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of cyclodextrin/oxaliplatin (or oxaliplatin salt or acid) in the complex is: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In additional embodiments, the cyclodextrin/oxaliplatin (or oxaliplatin salt or acid) complex is encapsulated in a liposome (e.g., as described herein or otherwise known in the art).

In additional embodiments, the disclosure provides a complex comprising cyclodextrin and a platinum-based chemotherapeutic agent selected from: nedaplatin, heptaplatin, lobaplatin, stratoplatin, paraplatin, platinol, cycloplatin, dexormaplatin, spiroplatin, picoplatin, triplatin, tetraplatin, iproplatin, ormaplatin, zeniplatin, platinum-triamine, traplatin, enloplatin, JM216, NK121, CI973, DWA 2114R, NDDP, and dedaplatin, or a salt or acid thereof. In other embodiments, the cyclodextrin/platinum-based chemotherapeutic agent complex comprises an analog of nedaplatin, heptaplatin, lobaplatin, stratoplatin, paraplatin, platinol, cycloplatin, dexormaplatin, spiroplatin, picoplatin, triplatin, tetraplatin, iproplatin, ormaplatin, zeniplatin, platinum-triamine, traplatin, enloplatin, JM216, NK121, CI973, DWA 2114R, NDDP, or dedaplatin, or a salt or acid thereof. In some embodiments, the molar ratio of cyclodextrin/oxaliplatin (or oxaliplatin salt or acid) in the complex is in the range 1-10:1. In some embodiments, the molar ratio of cyclodextrin/platinum-based chemotherapeutic agent (or salt or acid or analog thereof) in the complex is 1:1, 2:1, 3:1, 4:1, 5:1, 6:1, 7:1, or 10:1. In some embodiments, the molar ratio of cyclodextrin/platinum-based chemotherapeutic agent (or salt or acid or analog thereof) in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of cyclodextrin/platinum-based chemotherapeutic agent (or salt or acid or analog thereof) in the complex is: 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In additional embodiments, the cyclodextrin/platinum-based chemotherapeutic agent (or salt or acid or analog thereof) complex is encapsulated in a liposome (e.g., as described herein or otherwise known in the art).

In some embodiments, the disclosure provides a composition comprising a cyclodextrin/taxane-based chemotherapeutic agent complex. In some embodiments, the taxane-based chemotherapeutic agent is selected from: paclitaxel (PTX), docetaxel (DTX), larotaxel (LTX), and cabazitaxel (CTX), or a salt or acid thereof. In some embodiments, the molar ratio of cyclodextrin/taxane-based agent in the complex is in the range 1-10:1. In some embodiments, the molar ratio of cyclodextrin/taxane-based agent in the complex is 1:1, 2:1, 3:1, 4:1, 5:1, 6:1, 7:1, or 10:1. In some embodiments, the molar ratio of cyclodextrin/taxane-based agent in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of cyclodextrin/taxane-based agent in the complex is: 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In additional embodiments, the cyclodextrin/taxane-based agent complex is encapsulated in a liposome (e.g., as described herein or otherwise known in the art).

In additional embodiments, the disclosure provides a complex comprising cyclodextrin and paclitaxel (PTX), or a salt or acid thereof. In other embodiments, the cyclodextrin/taxane-based chemotherapeutic agent complex comprises an analog of paclitaxel (PTX), or a salt or acid thereof. In some embodiments, the molar ratio of cyclodextrin/paclitaxel (or paclitaxel salt or acid) in the complex is in the range 1-10:1. In some embodiments, the molar ratio of cyclodextrin/paclitaxel (or paclitaxel salt or acid) in the complex is 1:1, 2:1, 3:1, 4:1, 5:1, 6:1, 7:1, or 10:1. In some embodiments, the molar ratio of cyclodextrin/paclitaxel (or paclitaxel salt or acid) in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of cyclodextrin/paclitaxel (or paclitaxel salt or acid) in the complex is: 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In additional embodiments, the cyclodextrin/paclitaxel (or paclitaxel salt or acid) complex is encapsulated in a liposome (e.g., as described herein or otherwise known in the art).

In additional embodiments, the disclosure provides a complex comprising cyclodextrin and docetaxel (DTX), or a salt or acid thereof. In other embodiments, the cyclodextrin/taxane-based chemotherapeutic agent complex comprises an analog of docetaxel (DTX), or a salt or acid thereof. In some embodiments, the molar ratio of cyclodextrin/docetaxel (or docetaxel salt or acid) in the complex is in the range 1-10:1. In some embodiments, the molar ratio of cyclodextrin/docetaxel (or docetaxel salt or acid) in the complex is 1:1, 2:1, 3:1, 4:1, 5:1, 6:1, 7:1, or 10:1. In some embodiments, the molar ratio of cyclodextrin/docetaxel (or docetaxel salt or acid) in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of cyclodextrin/docetaxel (or docetaxel salt or acid) in the complex is: 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In additional embodiments, the cyclodextrin/docetaxel (or docetaxel salt or acid) complex is encapsulated in a liposome (e.g., as described herein or otherwise known in the art).

In additional embodiments, the disclosure provides a complex comprising cyclodextrin and larotaxel (LTX), or a salt or acid thereof. In other embodiments, the cyclodextrin/taxane-based chemotherapeutic agent complex comprises an analog of larotaxel (LTX), or a salt or acid thereof. In some embodiments, the molar ratio of cyclodextrin/larotaxel (or larotaxel salt or acid) in the complex is in the range 1-10:1. In some embodiments, the molar ratio of cyclodextrin/larotaxel (or larotaxel salt or acid) in the complex is 1:1, 2:1, 3:1, 4:1, 5:1, 6:1, 7:1, or 10:1. In some embodiments, the molar ratio of cyclodextrin/larotaxel (or larotaxel salt or acid) in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of cyclodextrin/larotaxel (or larotaxel salt or acid) in the complex is: 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In additional embodiments, the cyclodextrin/larotaxel (or larotaxel salt or acid) complex is encapsulated in a liposome (e.g., as described herein or otherwise known in the art).

In additional embodiments, the disclosure provides a complex comprising cyclodextrin and cabazitaxel (CTX), or a salt or acid thereof. In other embodiments, the cyclodextrin/taxane-based chemotherapeutic agent complex comprises an analog of cabazitaxel (CTX), or a salt or acid thereof. In some embodiments, the molar ratio of cyclodextrin/cabazitaxel (or cabazitaxel salt or acid) in the complex is in the range 1-10:1. In some embodiments, the molar ratio of cyclodextrin/cabazitaxel (or cabazitaxel salt or acid) in the complex is 1:1, 2:1, 3:1, 4:1, 5:1, 6:1, 7:1, or 10:1. In some embodiments, the molar ratio of cyclodextrin/cabazitaxel (or cabazitaxel salt or acid) in the complex is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:(21-50), or 1:>50. In some embodiments, the molar ratio of a cyclodextrin/cabazitaxel (or cabazitaxel salt or acid) in the complex is: 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, (21-50):1, or >50:1. In additional embodiments, the cyclodextrin/cabazitaxel (or cabazitaxel salt or acid) complex is encapsulated in a liposome (e.g., as described herein or otherwise known in the art).

The cyclodextrin of the cyclodextrin/therapeutic agent complex can be derivatized or underivatized. In some embodiments, the cyclodextrin is derivatized. In further embodiments, the cyclodextrin is a derivatized beta-cyclodextrin (e.g., a hydroxypropyl beta-cyclodextrin (HP-beta-CD), and a sulfobutyl ether beta-CD (SBE)-beta-cyclodextrin)). In some embodiments, the cyclodextrin of the cyclodextrin/therapeutic agent complex is a derivatized beta-cyclodextrin comprising: 1, 2, 3, 4, 5, 6, 7, 8, 9, 10, or more 2-hydroxylpropyl-3-group substitutions of hydroxy groups; or 1, 2, 3, 4, 5, 6, 7, 8, 9, 10, or more sulfoalkyl ether group substitutions of hydroxy groups. In further embodiments, the cyclodextrin of the cyclodextrin/therapeutic agent complex is a derivatized beta-cyclodextrin comprising: 1, 2, 3, 4, 5, 6, 7, 8, 9, 10, or more sulfobutyl ether group substitutions of hydroxy groups.

In some embodiments, the cyclodextrin of the cyclodextrin/therapeutic agent complex contained in the PANTIFOL liposome composition is a derivatized cyclodextrin of Formula CD-1:

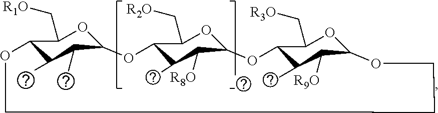

wherein: n is 4, 5, or 6; and wherein R1, R2, R3, R4, R5, R6, R7, R8, and R9 are each, independently, —H, a straight chain or branched C1-C8- alkylene group, a 2-hydroxylpropyl-3-group; or an optionally substituted straight-chain or branched C1-C6 group, wherein at least one of R1, R2, R3, R4, R5, R6, R7, R8 and R9 is a straight-chain or branched C1-C8- alkylene group or a 2-hydroxylpropyl-3-group.

In some embodiments, the cyclodextrin of the cyclodextrin/therapeutic agent complex contained in the PANTIFOL liposome composition is a derivatized cyclodextrin of Formula CD-2:

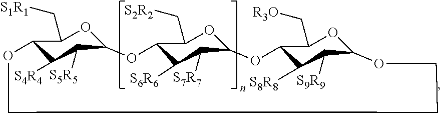

wherein: n is 4, 5, or 6; and wherein R1, R2, R3, R4, R5, R6, R7, R8, and R9 are each, independently, —O— or a —O—(C2-C6 alkylene)-SO3- group; wherein at least one of R1 and R2 is independently a —O—(C2-C6 alkylene)-SO3- group; and S1, S2, S3, S4, S5, S6, S7, S8, and S9 are each, independently, a —H or a H or a pharmaceutically acceptable cation. In further embodiments, the wherein the pharmaceutically acceptable cation is selected from: an alkali metal such as Li+, Na+, or K+; an alkaline earth metal such as Ca+2, or Mg+2, and ammonium ions and amine cations such as the cations of (C1-C6)-alkylamines, piperidine, pyrazine, (C1-C6)-alkanolamine and (C4-C8)-cycloalkanolamine.

In some embodiments, the PANTIFOL liposome comprises between 100 to 100,000 of the cyclodextrin/therapeutic agent complexes.

In some embodiments, a cyclodextrin derivative of the PANTIFOL/cyclodextrin complex and/or cyclodextrin/therapeutic agent complex is a cyclodextrin disclosed in U.S. Pat. Nos. 6,133,248, 5,874,418, 6,046,177, 5,376,645, 5,134,127, 7,034,013, 6,869,939; and Intl. Appl. Publ. No. WO 02005/117911, the contents each of which is herein incorporated by reference in its priority.

In some embodiments, the cyclodextrin derivative of the cyclodextrin/therapeutic agent complex is a sulfoalkyl ether cyclodextrin. In some embodiments, the cyclodextrin derivative of complex is a sulfobutyl ether-3-cyclodextrin such as CAPTISOL® (CyDex Pharma. Inc., Lenexa, Kans. Methods for preparing sulfobutyl ether-3-cyclodextrin and other sulfoalkyl ether cyclodextrins are known in the art.

In some embodiments, the cyclodextrin derivative of the cyclodextrin/therapeutic agent complex is a compound of Formula CD-3:

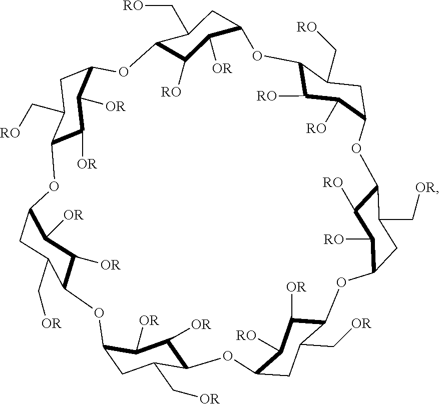

wherein R equals:

(a) (H)_{21-X} or (—(CH₂)₄—SO₃Na)x, and x=1.0-10.0, 1.0-5.0, 6.0-7.0, or 8.0-10.0;

(b) (H)_{21-X} or (—(CH₂CH(OH)CH₃)x, and x=1.0-10.0, 1.0-5.0, 6.0-7.0, or 8.0-10.0;

(c) (H)_{21-X} or (sulfoalkyl ethers)x, and x=1.0-10.0, 1.0-5.0, 6.0-7.0, or 8.0-10.0; or

(d) (H)_{21-X} or (—(CH₂)₄—SO₃Na)x, and x=1.0-10.0, 1.0-5.0, 6.0-7.0, or 8.0-10.0.

Additional cyclodextrins and cyclodextrin/platinum-based therapeutic complexes that can be contained in the PANTIFOL liposomes and used according to the disclosed methods is disclosed in U.S. Appl. No. 62/583,432, the contents of which is herein incorporated by reference it its entirety.

In some embodiments, the PANTIFOL liposome comprises a complex of a cyclodextrin and a platinum-based chemotherapeutic agent, or a salt thereof. In some embodiments, the platinum-based chemotherapeutic agent is cisplatin or a cisplatin analog. In some embodiments, the platinum-based chemotherapeutic agent is carboplatin. In additional embodiments, the liposome composition comprises a platinum-based chemotherapeutic agent is selected from: carboplatin, cisplatin, oxaliplatin, satraplatin, picoplatin, nedaplatin, triplatin, tetraplatin, lipoplatin, lobaplatin, ormaplatin, zeniplatin, platinum-triamine, traplatin, enloplatin, JM-216, 254-S, NK 121, CI-973, DWA 2114R, NDDP, and dedaplatin. In some embodiments, the PANTIFOL liposome comprises between 100 to 100,000 platinum-based chemotherapeutic agent/CD complexes. In additional embodiments, the liposome composition comprises liposomes that have a diameter in the range of 20 nm to 500 nm or 20 nm to 200 nm, or any range therein between. In some embodiments, liposomes in the composition comprise between 100 to 100,000 platinum.

### Targeted Liposomes

In some embodiments, the disclosure provides a liposomal polyglutamated Antifolate composition wherein the liposome comprises a αPANTIFOL and/or gamma polyglutamated Antifolate of the present disclosure and a targeting moiety attached to one or both of a PEG and the exterior of the liposome, and wherein the targeting moiety has a specific affinity for a surface antigen on a target cell of interest. Such liposomes may generally be referred to herein as “targeted liposomes”, e.g., liposomes including one or more targeting moieties or biodistribution modifiers on the surface of, or otherwise attached to, the liposomes. The targeting moiety of the targeted liposomes can be any moiety or agent that is capable of specifically binding a desired target (e.g., an antigen target expressed on the surface of a target cell of interest). In one embodiment, the targeted liposome specifically and preferentially binds to a target on the surface of a target cell of interest that internalizes the targeted liposome into which the liposome encapsulated αPANTIFOL and/or gamma polyglutamated Antifolate (e.g., gamma pentaglutamated Antifolate or gamma hexaglutamated Antifolate) exerts its cytotoxic effect. In further embodiments, the target cell is a cancer cell, a tumor cell or a metastatic cell. In some embodiments, the targeted liposome is pegylated.

The term “attach” or “attached” refers, for example, to any type of bonding such as covalent bonding, ionic bonding (e.g., avidin-biotin) bonding by hydrophobic interactions, and bonding via functional groups such as maleimide, or linkers such as PEG. For example, a detectable marker, a steric stabilizer, a liposome, a liposomal component, an immunostimulating agent may be attached to each other directly, by a maleimide functional group, or by a PEG-malemide group.

The composition and origination of the targeting moiety is non-limiting to the scope of this disclosure. In some embodiments, the targeting moiety attached to the liposome is a polypeptide or peptidomimetic ligand. Peptide and peptidomimetic targeting moieties include those having naturally occurring or modified peptides, e.g., D or L peptides; gamma, beta, or gamma peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic is a molecule capable of folding into a defined three-dimensional structure similar to a natural peptide. In some embodiments, the peptide or peptidomimetic targeting moiety is 2-50 amino acids long, e.g., about 5, 10, 15, 20, 25, 30, 35, 40, 45, or 50 amino acids long

In some embodiments, the targeting moiety polypeptide is at least 40 amino acid residues in length. In other embodiments, the targeting moiety polypeptide is at least 50, 60, 75, 100, 125, 150, 175, 200, 250, or 300 amino acid residues in length.

In additional embodiments, the targeting moiety polypeptide such as an antibody or an antigen-binding antibody fragment that binds a target antigen with an equilibrium dissociation constant (Kd) in a range of 0.5×10⁻¹⁰ to 10×10⁻⁶ as determined using BIACORE® analysis.

In some embodiments, the targeting moiety is an antibody or an antibody derivative. In other embodiments, the binding domain of the targeting moiety polypeptide is not derived from the antigen binding domain of an antibody. In some embodiments, the targeting moiety is a polypeptide derived from a binding scaffold selected from a DARPin, affilin, and armadillo repeat, D domain (see, e.g., WO 2016/164308), Z-domain (Affibody), adnectin, lipocalin, affilin, anticalin, knottin, fynomer, atrimer, kunitz domain (see, e.g., WO 2004/063337), CTLA4, or avimer (see, e.g., U.S. Publ. Nos. 2004/0175756, 2005/0053973, 2005/0048512, and 2006/0008844).

In additional embodiments, the targeting moiety is an antibody or a derivative of the antigen binding domain of an antibody that has specific affinity for an epitope on a cell surface antigen of interest expressed on the surface of a target cell. In some embodiments, the targeting moiety is a full-length antibody. In some embodiments, the targeting moiety is an antigen binding portion of an antibody. In some embodiments, the targeting moiety is an scFv. In other embodiments, the targeting moiety is a Fab. In some embodiments, the targeting moiety comprises a binding domain derived from the antigen binding domain of an antibody (e.g., an scFv, Fab, Fab′, F(ab′)2, an Fv fragment, a disulfide-linked Fv (sdFv), a Fd fragment consisting of VH and CH1 domains, an scFv, a minibody, a BiTE, a Tandab, a diabody ((VL-VH)2 or (VH-VL)2), a single domain antibody (e.g., an sdAb such as a nanobody (either VL or VH)), and a camelid VHH domain). In some embodiments, the targeting moiety comprises one or more complementarity determining regions (CDRs) of antibody origin. Examples of suitable antibody-based targeting moieties for the disclosed targeted liposomes include a full-length human antibody, a humanized antibody, a chimeric antibody, an antigen binding fragment of an antibody, a single chain antibody, a single-domain antibody, a bi-specific antibody, a synthetic antibody, a pegylated antibody and a multimeric antibody. The antibody of the provided targeted liposomes can have a combination of the above characteristics. For example, a humanized antibody can be an antigen binding fragment and can be pegylated and multimerized as well.

The term “humanized antibody” refers to forms of non-human (e.g., murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, and hamster) that have the desired specificity, affinity, and capability (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. Nos. 5,225,539 and 5,639,641.

In further embodiments, the targeting moiety has specific affinity for an epitope on a surface antigen of a target cell of interest. In some embodiments, the target cell is a cancer cell. In some embodiments, the target cell is a tumor cell. In other embodiments, the target cell is an immune cell.

In some embodiments, the targeting moiety has specific affinity for an epitope expressed on a tumor cell surface antigen. The term “tumor cell surface antigen” refers to an antigen that is common to a specific hyperproliferative disorder such as cancer. In some embodiments, the targeting moiety has specific affinity for an epitope of a tumor cell surface antigen that is a tumor associated antigen (TAA). A TAA is an antigen that is found on both tumor and some normal cells. A TAA may be expressed on normal cells during fetal development when the immune system is immature and unable to respond or may be normally present at extremely low levels on normal cells but which are expressed at much higher levels on tumor cells. Because of the dynamic nature of tumors, in some instances, tumor cells may express unique antigens at certain stages, and at others also express antigens that are also expressed on non-tumor cells. Thus, inclusion of a certain marker as a TAA does not preclude it being considered a tumor specific antigen. In some embodiments, the targeting moiety has specific affinity for an epitope of a tumor cell surface antigen that is a tumor specific antigen (TSA). A TSA is an antigen that is unique to tumor cells and does not occur on other cells in the body. In some embodiments, the targeting moiety has specific affinity for an epitope of a tumor cell surface antigen expressed on the surface of a cancer including but not limited to primary or metastatic melanoma, thymoma, lymphoma, sarcoma, lung cancer (e.g., NSCLC or SCLC), liver cancer, non-Hodgkin's lymphoma, Hodgkin's lymphoma, leukemias, multiple myeloma, glioblastoma, neuroblastoma, uterine cancer, cervical cancer, renal cancer, thyroid cancer, bladder cancer, kidney cancer, mesothelioma, and adenocarcinomas such as breast cancer, prostate cancer, ovarian cancer, pancreatic cancer, colon cancer and other cancers known in the art In some embodiments, the targeting moiety has specific affinity for an epitope of a cell surface antigen expressed on the surface of a cell in the tumor microenvironment (e.g., and antigen such as VEGFR and TIE1, or TIE2 expressed on endothelial cells and macrophage, respectively, or an antigen expressed on tumor stromal cells such as cancer-associated fibroblasts (CAFs) tumor infiltrating T cells and other leukocytes, and myeloid cells including mast cells, eosinophils, and tumor-associated macrophages (TAM).

In some embodiments, the targeted liposome PANTIFOL composition (e.g., TLp-PANTIFOL or TPLp-PANTIFOL) comprises a targeting moiety that has specific affinity for an epitope of a cancer or tumor cell surface antigen that is preferentially/differentially expressed on a target cell such as a cancer cell or tumor cell, compared to normal or non-tumor cells, that is present on a tumor cell but absent or inaccessible on a non-tumor cell. For example, in some situations, the tumor antigen is on the surface of both normal cells and malignant cancer cells but the tumor epitope is only exposed in a cancer cell. As a further example, a tumor cell surface antigen may experience a confirmation change in a cancerous state that causes a cancer cell specific epitope to be present. A targeting moiety with specific affinity to an epitope on a targetable tumor cell surface antigen described herein or otherwise known in the art is useful and is encompassed by the disclosed compositions and methods. In some embodiments, the tumor cell with the tumor cell surface antigen is a cancer cell. Examples of such tumor cell surface antigens include, without limitation folate receptor alpha, folate receptor beta and folate receptor delta.

In further embodiments, the targeting moiety comprises a polypeptide targeting moiety such as an antibody or an antigen-binding antibody fragment and the targeting moiety has binding specificity for a folate receptor. In some embodiments, the targeting moiety binds a folate receptor with an equilibrium dissociation constant (Kd) in a range of 0.5×10⁻¹⁰ to 10×10⁻⁶ as determined using BIACORE® analysis. In some embodiments, the folate receptor bound by the targeting moiety is one or more folate receptors selected from: folate receptor alpha (FR-α), folate receptor beta (FR-β), and folate receptor delta (FR-δ). In a further embodiment, the targeting moiety has specific affinity for at least two antigens selected from folate receptor alpha, folate receptor beta, and folate receptor delta. In another embodiment, the targeting moiety has specific affinity for folate receptor alpha; folate receptor beta; and folate receptor delta.

In some embodiments, the targeting moiety has a specific affinity for an epitope of a cell surface antigen that internalizes the targeting moiety upon binding. Numerous cell surface antigens that internalize binding partners such as antibodies upon binding are known in the art and are envisioned to be binding targets for the targeting moieties expressed on the targeted liposome PANTIFOL compositions (e.g., TLp-PANTIFOL or TPLp-PANTIFOL) disclosed herein.

In some embodiments, the targeting moiety has a specific affinity for an epitope of a cell surface antigen selected from: GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TMEFF2, endoglin, PSMA, CD98, CD56, CanAg, and CALLA.

In some embodiments, the targeting moiety has a specific affinity for an epitope of a cell surface antigen selected from mannose-6-phosphate receptor, transferrin receptor, and a cell adhesion molecule (CAM). In further embodiments, the targeting moiety has a specific affinity for an epitope of a CAM is selected from the group consist of: intercellular adhesion molecule (ICAM), platelet-endothelial cell adhesion molecule (PECAM), activated leukocyte cell adhesion molecule (ALCAM), B-lymphocyte cell adhesion molecule (BL-CAM), vascular cell adhesion molecule (VCAM), mucosal vascular addressin cell adhesion molecule (MAdCAM), CD44, LFA-2, LFA-3, and basigin.

A discussed herein, folate receptors (FRs) are distinct from reduced folate carriers (RFCs) and exploit different pathways for bringing folates and antifolates into cells. In some embodiments, the targeting moiety specifically binds a folate receptor. In further embodiments, the targeting moiety specifically binds a folate receptor selected from folate receptor alpha, folate receptor beta and folate receptor delta. Antibodies to folate receptor alpha can routinely be generated using techniques known in the art. Moreover, the sequences of numerous anti-folate receptor antibodies are in the public domain and/or commercially available and are readily obtainable.

Murine antibodies against folate receptor are examples of antibodies that can be used as targeting moieties of the disclosed targeted liposome is a murine antibody against folate receptor. The sequence of these antibodies are known and are described, for example, in U.S. Pat. Nos. 5,646,253; 8,388,972; 8,871,206; and 9,133,275, and Intl. Appl. Nos. PCT/US2011/056966, and PCT/US2012/046672. For example, based on the sequences already in the public domain, the gene for the antibodies can be synthesized and placed into a transient expression vector and the antibody was produced in HEK-293 transient expression system. The antibody can be a complete antibody, a Fab, or any of the various antibody variations discussed herein or otherwise known in the art.

In some embodiments, the targeted liposome (e.g., TL-PANTIFOL or TPL-PANTIFOL) contains from 1 to 1,000, or more than 1,000, targeting moieties on its surface. In some embodiments, the targeted liposome contains from 30 to 1,000, 30 to 500, 30 to 250 or 30-200, targeting moieties, or any range therein between. In some embodiments, the targeted liposome (e.g., TL-PANTIFOL or TPL-PANTIFOL) contains from 30 to 1,000, or more than 1,000, targeting moieties on its surface. In some embodiments, the targeted liposome contains from 30 to 500, 30 to 250 or 30-200, targeting moieties. In some embodiments, the targeted liposome contains less than 220 targeting moieties, less than 200 targeting moieties, or less than 175 targeting moieties. In some embodiments, the targeting moiety is non-covalently bonded to the outside of the liposome (e.g., via ionic interaction or a GPI anchor). In some embodiments, the targeted liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the molecules on the outside of the targeted liposome (e.g., TL-PANTIFOL or TPL-PANTIFOL) include a lipid, a targeting moiety, a steric stabilizer (e.g., a PEG), a maleimide, and a cholesterol. In some embodiments, the targeting moiety is covalently bound via a maleimide functional group. In some embodiments, the targeting moiety is covalently bound to a liposomal component or a steric stabilizer such as a PEG molecule. In some embodiments, all the targeting moieties of the liposome are bound to one component of the liposome such as a PEG. In other embodiments, the targeting moieties of the targeted liposome are bound to different components of the liposome. For example, some targeting moieties may be bound to the lipid components or cholesterol, some targeting moieties may be bound to the steric stabilizer (e.g., PEG) and still other targeting moieties may be bound to a detectable marker or to another targeting moiety. In some embodiments, the outside of the targeted liposome (e.g., TL-PANTIFOL or TPL-PANTIFOL) further comprises one or more of an immunostimulatory agent, a detectable marker and a maleimide disposed on at least one of the PEG and the exterior of the liposome. In some embodiments, the targeted liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the targeted liposome (e.g., TL-PANTIFOL or TPL-PANTIFOL) is anionic or neutral. In some embodiments, the targeted anionic or neutral liposome has a diameter in the range of 20 nm to 500 nm or 20 nm to 200 nm, or any range therein between. In further embodiments, the targeted anionic or neutral liposome has a diameter in the range of 80 nm to 120 nm, or any range therein between. In some embodiments, the targeted liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In other embodiments, the targeted liposome (e.g., TL-PANTIFOL or TPL-PANTIFOL) is cationic. In some embodiments, the targeted anionic or neutral liposome has a diameter in the range of 20 nm to 500 nm or 20 nm to 200 nm, or any range therein between. In further embodiments, the targeted anionic or neutral liposome has a diameter in the range of 80 nm to 120 nm, or any range therein between. In some embodiments, the targeted liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In additional embodiments, the liposomal composition comprising the targeted liposome (e.g., TL-PANTIFOL or TPL-PANTIFOL) comprises 30-70%, 30-60%, or 30-50% liposome entrapped αPANTIFOL and/or gamma polyglutamated Antifolate of the present disclosure, or any range therein between. In some embodiments, the liposomal composition comprising the targeted liposome comprises at least 1%, 5%, 10%, 15%, 20%, 25%, 30%, 35%, 40%, 45%, 50%, 55%, 60%, 65%, 70%, 75%, or more than 75%, of the starting material of αPANTIFOL and/or gamma polyglutamated Antifolate is encapsulated (entrapped) in the targeted liposomes liposomes.

In some embodiments, the targeted liposomal compositions comprise 30-70%, 30-60%, or 30-50%, w/w of the tetraglutamated Antifolate the present disclosure, or any range therein between. In some embodiments, the targeted liposomes comprise at least 1%, 5%, 10%, 15%, 20%, 25%, 30%, 35, 40%, 45%, 50%, 55%, 60%, 65%, 70%, 75%, or more than 75%, w/w of the tetraglutamated Antifolate. In some embodiments, during the process of preparing the targeted liposomes, at least 1%, 5%, 10%, 15%, 20%, 25%, 30%, 35%, 40%, 45%, 50%, 55%, 60%, 65%, 70%, 75%, or more than 75%, of the starting material of tetraglutamated Antifolate is encapsulated (entrapped) in the targeted liposomes. In some embodiments, the tetraglutamated Antifolate is gamma tetraglutamated Antifolate. In some embodiments, the tetraglutamated Antifolate is alpha tetraglutamated Antifolate.

In some embodiments, the targeted liposomal compositions comprise 30-70%, 30-60%, or 30-50%, w/w of the pentaglutamated Antifolate the present disclosure, or any range therein between In some embodiments, the targeted liposomes comprise at least 1%, 5%, 10%, 15%, 20%, 25%, 30%, 35, 40%, 45%, 50%, 55%, 60%, 65%, 70%, 75%, or more than 75%, w/w of the pentaglutamated Antifolate. In some embodiments, during the process of preparing the targeted liposomes, at least 1%, 5%, 10%, 15%, 20%, 25%, 30%, 35%, 40%, 45%, 50%, 55%, 60%, 65%, 70%, 75%, or more than 75%, of the starting material of pentaglutamated Antifolate is encapsulated (entrapped) in the targeted liposomes. In some embodiments, the pentaglutamated Antifolate is gamma pentaglutamated Antifolate. In some embodiments, the pentaglutamated Antifolate is alpha pentaglutamated Antifolate.

In some embodiments, the targeted liposomal compositions comprise 30-70%, 30-60%, or 30-50%, w/w of the hexaglutamated Antifolate the present disclosure, or any range therein between In some embodiments, the targeted liposomes comprise at least 1%, 5%, 10%, 15%, 20%, 25%, 30%, 35, 40%, 45%, 50%, 55%, 60%, 65%, 70%, 75%, or more than 75%, w/w of the hexaglutamated Antifolate. In some embodiments, during the process of preparing the targeted liposomes, at least 1%, 5%, 10%, 15%, 20%, 25%, 30%, 35%, 40%, 45%, 50%, 55%, 60%, 65%, 70%, 75%, or more than 75%, of the starting material of hexaglutamated Antifolate is encapsulated (entrapped) in the targeted liposomes. In some embodiments, the hexaglutamated Antifolate is gamma hexaglutamated Antifolate. In some embodiments, the hexaglutamated Antifolate is alpha hexaglutamated Antifolate.

Methods and techniques for covalently associating polypeptide targeting moieties with a liposome surface molecule are known in the art and can readily be applied to prepare the TL-PANTIFOL or TPL-PANTIFOL liposome compositions.

Chemical binding of non-proteinaceous targeting moieties and other compositions to the liposomal surface may be employed. Thus, a non-proteinaceous moiety, may be covalently or non-covalently linked to, embedded or adsorbed onto the liposome using any linking or binding method and/or any suitable chemical linker known in the art. The exact type and chemical nature of such cross-linkers and cross-linking methods is preferably adapted to the type of affinity group used and the nature of the liposome. Methods for binding or adsorbing or linking the targeting moiety are also well known in the art. For example, in some embodiments, the targeting moiety may be attached to a group at the interface via, but not limited to, polar groups such as amino, SH, hydroxyl, aldehyde, formyl, carboxyl, His-tag or other polypeptides. In addition, the targeting moiety may be attached via, but not limited to, active groups such as succinimidyl succinate, cyanuric chloride, tosyl activated groups, imidazole groups, CNBr, NHS, Activated CH, ECH, EAH, Epoxy, Thiopropyl, Activated Thiol, etc., Moreover, the targeting moiety may be attached via, but not limited to, hydrophobic bonds (Van Der Waals) or electrostatic interactions that may or may not include cross-linking agents (e.g., bivalent anions, poly-anions, poly-cations etc.).

### Manufacture of Liposomes

In some embodiments, the disclosure provides a method of making a liposomal composition disclosed herein. In one embodiment, the method includes forming a mixture comprising: (1) a liposomal component; and (2) a gamma polyglutamated (e.g., pentaglutamated or hexaglutamated) Antifolate of the present disclosure in aqueous solution. In one embodiment, the method includes forming a mixture comprising: (1) a liposomal component; and (2) an alpha polyglutamated (e.g., pentaglutamated or hexaglutamated) Antifolate of the present disclosure in aqueous solution. In further embodiments, the mixture comprises a pegylated liposomal component. The mixture is then homogenized to form liposomes in the aqueous solution. Further, the mixture can be extruded through a membrane to form liposomes enclosing the alpha or gamma polyglutamated Antifolate in an aqueous solution. It is understood the liposomal components of this disclosure can comprise any lipid (including cholesterol) including functionalized lipids and lipids attached to targeting moieties, detectable labels, and steric stabilizers, or any subset of all of these. It is further noted that the bioactive alpha or gamma polyglutamated Antifolate in aqueous solution can comprise any reagents and chemicals discussed herein or otherwise known in the art for the interior or exterior of the liposome including, for example, buffers, salts, and cryoprotectants.

In some embodiments, the disclosure provides a method of making a targeted pegylated liposomal gamma polyglutamated Antifolate (targeted-PLp-γPANTIFOL) or non-targeted PLp-γPANTIFOL disclosed herein. In one embodiment, the method includes forming a mixture comprising: (1) a liposomal component; (2) a gamma polyglutamated (e.g., pentaglutamated or hexaglutamated) Antifolate of the present disclosure in aqueous solution; and (3) the targeting moiety. In some embodiments, the disclosure provides a method of making a targeted pegylated liposomal alpha polyglutamated Antifolate (targeted-PLp-αPANTIFOL) or non-targeted PLp-αPANTIFOL disclosed herein. In one embodiment, the method includes forming a mixture comprising: (1) a liposomal component; (2) an alpha polyglutamated (e.g., pentaglutamated or hexaglutamated) Antifolate of the present disclosure in aqueous solution; and (3) the targeting moiety. The mixture is then homogenized to form liposomes in the aqueous solution. Further, the mixture may be extruded through a membrane to form liposomes enclosing the targeted alpha or gamma polyglutamated Antifolate in an aqueous solution. It is understood that the targeted pegylated liposomal components can comprise any lipid (including cholesterol) including functionalized lipids and lipids attached to targeting moieties, detectable labels, and steric stabilizers, or any subset of all of these. It is further noted that the targeted pegylated liposome can comprise any reagents and chemicals discussed herein or otherwise known in the art for the interior or exterior of the liposome including, for example, buffers, salts, and cryoprotectants.

The above methods optionally further comprise the step of lyophilizing the composition after the removing step to form a lyophilized composition. As stated above, targeted-PTPLA or non-targeted-PTPLA in aqueous solution may comprise a cryoprotectant described herein or otherwise known in the art. If the composition is to be lyophilized, a cryoprotectant may be preferred.

Additionally, after the lyophilizing step, the method optionally further comprises the step of reconstituting the lyophilized composition by dissolving the composition in a solvent after the lyophilizing step. Methods of reconstitution are known in the art. One preferred solvent is water. Other preferred solvents include saline solutions and buffered solutions.

While certain exemplary embodiments, are discussed herein, it is understood that liposomes can be made by any method that is known in the art. See, for example, G. Gregoriadis (editor), Liposome Technology, vol. 1-3, 1st edition, 1983; 2nd edition, 1993, CRC Press, 45 Boca Raton, Fla. Examples of methods suitable for making liposome compositions include extrusion, reverse phase evaporation, sonication, solvent (e.g., ethanol) injection, microfluidization, detergent dialysis, ether injection, and dehydration/rehydration. The size of liposomes can routinely be controlled by controlling the pore size of membranes used for low pressure extrusions or the pressure and number of passes utilized in microfluidization or any other suitable methods known in the art.

In general, the polyglutamated Antifolate of the present disclosure is contained inside, that is, in the inner (interior) space of the liposomes. In one embodiment, substituted ammonium is partially or substantially completely removed from the outer medium surrounding the liposomes. Such removal can be accomplished by any suitable means known in the art (e.g., dilution, ion exchange chromatography, size exclusion chromatography, dialysis, ultrafiltration, and precipitation). Accordingly, the methods of making liposomal compositions set forth above or otherwise known in the art can optionally further comprise the step of removing polyglutamated Antifolate in aqueous solution outside of the liposomes after forming the liposomes, for example, by the homogenization or by the extruding step.

In other embodiments, the disclosure provides a targeted pegylated liposomal polyglutamated Antifolate (TPLp-PANTIFOL) that selectively targets folate receptors comprising: a liposome including an interior space, a polyglutamated Antifolate disposed within the interior space, a steric stabilizer molecule attached to an exterior of the liposome, and a targeting moiety comprising a protein with specific affinity for at least one folate receptor, said targeting moiety attached to at least one of the steric stabilizer and the exterior of the liposome. The components of this embodiment, may be the same as described for other embodiments, of this disclosure. For example, the targeted pegylated liposomal polyglutamated Antifolate and the steric stabilizer which may be PEG, are as described in other parts of this disclosure.

In some embodiments, the disclosure provides a method of preparing a targeted composition comprising a pegylated liposome including an entrapped and/or encapsulated polyglutamated Antifolate; a targeting moiety an amino acid chain, the amino acid chain comprising a plurality of amino acids, the targeting moiety having a specific affinity for at least one type of folate receptor, the specific affinity being defined to include an equilibrium dissociation constant (Kd) in a range of 0.5×10⁻¹⁰ to 10×10⁻⁶ moles [0.05 nM to 10 μM] for at least one type folate receptor, the targeting moiety attached to one or both of a PEG and an exterior of the liposome, the method comprising: forming a mixture comprising: liposomal components and a polyglutamated Antifolate in solution; homogenizing the mixture to form liposomes in the solution; processing the mixture to form liposomes entrapping and/or encapsulating polyglutamated Antifolate; and providing a targeting moiety on a surface of the liposomes entrapping and/or encapsulating the polyglutamated Antifolate, the targeting moiety having specific affinity for at least one of folate receptor alpha (FR-α), folate receptor beta (FR-β) and folate receptor delta (FR-δ). In some embodiments, the method comprising: forming a mixture comprising: liposomal components and polyglutamated Antifolate in solution; forming liposomes entrapping and/or encapsulating polyglutamated Antifolate, for example by homogenizing or otherwise processing the mixture to form liposomes; and providing a targeting moiety on a surface of the liposomes entrapping and/or encapsulating the polyglutamated Antifolate, the targeting moiety having specific affinity for at least one of folate receptor alpha (FR-α), folate receptor beta (FR-β) and folate receptor delta (FR-δ). In some embodiments, the processing includes one or more of: thin film hydration, extrusion, in-line mixing, ethanol injection technique, freezing-and-thawing technique, reverse-phase evaporation, dynamic high pressure microfluidization, microfluidic mixing, double emulsion, freeze-dried double emulsion, 3D printing, membrane contactor method, and stirring, and once the particles have been formed, the particles can have their sizes further modified by one or more of extrusion and sonication. In some embodiments, during the process of preparing the liposomes at least 1%, 5%, 10%, 15%, 20%, 25%, 30%, 35%, 40%, 45%, 50%, 55%, 60%, 65%, 70%, 75%, or more than 75%, of the starting material of polyglutamated Antifolate is encapsulated (entrapped) in the targeted liposomes. In some embodiments, the liposomes are anionic or neutral. In some embodiments, the targeting moiety has the specific affinity for one or more of: folate receptor alpha (FR-α), folate receptor beta (FR-β) and folate receptor delta (FR-δ). In further embodiments, the targeting moiety has the specific affinity for folate receptor alpha (FR-α) and folate receptor beta (FR-β). In additional embodiments, the targeting moiety has the specific affinity for an epitope on a tumor cell surface antigen that is present on a tumor cell but absent or inaccessible on a non-tumor cell.

Liposomes can also be prepared to target particular cells, organs, or cell organelles by varying phospholipid composition or by inserting receptors or counter-receptors into the liposomes. For example, liposomes, prepared with a high content of a nonionic surfactant, have been used to target the liver. (See, e.g., Japanese Patent 04-244,018 to Hayakawa et al.; Kato et al., Biol. Pharm. Bull. 16:960, 1993.) A liposomal formulation of dipalmitoylphosphatidylcholine (DPPC) with a soybean-derived sterylglucoside mixture (SG) and cholesterol (Ch) has also been shown to target the liver. (See e.g., Shimizu et al., Biol. Pharm. Bull. 20:881 (1997)).

### Antibody Delivery Vehicles

In additional embodiments, the disclosure provides an antibody delivery vehicle (e.g., ADC). In some embodiments, the disclosure provides an immunoconjugate having the Formula (A)-(L)-(PANTIFOL), wherein: (A) is an antibody or antigen binding fragment of an antibody; (L) is a linker; and (PANTIFOL) is a PANTIFOL composition described herein; and wherein said linker (L) links (A) to (PANTIFOL). In some embodiments, the PANTIFOL is a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the antibody or antigen binding antibody fragment has specific affinity for an epitope of a cell surface antigen on a cell of interest (e.g., an epitope and/or antigen described herein). In certain embodiments, the antibody binds to an antigen target that is expressed in or on the cell membrane (e.g., on the cell surface) of a cancer/tumor and the antibody is internalized by the cell after binding to the (antigen) target, after which the PANTIFOL is released intracellularly. In some embodiments, the antibody is a full length antibody.

The antibody or antigen binding antibody fragment of the (A)-(L)-(PANTIFOL) immunoconjugate can be an IgA, IgD, IgE, IgG or IgM antibody. The different classes of immunoglobulins have different and well-known subunit structures and three-dimensional configurations. In certain embodiments, the antibody is an IgG antibody. In some embodiments, the antibody is an IgG1, IgG2, IgG3 or IgG4 antibody. In certain embodiments, the antibody is an IgG1 antibody.

In some embodiments, (A) is an antigen binding fragment of an antibody. In some embodiments, (A) is an antigen binding fragment of an antibody.

A “linker” is any chemical moiety that is capable of linking a compound, usually a drug, such as a PANTIFOL, to an antibody or antigen binding fragment of an antibody in a stable, covalent manner. The linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are known in the art and include, for example, disulfide groups, thioether groups, acid labile groups, photolabile groups, peptidase labile groups and esterase labile groups. Linkers also include charged linkers, and hydrophilic forms thereof.

In some embodiments, the linker is selected from a cleavable linker, a non-cleavable linker, a hydrophilic linker, and a dicarboxylic acid-based linker. In another embodiment, the linker is a non-cleavable linker. In another embodiment, the linker is selected from the group consisting: N-succinimidyl 4-(2-pyridyldithio) pentanoate (SPP); N-succinimidyl 4-(2-pyridyldithio)butanoate (SPDB) or N-succinimidyl 4-(2-pyridyldithio)-2-sulfobutanoate (sulfo-SPDB); N-succinimidyl 4-(maleimidomethyl) cyclohexane-carboxylate (SMCC); N-sulfosuccinimidyl 4-(maleimidomethyl) cyclohex-anecarboxylate (sulfoSMCC); N-succinimidyl-4-(iodoacetyl)-aminobenzoate (SIAB); and N-succinimidyl-RN-maleimidopropionamido)-tetraethyleneglycollester (NHS-PEG4-ma-leimide). In a further embodiment, the linker is N-succinimidyl-[(N-maleimido-propionamido)-tetraethyleneglycol] ester (NHS-PEG4-maleimide).

In some embodiments, the polyglutamated Antifolate is attached (coupled) to the antibody or antigen binding antibody fragment of the immunoconjugate directly, or through a linker using techniques known in the art. Such attachment of one or more PANTIFOL can include many chemical mechanisms, such as covalent binding, affinity binding, intercalation, coordinate binding and complexation. Covalent binding of the PANTIFOL and antibody or antigen binding antibody fragment can be achieved by direct condensation of existing side chains or by the incorporation of external bridging molecules. Many bivalent or polyvalent agents are useful in associating polypeptides to other proteins with coupling agents such as carbodiimides, diisocyanates, glutaraldehyde, diazobenzenes, and hexamethylene diamines. This list is not intended to be exhaustive of the various coupling agents known in the art but, rather, is exemplary of the more common coupling agents. In some embodiments, the antibody or antigen binding antibody fragment is derivatized and then attached to the polyglutamated Antifolate. Alternatively, the PANTIFOL can be derivatized and attached to the antibody or antigen binding antibody fragment using techniques known in the art.

In some embodiments, the immunoconjugate comprises an antibody or an antigen-binding fragment of an antibody and PANTIFOL containing 4, 5, 2-10, 4-6, or more than 5, glutamyl groups (including the glutamyl group of the Antifolate). In some embodiments, the immunoconjugate comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the antibody delivery vehicle composition comprises a polyglutamated Antifolate and an antibody or an antigen binding antibody fragment that has specific affinity for an epitope on a cell surface antigen selected from: GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P Cadherin, Fibronectin Extra-domain B (ED-B), VEGFR2 (CD309), Tenascin, Collagen IV, Periostin, endothelin receptor, HER2, HER3, ErbB4, EGFR, EGFRvIII, FGFR1, FGFR2, FGFR3, FGFR4, FGFR6, IGFR-1, FZD1, FZD2, FZD3, FZD4, FZD5, FZD6, FZD7, FZD8, FZD9, FZD10, SMO, CD2, CD3, CD4, CD5, CD6, CD8, CD11, CD11a, CD15, CD18, CD19, CD20, CD22, CD26, CD27L, CD28, CD30, CD33, CD34, CD37, CD38, CD40, CD40L, CD44, CD56, CD70, CD74, CD79, CD79b, CD98, CD105, CD133, CD138, cripto, IGF-1R, IGF-2R, EphA1 an EphA receptor, an EphB receptor, EphA2, EphA3, EphA4, EphA5, EphA6, EphA7, EphA8, EphA1, EphB1, EphB2, EphB3, EphB4, EphB6, an integrin (e.g., integrin αvβ3, αvβ5, or αvβ6), a C242 antigen, Apo2, PSGR, NGEP, PSCA, TMEFF2, endoglin, PSMA, CanAg, CALLA, c-Met, VEGFR-1, VEGFR-2, DDR1, PDGFR alpha., PDGFR beta, TrkA, TrkB, TrkC, UFO, LTK, ALK, Tie1, Tie2, PTK7, Ryk, TCR, NMDAR, LNGFR, and MuSK. In some embodiments, the delivery vehicle comprises a targeting moiety that has specific affinity for an epitope on a cell surface antigen derived from, or determined to be expressed on, a specific subject's cancer (tumor) such as a neoantigen. In some embodiments, the antibody delivery vehicle composition comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the antibody delivery vehicle composition comprises a polyglutamated Antifolate and an antibody or an antigen binding antibody fragment that has specific affinity for an epitope on an antigen selected from mannose-6-phosphate receptor, transferrin receptor, and a cell adhesion molecule (CAM). In further embodiments, the targeting moiety has a specific affinity for an epitope of a CAM is selected from the group consist of: intercellular adhesion molecule (ICAM), platelet-endothelial cell adhesion molecule (PECAM), activated leukocyte cell adhesion molecule (ALCAM), B-lymphocyte cell adhesion molecule (BL-CAM), vascular cell adhesion molecule (VCAM), mucosal vascular addressin cell adhesion molecule (MAdCAM), CD44, LFA-2, LFA-3, and basigin. In some embodiments, the antibody delivery vehicle composition comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the antibody delivery vehicle composition comprises 1, 2, 3, 4, 5, 5-10, or greater than 10 polyglutamated Antifolate. In some embodiments, the antibody delivery vehicle composition comprises 1, 2, 3, 4, 5, 5-10, or greater than 10, pentaglutamated Antifolate. In some embodiments, the antibody delivery vehicle composition comprises 1, 2, 3, 4, 5, 5-10, or greater than 10, hexaglutamated Antifolate. In some embodiments, the antibody delivery vehicle composition comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

### Pharmaceutical Compositions and Administration

In some embodiments, the liposome composition is provided as a pharmaceutical composition containing the liposome (e.g., described herein) and a carrier, e.g., a pharmaceutically acceptable carrier. Examples of pharmaceutically acceptable carriers contained in the provided pharmaceutical compositions include normal saline, isotonic dextrose, isotonic sucrose, Ringer's solution, and Hanks' solution. In some embodiments, a buffer substance is added to maintain an optimal pH for storage stability of the pharmaceutical composition. In some embodiments, the pH of the pharmaceutical composition is between 6.0 and 7.5. In some embodiments, the pH is between 6.3 and 7.0. In further embodiments, the pH is 6.5. Ideally the pH of the pharmaceutical composition allows for both stability of liposome membrane lipids and retention of the entrapped entities. Histidine, hydroxyethylpiperazine-ethylsulfonate (HEPES), morpholipoethylsulfonate (MES), succinate, tartrate, and citrate, typically at 2-20 mM concentration, are exemplary buffer substances. Other suitable carriers include, e.g., water, buffered aqueous solution, 0.4% NaCl, and 0.3% glycine. Protein, carbohydrate, or polymeric stabilizers and tonicity adjusters can be added, e.g., gelatin, albumin, dextran, or polyvinylpyrrolidone. The tonicity of the composition can be adjusted to the physiological level of 0.25-0.35 mol/kg with glucose or a more inert compound such as lactose, sucrose, mannitol, or dextrin. These compositions can routinely be sterilized using conventional, sterilization techniques known in the art (e.g., by filtration). The resulting aqueous solutions can be packaged for use or filtered under aseptic conditions and lyophilized, the lyophilized preparation being combined with a sterile aqueous medium prior to administration.

The provided pharmaceutical liposome compositions can also contain other pharmaceutically acceptable auxiliary substances as required to approximate physiological conditions, such as pH adjusting and buffering agents, and tonicity adjusting agents, for example, sodium acetate, sodium lactate, sodium chloride, potassium chloride, and calcium chloride. Additionally, the liposome suspension may include lipid-protective agents which protect lipids against free-radical and lipid-peroxidative damages on storage. Lipophilic free-radical quenchers, such as gamma-tocopherol and water-soluble iron-specific chelators, such as ferrioxamine, are suitable.

The liposome concentration in the provided fluid pharmaceutical formulations can vary widely depending upon need, e.g., from less than about 0.05% usually or at least about 2-10% to as much as 30-50% by weight and will be selected primarily by fluid volumes, and viscosities, in accordance with the particular mode of administration selected. For example, the concentration may be increased to lower the fluid load associated with treatment. This may be particularly desirable in patients having atherosclerosis-associated congestive heart failure or severe hypertension. Alternatively, liposome pharmaceutical compositions composed of irritating lipids may be diluted to low concentrations to lessen inflammation at the site of administration.

Some embodiments, relate to a method of delivering a targeted pegylated liposomal formulation of polyglutamated Antifolate, to a tumor expressing folate receptor on its surface. An exemplary method comprises the step of administering a liposome pharmaceutical composition provided herein in an amount to deliver a therapeutically effective dose of the targeted pegylated liposomal polyglutamated Antifolate to the tumor.

The amount of liposome pharmaceutical composition administered will depend upon the particular polyglutamated Antifolate entrapped inside the liposomes, the disease state being treated, the type of liposomes being used, and the judgment of the clinician. Generally, the amount of liposome pharmaceutical composition administered will be sufficient to deliver a therapeutically effective dose of the particular therapeutic entity.

The quantity of liposome pharmaceutical composition necessary to deliver a therapeutically effective dose can be determined by routine in vitro and in vivo methods, common in the art of drug testing. See, for example, D. B. Budman, A. H. Calvert, E. K. Rowinsky (editors). Handbook of Anticancer Drug Development, LWW, 2003. Therapeutically effective dosages for various therapeutic compositions are known to those skilled in the art. In some embodiments, a therapeutic entity delivered via the pharmaceutical liposome composition and provides at least the same or higher activity than the activity obtained by administering the same amount of the therapeutic entity in its routine non-liposome formulation. Typically, the dosages for the liposome pharmaceutical composition is in a range for example, between about 0.005 and about 5000 mg of the therapeutic entity per square meter of body surface area most often, between about 0.1 and about 1000 mg therapeutic entity per square meter of body surface area.

For example, if the subject has a tumor, an effective amount may be that amount of the agent (e.g., gamma polyglutamated Antifolate composition or alpha polyglutamated Antifolate composition) that reduces the tumor volume or load (as for example determined by imaging the tumor). Effective amounts can also routinely be assessed by the presence and/or frequency of cancer cells in the blood or other body fluid or tissue (e.g., a biopsy). If the tumor is impacting the normal functioning of a tissue or organ, then the effective amount can routinely be assessed by measuring the normal functioning of the tissue or organ. In some instances, the effective amount is the amount required to lessen or eliminate one or more, and preferably all, symptoms.

Pharmaceutical compositions comprising the polyglutamated Antifolate compositions (e.g., liposomes containing a pentaglutamated or hexaglutamated Antifolate) are also provided. Pharmaceutical compositions are sterile compositions that comprise a sample liposome and preferably polyglutamated Antifolate, preferably in a pharmaceutically acceptable carrier.

Unless otherwise stated herein, a variety of administration routes are available. The particular mode selected will depend, upon the particular active agent selected, the particular condition being treated, and the dosage required for therapeutic efficacy. The provided methods can be practiced using any known mode of administration that is medically acceptable and in accordance with good medical practice. In some embodiments, the administration route is an injection. In further embodiments, the injection is by a parenteral route elected from an intramuscular, subcutaneous, intravenous, intraarterial, intraperitoneal, intraarticular, intraepidural, intrathecal, intravenous, intramuscular, or intra sternal injection. In some embodiments, the administration route is an infusion. In additional embodiments, the administration route is oral, nasal, mucosal, sublingual, intratracheal, ophthalmic, rectal, vaginal, ocular, topical, transdermal, pulmonary, or inhalation.

Therapeutic compositions containing PANTIFOL compositions such as the liposomal PANTIFOL compositions described herein can be conventionally administered intravenously, as by injection of a unit dose, for example. The term “unit dose” when used in reference to a therapeutic composition provided herein refers to physically discrete units suitable as unitary dosage for the subject, each unit containing a predetermined quantity of active material calculated to produce the desired therapeutic effect in association with the required diluent; e.g., carrier, or vehicle. In a specific embodiment, therapeutic compositions containing an Adapter are administered subcutaneously.

In some embodiments, the PANTIFOL composition is administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration are also variable but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

The PANTIFOL composition are formulated, dosed, and administered in a fashion consistent with good medical practice. Factors for consideration in this context include the particular disorder being treated, the particular patient being treated, the clinical condition of the individual patient, the cause of the disorder, the site of delivery of the agent, the method of administration, the scheduling of administration, and other factors known to medical practitioners. The dosage ranges for the administration of PANTIFOL composition are those large enough to produce the desired effect in which the disease symptoms mediated by the target molecule are ameliorated. The dosage should not be so large as to cause adverse side effects, such as, hyperviscosity syndromes, pulmonary edema, congestive heart failure, and other adverse side effects known in the art. Generally, the dosage will vary with the age, weight, height, body surface area, state of health (e.g., renal and liver function), condition, sex and extent of the disease in the patient and can routinely be determined by one of ordinary skill in the art. The dosage can be adjusted by the individual physician in the event of any complication.

The dosage schedule and amounts effective for therapeutic and prophylactic uses, i.e., the “dosing regimen,” will depend upon a variety of factors, including the cause, stage and severity of the disease or disorder, the health, physical status, age of the subject being treated, and the site and mode of the delivery of the PANTIFOL composition. Therapeutic efficacy and toxicity of the PANTIFOL composition can be determined by standard pharmaceutical, pharmacological, and toxicological procedures in cell cultures or experimental animals. Data obtained from these procedures can likewise be used in formulating a range of dosages for human use. Moreover, therapeutic index (i.e., the dose therapeutically effective in 50 percent of the population divided by the dose lethal to 50 percent of the population (ED50/LD50)) can readily be determined using known procedures. The dosage is preferably within a range of concentrations that includes the ED50 with little or no toxicity, and may vary within this range depending on the dosage form employed, sensitivity of the patient, and the route of administration.

The dosage regimen also takes into consideration pharmacokinetics parameters known in the art, such as, drug absorption rate, bioavailability, metabolism and clearance (see, e.g., Hidalgo-Aragones, J. Steroid Biochem. Mol. Biol. 58:611-617 (1996); Groning et al., Pharmazie 51:337-341 (1996); Fotherby, Contraception 54:59-69 (1996); and Johnson et al., J. Pharm. Sci. 84:1144-1146 (1995)). It is well within the state of the art for the clinician to determine the dosage regimen for each subject being treated. Moreover, single or multiple administrations of the PANTIFOL composition can be administered depending on the dosage and frequency as required and tolerated by the subject. The duration of prophylactic and therapeutic treatment will vary depending on the particular disease or condition being treated. Some diseases are amenable to acute treatment whereas others require long-term, chronic therapy. The PANTIFOL composition can be administered serially, or simultaneously with the additional therapeutic agent.

In some embodiments, the PANTIFOL composition is administered in a liposomal composition at a dose of between 0.005 and 5000 mg of PANTIFOL per square meter of body surface area, or any range therein between. In further embodiments, the PANTIFOL composition is administered in a liposomal composition at a dose of between 0.1 and 1000 mg PANTIFOL/meter squared of body surface area, or any range therein between.

In some embodiments, the PANTIFOL composition is administered in an immunoconjugate composition at a dose of 1 mg/kg to 500 mg/kg, 1 mg/kg to 250 mg/kg, 1 mg/kg to 200 mg/kg, 1 mg/kg to 150 mg/kg, 1 mg/kg to 100 mg/kg, 1 mg/kg to 50 mg/kg, 1 mg/kg to 25 mg/kg, 1 mg/kg to 20 mg/kg, 1 mg/kg to 15 mg/kg, 1 mg/kg to 10 mg/kg, or 1 mg/kg to 5 mg/kg, or any range therein between.

In another embodiment, the PANTIFOL composition is administered in combination with one or more additional therapeutics.

In some embodiment, the PLp-PANTIFOL and/or targeted-PLp-PANTIFOL is prepared as an infusion composition, an injection composition, a parenteral composition, or a topical composition. In further embodiments, the injection includes one or more of: intraperitoneal injection, direct intratumor injection, intra-arterial injection, and intravenous injection, subcutaneous injection, intramuscular injection, delivery via transcutaneous and intranasal route. In a further embodiment, the PLp-PANTIFOL and/or targeted-PLp-PANTIFOL is a liquid solution or a suspension. However, solid forms suitable for solution in, or suspension in, liquid vehicles prior to injection are also provided herein. In some embodiments, the targeted pegylated liposomal polyglutamated Antifolate composition is formulated as an enteric-coated tablet or gel capsule according to methods known in the art.

In some embodiments, the targeted pegylated liposomal polyglutamated Antifolate formulations are administered to a tumor of the central nervous system using a slow, sustained intracranial infusion of the liposomes directly into the tumor (e.g., a convection-enhanced delivery (CED)). See, Saito et al., Cancer Research 64:2572-2579 (2004); Mamot et al., J. Neuro-Oncology 68:1-9 (2004). In other embodiments, the formulations are directly applied to tissue surfaces. Sustained release, pH dependent release, and other specific chemical or environmental condition-mediated release administration of the pegylated liposomal polyglutamated Antifolate formulations (e.g., depot injections and erodible implants) are also provided. Examples of such release-mediating compositions are further described herein or otherwise known in the art.

For administration by inhalation, the compositions can be conveniently delivered in the form of an aerosol spray presentation from pressurized packs or a nebulizer, with the use of a suitable propellant, e.g., dichlorodifluoromethane, trichlorofluoromethane, ichlorotetrafluoroethane, carbon dioxide or other suitable gas. In the case of a pressurized aerosol, the dosage unit can be determined by providing a valve to deliver a metered amount.

When it is desirable to deliver the compositions systemically, they can be formulated for parenteral administration by injection, e.g., by bolus injection or continuous infusion. Formulations for injection can be presented in unit dosage form, e.g., in ampoules or in multi-dose containers. Pharmaceutical parenteral formulations include aqueous solutions of the ingredients. Aqueous injection suspensions can contain substances which increase the viscosity of the suspension, such as sodium carboxymethyl cellulose, sorbitol, or dextran. Alternatively, suspensions of liposomes can be prepared as oil-based suspensions. Suitable lipophilic solvents or vehicles include fatty oils such as sesame oil, or synthetic fatty acid esters, such as ethyl oleate or triglycerides.

Alternatively, the non-targeted or targeted pegylated liposomal polyglutamated Antifolate can be in powder form or lyophilized form for constitution with a suitable vehicle, e.g., sterile pyrogen-free water, before use.

The provided compositions (e.g., alpha and/or gamma polyglutamated Antifolate and liposomes containing the alpha and/or gamma polyglutamated Antifolate) can also be formulated in rectal or vaginal compositions such as suppositories or retention enemas, e.g., containing conventional suppository bases such as cocoa butter or other glycerides.

### Methods of Use and Treatment

In additional embodiments, the disclosure provides methods of using polyglutamated Antifolate (PANTIFOL) compositions such as αPANTIFOL or γPANTIFOL compositions. In some embodiments, the gamma γPANTIFOL compositions are used to treat a disease or disorder. In some embodiments, the alpha αPANTIFOL compositions are used to treat a disease or disorder.

In some embodiments, the disclosure provides a method of killing a cell that comprises contacting the cell with a composition comprising a polyglutamated Antifolate (e.g., a γPANTIFOL disclosed herein or αPANTIFOL disclosed herein). In some embodiments, the polyglutamated Antifolate is a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the contacted cell is a mammalian cell. In further embodiments, the contacted cell is a human cell. In some embodiments, the contacted cell is a hyperproliferative cell. In further embodiments, the hyperproliferative cell is a cancer cell. In further embodiments, the cancer cell is a primary cell or a cell from a cell line obtained/derived from a cancer selected from: a non-hematologic malignancy including such as for example, lung cancer, pancreatic cancer, breast cancer, ovarian cancer, prostate cancer, head and neck cancer, gastric cancer, gastrointestinal cancer, colorectal cancer, esophageal cancer, cervical cancer, liver cancer, kidney cancer, biliary duct cancer, gallbladder cancer, bladder cancer, sarcoma (e.g., osteosarcoma), brain cancer, central nervous system cancer, and melanoma; and a hematologic malignancy such as for example, a leukemia, a lymphoma and other B cell malignancies, myeloma and other plasma cell dysplasias or dyscrasias. In some embodiments, the contacted cancer cell is a primary cell or a cell from a cell line obtained/derived from lung cancer (e.g., NSCLC or mesothelioma). In some embodiments, the contacted cancer cell is a primary cell or a cell from a cell line obtained/derived from breast cancer (e.g., HER2++ or triple negative breast cancer). In some embodiments, the contacted cancer cell is a primary cell or a cell from a cell line obtained/derived from colorectal cancer. In some embodiments, the contacted cancer cell is a primary cell or a cell from a cell line obtained/derived from ovarian cancer. In some embodiments, the contacted cancer cell is a primary cell or a cell from a cell line obtained/derived from endometrial cancer. In some embodiments, the contacted cancer cell is a primary cell or a cell from a cell line obtained/derived from pancreatic cancer. In some embodiments, the contacted cancer cell is a primary cell or a cell from a cell line obtained/derived from liver cancer. In some embodiments, the contacted cancer cell is a primary cell or a cell from a cell line obtained/derived from head and neck cancer. In some embodiments, the contacted cancer cell is a primary cell or a cell from a cell line obtained/derived from osteosarcoma. In some embodiments, the method is performed in vivo. In other embodiments, the method is performed in vitro. In some embodiments, the PANTIFOL composition contains 4, 5, 2-10, 4-6, or more than 5, γ-glutamyl groups or α-glutamyl groups. In some embodiments, the PANTIFOL composition comprises gamma pentaglutamated Antifolate or alpha pentaglutamated Antifolate. In some embodiments, the PANTIFOL composition comprises gamma hexaglutamated Antifolate or alpha hexaglutamated Antifolate. In some embodiments, the PANTIFOL composition comprises L gamma polyglutamated Antifolate or L alpha polyglutamated Antifolate. In some embodiments, the PANTIFOL composition comprises D gamma polyglutamated Antifolate or D alpha polyglutamated Antifolate. In some embodiments, the PANTIFOL composition comprises L and D gamma polyglutamated Antifolate or L and D alpha polyglutamated Antifolate.

In additional embodiments, the disclosure provides a method of killing a cell that comprises contacting the cell with a liposome containing polyglutamated Antifolate (e.g., an Lp-PANTIFOL such as, PLp-PANTIFOL, NTLp-PANTIFOL, NTPLp-PANTIFOL, TLp-PANTIFOL or TPLp-PANTIFOL disclosed herein). In some embodiments, the liposomal composition comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the liposome is pegylated (e.g., PLp-PANTIFOL and NTPLp-PANTIFOL). In some embodiments, the liposome comprises a targeting moiety on its surface that specifically binds an antigen on the surface of the cell (e.g., TLp-PANTIFOL and TPLp-PANTIFOL). In further embodiments, the liposome is pegylated and comprises a targeting moiety on its surface that specifically binds an antigen on the surface of the cell (e.g., TPLp-PANTIFOL). In some embodiments, the contacted cell is a mammalian cell. In further embodiments, the contacted cell is a human cell. In additional embodiments, the contacted cell is a hyperproliferative cell. In further embodiments, the hyperproliferative cell is a cancer cell. In further embodiments, the contacted cancer cell is a primary cell or a cell from a cell line obtained/derived from a cancer selected from: lung cancer (e.g., non-small cell), pancreatic cancer, breast cancer, ovarian cancer, prostate cancer, head and neck cancer, gastric cancer, gastrointestinal cancer, colorectal cancer, esophageal cancer, cervical cancer, liver cancer, kidney cancer, biliary duct cancer, gallbladder cancer, bladder cancer, sarcoma (e.g., osteosarcoma), brain cancer, central nervous system cancer, melanoma, myeloma, a leukemia and a lymphoma. In some embodiments, the contacted cancer cell is a primary cell or a cell from a cell line obtained/derived from lung cancer (e.g., NSCLC or mesothelioma). In some embodiments, the contacted cancer cell is a primary cell or a cell from a cell line obtained/derived from breast cancer (e.g., HER2++ or triple negative breast cancer). In some embodiments, the contacted cancer cell is a primary cell or a cell from a cell line obtained/derived from colorectal cancer. In some embodiments, the contacted cancer cell is a primary cell or a cell from a cell line obtained/derived from ovarian cancer. In some embodiments, the contacted cancer cell is a primary cell or a cell from a cell line obtained/derived from endometrial cancer. In some embodiments, the contacted cancer cell is a primary cell or a cell from a cell line obtained/derived from pancreatic cancer. In some embodiments, the contacted cancer cell is a primary cell or a cell from a cell line obtained/derived from liver cancer. In some embodiments, the contacted cancer cell is a primary cell or a cell from a cell line obtained/derived from head and neck cancer. In some embodiments, the contacted cancer cell is a primary cell or a cell from a cell line obtained/derived from osteosarcoma. In some embodiments, the method is performed in vivo. In other embodiments, the method is performed in vitro. In some embodiments, the liposome contains a PANTIFOL containing 4, 5, 2-10, 4-6, or more than 5, γ-glutamyl groups or α-glutamyl groups. In some embodiments, the liposome comprises L gamma polyglutamated Antifolate or L alpha polyglutamated Antifolate. In some embodiments, the liposome composition comprises 2, 3, 4, 5, or more than 5, L-gamma glutamyl groups or L-alpha glutamyl groups. In some embodiments, the liposome comprises D gamma polyglutamated Antifolate or D alpha polyglutamated Antifolate. In some embodiments, the liposome comprises 2, 3, 4, 5, or more than 5, D-gamma glutamyl groups or D-alpha glutamyl groups. In some embodiments, the administered liposome comprises 2, 3, 4, 5, or more than 5, L-gamma glutamyl groups or L-alpha glutamyl groups. In some embodiments, the liposome comprises L and D gamma polyglutamated Antifolate or L and D alpha polyglutamated Antifolate. In some embodiments, the liposome comprises 2, 3, 4, 5, or more than 5, L-gamma glutamyl groups and 2, 3, 4, 5, or more than 5, D-gamma glutamyl groups. In some embodiments, the liposome comprises 2, 3, 4, 5, or more than 5, L-alpha glutamyl groups and 2, 3, 4, 5, or more than 5, D-alpha glutamyl groups.

In some embodiments, the disclosure provides a method of killing a hyperproliferative cell that comprises contacting a hyperproliferative cell with a delivery vehicle (e.g., a liposome or antibody) comprising polyglutamated Antifolate (e.g., a PANTIFOL disclosed herein). In some embodiments, the delivery vehicle comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In some embodiments, the delivery vehicle is non-targeted. In other embodiments, the delivery vehicle is targeted and comprises a targeting moiety on its surface that has specific affinity for an epitope on an antigen on the surface of the hyperproliferative cell. In further embodiments, the delivery vehicle comprises a targeting moiety that has specific affinity for an epitope on an antigen on the surface of the hyperproliferative cell selected from GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TMEFF2, endoglin, PSMA, CD98, CD56, CanAg, and CALLA. In some embodiments, the method is performed in vivo. In some embodiments, the method is performed in vitro. In some embodiments, the delivery vehicle comprises a PANTIFOL consisting of 4, 5, 2-10, 4-6, or more than 5, glutamyl groups. In some embodiments, the delivery vehicle comprises gamma tetraglutamated Antifolate or alpha tetraglutamated Antifolate. In some embodiments, the delivery vehicle comprises gamma pentaglutamated Antifolate or alpha pentaglutamated Antifolate. In other embodiments, the delivery vehicle comprises gamma hexaglutamated Antifolate or alpha hexaglutamated Antifolate. In some embodiments, the delivery vehicle comprises L gamma polyglutamated Antifolate or L alpha polyglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups.

In certain particular embodiments, the method of a killing a hyperproliferative cell is performed using a liposome delivery vehicle that comprises polyglutamated Antifolate (e.g., an Lp-PANTIFOL such as, PLp-PANTIFOL, NTLp-PANTIFOL, NTPLp-PANTIFOL, TLp-PANTIFOL or TPLp-PANTIFOL disclosed herein). In some embodiments, the delivery vehicle comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the delivery vehicle is a non-targeted liposome. In some embodiments, the delivery vehicle comprises a targeting moiety on its surface that has specific affinity for an epitope on an antigen on the surface of the hyperproliferative cell (e.g., TLp-PANTIFOL and TPLp-PANTIFOL). In some embodiments, the delivery vehicle is a liposome comprising a targeting moiety on its surface that has specific affinity for an epitope on an antigen on the surface of the hyperproliferative cell. In further embodiments, the targeting moiety has specific affinity for an epitope on an antigen selected from GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TMEFF2, endoglin, PSMA, CD98, CD56, CanAg, and CALLA. In some embodiments, the liposome is pegylated (e.g., PLp-PANTIFOL, and NTPLp-PANTIFOL). In further embodiments, the liposome is pegylated and comprises a targeting moiety on its surface that has specific affinity for an epitope on an antigen on the surface of the hyperproliferative cell (e.g., TPLp-PANTIFOL). In other embodiments, the embodiments, the liposome is unpegylated. In some embodiments, the liposome is unpegylated and the liposome comprises a targeting moiety on its surface that has specific affinity for an epitope on an antigen on the surface of the hyperproliferative cell (e.g., TPLp-PANTIFOL). In some embodiments, the liposome comprises a PANTIFOL consisting of 4, 5, 2-10, 4-6, or more than 5, glutamyl groups. In some embodiments, the liposome comprises gamma tetraglutamated Antifolate or alpha tetraglutamated Antifolate. In some embodiments, the liposome comprises gamma pentaglutamated Antifolate or alpha pentaglutamated Antifolate. In other embodiments, the liposome comprises gamma hexaglutamated Antifolate or alpha hexaglutamated Antifolate. In some embodiments, the liposome comprises L gamma polyglutamated Antifolate or L alpha polyglutamated Antifolate. In some embodiments, the liposome comprises 2, 3, 4, 5, or more than 5, L-gamma glutamyl groups or L-alpha glutamyl groups. In some embodiments, the liposome comprises D gamma polyglutamated Antifolate or D alpha polyglutamated Antifolate. In some embodiments, the liposome comprises 2, 3, 4, 5, or more than 5, D-gamma glutamyl groups or D-alpha glutamyl groups. In some embodiments, the liposome comprises L and D gamma polyglutamated Antifolate or L and D alpha polyglutamated Antifolate. In some embodiments, the liposome comprises 2, 3, 4, 5, or more than 5, L-gamma glutamyl groups and 2, 3, 4, 5, or more than 5, D-gamma glutamyl groups. In some embodiments, the liposome comprises 2, 3, 4, 5, or more than 5, L-alpha glutamyl groups and 2, 3, 4, 5, or more than 5, D-alpha glutamyl groups.

In additional embodiments, the disclosure provides a method of inhibiting the proliferation of a cancer cell that comprises contacting the cancer cell with a delivery vehicle (e.g., a liposome or antibody) comprising polyglutamated Antifolate (e.g., a γPANTIFOL disclosed herein). In some embodiments, the delivery vehicle comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In some embodiments, the delivery vehicle is non-targeted. In some embodiments, the delivery vehicle is targeted and comprises a targeting moiety on its surface that has specific affinity for an epitope on an antigen on the surface of the cancer cell. In further embodiments, the delivery vehicle comprises a targeting moiety that has specific affinity for an epitope on a cell surface antigen selected from GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TMEFF2, endoglin, PSMA, CD98, CD56, CanAg, and CALLA. In some embodiments, the delivery vehicle is an antibody that has specific affinity for an epitope on an antigen on the surface of the cancer cell. In some embodiments, the contacted cancer cell is a mammalian cell. In further embodiments, the contacted cancer cell is a human cell. In additional embodiments, the contacted cancer cell is a primary cell or a cell from a cell line obtained/derived from a cancer selected from: lung cancer (e.g., non-small cell), pancreatic cancer, breast cancer, ovarian cancer, prostate cancer, head and neck cancer, gastric cancer, gastrointestinal cancer, colorectal cancer, esophageal cancer, cervical cancer, liver cancer, kidney cancer, biliary duct cancer, gallbladder cancer, bladder cancer, sarcoma (e.g., osteosarcoma), brain cancer, central nervous system cancer, melanoma, myeloma, a leukemia and a lymphoma. In some embodiments, the contacted cancer cell is a primary cell or a cell from a cell line obtained/derived from lung cancer (e.g., NSCLC or mesothelioma). In some embodiments, the contacted cancer cell is a primary cell or a cell from a cell line obtained/derived from breast cancer (e.g., HER2++ or triple negative breast cancer). In some embodiments, the contacted cancer cell is a primary cell or a cell from a cell line obtained/derived from colorectal cancer. In some embodiments, the contacted cancer cell is a primary cell or a cell from a cell line obtained/derived from ovarian cancer. In some embodiments, the contacted cancer cell is a primary cell or a cell from a cell line obtained/derived from endometrial cancer. In some embodiments, the contacted cancer cell is a primary cell or a cell from a cell line obtained/derived from pancreatic cancer. In some embodiments, the contacted cancer cell is a primary cell or a cell from a cell line obtained/derived from liver cancer. In some embodiments, the contacted cancer cell is a primary cell or a cell from a cell line obtained/derived from head and neck cancer. In some embodiments, the contacted cancer cell is a primary cell or a cell from a cell line obtained/derived from osteosarcoma. In some embodiments, the method is performed in vivo. In some embodiments, the method is performed in vitro. In some embodiments, the delivery vehicle is an antibody that has specific affinity for an epitope on one of the above-listed cell surface antigens. In other embodiments, the targeting vehicle is a liposome that comprises a targeting moiety that has specific affinity for an epitope on the surface of the cancer cell. In other embodiments, the targeting vehicle is a liposome that comprises a targeting moiety that has specific affinity for an epitope on one of the above-listed cell surface antigens. In some embodiments, the delivery vehicle is a liposome that is pegylated. In other embodiments, the delivery vehicle is a liposome that is not pegylated. In some embodiments, the delivery vehicle comprises a PANTIFOL composition containing 4, 5, 2-10, 4-6, or more than 5, γ-glutamyl groups or α-glutamyl groups. In some embodiments, the delivery vehicle comprises gamma tetraglutamated Antifolate or alpha tetraglutamated Antifolate. In some embodiments, the delivery vehicle comprises gamma pentaglutamated Antifolate or alpha pentaglutamated Antifolate. In other embodiments, the delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups.

In further embodiments, the disclosure provides a method of inhibiting the proliferation of a cancer cell that comprises contacting the cancer cell with a liposome comprising polyglutamated Antifolate (e.g., a PANTIFOL disclosed herein). In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the liposome is non-targeted. In some embodiments, the liposome is targeted and comprises a targeting moiety on its surface that has specific affinity for an epitope on an antigen on the surface of the cancer cell. In further embodiments, the liposome comprises a targeting moiety that has specific affinity for an epitope on a cell surface antigen selected from: GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TMEFF2, endoglin, PSMA, CD98, CD56, CanAg, and CALLA. In some embodiments, the contacted cancer cell is a mammalian cell. In further embodiments, the contacted cancer cell is a human cell. In additional embodiments, the contacted cancer cell is a primary cell or a cell from a cell line obtained/derived from a cancer selected from: lung cancer (e.g., non-small cell), pancreatic cancer, breast cancer, ovarian cancer, prostate cancer, head and neck cancer, gastric cancer, gastrointestinal cancer, colorectal cancer, esophageal cancer, cervical cancer, liver cancer, kidney cancer, biliary duct cancer, gallbladder cancer, bladder cancer, sarcoma (e.g., osteosarcoma), brain cancer, central nervous system cancer, melanoma, myeloma, a leukemia and a lymphoma. In some embodiments, the contacted cancer cell is a primary cell or a cell from a cell line obtained/derived from lung cancer (e.g., NSCLC or mesothelioma). In some embodiments, the contacted cancer cell is a primary cell or a cell from a cell line obtained/derived from breast cancer (e.g., HER2++ or triple negative breast cancer). In some embodiments, the contacted cancer cell is a primary cell or a cell from a cell line obtained/derived from colorectal cancer. In some embodiments, the contacted cancer cell is a primary cell or a cell from a cell line obtained/derived from ovarian cancer. In some embodiments, the contacted cancer cell is a primary cell or a cell from a cell line obtained/derived from endometrial cancer. In some embodiments, the contacted cancer cell is a primary cell or a cell from a cell line obtained/derived from pancreatic cancer. In some embodiments, the contacted cancer cell is a primary cell or a cell from a cell line obtained/derived from liver cancer. In some embodiments, the contacted cancer cell is a primary cell or a cell from a cell line obtained/derived from head and neck cancer. In some embodiments, the contacted cancer cell is a primary cell or a cell from a cell line obtained/derived from osteosarcoma. In some embodiments, the method is performed in vivo. In some embodiments, the method is performed in vitro. In other embodiments, the targeting vehicle is a liposome that comprises a targeting moiety that has specific affinity for an epitope on one of the above-listed cell surface antigens. In some embodiments, the liposome is pegylated. In other embodiments, the liposome that is not pegylated. In some embodiments, the liposome comprises a PANTIFOL consisting of 4, 5, 2-10, 4-6, or more than 5, glutamyl groups. In some embodiments, the liposome comprises gamma tetraglutamated Antifolate or alpha tetraglutamated Antifolate. In some embodiments, the liposome comprises gamma pentaglutamated Antifolate or alpha pentaglutamated Antifolate. In other embodiments, the liposome comprises gamma hexaglutamated Antifolate or alpha hexaglutamated Antifolate. In some embodiments, t the liposome comprises L gamma polyglutamated Antifolate or L alpha polyglutamated Antifolate. In some embodiments, the liposome comprises 2, 3, 4, 5, or more than 5, L-gamma glutamyl groups or L-alpha glutamyl groups. In some embodiments, the liposome comprises D gamma polyglutamated Antifolate or D alpha polyglutamated Antifolate. In some embodiments, the liposome comprises 2, 3, 4, 5, or more than 5, D-gamma glutamyl groups or D-alpha glutamyl groups. In some embodiments, the liposome comprises L and D gamma polyglutamated Antifolate or L and D alpha polyglutamated Antifolate. In some embodiments, the liposome comprises 2, 3, 4, 5, or more than 5, L-gamma glutamyl groups or L-alpha glutamyl groups. In some embodiments, the liposome comprises 2, 3, 4, 5, or more than 5, L-gamma glutamyl groups and 2, 3, 4, 5, or more than 5, D-gamma glutamyl groups. In some embodiments, the liposome comprises 2, 3, 4, 5, or more than 5, L-alpha glutamyl groups and 2, 3, 4, 5, or more than 5, D-alpha glutamyl groups.

In additional embodiments, the disclosure provides a method for treating a hyperproliferative disorder that comprises administering an effective amount of a delivery vehicle (e.g., antibody or liposome) comprising polyglutamated Antifolate (e.g., a PANTIFOL disclosed herein) to a subject having or at risk of having a hyperproliferative disorder. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In some embodiments, the administered delivery vehicle is pegylated. In some embodiments, the administered delivery vehicle is not pegylated. In additional embodiments, the administered delivery vehicle comprises a targeting moiety that has a specific affinity for an epitope of antigen on the surface of the hyperproliferative cell. In additional embodiments, the delivery vehicle comprises a targeting moiety that specifically binds a cell surface antigen selected from: GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TMEFF2, endoglin, PSMA, CD98, CD56, CanAg, and CALLA. In some embodiments, the targeting moiety is an antibody or an antigen binding antibody fragment. In some embodiments, the administered delivery vehicle does not comprise a targeting moiety that has a specific affinity for an epitope on a cell surface antigen of the hyperproliferative cell. In some embodiments, the delivery vehicle comprises a PANTIFOL consisting of 4, 5, 2-10, 4-6, or more than 5, glutamyl groups. In some embodiments, the delivery vehicle comprises gamma tetraglutamated Antifolate or alpha tetraglutamated Antifolate. In some embodiments, the delivery vehicle comprises gamma pentaglutamated Antifolate or alpha pentaglutamated Antifolate. In other embodiments, the delivery vehicle comprises gamma hexaglutamated Antifolate or alpha hexaglutamated Antifolate. In some embodiments, the delivery vehicle comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups. In some embodiments, the hyperproliferative disorder is cancer. In some embodiments, the hyperproliferative disorder is an autoimmune disease (e.g., rheumatoid arthritis). In some embodiments, the hyperproliferative disorder is a benign or malignant tumor; leukemia, hematological, or lymphoid malignancy. In other embodiments, the hyperproliferative disorder selected from a neuronal, glial, astrocytal, hypothalamic, glandular, macrophagal, epithelial, stromal, blastocoelic, inflammatory, angiogenic and immunologic disorder, including an autoimmune disease.

In additional embodiments, the disclosure provides a method for treating a hyperproliferative disorder that comprises administering an effective amount of a liposome comprising polyglutamated Antifolate (e.g., an Lp-PANTIFOL such as, PLp-PANTIFOL, NTLp-PANTIFOL, NTPLp-PANTIFOL, TLp-PANTIFOL, or TPLp-PANTIFOL) to a subject having or at risk of having a hyperproliferative disorder. In some embodiments, the liposome is pegylated. In some embodiments, the liposome is not pegylated. In additional embodiments, the liposome comprises a targeting moiety that has a specific affinity for an epitope of antigen on the surface of the hyperproliferative cell. In additional embodiments, the liposome comprises a targeting moiety that specifically binds a cell surface antigen selected from: GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TMEFF2, endoglin, PSMA, CD98, CD56, CanAg, and CALLA. In some embodiments, the targeting moiety is an antibody or an antigen binding antibody fragment. In some embodiments, the liposome does not comprise a targeting moiety that has a specific affinity for an epitope on a cell surface antigen of the hyperproliferative cell. In some embodiments, the liposome comprises a PANTIFOL consisting of 4, 5, 2-10, 4-6, or more than 5, glutamyl groups. In some embodiments, the liposome comprises gamma tetraglutamated Antifolate or alpha tetraglutamated Antifolate. In some embodiments, the liposome comprises gamma pentaglutamated Antifolate or alpha pentaglutamated Antifolate. In other embodiments, the liposome comprises gamma hexaglutamated Antifolate or alpha hexaglutamated Antifolate. In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the liposome comprises 2, 3, 4, 5, or more than 5, L-gamma glutamyl groups or L-alpha glutamyl groups. In some embodiments, the liposome comprises D gamma polyglutamated Antifolate or D alpha polyglutamated Antifolate. In some embodiments, the liposome comprises 2, 3, 4, 5, or more than 5, D-gamma glutamyl groups or D-alpha glutamyl groups. In some embodiments, the liposome comprises L and D gamma polyglutamated Antifolate or L and D alpha polyglutamated Antifolate. In some embodiments, t the liposome comprises 2, 3, 4, 5, or more than 5, L-gamma glutamyl groups or L-alpha glutamyl groups. In some embodiments, the liposome comprises 2, 3, 4, 5, or more than 5, L-gamma glutamyl groups and 2, 3, 4, 5, or more than 5, D-gamma glutamyl groups. In some embodiments, the liposome comprises 2, 3, 4, 5, or more than 5, L-alpha glutamyl groups and 2, 3, 4, 5, or more than 5, D-alpha glutamyl groups. In some embodiments, the hyperproliferative disorder is cancer. In some embodiments, the hyperproliferative disorder is an autoimmune disease (e.g., rheumatoid arthritis). In some embodiments, the hyperproliferative disorder is a benign or malignant tumor; leukemia, hematological, or lymphoid malignancy. In other embodiments, the hyperproliferative disorder is selected from a neuronal, glial, astrocytal, hypothalamic, glandular, macrophagal, epithelial, stromal, blastocoelic, inflammatory, angiogenic and immunologic disorder, including an autoimmune disease.

Exemplary hyperproliferative disorders that can be treated according to the disclosed methods include, but are not limited to, disorders associated with benign, pre-malignant, and malignant cellular proliferation, including but not limited to, neoplasms and tumors (e.g., histiocytoma, glioma, astrocytoma, osteoma), cancers (e.g., lung cancer, small cell lung cancer, gastrointestinal cancer, bowel cancer, colorectal cancer, breast carcinoma, ovarian carcinoma, prostate cancer, testicular cancer, liver cancer, kidney cancer, bladder cancer, pancreatic cancer, brain cancer, sarcoma (e.g., osteosarcoma, Kaposi's sarcoma), and melanoma), leukemias, psoriasis, bone diseases, fibroproliferative disorders (e.g., of connective tissues), and atherosclerosis.

In additional embodiments, the disclosure provides a method for treating cancer that comprises administering an effective amount of a delivery vehicle (e.g., antibody or liposome) comprising polyglutamated Antifolate (e.g., a PANTIFOL disclosed herein) to a subject having or at risk of having cancer. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In some embodiments, the administered delivery vehicle is pegylated. In some embodiments, the administered delivery vehicle is not pegylated. In additional embodiments, the administered delivery vehicle comprises a targeting moiety that has a specific affinity for an epitope of antigen on the surface of a cancer cell. In some embodiments, the targeting moiety is an antibody or an antigen binding antibody fragment. In some embodiments, the administered delivery vehicle comprises PANTIFOL containing 4, 5, 2-10, 4-6, or more than 5, glutamyl groups. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups. In some embodiments, the cancer is selected from: lung (e.g., non-small lung cancer), pancreatic, breast cancer, ovarian, lung, prostate, head and neck, gastric, gastrointestinal, colon, esophageal, cervical, kidney, biliary duct, gallbladder, and a hematologic malignancy (e.g., a leukemia or lymphoma). In some embodiments, the cancer is lung cancer (e.g., NSCLC or mesothelioma). In some embodiments, the cancer is breast cancer (e.g., HER2++ or triple negative breast cancer). In some embodiments, the cancer is colorectal cancer. In some embodiments, the cancer is ovarian cancer. In some embodiments, the cancer is endometrial cancer. In some embodiments, the cancer is pancreatic cancer. In some embodiments, the cancer is liver cancer. In some embodiments, the cancer is head and neck cancer. In some embodiments, the cancer is osteosarcoma.

In additional embodiments, the disclosure provides a method for treating, reducing, or inhibiting metastasis that comprises administering an effective amount of a delivery vehicle (e.g., antibody or liposome) comprising polyglutamated Antifolate (e.g., a PANTIFOL disclosed herein) to a subject having or at risk of having cancer. In some embodiments, the disclosed methods provide among other things, (1) reducing or inhibiting growth, proliferation, survival, mobility or invasiveness of a primary tumor, cancer or neoplasia; (2) reducing or inhibiting growth, proliferation, survival, mobility or invasiveness of a primary tumor, cancer or neoplasia that potentially or does develop metastases; (3) reducing or inhibiting formation or establishment of metastases arising from a primary tumor, cancer or neoplasia to one or more other sites, locations, regions or systems distinct from the primary tumor, cancer or neoplasia; (4) reducing or inhibiting growth or proliferation of a metastasis at one or more other sites, locations, regions or systems distinct from the primary tumor, cancer or neoplasia after a metastasis has formed or has been established; and/or (5) reducing or inhibiting formation or establishment of additional metastasis after the metastasis has been formed or established. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In some embodiments, the administered delivery vehicle is pegylated. In some embodiments, the administered delivery vehicle is not pegylated. In additional embodiments, the administered delivery vehicle comprises a targeting moiety that has a specific affinity for an epitope of antigen on the surface of a cancer cell. In some embodiments, the targeting moiety is an antibody or an antigen binding antibody fragment. In some embodiments, the administered delivery vehicle comprises PANTIFOL containing 4, 5, 2-10, 4-6, or more than 5, glutamyl groups. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups. In some embodiments, the cancer is selected from: lung (e.g., non-small lung cancer), pancreatic, breast cancer, ovarian, lung, prostate, head and neck, gastric, gastrointestinal, colon, esophageal, cervical, kidney, biliary duct, gallbladder, and a hematologic malignancy (e.g., a leukemia or lymphoma). In some embodiments, the cancer is lung cancer (e.g., NSCLC or mesothelioma). In some embodiments, the cancer is breast cancer (e.g., HER2++ or triple negative breast cancer). In some embodiments, the cancer is colorectal cancer. In some embodiments, the cancer is ovarian cancer. In some embodiments, the cancer is endometrial cancer. In some embodiments, the cancer is pancreatic cancer. In some embodiments, the cancer is liver cancer. In some embodiments, the cancer is head and neck cancer. In some embodiments, the cancer is osteosarcoma.

In additional embodiments, the disclosure provides a method for treating cancer that comprises administering an effective amount of a delivery vehicle (e.g., antibody or liposome) comprising polyglutamated Antifolate (e.g., a PANTIFOL disclosed herein) to a subject having or at risk of having cancer. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In some embodiments, the administered delivery vehicle is pegylated. In some embodiments, the administered delivery vehicle is not pegylated. In additional embodiments, the administered delivery vehicle comprises a targeting moiety that has a specific affinity for an epitope of antigen on the surface of a cancer cell. In additional embodiments, the delivery vehicle comprises a targeting moiety that specifically binds a cell surface antigen selected from: GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TMEFF2, endoglin, PSMA, CD98, CD56, CanAg, and CALLA. In some embodiments, the targeting moiety is an antibody or an antigen binding antibody fragment. In some embodiments, the administered delivery vehicle comprises PANTIFOL containing 4, 5, 2-10, 4-6, or more than 5, glutamyl groups. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups. In some embodiments, the cancer is selected from: lung (e.g., non-small lung cancer), pancreatic, breast cancer, ovarian, lung, prostate, head and neck, gastric, gastrointestinal, colon, esophageal, cervical, kidney, biliary duct, gallbladder, and a hematologic malignancy (e.g., a leukemia or lymphoma). In some embodiments, the cancer is lung cancer (e.g., NSCLC or mesothelioma). In some embodiments, the cancer is breast cancer (e.g., HER2++ or triple negative breast cancer). In some embodiments, the cancer is colorectal cancer. In some embodiments, the cancer is ovarian cancer. In some embodiments, the cancer is endometrial cancer. In some embodiments, the cancer is pancreatic cancer. In some embodiments, the cancer is liver cancer. In some embodiments, the cancer is head and neck cancer. In some embodiments, the cancer is osteosarcoma.

In additional embodiments, the disclosure provides a method for treating cancer that comprises administering an effective amount of a liposome comprising polyglutamated Antifolate (e.g., an Lp-PANTIFOL such as, PLp-PANTIFOL, NTLp-PANTIFOL, NTPLp-PANTIFOL, TLp-PANTIFOL, or TPLp-PANTIFOL) to a subject having or at risk of having cancer. In some embodiments, the liposome is pegylated. In some embodiments, the liposome is not pegylated. In additional embodiments, the liposome comprises a targeting moiety that has a specific affinity for an epitope of antigen on the surface of a cancer cell. In additional embodiments, the liposome comprises a targeting moiety that specifically binds a cell surface antigen selected from: GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TMEFF2, endoglin, PSMA, CD98, CD56, CanAg, and CALLA. In some embodiments, the cancer is selected from: lung (e.g., non-small lung cancer), pancreatic, breast cancer, ovarian, lung, prostate, head and neck, gastric, gastrointestinal, colon, esophageal, cervical, kidney, biliary duct, gallbladder, and a hematologic malignancy (e.g., a leukemia or lymphoma). In some embodiments, the targeting moiety is an antibody or an antigen binding antibody fragment. In some embodiments, the liposome comprises PANTIFOL containing 4, 5, 2-10, 4-6, or more than 5, γ-glutamyl groups or α-glutamyl groups. In some embodiments, the liposome comprises gamma tetraglutamated Antifolate or alpha tetraglutamated Antifolate. In some embodiments, the liposome comprises gamma pentaglutamated Antifolate or alpha pentaglutamated Antifolate. In other embodiments, the liposome comprises gamma hexaglutamated Antifolate or alpha hexaglutamated Antifolate. In some embodiments, the liposome comprises L gamma polyglutamated Antifolate or L alpha polyglutamated Antifolate. In some embodiments, the liposome comprises a γPANTIFOL containing 1, 2, 3, 4, 5, 6, 7, 8, 9, 10, or more than 10, γ-glutamyl groups in the L-form. In some embodiments, the liposome comprises a αPANTIFOL containing 1, 2, 3, 4, 5, 6, 7, 8, 9, 10, or more than 10, α-glutamyl groups in the L-form. In some embodiments, the liposome comprises D gamma polyglutamated Antifolate or D alpha polyglutamated Antifolate. In some embodiments, the liposome comprises a γPANTIFOL containing 1, 2, 3, 4, 5, 6, 7, 8, 9, 10, or more than 10, γ-glutamyl groups in the D-form. In some embodiments, the liposome comprises a αPANTIFOL containing 1, 2, 3, 4, 5, 6, 7, 8, 9, 10, or more than 10, α-glutamyl groups in the D-form. In some embodiments, the liposome comprises L and D gamma polyglutamated Antifolate or L and D alpha polyglutamated Antifolate. In some embodiments, the liposome comprises a γPANTIFOL containing 2, 3, 4, 5, or more than 5, γ-glutamyl groups in the L-form, and 1, 2, 3, 4, 5 or more than 5, γ-glutamyl groups in the D-form. In some embodiments, the administered delivery vehicle comprises 2, 3, 4, 5, or more than 5, L-alpha glutamyl groups and 1, 2, 3, 4, 5, or more than 5, D-alpha glutamyl groups. In additional embodiments, the disclosure provides a method for treating cancer that comprises administering to a subject having or at risk of having cancer, an effective amount of a liposomal composition containing a liposome that comprises gamma polyglutamated Antifolate and a targeting moiety that has a specific affinity for an epitope of antigen on the surface of the cancer. In some embodiments, the liposome comprises a targeting moiety that specifically binds a cell surface antigen selected from: GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TME1-1-2, endoglin, PSMA, CD98, CD56, CanAg, and CALLA. In some embodiments, the liposomal composition is administered to treat a cancer selected from: lung cancer, pancreatic, breast cancer, ovarian cancer, lung cancer, prostate cancer, head and neck cancer, gastric cancer, gastrointestinal cancer, colon cancer, esophageal cancer, cervical cancer, kidney cancer, biliary duct cancer, gallbladder cancer, and a hematologic malignancy. In some embodiments, the administered liposomal composition comprises pegylated liposomes (e.g., TPLp-γPANTIFOL). In some embodiments, the administered liposomal composition comprises liposomes that are not pegylated. In some embodiments, liposomes of the administered liposomal composition comprises PANTIFOL containing 4, 5, 2-10, 4-6, or more than 5, γ-glutamyl groups or α-glutamyl groups. In some embodiments, liposomes of the administered liposomal composition comprise gamma tetraglutamated Antifolate or alpha tetraglutamated Antifolate. In some embodiments, liposomes of the administered liposomal composition comprise gamma pentaglutamated Antifolate or alpha pentaglutamated Antifolate. In other embodiments, liposomes of the administered liposomal composition comprises gamma hexaglutamated Antifolate or alpha hexaglutamated Antifolate. In some embodiments, a liposome of the liposomal composition comprises a PANTIFOL containing 2, 3, 4, 5, 6, 7, 8, 9, 10, or more than 10, γ-glutamyl groups or α-glutamyl groups in the L-form. In some embodiments, a liposome of the liposomal composition comprises D gamma polyglutamated Antifolate or D alpha polyglutamated Antifolate. In some embodiments, a liposome of the liposomal composition comprises a PANTIFOL containing 1, 2, 3, 4, 5, 6, 7, 8, 9, 10, or more than 10, γ-glutamyl groups or α-glutamyl groups in the D-form. In some embodiments, the liposome comprises L and D gamma polyglutamated Antifolate or L and D alpha polyglutamated Antifolate. In some embodiments, a liposome of the liposomal composition comprises γPANTIFOL containing 2, 3, 4, 5, or more than 5, γ-glutamyl groups in the L-form, and 1, 2, 3, 4, 5 or more than 5, γ-glutamyl groups in the D-form. In some embodiments, a liposome of the liposomal composition comprises αPANTIFOL containing 2, 3, 4, 5, or more than 5, α-glutamyl groups in the L-form, and 1, 2, 3, 4, 5 or more than 5, α-glutamyl groups in the D-form.

In some embodiments, the liposome comprises a targeting moiety that has specific affinity for an epitope of a tumor specific antigen (TSA) or tumor associated antigen (TAA). In some embodiments, the liposome comprises a targeting moiety that has specific affinity for an epitope of an antigen selected from: a tumor differentiation antigen (e.g., MART1/MelanA, gp100 (Pmel 17), tyrosinase, TRP1, and TRP2), a tumor-specific multilineage antigen (e.g., MAGE1, MAGE3, BAGE, GAGE1, GAGE2, and p15), an overexpressed embryonic antigen (e.g., carcinoembryonic antigen (CEA)), an overexpressed oncogene or mutated tumor-suppressor gene product (e.g., p53, Ras, and HER2/neu), a unique tumor antigen resulting from chromosomal translocations (e.g., BCR-ABL, E2A-PRL, H4-RET, IGH-IGK, and MYL-RAR), a viral antigen (e.g., Epstein Barr virus antigen EBVA, human papillomavirus (HPV) antigen E6 or E7), GP 100), prostatic acid phosphatase (PAP), prostate-specific antigen (PSA), PTGER4, ITGA4, CD37, CD52, CD62L (L-selectin), CXCR4, CD69, EVI2B (CD361), SLC39A8, MICB, LRRC70, CLELC2B, HMHA1, LST1, and CMTM6 (CKLFSF6). In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the liposome comprises a targeting moiety that has specific affinity for an epitope of a hematologic tumor antigen. In further embodiments, the targeting moiety has specific affinity for an epitope of a hematologic tumor antigen selected from: CD19, CD20, CD22, CD30, CD138, CD33, CD34, CD38, CD123, CS1, ROR1, LewisY, Ig kappa light chain, TCR, BCMA, TACI, BAFFR (CD268), CALLA, and a NKG2DL ligand). In some embodiments, the liposome comprises a targeting moiety that has specific affinity for an epitope of a B-cell lymphoma-specific idiotype immunoglobulin, or a B-cell differentiation antigen (e.g., CD19, CD20, and CD37). In some embodiments, the liposome comprises a targeting moiety that has specific affinity for an epitope of an antigen on a multiple myeloma cell (e.g., CS-1, CD38, CD138, MUC1, HM1.24, CYP1B1, SP17, PRAME, Wilms' tumor 1 (WT1), and heat shock protein gp96) or an antigen on myeloid cells (e.g., TSLPR and IL-7R). In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the liposome comprises a targeting moiety that has specific affinity for an epitope of a solid tumor antigen. In further embodiments, the targeting moiety has specific affinity for an epitope of a hematologic tumor antigen selected from: disialoganglioside (GD2), o-acetyl GD2, EGFRvIII, ErbB2, VEGFR2, FAP, mesothelin, IL13Ra2 (glioma), cMET, PSMA, L1CAM, CEA, and EGFR. In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the liposome comprises a targeting moiety that has specific affinity for an epitope of an antigen selected from: GONMB, TACSTD2 (TROP2), CEACAM5, EPCAM, a folate receptor (e.g., folate receptor-γ, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin γvβ3, γvβ5, or γvβ6), a C242 antigen, Apo2, PSGR, NGEP, PSCA, TMEFF2, endoglin, PSMA, CD98, CD56, CanAg, and CALLA. In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the liposome comprises a targeting moiety that has specific affinity for an epitope of an antigen selected from: CD137, PDL1, CTLA4, CD47, KIR, TNFRSF10B (DR5), TIM3, PD1, cMet, Glycolipid F77, EGFRvIII, HLAA2 (NY-ESO-1), LAGS, CD134 (OX40), HVEM, BTLA, TNFRSF25 (DR3), CD133, MAGE A3, PSCA, MUC1, CD44v6, CD44v6/7, CD44v7/8, IL11Ra, ephA2, CAIX, MNCAIX, CSPG4, MUC16, EPCAM (EGP2), TAG72, EGP40, ErbB receptor family, ErbB2 (HER2), ErbB3/4, RAGE1, GD3, FAR, LewisY, NCAM, HLAA1/MAGE1, MAGEA1, MAGEA3, MAGE-A4, B7H3, WT1, MelanA (MART1), HPV E6, HPV E7, thyroglobulin, tyrosinase, PSA, CLL1GD3, Tn Ag, FLT3, KIT, PRSS21, CD24, PDGFR-beta, SSEA4, prostase, PAP, ELF2M, ephB2, IGF1, IGFII, IGFI receptor, LMP2, gp100, bcr-abl, Fucosyl GM1, sLe, GM3, TGSS, folate receptor beta, TEM1 (CD248), TEM7R, CLDN6, TSHR, GPRCSD, CXORF61, CD97, CD7a, HLE, CD179a, ALK, Plysialic acid, PLAC1, GloboH, NY-BR-1, UPK2, HAVCR1, ADRB3, PANX3, GPR20, LY6K, OR51E2, TARP, LAGEla, legumain, E7, ETV6-AML, sperm protein 17, XAGE1, Tie 2, MAD-CT1, MAD-CT2, Fos-related antigen 1, p53, p53 mutant, prostein, survivin, telomerase, PCTA1 (Galectin 8), Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP2, CYP1B1, BORIS, SART3, PAXS, OY-TES1, LCK, AKAP4, SSX2, reverse transcriptase, RU1, RU2, intestinal carboxyl esterase, neutrophil elastase, mut hsp70-2, CD79a, CD79b, CD72, LAIR1, FCAR, LILRA2, CD300LF, CLEC12A, BST2, EMR2, LY75, GPC3, FCRLS, IGLL1, TSP-180, MAGE4, MAGE5, MAGE6, VEGFR1, IGF1R, hepatocyte growth factor receptor, p185ErbB2, p180ErbB-3, nm-23H1, CA 19-9, CA 72-4, CAM 17.1, NuMa, K-ras, beta-Catenin, CDK4, Muml, p15, p16, 43-9F, 5T4, 791Tgp72, β-human chorionic gonadotropin, BCA225, BTAA, CA125, CA15-3, CA 27.29 (BCAA), CA195, CA242, CA-50, CAM43, CD68, CO-029, FGFS, G250, HTgp-175, M344, MA50, MG7-Ag, MOV18, NB/70K, NY-CO1, RCAS1, SDCCAG16, M2BP, TAAL6, TLP, and TPS, glioma-associated antigen, gamma-fetoprotein (AFP), p26 fragment of AFP, lectin-reactive AFP, and TLR4. In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the liposome comprises a targeting moiety that has specific affinity for an epitope of an antigen selected from: PDGFRA, VEGFR1, VEGFR3, neuropilin 1 (NRP1), neuropilin 2 (NRP2), betacellulin, PLGF, RET (rearranged during transfection), TIEL TIE2 (TEK), CA125, CD3, CD4, CD7, CD10, CD13, CD25 CD32, CD32b, CD44 (e.g., CD44v6), CD47, CD49e (integrin gamma 5), CD54 (ICAM), CD55, CD64, CD74, CD80, CD90, CD200, CD147, CD166, CD200, ESA, SHH, DHH, IHH, patched 1 (PTCH1), smoothened (SMO), WNT1, WNT2B, WNT3A, WNT4, WNT4A, WNTSA, WNTSB, WNT7B, WNT8A, WNT10A, WNT10B, WNT16B, LKPS, LRPS, LRP6, FZD1, FZD2, FZD4, FZD5, FZD6, FZD7, FZD8, Notch, Notch1, Notch3, Notch4, DLL4, Jagged, Jagged1, Jagged2, Jagged3, TNFRSF1A (TNFR1, p55, p60), TNFRSF1B (TNFR2), TNFRSF6 (Fas, CD95), TNFRSF6B (DcR3), TNFRSF7 (CD27), TNFSF9 (41BB Ligand), TNFRSF8 (CD30), TNFRSF10A (TRAILR1, DR4), TNFRSF11A (RANK), TNFRSF12 (TWEAKR), TNFRSF19L (KELT), TNFRSF19 (TROY), TNFRSF21 (DR6), ILIRI, 1L1R2, IL2R, IL5R, IL6R, 1L8R, IL10R, IL12R, IL13R, IL15R, IL18R, IL19R, IL21R, IL23R, XAG1, XAG3, REGIV, FGFR1, FGFR2, FGFR3, ALK, ALK1, ALK7, ALCAM, Axl, TGFb, TGFb2, TGFb3, TGFBR1, IGFIIR, BMPRI, N-cadherin, E-cadherin, VE-cadherin, ganglioside GM2, ganglioside GD3, PSGR, DCC, CDCP1, CXCR2, CXCR7, CCR3, CCR4, CCR5, CCR7, CCR10, Claudin1, Claudin2, Claudin3, Claudin4, TMEFF2, neuregulin, MCSF, CSF, CSFR (fms), GCSF, GCSFR, BCAM, BRCA1, BRCA2, HLA-DR, ABCC3, ABCB5, HM 1.24, LFA1, LYNX, S100A8, S100A9, SCF, Von Willebrand factor, Lewis Y6 receptor, CA G250 (CA9), CRYPTO, VLA5, HLADR, MUC18, mucin CanAg, EGFL7, integrin avb3, integrin γ5β activin B1 gamma, leukotriene B4 receptor (LTB4R), neurotensin NT receptor (NTR), 5T4 oncofetal antigen, Tenascin C, MMP, MMP2, MMP7, MMP9, MMP12, MMP14, MMP26, cathepsin G, SULF1, SULF2, MET, CA9, TM4SF1, syndecan (SDC1), Ephrin B4, TEM1, TGFbeta 1, and TGFBRII. In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the liposome comprises a targeting moiety that has specific affinity for an epitope of an antigen associated with a disorder of the immune system (e.g., an autoimmune disorder and an inflammatory disorder), or is associated with regulating an immune response. In some embodiments, the targeting moiety has specific affinity for an epitope of a cell surface antigen expressed on the surface of a macrophage (expressing CD44).

In some embodiments, the liposome comprises a targeting moiety that has specific affinity for an epitope of an immunoinhibitory target. In another embodiment, the AD is an epitope of an immunoinhibitory target selected from: IL1Ra, IL6R, CD26L, CD28, CD80, FcGamma RIIB. In another embodiment, the AD in the Adapter is an epitope of an immunostimulatory target selected from: CD25, CD28, CTLA4, PD1, B7H1 (PDL1), B7H4 TGFbeta, TNFRSF4 (OX40), TNFRSF5 (CD40), TNFRSF9 (41BB, CD137), TNFRSF14 (HVEM), TNFRSF25 (DR3), and TNFRSF18 (GITR).

In some embodiments, the liposome comprises a targeting moiety that has specific affinity for an epitope of an antigen selected from: IL1Rb, C3AR, C5AR, CXCR1, CXCR2, CCR1, CCR3, CCR7, CCR8, CCR9, CCR10, ChemR23, MPL, GP130, TLR2, TLR3, TLR4, TLR5, TLR7, TLR8, TLR9, TREM1, TREM2, CD49a (integrin gamma 1), integrin a5b3, gamma4 integrin subunit, A4B7 integrin, cathepsin G, TNFRSF3 (LTBR), TNFRSF6 (Fas, CD95), TNFRSF6B (DcR3), TNFRSF8 (CD30), TNFRSF11A (RANK), TNFRSF16 (NGFR), TNFRSF19L (RELT), TNFRSF19 (TROY), TNFRSF21 (DR6), CD14, CD23, CD36, CD36L, CD39, CD91, CD153, CD164, CD200, CD200R, B71 (CD80), B72 (CD86), B7h, B7DC (PDL2), ICOS, ICOSL, MHC, CD, B7H2, B7H3, B7x, SLAM, KIM1, SLAMF2, SLAMF3, SLAMF4, SLAMF5, SLAMF6, SLAMF7, TNFRSF1A (TNFR1, p55, p60), TNFRSF1B (TNFR2), TNFRSF7 (CD27), TNFRSF12 (TWEAKR), TNFRSF5 (CD40), IL1R, IL2R, IL4Ra, IL5R, IL6RIL15R, IL17R, IL17Rb, IL17RC, IL22RA, IL23R, TSLPR, B7RP1, cKit, GMCSF, GMCSFR, CD2, CD4, CD11a, CD18, CD30, CD40, CD86, CXCR3, CCR2, CCR4, CCR5, CCR8, RhD, IgE, and Rh.

In additional embodiments, the disclosure provides a method for treating cancer that comprises administering an effective amount of a liposomal composition to a subject having or at risk of having a cancer that expresses folate receptor on its cell surface, wherein the liposomal composition comprises liposomes that comprise (a) polyglutamated Antifolate (e.g., γPANTIFOL or αPANTIFOL) and (b) a targeting moiety that has specific binding affinity for a folate receptor. In some embodiments, the targeting moiety has specific binding affinity for folate receptor alpha (FR-α), folate receptor beta (FR-β), and/or folate receptor delta (FR-δ). In some embodiments, the targeting moiety has a specific binding affinity for folate receptor alpha (FR-α), folate receptor beta (FR-β), and/or folate receptor delta (FR-δ). In some embodiments, the targeting moiety has a specific binding affinity for folate receptor alpha (FR-α) and folate receptor beta (FR-β). In some embodiments, the administered liposomal composition comprises pegylated liposomes (e.g., TPLp-PANTIFOL). In some embodiments, the administered liposomal composition comprises liposomes that are not pegylated. In some embodiments, liposomes of the administered liposomal composition comprises PANTIFOL containing 4, 5, 2-10, 4-6, or more than 5, γ-glutamyl groups or α-glutamyl groups. In some embodiments, a liposome of the liposomal composition comprises a γPANTIFOL containing 1, 2, 3, 4, 5, 6, 7, 8, 9, 10, or more than 10, γ-glutamyl groups or α-glutamyl groups in the D-form. In some embodiments, a liposome of the liposomal composition comprises L and D gamma polyglutamated Antifolate or L and D alpha polyglutamated Antifolate. In some embodiments, a liposome of the liposomal composition comprises a γPANTIFOL containing 2, 3, 4, 5, or more than 5, γ-glutamyl groups in the L-form, and 1, 2, 3, 4, 5 or more than 5, γ-glutamyl groups in the D-form. In some embodiments, a liposome of the liposomal composition comprises αPANTIFOL containing 2, 3, 4, 5, or more than 5, α-glutamyl groups in the L-form, and 1, 2, 3, 4, 5 or more than 5, α-glutamyl groups in the D-form. In some embodiments, a liposome of the liposomal composition comprises tetraglutamated Antifolate. In some embodiments, a liposome of the liposomal composition comprises pentaglutamated Antifolate. In some embodiments, a liposome of the liposomal composition comprises hexaglutamated Antifolate.

In some embodiments, liposomes of the administered liposomal composition comprise gamma tetraglutamated Antifolate or alpha tetraglutamated Antifolate. In some embodiments, liposomes of the administered liposomal composition comprise gamma pentaglutamated Antifolate or alpha pentaglutamated Antifolate. In some embodiments, liposomes of the administered liposomal composition comprises gamma hexaglutamated Antifolate or alpha hexaglutamated Antifolate. In some embodiments, the liposomal composition is administered to treat an epithelial tissue malignancy. In some embodiments, the liposomal composition is administered to treat a cancer selected from: lung cancer, pancreatic, breast cancer, ovarian cancer, lung cancer, prostate cancer, head and neck cancer, gastric cancer, gastrointestinal cancer, colon cancer, esophageal cancer, cervical cancer, kidney cancer, biliary duct cancer, gallbladder cancer, and a hematologic malignancy. In some embodiments, the liposomal composition is administered to treat a cancer selected from: breast cancer, advanced head and neck cancer, lung cancer, stomach cancer, osteosarcoma, Non-Hodgkin's lymphoma (NHL), acute lymphoblastic leukemia (ALL), mycosis fungoides (cutaneous T-cell lymphoma) choriocarcinoma, chorioadenoma, nonleukemic meningeal cancer, soft tissue sarcoma (desmoid tumors, aggressive fibromatosis), bladder cancer, and central nervous system (CNS) cancer. In some embodiments, the liposomal composition is administered to treat lung cancer (e.g., NSCLC or mesothelioma). In some embodiments, the liposomal composition is administered to treat breast cancer (e.g., HER2++ or triple negative breast cancer). In some embodiments, the liposomal composition is administered to treat colorectal cancer. In some embodiments, the liposomal composition is administered to treat ovarian cancer. In some embodiments, the liposomal composition is administered to treat endometrial cancer. In some embodiments, the liposomal composition is administered to treat pancreatic cancer. In some embodiments, the liposomal composition is administered to treat liver cancer. In some embodiments, the liposomal composition is administered to treat head and neck cancer. In some embodiments, the liposomal composition is administered to treat osteosarcoma.

In some embodiments, the disclosure provides a method for treating lung cancer (e.g., non-small lung cancer) that comprises administering an effective amount of a delivery vehicle (e.g., an antibody or liposome) comprising polyglutamated Antifolate (e.g., a PANTIFOL disclosed herein) to a subject having or at risk of having lung cancer. In particular embodiments, the, the cancer is non-small cell lung cancer. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In some embodiments, the administered delivery vehicle is pegylated. In some embodiments, the administered delivery vehicle is not pegylated. In additional embodiments, the delivery vehicle comprises a targeting moiety on its surface that has specific affinity for an epitope on an antigen on the surface of a lung cancer (e.g., non-small cell lung cancer) cell. In further embodiments, the delivery vehicle comprises a targeting moiety that has specific affinity for an epitope on an antigen selected from Mucin 1, Nectin 4, NaPi2b, CD56, EGFR, and SC-16. In some embodiments, the targeting moiety is an antibody or a fragment of an antibody. In additional embodiments, the delivery vehicle is a liposome, and the liposome comprises a targeting moiety that has specific affinity for an epitope on an antigen selected from Mucin 1, Nectin 4, NaPi2b, CD56, EGFR, and SC-16. In further embodiments, the delivery vehicle is a pegylated liposome that comprises a targeting moiety that has specific affinity for an epitope on an antigen selected from: Mucin 1, Nectin 4, NaPi2b, CD56, EGFR, and SC-16. In some embodiments, the administered delivery vehicle comprises PANTIFOL containing 4, 5, 2-10, 4-6, or more than 5, glutamyl groups. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups. In some embodiments, the administered delivery vehicle comprises gamma tetraglutamated Antifolate or alpha tetraglutamated Antifolate. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate.

In some embodiments, the disclosure provides a method for treating pancreatic cancer that comprises administering an effective amount of a delivery vehicle (e.g., an antibody (ADC) or liposome) comprising polyglutamated Antifolate (e.g., a PANTIFOL disclosed herein) to a subject having or at risk of having pancreatic cancer. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In some embodiments, the administered delivery vehicle is pegylated. In some embodiments, the administered delivery vehicle is not pegylated. In additional embodiments, the delivery vehicle comprises a targeting moiety on its surface that has specific affinity for an epitope on an antigen on the surface of a pancreatic cancer cell. In further embodiments, the delivery vehicle comprises a targeting moiety that has specific affinity for an epitope on an antigen selected from TACSTD2 (TROP2), Mucin 1, mesothelin, Guanylyl cyclase C (GCC), SLC44A4, and Nectin 4. In further embodiments, the delivery vehicle is a liposome, and the liposome comprises a targeting moiety has specific affinity for an epitope on an antigen selected from TACSTD2 (TROP2), Mucin 1, Mesothelin, Guanylyl cyclase C (GCC), SLC44A4, and Nectin 4. In some embodiments, the administered delivery vehicle comprises γPANTIFOL containing 4, 5, 2-10, 4-6, or more than 5, γ-glutamyl groups or α-glutamyl groups. In some embodiments, the administered delivery vehicle comprises gamma tetraglutamated Antifolate. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups.

In additional embodiments, the disclosure provides a method for treating breast cancer (e.g., triple negative breast cancer (estrogen receptor-, progesterone receptor-, and HER2)) that comprises administering an effective amount of a delivery vehicle (e.g., an antibody or liposome) comprising polyglutamated Antifolate (e.g., a PANTIFOL disclosed herein) to a subject having or at risk of having breast cancer. In some embodiments, the administered delivery vehicle is a liposome that comprises gamma polyglutamated Antifolate. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In some embodiments, the administered delivery vehicle is pegylated. In some embodiments, the administered delivery vehicle is not pegylated. In additional embodiments, the delivery vehicle comprises a targeting moiety on its surface that has specific affinity for an epitope on an antigen on the surface of a breast cancer cell. In further embodiments, the delivery vehicle comprises a targeting moiety that has specific affinity for an epitope on an antigen selected from: LIV-1 (ZIP6), EGFR, HER2, HER3, Mucin 1, GONMB, and Nectin 4. In some embodiments, the targeting moiety is an antibody or a fragment of an antibody. In additional embodiments, the delivery vehicle is a liposome, and the liposome comprises a targeting moiety that has specific affinity for an epitope on an antigen selected from: LIV-1 (ZIP6), EGFR, HER2, HER3, Mucin 1, GONMB, and Nectin 4. In some embodiments, the administered delivery vehicle comprises PANTIFOL containing 4, 5, 2-10, 4-6, or more than 5, γ-glutamyl groups or α-glutamyl groups. In some embodiments, the administered delivery vehicle comprises gamma tetraglutamated Antifolate or alpha tetraglutamated Antifolate. In some embodiments, the administered delivery vehicle comprises gamma pentaglutamated Antifolate or alpha pentaglutamated Antifolate. In some embodiments, the administered delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups.

In some embodiments, the disclosure provides a method for treating a hematological cancer that comprises administering an effective amount of a delivery vehicle (e.g., an antibody or liposome) comprising polyglutamated Antifolate (e.g., a PANTIFOL disclosed herein) to a subject having or at risk of having a hematological cancer. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In some embodiments, the administered delivery vehicle is pegylated. In some embodiments, the administered delivery vehicle is not pegylated. In additional embodiments, the delivery vehicle comprises a targeting moiety on its surface that has specific affinity for an epitope on an antigen on the surface of a hematological cancer cell. In further embodiments, the delivery vehicle comprises a targeting moiety that has specific affinity for an epitope on an antigen selected from: CD30, CD79b, CD19, CD138, CD74, CD37, CD19, CD22, CD33, CD34, and CD98. In further embodiments, the delivery vehicle is a liposome, and the liposome comprises a targeting moiety has specific affinity for an epitope on an antigen selected from: CD30, CD79b, CD19, CD138, CD74, CD37, CD19, CD22, CD33, CD34, and CD98. In some embodiments, the administered delivery vehicle comprises PANTIFOL containing 4, 5, 2-10, 4-6, or more than 5, γ-glutamyl groups or α-glutamyl groups. In some embodiments, the administered delivery vehicle comprises gamma tetraglutamated Antifolate or alpha tetraglutamated Antifolate. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups.

In some embodiments, the disclosure provides a method for treating a subject having or at risk of having a cancer that is distinguishable by the expression of an antigen on its cell surface. Thus, in some embodiments, the disclosure provides a method for treating cancer that comprises administering to a subject having or at risk of having a cancer, an effective amount of a delivery vehicle (e.g., an antibody or liposome) comprising a targeting moiety that has specific affinity for an epitope on a surface antigen of the cancer and polyglutamated Antifolate (e.g., a PANTIFOL disclosed herein). In some embodiments, the administered delivery vehicle comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the administered delivery vehicle is pegylated. In some embodiments, the targeting moiety is an antibody or a fragment of an antibody. In additional embodiments, the delivery vehicle is a liposome. In some embodiments, the administered delivery vehicle comprises PANTIFOL consisting of 4, 5, 2-10, 4-6, or more than 5, γ-glutamyl groups or α-glutamyl groups. In some embodiments, the administered delivery vehicle comprises gamma tetraglutamated Antifolate or alpha tetraglutamated Antifolate. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups.

In some embodiments, the disclosed compositions (e.g., liposomes containing alpha or gamma polyglutamated Antifolate) are administered to subjects having or at risk of having a cancer, a solid tumor, and/or a metastasis that is distinguishable by the expression of a tumor specific antigen or tumor associated antigen on its cell surface. Thus, in some embodiments, the disclosure provides a method for treating cancer that comprises administering an effective amount of a delivery vehicle (e.g., liposome) comprising a targeting moiety and polyglutamated Antifolate (e.g., a PANTIFOL disclosed herein) to a subject having or at risk of having a cancer, solid tumor, and/or metastasis that is distinguishable by the expression of a tumor specific antigen or tumor associated antigen on its cell surface cancer, and wherein the targeting moiety has specific binding affinity for an epitope on an tumor specific antigen or tumor associated antigen. In some embodiments, the administered delivery vehicle is a liposome. In further embodiments, the liposome is pegylated. In additional embodiments, the delivery vehicle comprises a targeting moiety that has specific affinity for an epitope on a cell surface antigen expressed on the surface of a cancer, a solid tumor, and/or a metastatic cell. e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P Cadherin, Fibronectin Extra-domain B (ED-B), VEGFR2 (CD309), Tenascin, Collagen IV, Periostin, endothelin receptor, HER2, HER3, ErbB4, EGFR, EGFRvIII, FGFR1, FGFR2, FGFR3, FGFR4, FGFR6, IGFR-1, FZD1, FZD2, FZD3, FZD4, FZD5, FZD6, FZD7, FZD8, FZD9, FZD10, SMO, CD2, CD3, CD4, CD5, CD6, CD8, CD11, CD11a, CD15, CD18, CD19, CD20, CD22, CD26, CD27L, CD28, CD30, CD33, CD34, CD37, CD38, CD40, CD44, CD56, CD70, CD74, CD79, CD79b, CD98, CD105, CD133, CD138, cripto, IGF-1R, IGF-2R, EphA1 an EphA receptor, an EphB receptor, EphA1, EphA2, EphA3, EphA4, EphA5, EphA6, EphA7, EphA8, EphB1, EphB2, EphB3, EphB4, EphB6, an integrin (e.g., integrin αvβ3, αvβ5, or αvβ6), a C242 antigen, Apo2, PSGR, NGEP, PSCA, TMEFF2, endoglin, PSMA, CanAg, CALLA, c-Met, VEGFR-1, VEGFR-2, DDR1, PDGFR alpha., PDGFR beta, TrkA, TrkB, TrkC, UFO, LTK, ALK, Tie1, Tie2, PTK7, Ryk, TCR, NMDAR, LNGFR, and MuSK. In some embodiments, the delivery vehicle comprises a targeting moiety that has specific affinity for an epitope on a cell surface antigen(s) derived from, or determined to be expressed on, a specific subject's cancer (tumor) such as a neoantigen. In some embodiments, the administered delivery vehicle comprises PANTIFOL containing 4, 5, 2-10, 4-6, or more than 5, γ-glutamyl groups or α-glutamyl groups. In some embodiments, the delivery vehicle comprises a PANTIFOL containing 2, 3, 4, 5, 6, 7, 8, 9, 10, or more than 10, γ-glutamyl groups or α-glutamyl groups in the L-form. In some embodiments, the delivery vehicle comprises a PANTIFOL containing 1, 2, 3, 4, 5, 6, 7, 8, 9, 10, or more than 10, γ-glutamyl groups or α-glutamyl groups in the L-form. In some embodiments, the delivery vehicle comprises D gamma polyglutamated Antifolate or D alpha polyglutamated Antifolate. In some embodiments, the delivery vehicle comprises a PANTIFOL containing 1, 2, 3, 4, 5, 6, 7, 8, 9, 10, or more than 10, γ-glutamyl groups or α-glutamyl groups in the D-form. In some embodiments, the delivery vehicle comprises L and D gamma polyglutamated Antifolate or L and D alpha polyglutamated Antifolate. In some embodiments, the delivery vehicle comprises a γPANTIFOL containing 2, 3, 4, 5, or more than 5, γ-glutamyl groups in the L-form, and 1, 2, 3, 4, 5 or more than 5, γ-glutamyl groups in the D-form. In some embodiments, the delivery vehicle comprises a αPANTIFOL containing 2, 3, 4, 5, or more than 5, α-glutamyl groups in the L-form, and 1, 2, 3, 4, 5 or more than 5, α-glutamyl groups in the D-form. In some embodiments, the administered delivery vehicle comprises gamma tetraglutamated Antifolate or alpha tetraglutamated Antifolate. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate.

In additional embodiments, the targeting moiety has specific affinity for an epitope on an antigen selected from: GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TMEFF2, endoglin, PSMA, CD98, CD56, CanAg, and CALLA.

In further embodiments, the delivery vehicle is a liposome, and the liposome comprises a targeting moiety that specifically binds a cell surface antigen selected from: GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TMEFF2, endoglin, PSMA, CD98, CD56, CanAg, and CALLA. In some embodiments, the administered delivery vehicle comprises PANTIFOL containing 4, 5, 2-10, 4-6, or more than 5, glutamyl groups. In some embodiments, the liposome comprises gamma pentaglutamated Antifolate or alpha pentaglutamated Antifolate. In other embodiments, the liposome comprises gamma hexaglutamated Antifolate or alpha hexaglutamated Antifolate. In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the a liposome comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the liposome comprises D gamma polyglutamated Antifolate or D alpha polyglutamated Antifolate. In some embodiments, the liposome comprises 2, 3, 4, 5, or more than 5, D-gamma glutamyl groups or D-alpha glutamyl groups. In some embodiments, the liposome comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups.

In further embodiments, the disclosure provides a method for treating cancer that comprises administering an effective amount of a delivery vehicle (e.g., an antibody or liposome) comprising a targeting moiety on its surface that specifically binds a folate receptor, and a polyglutamated Antifolate (e.g., a PANTIFOL disclosed herein) to a subject having or at risk of having a cancer that contains cells expressing the folate receptor on their cell surface. In some embodiments, the targeting moiety is an antibody, or an antigen binding fragment of an antibody. In further embodiments, the targeting moiety has specific affinity for folate receptor alpha, folate receptor beta or folate receptor delta. As disclosed herein, the folate receptor targeted pegylated liposomes containing gamma polyglutamated Antifolate are able to deliver high quantities of gamma polyglutamated Antifolate to cancer cells and particularly cancer cells that express folate receptors, compared to normal cells (i.e., cells that unlike cancer cells do not actively take up liposomes, and/or do not express folate receptors). Any cancers that express folate receptors may be treated according to the disclosed methods. It should be noted that some cancers may express folate receptors in an early stage while the majority of cancers may express folate receptors at late stages. In some embodiments, the administered delivery vehicle is a liposome. In further embodiments, the liposome is pegylated. In some embodiments, the administered delivery vehicle comprises PANTIFOL containing 4, 5, 2-10, 4-6, or more than 5, glutamyl groups. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups.

In additional embodiments, the disclosure provides a method for cancer maintenance therapy that comprises administering an effective amount of a liposomal composition comprising liposomes that contain polyglutamated Antifolate (e.g., a PANTIFOL disclosed herein) to a subject that is undergoing or has undergone cancer therapy. In some embodiments, the administered liposomal composition is a PLp-PANTIFOL, NTLp-PANTIFOL, NTPLp-PANTIFOL, TLp-PANTIFOL or TPLp-PANTIFOL. In some embodiments, the administered liposomal composition comprises pegylated liposomes (e.g., PLp-PANTIFOL, NTPLp-PANTIFOL, or TPLp-PANTIFOL). In some embodiments, the administered liposomal composition comprises a targeting moiety that has specific affinity for an epitope on a surface antigen of a cancer cell (e.g., TLp-PANTIFOL or TPLp-PANTIFOL). In some embodiments, the administered liposomal composition comprises liposomes that are pegylated and comprise a targeting moiety (e.g., TPLp-PANTIFOL). In some embodiments, the administered liposomal composition comprises liposomes that are targeted and liposomes that do not comprise a targeting moiety (e.g., are not targeted). In some embodiments, the administered liposomal composition comprises liposomes that are pegylated and liposomes that are not pegylated. In some embodiments, liposomes of the administered liposomal composition comprise polyglutamated Antifolate that contains 4, 5, 2-10, 4-6, or more than 5, γ-glutamyl groups or α-glutamyl groups. In some embodiments, the administered liposomal composition comprises gamma tetraglutamated Antifolate or alpha tetraglutamated Antifolate. In some embodiments, the administered liposomal composition comprises gamma pentaglutamated Antifolate or alpha pentaglutamated Antifolate. In other embodiments, the administered liposomal composition comprises gamma hexaglutamated Antifolate or alpha hexaglutamated Antifolate. In some embodiments, the administered liposomal composition comprises L gamma polyglutamated Antifolate or L alpha polyglutamated Antifolate. In some embodiments, the administered liposomal composition comprises L gamma polyglutamated Antifolate or L alpha polyglutamated Antifolate. In some embodiments, the administered liposomal composition comprises 2, 3, 4, 5, or more than 5, L-gamma glutamyl groups or L-alpha glutamyl groups. In some embodiments, the administered liposomal composition comprises D gamma polyglutamated Antifolate or D alpha polyglutamated Antifolate. In some embodiments, the administered liposomal composition comprises 2, 3, 4, 5, or more than 5, D-gamma glutamyl groups or D-alpha glutamyl groups. In some embodiments, the administered liposomal composition comprises L and D gamma polyglutamated Antifolate or L and D alpha polyglutamated Antifolate. In some embodiments, the administered liposomal composition comprises 2, 3, 4, 5, or more than 5, L-gamma glutamyl groups or L-alpha glutamyl groups. In some embodiments, the administered liposomal composition comprises 2, 3, 4, 5, or more than 5, L-gamma glutamyl groups and 2, 3, 4, 5, or more than 5, D-gamma glutamyl groups. In some embodiments, the administered liposomal composition comprises 2, 3, 4, 5, or more than 5, L-alpha glutamyl groups and 2, 3, 4, 5, or more than 5, D-alpha glutamyl groups.

In some embodiments, the cancer treated by one or more of the methods disclosed herein is a solid tumor lymphoma. Examples of solid tumor lymphoma include Hodgkin's lymphoma, Non-Hodgkin's lymphoma, and B cell lymphoma.

In some embodiments, the cancer treated by one or more of the methods disclosed herein is bone cancer, brain cancer, breast cancer, colorectal cancer, connective tissue cancer, cancer of the digestive system, endometrial cancer, esophageal cancer, eye cancer, cancer of the head and neck, gastric cancer, intra-epithelial neoplasm, melanoma neuroblastoma, Non-Hodgkin's lymphoma, non-small cell lung cancer, prostate cancer, retinoblastoma, or rhabdomyosarcoma.

In some embodiments, the disclosure provides a method for treating cancer that comprises administering an effective amount of a composition comprising a delivery vehicle and a gamma polyglutamated Antifolate to a subject having or at risk of having cancer. In some embodiments, the administered composition comprises a pegylated delivery vehicle. In some embodiments, the administered composition comprises a targeting moiety that has a specific affinity for an epitope of antigen on the surface of a target cell of interest such as a cancer cell. In some embodiments, the delivery vehicle comprises an antibody or an antigen binding antibody fragment. In some embodiments, the composition is administered to treat a cancer selected from: lung cancer, pancreatic cancer, breast cancer, ovarian cancer, prostate cancer, head and neck cancer, gastric cancer, gastrointestinal cancer, colorectal cancer, esophageal cancer, cervical cancer, liver cancer, kidney cancer, biliary duct cancer, gallbladder cancer, bladder cancer, sarcoma (e.g., osteosarcoma), brain cancer, central nervous system cancer, melanoma, myeloma, a leukemia and a lymphoma. In some embodiments, the composition is administered to treat a cancer selected from: breast cancer, advanced head and neck cancer, lung cancer, stomach cancer, osteosarcoma, Non-Hodgkin's lymphoma (NHL), acute lymphoblastic leukemia (ALL), mycosis fungoides (cutaneous T-cell lymphoma) choriocarcinoma, chorioadenoma, nonleukemic meningeal cancer, soft tissue sarcoma (desmoid tumors, aggressive fibromatosis), bladder cancer, and central nervous system (CNS) cancer. In some embodiments, the cancer is lung cancer (e.g., NSCLC or mesothelioma). In some embodiments, the cancer is breast cancer (e.g., HER2++ or triple negative breast cancer). In some embodiments, the cancer is colorectal cancer. In some embodiments, the cancer is ovarian cancer. In some embodiments, the cancer is endometrial cancer. In some embodiments, the cancer is pancreatic cancer. In some embodiments, the cancer is liver cancer. In some embodiments, the cancer is head and neck cancer. In some embodiments, the cancer is osteosarcoma.

In some embodiments, the administered composition contains 4, 5, 2-10, 4-6, or more than 5, glutamyl groups. In some embodiments, the administered delivery vehicle comprises PANTIFOL containing 4, 5, 2-10, 4-6, or more than 5, glutamyl groups. In some embodiments, the administered delivery vehicle comprises gamma tetraglutamated Antifolate or alpha tetraglutamated Antifolate. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups. In some embodiments, the administered delivery vehicle is an immunoconjugate. In some embodiments, the administered delivery vehicle comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In additional embodiments, the disclosure provides a method for treating cancer that comprises administering an effective amount of a liposomal composition comprising liposomes that contain polyglutamated Antifolate (e.g., Lp-PANTIFOL, PLp-PANTIFOL, NTLp-PANTIFOL, NTPLp-PANTIFOL, TLp-PANTIFOL or TPLp-PANTIFOL) to a subject having or at risk of having cancer. In some embodiments, the liposomal composition comprises αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the liposomal composition is administered to treat a cancer selected from: lung cancer, pancreatic cancer, breast cancer, ovarian cancer, prostate cancer, head and neck cancer, gastric cancer, gastrointestinal cancer, colorectal cancer, esophageal cancer, cervical cancer, liver cancer, kidney cancer, biliary duct cancer, gallbladder cancer, bladder cancer, sarcoma (e.g., osteosarcoma), brain cancer, central nervous system cancer, melanoma, myeloma, a leukemia and a lymphoma. In some embodiments, the liposomal composition is administered to treat lung cancer (e.g., NSCLC or mesothelioma). In some embodiments, the liposomal composition is administered to treat breast cancer (e.g., HER2++ or triple negative breast cancer). In some embodiments, the liposomal composition is administered to treat colorectal cancer. In some embodiments, the liposomal composition is administered to treat ovarian cancer. In some embodiments, the liposomal composition is administered to treat endometrial cancer. In some embodiments, the liposomal composition is administered to treat pancreatic cancer. In some embodiments, the liposomal composition is administered to treat liver cancer. In some embodiments, the liposomal composition is administered to treat head and neck cancer. In some embodiments, the liposomal composition is administered to treat osteosarcoma. In some embodiments, the administered liposomal composition comprises pegylated liposomes (e.g., PLp-PANTIFOL, NTPLp-PANTIFOL, or TPLp-PANTIFOL). In some embodiments, liposomes of the administered liposomal composition comprise a PANTIFOL containing 4, 5, 2-10, 4-6, or more than 5, glutamyl groups. In some embodiments, liposomes of the administered liposomal composition comprise a gamma tetraglutamated Antifolate or alpha tetraglutamated Antifolate. In some embodiments, liposomes of the administered liposomal composition comprise a gamma pentaglutamated Antifolate or alpha pentaglutamated Antifolate. In other embodiments, liposomes of the administered liposomal composition comprises a gamma hexaglutamated Antifolate or alpha hexaglutamated Antifolate.

In additional embodiments, the disclosure provides a method for treating cancer that comprises administering an effective amount of a liposomal composition that comprises targeted liposomes (e.g., TLp-PANTIFOL or TPLp-PANTIFOL) to a subject having or at risk of having cancer, wherein the liposomal composition comprises liposomes that comprise a polyglutamated Antifolate (Lp-PANTIFOL) and further comprise a targeting moiety having a specific affinity for a surface antigen (epitope) on the cancer. In some embodiments, the liposomal composition comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In additional embodiments, the disclosure provides a method for treating cancer that comprises administering an effective amount of a liposomal composition comprising liposomes that contain polyglutamated Antifolate (e.g., Lp-PANTIFOL, PLp-PANTIFOL, NTLp-PANTIFOL, NTPLp-PANTIFOL, TLp-PANTIFOL or TPLp-PANTIFOL) to a subject having or at risk of having cancer. In some embodiments, the liposomal composition is administered to treat a cancer selected from: lung cancer, pancreatic cancer, breast cancer, ovarian cancer, prostate cancer, head and neck cancer, gastric cancer, gastrointestinal cancer, colorectal cancer, esophageal cancer, cervical cancer, liver cancer, kidney cancer, biliary duct cancer, gallbladder cancer, bladder cancer, sarcoma (e.g., osteosarcoma), brain cancer, central nervous system cancer, melanoma, myeloma, a leukemia and a lymphoma. In some embodiments, the liposomal composition is administered to treat a cancer selected from: breast cancer, advanced head and neck cancer, lung cancer, stomach cancer, osteosarcoma, Non-Hodgkin's lymphoma (NHL), acute lymphoblastic leukemia (ALL), mycosis fungoides (cutaneous T-cell lymphoma) choriocarcinoma, chorioadenoma, nonleukemic meningeal cancer, soft tissue sarcoma (desmoid tumors, aggressive fibromatosis), bladder cancer, and central nervous system (CNS) cancer. In some embodiments, the liposomal composition is administered to treat lung cancer (e.g., NSCLC or mesothelioma). In some embodiments, the liposomal composition is administered to treat breast cancer (e.g., HER2++ or triple negative breast cancer). In some embodiments, the liposomal composition is administered to treat colorectal cancer. In some embodiments, the liposomal composition is administered to treat ovarian cancer. In some embodiments, the liposomal composition is administered to treat endometrial cancer. In some embodiments, the liposomal composition is administered to treat pancreatic cancer. In some embodiments, the liposomal composition is administered to treat liver cancer. In some embodiments, the liposomal composition is administered to treat head and neck cancer. In some embodiments, the liposomal composition is administered to treat osteosarcoma.

In some embodiments, the administered liposomal composition comprises pegylated liposomes (e.g., PLp-PANTIFOL, NTPLp-PANTIFOL, or TPLp-PANTIFOL). In some embodiments, liposomes of the administered liposomal composition comprise a PANTIFOL containing 4, 5, 2-10, 4-6, or more than 5, glutamyl groups. In some embodiments, the administered liposomal composition comprises gamma tetraglutamated Antifolate or alpha tetraglutamated Antifolate. In some embodiments, liposomes of the administered liposomal composition comprises gamma pentaglutamated Antifolate or alpha pentaglutamated Antifolate. In other embodiments, liposomes of the administered liposomal composition comprises gamma hexaglutamated Antifolate or alpha hexaglutamated Antifolate. In some embodiments, the administered liposomal composition comprises L gamma polyglutamated Antifolate or L alpha polyglutamated Antifolate. In some embodiments, the administered liposomal composition comprises 2, 3, 4, 5, or more than 5, L-gamma glutamyl groups or L-alpha glutamyl groups. In some embodiments, the administered liposomal composition comprises D gamma polyglutamated Antifolate or D alpha polyglutamated Antifolate. In some embodiments, the administered liposomal composition comprises 2, 3, 4, 5, or more than 5, D-gamma glutamyl groups or D-alpha glutamyl groups. In some embodiments, the administered liposomal composition comprises L and D gamma polyglutamated Antifolate or L and D alpha polyglutamated Antifolate. In some embodiments, the administered liposomal composition comprises 2, 3, 4, 5, or more than 5, L-gamma glutamyl groups and 2, 3, 4, 5, or more than 5, D-gamma glutamyl groups. In some embodiments, the administered liposomal composition comprises 2, 3, 4, 5, or more than 5, L-alpha glutamyl groups and 2, 3, 4, 5, or more than 5, D-alpha glutamyl groups.

In additional embodiments, the disclosure provides a method for treating cancer that comprises administering an effective amount of a liposomal composition that comprises targeted liposomes (e.g., TLp-PANTIFOL or TPLp-PANTIFOL) to a subject having or at risk of having cancer, wherein the liposomal composition comprises liposomes that comprise polyglutamated Antifolate (Lp-PANTIFOL) and further comprise a targeting moiety having a specific affinity for a surface antigen (epitope) on the cancer. In some embodiments, the liposomal composition comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the liposomal composition is administered to treat a cancer selected from: lung cancer, pancreatic cancer, breast cancer, ovarian cancer, prostate cancer, head and neck cancer, gastric cancer, gastrointestinal cancer, colorectal cancer, esophageal cancer, cervical cancer, liver cancer, kidney cancer, biliary duct cancer, gallbladder cancer, bladder cancer, sarcoma (e.g., osteosarcoma), brain cancer, central nervous system cancer, melanoma, myeloma, a leukemia and a lymphoma. In some embodiments, the liposomal composition is administered to treat a cancer selected from: breast cancer, advanced head and neck cancer, lung cancer, stomach cancer, osteosarcoma, Non-Hodgkin's lymphoma (NHL), acute lymphoblastic leukemia (ALL), mycosis fungoides (cutaneous T-cell lymphoma) choriocarcinoma, chorioadenoma, nonleukemic meningeal cancer, soft tissue sarcoma (desmoid tumors, aggressive fibromatosis), bladder cancer, and central nervous system (CNS) cancer. In some embodiments, the liposomal composition is administered to treat lung cancer (e.g., NSCLC or mesothelioma). In some embodiments, the liposomal composition is administered to treat breast cancer (e.g., HER2++ or triple negative breast cancer). In some embodiments, the liposomal composition is administered to treat colorectal cancer. In some embodiments, the liposomal composition is administered to treat ovarian cancer. In some embodiments, the liposomal composition is administered to treat endometrial cancer. In some embodiments, the liposomal composition is administered to treat pancreatic cancer. In some embodiments, the liposomal composition is administered to treat liver cancer. In some embodiments, the liposomal composition is administered to treat head and neck cancer. In some embodiments, the liposomal composition is administered to treat osteosarcoma. In some embodiments, the administered liposomal composition comprises pegylated liposomes (e.g., PLp-PANTIFOL, NTPLp-PANTIFOL, or TPLp-PANTIFOL). In some embodiments, liposomes of the administered liposomal composition comprise a PANTIFOL containing 4, 5, 2-10, 4-6, or more than 5, γ-glutamyl groups or α-glutamyl groups. In some embodiments, liposomes of the administered liposomal composition comprise gamma tetraglutamated Antifolate or alpha tetraglutamated Antifolate. In some embodiments, liposomes of the administered liposomal composition comprise gamma pentaglutamated Antifolate or alpha pentaglutamated Antifolate. In other embodiments, the liposomes of the administered liposomal composition comprise gamma hexaglutamated Antifolate or alpha hexaglutamated Antifolate. In some embodiments, the administered liposomal composition comprises L gamma polyglutamated Antifolate or L alpha polyglutamated Antifolate. In some embodiments, the administered liposomal composition comprises 2, 3, 4, 5, or more than 5, L-gamma glutamyl groups or L-alpha glutamyl groups. In some embodiments, the administered liposomal composition comprises D gamma polyglutamated Antifolate or D alpha polyglutamated Antifolate. In some embodiments, the administered liposomal composition comprises 2, 3, 4, 5, or more than 5, D-gamma glutamyl groups or D-alpha glutamyl groups. In some embodiments, the administered liposomal composition comprises L and D gamma polyglutamated Antifolate or L and D alpha polyglutamated Antifolate. In some embodiments, the administered liposomal composition comprises 2, 3, 4, 5, or more than 5, L-gamma glutamyl groups and 2, 3, 4, 5, or more than 5, D-gamma glutamyl groups. In some embodiments, the administered liposomal composition comprises 2, 3, 4, 5, or more than 5, L-alpha glutamyl groups and 2, 3, 4, 5, or more than 5, D-alpha glutamyl groups.

In further embodiments, the disclosure provides a method for treating cancer that comprises administering an effective amount of a liposomal composition that contains targeted liposomes (e.g., TLp-PANTIFOL or TPLp-PANTIFOL) to a subject having or at risk of having a cancer that expresses folate receptor on its cell surface, wherein the liposomal composition comprises liposomes that comprise (a) polyglutamated Antifolate (e.g., γPANTIFOL or αPANTIFOL) and (b) a targeting moiety that has specific binding affinity for the folate receptor. In some embodiments, the administered liposomal composition comprises pegylated liposomes (e.g., TPLp-PANTIFOL). In some embodiments, the liposomal composition comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the targeting moiety has a specific binding affinity for folate receptor alpha (FR-α), folate receptor beta (FR-β), and/or folate receptor delta (FR-δ). In some embodiments, the targeting moiety has a specific binding affinity for folate receptor alpha (FR-α), folate receptor beta (FR-β), and/or folate receptor delta (FR-δ). In some embodiments, the targeting moiety has a specific binding affinity for folate receptor alpha (FR-α) and folate receptor beta (FR-β). In some embodiments, the liposomal composition is administered to treat a cancer selected from: lung cancer, pancreatic, breast cancer, ovarian cancer, lung cancer, prostate cancer, head and neck cancer, gastric cancer, gastrointestinal cancer, colon cancer, esophageal cancer, cervical cancer, kidney cancer, biliary duct cancer, gallbladder cancer, and a hematologic malignancy. In some embodiments, the liposomal composition is administered to treat a cancer selected from: breast cancer, advanced head and neck cancer, lung cancer, stomach cancer, osteosarcoma, Non-Hodgkin's lymphoma (NHL), acute lymphoblastic leukemia (ALL), mycosis fungoides (cutaneous T-cell lymphoma) choriocarcinoma, chorioadenoma, nonleukemic meningeal cancer, soft tissue sarcoma (desmoid tumors, aggressive fibromatosis), bladder cancer, and central nervous system (CNS) cancer. In some embodiments, the liposomal composition is administered to treat lung cancer (e.g., NSCLC or mesothelioma). In some embodiments, the liposomal composition is administered to treat breast cancer (e.g., HER2++ or triple negative breast cancer). In some embodiments, the composition is administered to treat colorectal cancer. In some embodiments, the composition is administered to treat ovarian cancer. In some embodiments, the composition is administered to treat endometrial cancer. In some embodiments, the composition is administered to treat pancreatic cancer. In some embodiments, the composition is administered to treat liver cancer. In some embodiments, the composition is administered to treat head and neck cancer. In some embodiments, the composition is administered to treat osteosarcoma. In some embodiments, liposomes of the administered liposomal composition comprise a PANTIFOL containing 4, 5, 2-10, 4-6, or more than 5, γ-glutamyl groups or α-glutamyl groups. In some embodiments, liposomes of the administered liposomal composition comprise gamma tetraglutamated Antifolate or alpha tetraglutamated Antifolate. In some embodiments, liposomes of the administered liposomal composition comprise gamma pentaglutamated Antifolate or alpha pentaglutamated Antifolate. In other embodiments, liposomes of the administered liposomal composition comprise gamma hexaglutamated Antifolate or alpha hexaglutamated Antifolate. In some embodiments, the administered liposomal composition comprises L gamma polyglutamated Antifolate or L alpha polyglutamated Antifolate. In some embodiments, the administered liposomal composition comprises 2, 3, 4, 5, or more than 5, L-gamma glutamyl groups or L-alpha glutamyl groups. In some embodiments, the administered liposomal composition comprises D gamma polyglutamated Antifolate or D alpha polyglutamated Antifolate. In some embodiments, the administered liposomal composition comprises 2, 3, 4, 5, or more than 5, D-gamma glutamyl groups or D-alpha glutamyl groups. In some embodiments, the administered liposomal composition comprises L and D gamma polyglutamated Antifolate or L and D alpha polyglutamated Antifolate. In some embodiments, the administered liposomal composition comprises 2, 3, 4, 5, or more than 5, L-gamma glutamyl groups and 2, 3, 4, 5, or more than 5, D-gamma glutamyl groups. In some embodiments, the administered liposomal composition comprises 2, 3, 4, 5, or more than 5, L-alpha glutamyl groups and 2, 3, 4, 5, or more than 5, D-alpha glutamyl groups.

In some embodiments, the disclosure provides a method for treating a disorder of the immune system (e.g., an autoimmune disease such as inflammation and rheumatoid arthritis) that comprises administering an effective amount of a delivery vehicle (e.g., antibody or liposome) comprising polyglutamated Antifolate (e.g., a PANTIFOL disclosed herein) to a subject having or at risk of having a disorder of the immune system. In some embodiments, the autoimmune disease is rheumatoid arthritis. In some embodiments, the delivery vehicle comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In some embodiments, the administered delivery vehicle is pegylated. In some embodiments, the administered delivery vehicle is not pegylated. In additional embodiments, the administered delivery vehicle comprises a targeting moiety that has a specific affinity for an epitope of antigen on the surface of an immune cell associated with a disorder of the immune system. In some embodiments, the targeting moiety is an antibody or an antigen binding antibody fragment. In some embodiments, the administered delivery vehicle comprises a PANTIFOL containing 4, 5, 2-10, 4-6, or more than 5, γ-glutamyl groups or α-glutamyl groups. In some embodiments, the administered delivery vehicle comprises gamma tetraglutamated Antifolate or alpha tetraglutamated Antifolate. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups.

In some embodiments, the disclosure provides a method for treating an infectious disease (e.g., HIV malaria, and schistomiasis) that comprises administering an effective amount of a delivery vehicle (e.g., antibody or liposome) comprising polyglutamated Antifolate (e.g., a PANTIFOL disclosed herein) to a subject having or at risk of having an infectious disease. In some embodiments, the delivery vehicle comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In some embodiments, the delivery vehicle is a liposome. In some embodiments, the administered delivery vehicle is pegylated. In some embodiments, the administered delivery vehicle is not pegylated. In additional embodiments, the administered delivery vehicle comprises a targeting moiety that has a specific affinity for an epitope of antigen on the surface of a pathogen associated with an infectious disease. In some embodiments, the targeting moiety is an antibody or an antigen binding antibody fragment. In some embodiments, the administered delivery vehicle comprises PANTIFOL containing 4, 5, 2-10, 4-6, or more than 5, γ-glutamyl groups or α-glutamyl groups. In some embodiments, the administered delivery vehicle comprises gamma tetraglutamated Antifolate. In some embodiments, the administered delivery vehicle comprises gamma pentaglutamated Antifolate or alpha pentaglutamated Antifolate. In other embodiments, the administered delivery vehicle comprises gamma hexaglutamated Antifolate or alpha hexaglutamated Antifolate. In some embodiments, the administered delivery vehicle comprises L gamma polyglutamated Antifolate or L alpha polyglutamated Antifolate. In some embodiments, the administered delivery vehicle comprises 2, 3, 4, 5, or more than 5, L-gamma glutamyl groups or L-alpha glutamyl groups. In some embodiments, the administered delivery vehicle comprises D gamma polyglutamated Antifolate or D alpha polyglutamated Antifolate. In some embodiments, the administered delivery vehicle comprises 2, 3, 4, 5, or more than 5, D-gamma glutamyl groups or D-alpha glutamyl groups. In some embodiments, the administered delivery vehicle comprises L and D gamma polyglutamated Antifolate or L and D alpha polyglutamated Antifolate. In some embodiments, the administered delivery vehicle comprises 2, 3, 4, 5, or more than 5, L-gamma glutamyl groups and 2, 3, 4, 5, or more than 5, D-gamma glutamyl groups. In some embodiments, the administered delivery vehicle comprises 2, 3, 4, 5, or more than 5, L-alpha glutamyl groups and 2, 3, 4, 5, or more than 5, D-alpha glutamyl groups.

In some embodiments, the administered delivery vehicle is a liposome or an antibody. In some embodiments, the delivery vehicle comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the delivery vehicle is an antibody (e.g., a full-length IgG antibody, a bispecific antibody, or a scFv). In some embodiments, the delivery vehicle is a liposome (e.g., an Lp-PANTIFOL such as, PLp-PANTIFOL, NTLp-PANTIFOL, NTPLp-PANTIFOL, TLp-PANTIFOL, or TPLp-PANTIFOL). In further embodiments, the liposome is pegylated. In additional embodiments, the delivery vehicle comprises a targeting moiety on its surface that specifically binds an antigen on the surface of a target cell of interest. In further embodiments, the delivery vehicle comprises a targeting moiety that specifically binds a cell surface antigen selected from: GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TME1-1-2, endoglin, PSMA, CD98, CD56, CanAg, and CALLA.

In further embodiments, the delivery vehicle is a liposome, and the liposome comprises a targeting moiety that specifically binds a cell surface antigen selected from: GONMB, TACSTD2 (TROP2), CEACAM5, EPCAM, a folate receptor (e.g., folate receptor-α, folate receptor-β or folate receptor-δ), Mucin 1 (MUC-1), MUC-6, STEAP1, mesothelin, Nectin 4, ENPP3, Guanylyl cyclase C (GCC), SLC44A4, NaPi2b, CD70 (TNFSF7), CA9 (Carbonic anhydrase), 5T4 (TPBG), SLTRK6, SC-16, Tissue factor, LIV-1 (ZIP6), CGEN-15027, P-Cadherin, Fibronectin Extra-domain B (ED-B), VEGFR2 (CD309), Tenascin, Collagen IV, Periostin, endothelin receptor, HER2, HER3, EGFR, IGFR-1, EGFRvIII, CD2, CD3, CD4, CD5, CD6, CD11, CD11a, CD15, CD18, CD19, CD20, CD22, CD26, CD27L, CD30, CD33, CD34, CD37, CD38, CD40, CD44, CD56, CD70, CD74, CD79, CD79b, CD105, CD133, CD138, cripto, CD38, an EphA receptor, an EphB receptor, EphA2, an integrin (e.g., integrin αvβ3, αvβ5, or αvβ6), a C242 antigen, Apo2, PSGR, NGEP, PSCA, TMEFF2, endoglin, PSMA, CD98, CD56, CanAg, and CALLA. In some embodiments, the liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the disclosure provides for the use of a composition comprising a polyglutamated Antifolate for manufacture of a medicament for treatment of a hyperproliferative disease. In some embodiments, the composition comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the composition comprises a liposome that comprises a targeting moiety. In some embodiments, the polyglutamated Antifolate is a gamma polyglutamated Antifolate. In some embodiments, the gamma polyglutamated Antifolate comprise 5 or more glutamyl groups. In some embodiments, the gamma polyglutamated Antifolate is pentaglutamated or hexaglutamated. In some embodiments, the gamma polyglutamated Antifolate is in a liposome. In some embodiments, the polyglutamated Antifolate is an alpha polyglutamated Antifolate. In some embodiments, the alpha polyglutamated Antifolate comprise 5 or more glutamyl groups. In some embodiments, the alpha polyglutamated Antifolate is pentaglutamated or hexaglutamated. In some embodiments, the alpha polyglutamated Antifolate is in a liposome. In some embodiments, the hyperproliferative disease is cancer. In some embodiments, the cancer is selected from: lung (e.g., non-small lung cancer), pancreatic, breast cancer, ovarian, lung, prostate, head and neck, gastric, gastrointestinal, colon, esophageal, cervical, kidney, biliary duct, gallbladder, and a hematologic malignancy. In some embodiments, the cancer is selected from: breast cancer, advanced head and neck cancer, lung cancer, stomach cancer, osteosarcoma, Non-Hodgkin's lymphoma (NHL), acute lymphoblastic leukemia (ALL), mycosis fungoides (cutaneous T-cell lymphoma) choriocarcinoma, chorioadenoma, nonleukemic meningeal cancer, soft tissue sarcoma (desmoid tumors, aggressive fibromatosis), bladder cancer, and central nervous system (CNS) lymphoma. In some embodiments, the liposomal composition is administered to treat lung cancer (e.g., NSCLC or mesothelioma). In some embodiments, the liposomal composition is administered to treat breast cancer (e.g., HER2++ or triple negative breast cancer). In some embodiments, the composition is administered to treat colorectal cancer. In some embodiments, the composition is administered to treat ovarian cancer. In some embodiments, the composition is administered to treat endometrial cancer. In some embodiments, the composition is administered to treat pancreatic cancer. In some embodiments, the composition is administered to treat liver cancer. In some embodiments, the composition is administered to treat head and neck cancer. In some embodiments, the composition is administered to treat osteosarcoma. In some embodiments, the cancer is leukemia or lymphoma. In some embodiments, the hyperproliferative disease is an autoimmune disease. In some embodiments, the hyperproliferative disease is inflammation and rheumatoid arthritis.

The disclosed methods can be practiced in any subject that is likely to benefit from delivery of compositions contemplated herein (e.g., gamma polyglutamated Antifolate compositions such as liposome containing a gamma pentaglutamated or gamma hexaglutamated Antifolate, alpha polyglutamated Antifolate compositions such as liposome containing an alpha pentaglutamated or alpha hexaglutamated Antifolate). Mammalian subjects, and in particular, human subjects are preferred. In some embodiments, the subjects also include animals such as household pets (e.g., dogs, cats, rabbits, and ferrets), livestock or farm animals (e.g., cows, pigs, sheep, chickens and other poultry), horses such as thoroughbred horses, laboratory animals (e.g., mice, rats, and rabbits), and other mammals. In other embodiments, the subjects include fish and other aquatic species.

The subjects to whom the agents are delivered may be normal subjects. Alternatively, the subject may have or be at risk of developing a condition that can be diagnosed or that can benefit from delivery of one or more of the provided compositions. In some embodiments, such conditions include cancer (e.g., solid tumor cancers or non-solid cancer such as leukemias). In some embodiments, these conditions (e.g., cancers) involve cells that express an antigen that can be specifically bound by a targeted pegylated liposomal gamma polyglutamated Antifolate and/or alpha polyglutamated Antifolate disclosed herein. In further embodiments, these antigens specifically bind and internalize the targeted pegylated liposomal polyglutamated Antifolate into the cell. In some embodiments, the targeted pegylated liposomal gamma polyglutamated Antifolate and/or alpha polyglutamated Antifolate specifically binds a folate receptor (e.g., folate receptor alpha (FR-α), folate receptor beta (FR-β) and folate receptor delta (FR-δ)) expressed on the surface of the cancer cell.

Tests for diagnosing the conditions that can be treated with the provided compositions are known in the art and will be familiar to the medical practitioner. The determination of whether a cell type expresses folate receptors can be made using commercially available antibodies. These laboratory tests include without limitation microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunosorbent assays, urine screening tests, DNA probe hybridization, and serologic tests. The medical practitioner will generally also take a full history and conduct a complete physical examination in addition to running the laboratory tests listed above.

A subject having a cancer can, for example, be a subject that has detectable cancer cells. A subject at risk of developing a cancer can, for example, be a subject that has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (e.g., carcinogens) such as tobacco, asbestos, or other chemical toxins, and subjects previously treated for cancer and in apparent remission.

In some embodiments, the disclosure provides methods for selectively deliver a folate receptor targeted pegylated liposomal polyglutamated Antifolate to a tumor cell expressing a folate receptor on its surface at a rate that is higher (e.g., at least two-fold greater, at least three-fold greater, at least four-fold greater, or at least five-fold greater, than a cell not expressing folate receptor on its cell surface). In some embodiments, the pegylated liposome comprises L gamma polyglutamated Antifolate. In some embodiments, the pegylated liposome comprises L alpha polyglutamated Antifolate. In some embodiments, the pegylated liposome comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the pegylated liposome comprises 2, 3, 4, 5, or more than 5, L-gamma glutamyl groups or L-alpha glutamyl groups. In some embodiments, the pegylated liposome comprises D gamma polyglutamated Antifolate or D alpha polyglutamated Antifolate. In some embodiments, the pegylated liposome comprises 2, 3, 4, 5, or more than 5, D-gamma glutamyl groups D-alpha glutamyl groups. In some embodiments, the pegylated liposome comprises L and D gamma polyglutamated Antifolate or L and D alpha polyglutamated Antifolate. In some embodiments, the pegylated liposome comprises 2, 3, 4, 5, or more than 5, L-gamma glutamyl groups or L-alpha glutamyl groups. In some embodiments, the pegylated liposome comprises 2, 3, 4, 5, or more than 5, L-gamma glutamyl groups and 2, 3, 4, 5, or more than 5, D-gamma glutamyl groups. In some embodiments, the peglated liposome comprises 2, 3, 4, 5, or more than 5, L-alpha glutamyl groups and 2, 3, 4, 5, or more than 5, D-alpha glutamyl groups.

### Combination Therapy

In certain embodiments, in addition to administering polyglutamated Antifolate composition described herein, the method or treatment further comprises administering at least one additional therapeutic agent. An additional therapeutic agent can be administered prior to, concurrently with, and/or subsequently to, administration of the polyglutamated Antifolate composition. The additional therapeutic agent can be associated with a polyglutamated Antifolate delivery vehicle (e.g., coencapsulated with polyglutamated Antifolate in a liposome), present in a solution containing a polyglutamated Antifolate delivery vehicle, or in a separate formulation from the composition containing the polyglutamated Antifolate composition. Pharmaceutical compositions comprising a polypeptide or agent and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the polypeptide or agent(s). Combination therapy may decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments, the disclosure provides a method for treating cancer that comprises administering an effective amount of a polyglutamated Antifolate composition disclosed herein and a biologic. In some embodiments, the administered polyglutamated Antifolate is a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the polyglutamated Antifolate is administered in combination with a therapeutic antibody. In further embodiments, the polyglutamated Antifolate is administered in combination with an anti-CD antibody (e.g., rituximab) or an antibody that binds an immune checkpoint protein (e.g., CTLA4, PD1, PDL1, and TIM3). In further embodiments, the polyglutamated Antifolate is administered in combination with an fc-fusion protein (e.g., entanercept).

In some embodiments, the disclosure provides a method for treating disorder of the immune system that comprises administering an effective amount of a polyglutamated Antifolate composition disclosed herein and a biologic. In some embodiments, the administered polyglutamated Antifolate is a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the polyglutamated Antifolate is administered in combination with a therapeutic antibody. In further embodiments, the polyglutamated Antifolate is administered in combination with an anti-TNF antibody (e.g., adalimumab). In some embodiments, the polyglutamated Antifolate is administered in combination with an fc-fusion protein (e.g., entanercept).

In some embodiments of the methods described herein, the combination of a PANTIFOL compositions described herein and at least one additional therapeutic agent results in additive or synergistic results. In some embodiments, the combination therapy results in an increase in the therapeutic index of the PANTIFOL or agent. In some embodiments, the combination therapy results in an increase in the therapeutic index of the additional therapeutic agent(s). In some embodiments, the combination therapy results in a decrease in the toxicity and/or side effects of the PANTIFOL or agent. In some embodiments, the combination therapy results in a decrease in the toxicity and/or side effects of the additional therapeutic agent(s).

In some embodiments, in addition to administering polyglutamated Antifolate compositions described herein, the methods or treatments described herein further comprise administering at least one additional therapeutic agent selected from: an anti-tubulin agent, an auristatin, a DNA minor groove binder, a DNA replication inhibitor, an alkylating agent (e.g., platinum complexes such as cisplatin, mono(platinum), bis(platinum) and tri-nuclear platinum complexes and carboplatin), an anthracycline, an antibiotic, an anti-folate (e.g., a polyglutamatable antifolate or a non polyglutamatable anti-folate), an antimitotic (e.g., a vinca alkaloid, such as vincristine, vinblastine, vinorelbine, or vindesine), radiation sensitizer, a steroid, a taxane, a topoisomerase inhibitor (e.g., doxorubicin HCl, daunorubicin citrate, mitoxantrone HCl, actinomycin D, etoposide, topotecan HCl, teniposide (VM-26), and irinotecan), an anti-metabolite, a chemotherapy sensitizer, a duocarmycin, an etoposide, a fluorinated pyrimidine, an ionophore, a lexitropsin, a nitrosourea, a platinol, a purine antimetabolite, a PARP inhibitor, and a puromycin. In certain embodiments, the second therapeutic agent is an alkylating agent, an antimetabolite, an antimitotic, a topoisomerase inhibitor, or an angiogenesis inhibitor. In some embodiments, the administered polyglutamated Antifolate is a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

Therapeutic agents that may be administered in combination with the PANTIFOL compositions described herein include chemotherapeutic agents. Thus, in some embodiments, the methods or treatments described herein further comprise administering at least one involves the administration of a PANTIFOL composition described herein in combination with a chemotherapeutic agent or in combination with a cocktail of chemotherapeutic agents. In some embodiments, the administered polyglutamated Antifolate is a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. Treatment with a PANTIFOL composition can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4.sup.th Edition, 2008, M. C. Perry, Editor, Lippincott, Williams & Wilkins, Philadelphia, Pa.

Chemotherapeutic agents useful in the present invention include, but are not limited to, alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as Antifolate and 5-fluorouracil (5-FU); folic acid analogs such as denopterin, Antifolate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, such as paclitaxel (TAXOL®) and docetaxel (TAXOTERE®); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA®); anti-hormonal agents such as, tamoxifen, raloxifene, aromatase inhibiting 4(5)-imidazoles, 4-hydroxytamoxifen, trioxifene, keoxifene, LY117018, onapristone, and toremifene (FARESTON®); anti-androgens such as flutamide, nilutamide, bicalutamide, leuprolide, and goserelin; and pharmaceutically acceptable salts, acids or derivatives according to any of the above. In certain embodiments, the additional therapeutic agent is cisplatin. In certain embodiments, the additional therapeutic agent is carboplatin. In other embodiments, the additional therapeutic agent is oxaloplatin.

Additional therapeutic agents that may be administered in combination with the PANTIFOL compositions described herein include one or more immunotherapeutic agents.

In some embodiments PANTIFOL is administered in combination with an immunotherapeutic agent that inhibits one or more T cell-associated inhibitory molecules (e.g., CTLA4, PD1, Lymphocyte activation gene-3 (LAG-3, CD223), T cell immunoglobulin-3 (TIM-3), T cell immunoglobulin and ITIM domain (TIGIT), V-domain Ig suppressor of T cell activation (VISTA), B7 homolog 3 (B7-H3, CD276), B and T cell lymphocyte attenuator (BTLA, CD272), or Adenosine A2a receptor (A2aR) or CD73). In some embodiments, the PANTIFOL composition is administered separately from the immunotherapeutic agent. In some embodiments, the PANTIFOL composition is administered at the same time (e.g., concurrently or serially) as the immunotherapeutic agent. In some embodiments, the PANTIFOL and the immunotherapeutic agent are encapsulated in or otherwise associated with the same liposome.

In some embodiments, treatment methods provided herein comprise administering a PANTIFOL composition described herein in combination with a PD1 inhibitor. In some embodiments, the PANTIFOL composition is administered in combination with pembroluzumab. In some embodiments, the PANTIFOL composition is administered in combination with nivolumab. In some embodiments, the PANTIFOL composition is administered separately from the PD1 inhibitor. In some embodiments, the PANTIFOL composition is administered at the same time (e.g., concurrently or serially) as the PD1 inhibitor. In some embodiments, the PANTIFOL and the PD1 inhibitor are encapsulated in or otherwise associated with the same liposome.

In other embodiments, the PANTIFOL composition is administered in combination with a PDL1 inhibitor. In some embodiments, the PANTIFOL composition is administered in combination with atezolizumab. In some embodiments, the PANTIFOL composition is administered in combination with avelumab. In some embodiments, the PANTIFOL composition is administered in combination with durvalumab. In some embodiments, the PANTIFOL composition is administered in combination with PDR001. In some embodiments, the PANTIFOL composition is administered separately from the PDL-1 inhibitor. In some embodiments, the PANTIFOL composition is administered at the same time (e.g., concurrently or serially) as the PDL-1 inhibitor. In some embodiments, the PANTIFOL and the PDL-1 inhibitor are encapsulated in or otherwise associated with the same liposome.

In some embodiments, treatment methods provided herein comprise administering a PANTIFOL composition in combination with a therapeutic agent that inhibits activity of CTLA4 LAG3, TIM-3, TIGIT, VISTA, B7-H3, BTLA, A2aR or CD73. In some embodiments, treatment methods provided herein comprise administering a PANTIFOL composition described herein in combination with a CTLA4 inhibitor. In further embodiments, the PANTIFOL composition is administered in combination with ipilimumab. In some embodiments, treatment methods provided herein comprise administering a PANTIFOL composition in combination with a LAG3 inhibitor. In further embodiments, the PANTIFOL composition is administered in combination with TSR-033, MK-4280, LAG525, BMS-986106, or MGD013. In some embodiments, treatment methods provided herein comprise administering a PANTIFOL composition in combination with a TIM-3 inhibitor. In further embodiments, the PANTIFOL composition is administered in combination with MBG453 or MEDI9447. In some embodiments, treatment methods provided herein comprise administering a PANTIFOL composition in combination with a TIGIT inhibitor. In further embodiments, the PANTIFOL composition is administered in combination with BMS-986207 or OMP-31M32. In some embodiments, treatment methods provided herein comprise administering a PANTIFOL composition in combination with a VISTA inhibitor. In further embodiments, the PANTIFOL composition is administered in combination with JNJ-61610588 or CA-170. In some embodiments, treatment methods provided herein comprise administering a PANTIFOL composition in combination with a B7-H3 inhibitor. In further embodiments, the PANTIFOL composition is administered in combination with neoblituzumab, enoblituzumab, MGD009, or 8H9. In some embodiments, treatment methods provided herein comprise administering a PANTIFOL composition in combination with a BTLA inhibitor. In some embodiments, treatment methods provided herein comprise administering a PANTIFOL composition in combination with an A2aR or CD73 inhibitor. In further embodiments, the PANTIFOL composition is administered in combination with CPI444. In some embodiments, the PANTIFOL composition is administered separately from the immunotherapeutic agent. In some embodiments, the PANTIFOL composition is administered at the same time (e.g., concurrently or serially) as the immunotherapeutic agent. In some embodiments, the PANTIFOL and the immunotherapeutic agent are encapsulated in or otherwise associated with the same liposome.

In some embodiments, treatment methods provided herein comprise administering an PANTIFOL composition in combination with a therapeutic agent that inhibits activity of transforming growth factor (TGF)-β, phosphoinositide 3-kinase gamma (PI3Kγ), Killer immunoglobulin-like receptors (KIR, CD158), CD47, or Indoleamine 2,3-dioxygenase (IDO). In some embodiments, treatment methods provided herein comprise administering a PANTIFOL composition described herein in combination with a TGFβ antagonist. In further embodiments, the PANTIFOL composition is administered in combination with M7824 or Galusertinib (LY2157299). In some embodiments, treatment methods provided herein comprise administering a PANTIFOL composition described herein in combination with a PI3Kγ antagonist. In further embodiments, the PANTIFOL composition is administered in combination with IPI-549. In some embodiments, treatment methods provided herein comprise administering a PANTIFOL composition described herein in combination with a KIR antagonist. In further embodiments, the PANTIFOL composition is administered in combination with IPH4102 or lirilumab. In some embodiments, treatment methods provided herein comprise administering a PANTIFOL composition described herein in combination with a CD47 antagonist. In further embodiments, the PANTIFOL composition is administered in combination with Hu5F9-G4 or TTI-621. In some embodiments, treatment methods provided herein comprise administering a PANTIFOL composition described herein in combination with an IDO antagonist. In further embodiments, the PANTIFOL composition is administered in combination with BMS-986205, indoximod, or epacadostat. In some embodiments, the PANTIFOL composition is administered separately from the therapeutic agent. In some embodiments, the PANTIFOL composition is administered at the same time (e.g., concurrently or serially) as the therapeutic agent. In some embodiments, the PANTIFOL and the therapeutic agent are encapsulated in or otherwise associated with the same liposome.

In some embodiments, treatment methods provided herein comprise administering an PANTIFOL composition in combination with a therapeutic agent that is an agonist of OX40 (CD134), inducible co-stimulator (ICOS), Glucocorticoid-induced TNF receptor family-related protein (GITR), 4-1BB (CD137), CD40, CD27-CD70, or a Toll-like receptor (TLR). In some embodiments, treatment methods provided herein comprise administering a PANTIFOL composition described herein in combination with an OX40 agonist. In further embodiments, the PANTIFOL composition is administered in combination with GSK3174998, MOXR0916, 9B12, PF-04518600 (PF-8600), MEDI6383, MEDI0562, INCAGN01949, or GSK3174998. In some embodiments, treatment methods provided herein comprise administering a PANTIFOL composition described herein in combination with an ICOS agonist. In further embodiments, the PANTIFOL composition is administered in combination with JTX-2011, GSK3359609, or MEDI-570. In some embodiments, treatment methods provided herein comprise administering a PANTIFOL composition described herein in combination with a GITR agonist. In further embodiments, the PANTIFOL composition is administered in combination with TRX-518, AMG 228, BMS-986156, MEDI1873, MK-4166, INCAGN01876, or GWN32. In some embodiments, treatment methods provided herein comprise administering a PANTIFOL composition described herein in combination with a 4-1BB agonist. In further embodiments, the PANTIFOL composition is administered in combination with utomilumab or urelumab (PF-05082566). In some embodiments, treatment methods provided herein comprise administering a PANTIFOL composition described herein in combination with a CD40 agonist. In further embodiments, the PANTIFOL composition is administered in combination with CP-870893, APX005M, ADC-1013, lucatumumab, Chi Lob 7/4, dacetuzumab, SEA-CD40, or R07009789. In some embodiments, treatment methods provided herein comprise administering a PANTIFOL composition described herein in combination with a CD27-CD70 agonist. In further embodiments, the PANTIFOL composition is administered in combination with ARGX-110, or BMS-936561 (MDX-1203). In some embodiments, treatment methods provided herein comprise administering a PANTIFOL composition described herein in combination with a TLR agonist. In further embodiments, the PANTIFOL composition is administered in combination with MEDI9197, PG545 (pixatimod, pINN), or poly-ICLC. In some embodiments, the PANTIFOL composition is administered separately from the therapeutic agent. In some embodiments, the PANTIFOL composition is administered at the same time (e.g., concurrently or serially) as the therapeutic agent. In some embodiments, the PANTIFOL and the therapeutic agent are encapsulated in or otherwise associated with the same liposome.

In some embodiments, the disclosure provides a combination therapy wherein a polyglutamated Antifolate composition described herein is administered in combination with another DMARD. In some embodiments, the administered polyglutamated Antifolate is a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In further embodiments, the polyglutamated Antifolate composition is administered in combination sulfasalazine or hydroxychloroquine. In some embodiments, the disclosure provides a combination therapy wherein a polyglutamated Antifolate composition described herein is administered in combination with chloroquine.

In some embodiments, the disclosure provides a combination therapy wherein a polyglutamated Antifolate composition described herein is administered in combination with a steroid. In some embodiments, the administered polyglutamated Antifolate is a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In further embodiments, the polyglutamated Antifolate composition is administered in combination with prednisolone.

In some embodiments, the disclosure provides a combination therapy wherein a polyglutamated Antifolate composition described herein is administered in combination a biologic agent. In some embodiments, the administered polyglutamated Antifolate is a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof. In some embodiments, the biologic agent is a therapeutic antibody. In further embodiments, the therapeutic binds TNF-alpha or CD-20.

### Kits Comprising PANTIFOL Compositions

The disclosure also provides kits that comprise the PANTIFOL compositions described herein and that can be used to perform the methods described herein. In certain embodiments, a kit comprises at least one purified PANTIFOL composition in one or more containers. In some embodiments, the kit comprises a αPANTIFOL and/or γPANTIFOL of the present disclosure, such as a substantially pure γPANTIFOL of the present disclosure (e.g., Formula III-1-L, III-1-D, or a pharmaceutically acceptable salt thereof, or Formula IV-1-L or IV-1-D), or a substantially pure αPANTIFOL of the present disclosure (e.g., Formula III-1-L-Alpha, III-1-D-Alpha, or a pharmaceutically acceptable salt thereof, or Formula IV-1-L-Alpha or IV-1-D-Alpha), or a combination thereof.

In some embodiments, the kits contain all of the components necessary and/or sufficient to perform a detection assay, including all controls, directions for performing assays, and any necessary software for analysis and presentation of results. One skilled in the art will readily recognize that the disclosed agents can be readily incorporated into one of the established kit formats which are well known in the art.

In some embodiments, the kits include a dosage amount (e.g., as used for therapy or diagnosis) of at least one PANTIFOL compositions (e.g., a PANTIFOL liposome), or pharmaceutical formulation thereof, as disclosed herein. Kits may further comprise suitable packaging and/or instructions for use of the composition. Kits may also comprise a means for the delivery for the composition, or pharmaceutical formulation thereof, such as a syringe for injection or other device as described herein and known to those of skill in the art. One of skill in the art will readily recognize that the disclosed PANTIFOL compositions can be readily incorporated into one of the established kit formats which are well known in the art.

Further provided are kits that comprise a PANTIFOL compositions as well as at least one additional therapeutic agent. In certain embodiments, the second (or more) therapeutic agent is an anti-metabolite. In certain embodiments, the second (or more) therapeutic agent is a chemotherapeutic agent.

The following examples are intended to illustrate but not to limit the disclosure in any manner, shape, or form, either explicitly or implicitly. While they are typical of those that might be used, other procedures, methodologies, or techniques known to those skilled in the art may, be used without departing from the scope of the present disclosure.

### EXAMPLES

The various starting materials, intermediates, and compounds of the preferred embodiments can be isolated and purified where appropriate using conventional techniques such as precipitation, filtration, crystallization, evaporation, distillation, and chromatography. Characterization of these compounds can be performed using conventional methods such as by melting point, elemental analysis, optical rotation, mass spectrometry, NMR (nuclear magnetic resonance), and various other spectroscopic analyses. Exemplary embodiments of steps for performing the synthesis of products described herein are described in greater detail infra.

### Example 1. Synthesis of Compound J

### Step 1.

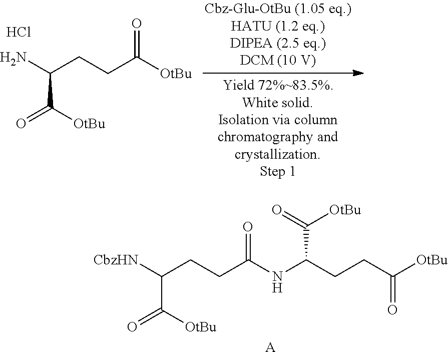

Charged dichloromethane (DCM) (20 kg, 10 V) and Cbz-Glu-OtBu (1.8 kg, 1.05 eq.) to a 30 L reactor. Cooled the reaction mass to 10° C. HATU (2.31 kg, 1.2 eq.) and diisopropylethylamine (DIPEA) (1.65 kg, 2.5 eq.) were added to the reaction mixture at 10° C. The reaction mixture was allowed to warm to room temperature and stirred for 30 min Glu(OtBu)-OtBu-HCl (1.5 kg, 1.0 eq.) was added to the reaction mixture at room temperature. The reaction mixture was stirred for 20 h at room temperature. Analysis by HPLC @210 nm indicated that Glu(OtBu)-OtBu-HCl was not detectable. 10% of aqueous citric acid (11.83 kg) was added and stirred for 10 min. Separated the layers and organic layer was washed with 10% aqueous citric acid (12.02 kg). Separated the layers and organic layer was washed with 10% aqueous citric acid (12.22 kg). Saturated aqueous sodium bicarbonate (12.11 kg) was slowly added and stirred for 10 min.

Separated the layers and organic layer was washed with saturated aqueous sodium bicarbonate (11.92 kg). Organic layer was washed with brine (13.7 kg). The organic layer was dried over anhydrous sodium sulfate and concentrated under vacuum. The residue was purified by column chromatography on silica gel (hexane/EtOAc=5:1). Fractions were concentrated to 2 V (˜3 L) under vacuum. Hexane (8.25 L, 8 V) was added and stirred for 30 min. Filtered the solid material to give wet solid. Dried the solid in air to afford 2.4 kg of white solid material with 99.5% purity and 81.8% yield.

### Step 2.

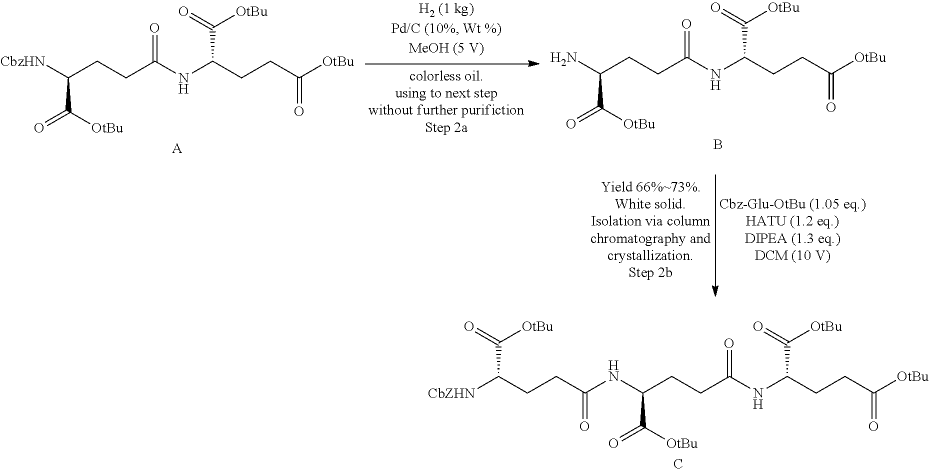

Charged MeOH (12 kg, 6 V) and Compound A (2.4 kg, 1.0 eq.) to a 30 L reactor. Nitrogen replacement and protection were carried out. 10% Pd/C (0.25 kg) was added at 10-20° C. The reaction mixture was stirred under hydrogen (H₂) atmosphere at 45° C. for 12 h. Analysis by HPLC @210 nm indicated that compound A was not detectable. Nitrogen replacement was carried out. The mixture was filtered. The filtrate was concentrated under vacuum to afford 1.83 kg of Compound B as a colorless oil with 98.8% purity and quantitative yield.

Compound B was converted into compound C via similar process as described in Step 1. Charged DCM (23.8 kg, 10 V), Cbz-Glu-OtBu (1.44 kg, 1.05 eq.) to a 30 L reactor. Cooled the reaction mass to 10° C. HATU (1.86 kg, 1.2 eq.) and DIPEA (0.68 kg, 1.3 eq.) were added to the reaction mixture at 10° C. The reaction mixture was allowed to warm to room temperature and stirred for 30 min. Compound-B (1.81 kg, 1.0 eq.) was added to the reaction mixture at room temperature. The reaction mixture was stirred for 20 h at room temperature. Analysis by HPLC @210 nm indicated that compound B was not detectable. 10% of aqueous citric acid (12.3 kg) was added and stirred for 10 min. Separated the layers and organic layer was washed with 10% aqueous citric acid (12.2 kg). Separated the layers and organic layer was washed with saturated aqueous sodium bicarbonate (12.2 kg). Organic layer was washed with brine (14.0 kg). The organic layer was dried over anhydrous sodium sulfate and concentrated under vacuum.

The residue was purified by column chromatography on silica gel (hexane/EtOAc=3:1). Fractions were concentrated to 2 V (˜2.9 L) under vacuum. Hexane (8.25 kg, 8 V) was added and stirred for 30 min. Filtered the solid material to give wet solid. Dried the solid in air to afford 2.25 kg of white solid material with 99.7% purity and 69% yield.

### Step 3.

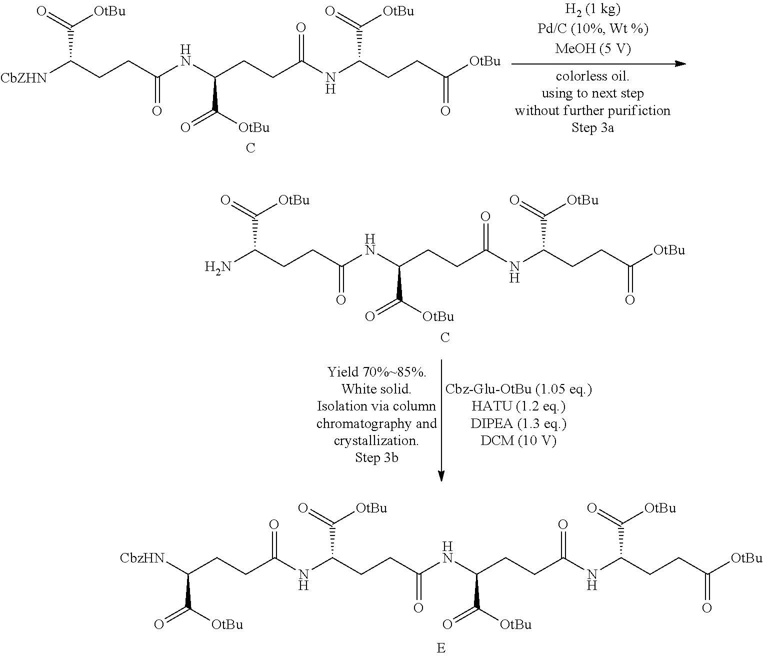

Compound C was converted into Compound D similar to the procedure described in Step 2. Charged MeOH (8.5 kg, 5 V) and Compound-C (2.1 kg, 1.0 eq.) to a 30 L reactor. Nitrogen replacement and protection were carried out. 10% Pd/C (0.22 kg) was added at 10˜20° C. The reaction mixture was stirred under H₂ atmosphere at 45° C. for 20 h. Analysis by HPLC @210 nm indicated that Compound C was not detectable. Nitrogen replacement was carried out. The mixture was filtered. The filtrate was concentrated under vacuum to afford 1.75 kg of Compound-D as a colorless oil with 99.7% purity and quantitative yield.

Compound D was converted into Compound E similar to the procedure described in Step 2. Charged DCM (23 kg, 10 V), Cbz-Glu-OtBu (62.2 g, 1.05 eq.) to a 2 L reactor. Cooled the reaction mass to 10° C. HATU (80.3 g, 1.2 eq.) and DIPEA (29.5 g, 1.3 eq.) were added to the reaction mixture at 10° C. The reaction mixture was allowed to warm to room temperature and stirred for 30 min. Compound D (110.5 g, 1.0 eq.) was added to the reaction mixture at room temperature. The reaction mixture was stirred for 20 h at room temperature. Analysis by HPLC @210 nm indicated that Compound D was not detectable. 10% of aqueous citric acid (400 mL) was added and stirred for 10 min. Separated the layers and organic layer was washed with 10% aqueous citric acid (400 mL). Separated the layers and organic layer was washed with saturated aqueous sodium bicarbonate (400 mL). Organic layer was washed with brine (400 mL). The organic layer was dried over anhydrous sodium sulfate and concentrate under vacuum.

The residue was purified by column chromatography on silica gel (hexane/EtOAc=3:1). Fractions were concentrated to 2.7 V (˜300 mL) under vacuum. Hexane (1450 mL, 13 V) was added and stirred for 30 min. Filtered the solid material to give wet solid. Dried the solid in air to afford Compound E, 115.1 g of white solid material with 99.6% purity and 70% yield.

### Step 4.

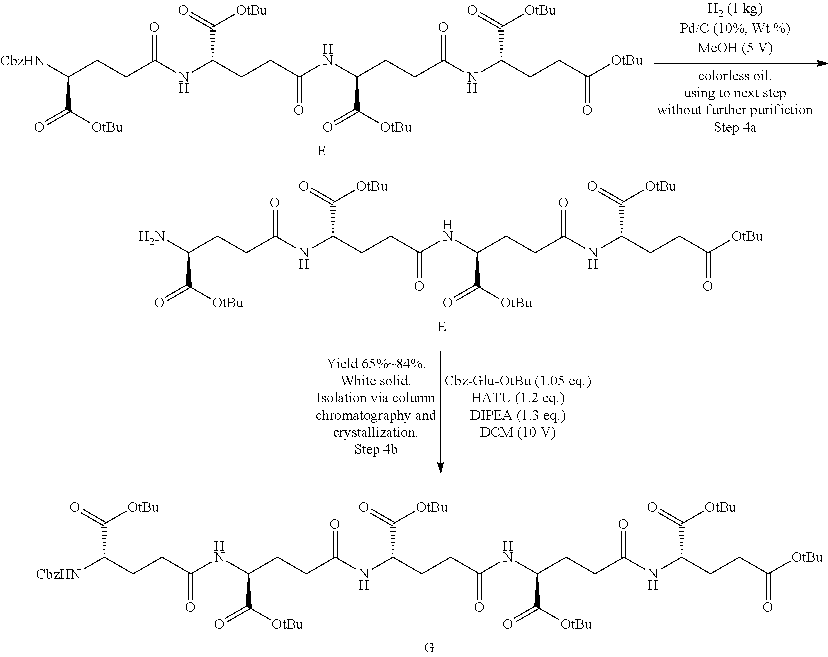 [SMILES: CC(C)(C)OC(=O)CC[C@H](NC(=O)CC[C@H](NC(=O)CC[C@H](NC(=O)CC[C@H](NC(=O)OCc1ccccc1)C(=O)OC(C)(C)C)C(=O)OC(C)(C)C)C(=O)OC(C)(C)C)C(=O)OC(C)(C)C ; CC(C)(C)OC(=O)CC[C@H](NC(=O)CC[C@H](NC(=O)CC[C@H](NC(=O)CC[C@H](N)C(=O)OC(C)(C)C)C(=O)OC(C)(C)C)C(=O)OC(C)(C)C)C(=O)OC(C)(C)C ; CC(C)(C)OC(=O)CC[C@H](NC(=O)CC[C@H](NC(=O)CC[C@H](NC(=O)CC[C@H](NC(=O)CC[C@H](NC(=O)OCc1ccccc1)C(=O)OC(C)(C)C)C(=O)OC(C)(C)C)C(=O)OC(C)(C)C)C(=O)OC(C)(C)C)C(=O)OC(C)(C)C]

Compound E was converted into Compound F via similar procedures described in steps 2 and 3. Charged MeOH (8.5 kg, 5 V) and Compound-E (2.15 kg, 1.0 eq.) to a 30 L reactor. Nitrogen replacement and protection. 10% Pd/C (0.22 kg) was added at 10˜20° C. The reaction mixture was stirred under H2 atmosphere at 45° C. for 20 h. Analysis by HPLC @210 nm indicated that Compound E was not detectable. Nitrogen replacement. The mixture was filtered. The filtrate was concentrated under vacuum to afford 1.84 kg of Compound-F as a colorless oil with 99.5% purity and quantitative yield.

Compound F was converted into Compound G via similar procedures described in steps 2 and 3. Charged DCM (24 kg, 10 V), Cbz-Glu-OtBu (0.8 kg, 1.05 eq.) to a 30 L reactor. Cooled the mixture to 10° C. HATU (1.03 kg, 1.2 eq.) and DIPEA (0.38 kg, 1.3 eq.) were added to the reaction mixture at 10° C. The reaction mixture was allowed to warm to room temperature and stirred for 30 min. Compound-F (1.84 kg, 1.0 eq.) was added to the reaction mixture at room temperature. The reaction mixture was stirred for 20 h at room temperature. Analysis by HPLC @210 nm indicated that Compound F was not detectable. 10% of aqueous citric acid (10.8 kg) was added and stirred for 10 min. Separated the layers and organic layer was washed with 10% aqueous citric acid (11 kg). Separated the layers and organic layer was washed with saturated aqueous sodium bicarbonate (10.8 kg). Organic layer was washed with brine (12.1 kg). The organic layer was dried over anhydrous sodium sulfate and concentrated under vacuum. The residue was purified by column chromatography on silica gel (hexane/EtOAc=2:1). Fractions were concentrated to 2 V (4 L) under vacuum. Hexane (20.3 kg, 15 V) was added and stirred for 30 min. Filtered the solid material to give wet solid. Dried the solid in air to afford Compound G, 2.16 kg of white solid material with 99.5% purity and 84% yield.

### Step 5.

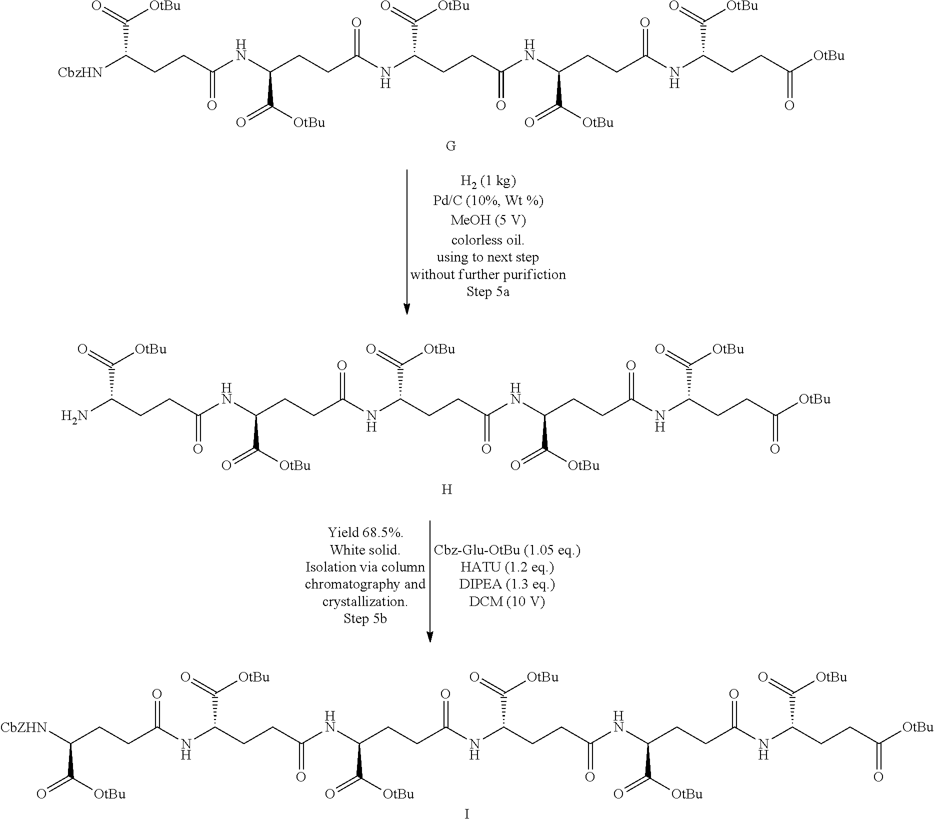

Compound G was converted into Compound H via similar procedures described above in steps 2-4. Charged MeOH (11.8 kg, 5 V) and Compound-G (2.12 kg, 1.0 eq.) to a 30 L reactor. Nitrogen replacement and protection. 10% Pd/C (0.23 kg) was added at 10˜20° C. The mixture was subjected to hydrogenolysis under H2 atmosphere at 45°. Analysis by HPLC @210 nm indicated that Compound G was not detectable. Nitrogen replacement. The mixture was filtered. The filtrate was concentrated under vacuum to afford 1.86 kg of Compound-H as a colorless oil with 98.1% purity and quantitative yield.

Compound H was converted into Compound I via similar procedures described above in steps 2-4. Charged DCM (25.9 kg, 10 V), Cbz-Glu-OtBu (0.66 kg, 1.05 eq.) to a 30 L reactor. Cooled the mixture to 10° C. HATU (0.84 kg, 1.2 eq.) and DIPEA (0.31 kg, 1.3 eq.) were added to the reaction mixture at 10° C. The reaction mixture was allowed to warm to room temperature and stirred for 30 min. Compound-H (1.86 kg, 1.0 eq.) was added to the reaction mixture at room temperature. The reaction mixture was stirred for 20 h at room temperature. Analysis by HPLC @210 nm indicated that Compound H was not detectable. 10% of aqueous citric acid (10.8 kg) was added and stirred for 10 min Separated the layers and organic layer was washed with 10% aqueous citric acid (10.7 kg). Saturated aqueous sodium bicarbonate (10.5 kg) was slowly added and stirred for 10 min. Organic layer was washed with brine (12 kg). The organic layer was dried over anhydrous sodium sulfate and concentrated under vacuum. The residue was purified by column chromatography on silica gel (hexane/EtOAc=2:1). Fractions were concentrated to 2V (4 L) under vacuum. Hexane (20.4 kg, 15V) was added and stirred for 30 min Filtered the solid material to give wet solid. Dried the solid in air to afford 2.09 kg of Compound-I as a white solid with 98.2% purity and 84.7% yield.

### Step 6

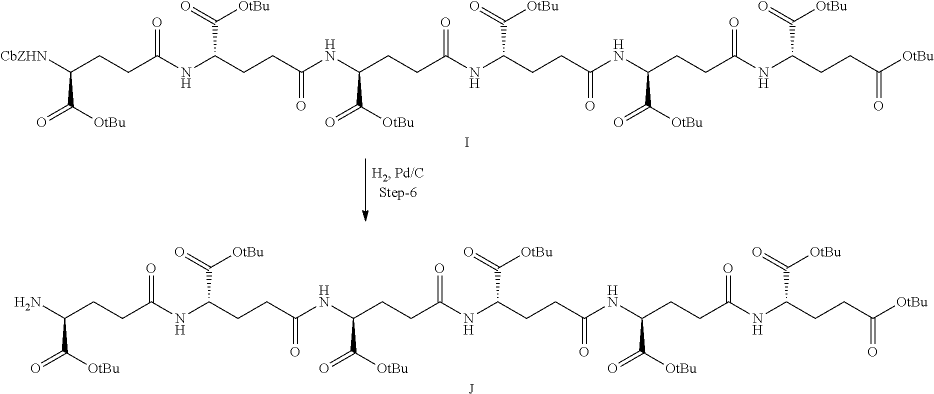

Compound I was converted into Compound J via similar procedures described above in steps 2-5. Charged MeOH (16.6 kg, 10V) and Compound-I (2.08 kg, 1.0 eq.) to a 30 L reactor. Nitrogen replacement and protection. 10% Pd/C (0.21 kg) was added at 10-20° C. The reaction mixture was subjected to hydrogenolysis under H₂ atmosphere at 45°. After analysis by HPLC @210 nm indicated that Compound I was not detectable in the reaction mixture, nitrogen replacement was carried out. The mixture was filtered. The filtrate was concentrated under vacuum. The residue was treated with isopropyl ether (3.9 kg) and filtered to afford 1.59 kg of Compound-J as a white solid with 98.4% purity and 85% yield.

HPLC Method 1 for Analyzing Compound-J:

| Instrument | Waters 2695-2998-SEDEX 85 | |
|---|---|---|
| Column | Agilent Eclipse plus C18, 3.5 μm, | |
| | 100 mm (L) × 4.6 mm (ID) | |
| Flow Rate | 1.0 | mL/min |
| Injection volume | 1.0 | μL |
| Detection | 210 | nm |
| Column Temp | 30° | C. |
| Sample run time | 15 | min |

| | |
|---|---|
| Sample solvent | MeOH |
| Needle wash | 10% MeOH |
| Equilibration | 10 min Chromatographic |
| time | |

### Conditions: Gradient Elution

| Time (min) | MPA (%) | MPB (%) | Elution |
|---|---|---|---|
| 0 | 50 | 50 | Initial conditions |
| 15.00 | 0 | 100 | Linear |

Mobile Phase A (MPA): 0.025% HCOOH/H₂O (v/v)
Mobile Phase B (MPB): 0.025% HCOOH/ACN (CH₃CN) (v/v)

### Example 2. Synthesis of Compound 100

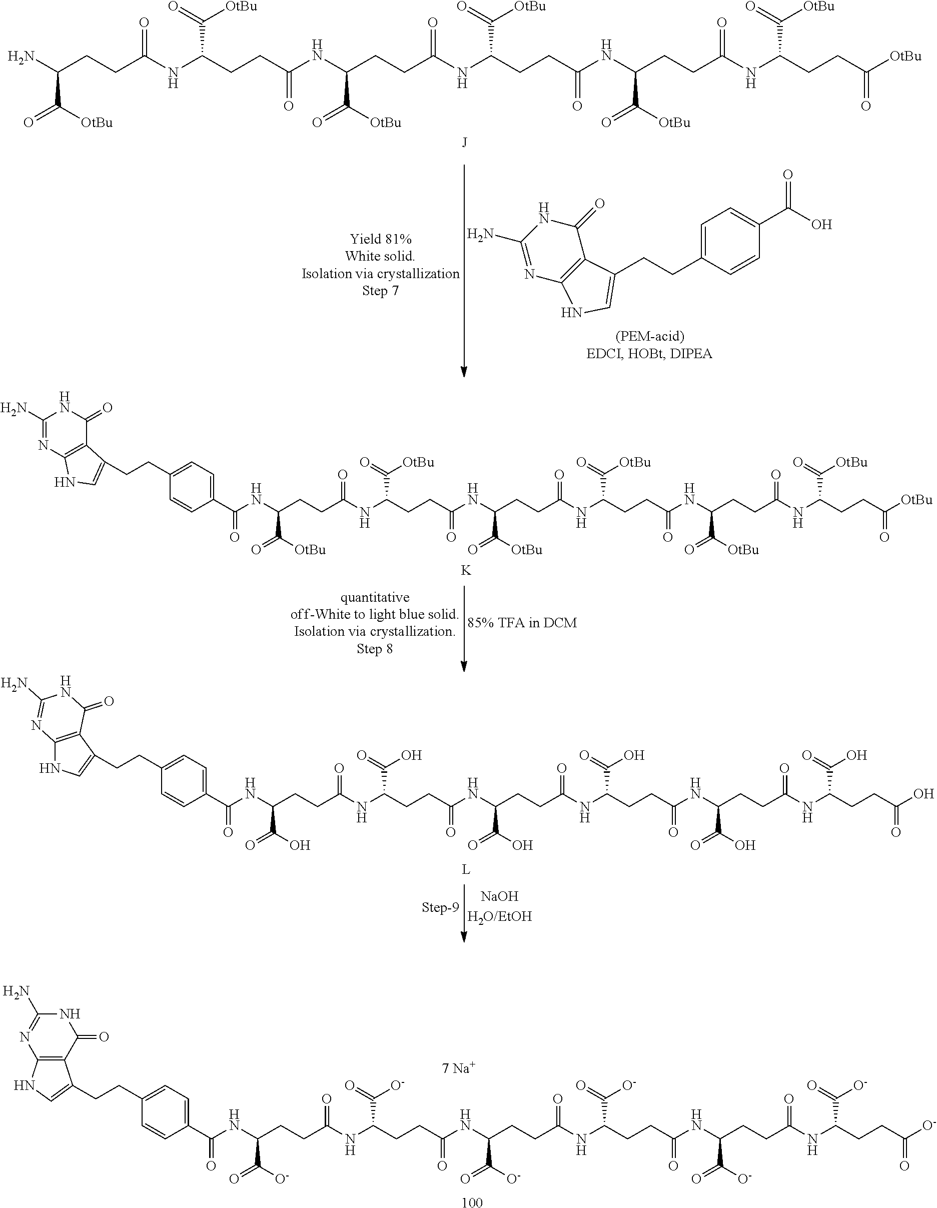

### Step 7.

Charged DMF (14.5 kg), PEM-acid (0.385 kg, 1.0 eq.) to a 30 L reactor. Cooled the mixture to 10° C. EDCI (0.27 kg, 1.1 eq.), HOBt (0.19 kg, 1.1 eq.) and DIPEA (0.21 kg, 1.2 eq.) were added to the reaction mixture at 10° C. The reaction mixture was allowed to warm to room temperature and stirred for 30 min. A solution of Compound-J (1.58 kg, 1.03 eq.) in DMF (6.3 kg) was added to the reaction mixture at room temperature. The reaction mixture was stirred for 20 h at room temperature. After analysis by HPLC @210 nm indicated that PEM-acid was not detectable, the mixture was diluted with EtOAc (22.2 kg). 10% of aqueous citric acid (12.5 kg) was added and stirred for 10 min. Separated the layers and the aqueous was re-extracted with EtOAc (8.5 kg×2). The three batches EtOAc layers were combined and washed with 10% aqueous citric acid (16.5 kg). Saturated aqueous sodium bicarbonate (16.4 kg) was slowly added and stirred for 10 min. Organic layer was washed with water (15.1 kg). The organic layer was dried over anhydrous sodium sulfate and concentrated under vacuum. The residue was dissolved in DCM (7.0 kg) and MeOH (0.28 kg) at 45° C. MTBE (14.0 kg) was added and the resulting mixture was stirred for 30 min. The mixture was cooled to room temperature. Filtered the solid material to give wet solid. Dried the solid in air to afford 1.61 kg of Compound-K as a white solid with 99.2% purity and 82.5% yield. The HPLC used for analyzing purity of Compound-K is the HPLC Method 1 shown in Example 1.

### Step 8.

Charged DCM (4.8 kg) and TFA (30.5 kg) to a 30 L reactor. Cooled the mixture to 10° C. Compound-K (1.6 kg, 1.0 eq.) was added. The reaction mixture was stirred at room temperature for 15 h. The mixture was concentrated under reduced pressure. The residue was treated with EtOAc (14.4 kg). Filtered the solid material to give wet solid. Dried the solid in drying oven to afford crude Compound-L.

Charged DCM (4.8 kg) and TFA (30.5 kg) to a 30 L reactor. Cooled the mixture to 10° C. The crude Compound-L was added. The reaction mixture was stirred at room temperature for 8 h. Analysis of a sample of the reaction mixture by HPLC @210 nm indicated that the purity of Compound-L was >98%. The mixture was concentrated under reduced pressure. The residue was treated with EtOAc (14.4 kg). Filtered the solid material to give wet solid. Dried the solid in drying oven to afford 1.22 kg of Compound-L as a solid with 98% purity and quantitative yield.

HPLC Method 2 was used for the analysis of purity of Compound-L:

| Instrument | Waters 2695-2998-SEDEX 85 | |
|---|---|---|
| Column | Agilent Eclipse plus C18, 3.5 μm, | |
| | 100 mm (L) × 4.6 mm (ID) | |
| Flow Rate | 1.0 | mL/min |
| Injection volume | 1.0 | μL |
| Detection | 210 | nm |
| Column Temp | 30° | C. |
| Sample run time | 15 | min |
| Sample solvent | CH3CN/H2O | |
| Needle wash | 10% MeOH | |
| Equilibration | 10 | min |
| time | | |

### Chromatographic Conditions: Gradient Elution

| Time (min) | MPA (%) | MPB (%) | Elution |
|---|---|---|---|
| 0 | 95 | 5 | Initial conditions |
| 15.00 | 65 | 35 | Linear |

MPA: 0.025% HCOOH/H2O (v/v)
MPB: 0.025% HCOOH/ACN (v/v)

### Step 9.

Charged water (2.5 kg) and NaOH (0.2 kg) to a 30 L reactor. Cooled the mixture to 0-5° C. Compound-L (1.21 kg) was added. The mixture was adjusted pH to 9 with 5 M aqueous NaOH (0.65 L). The reaction mixture was stirred at room temperature for 10 min. Monitored the pH, pH=9.

The resulting solution was added dropwise into stirring EtOH (52 kg) at room temperature. The mixture was adjusted pH to 9 with 5 M aqueous NaOH and stirred at room temperature for 30 min. Filtered the mixture and collected the solid. The solid was dried in drying oven to afford crude Compound 100 (1.35 kg).

The crude Compound 100 (1.35 kg) was dissolved in water (2.3 kg) at 0-10° C. The resulting solution was added dropwise into stirring EtOH (36 kg) at room temperature. The mixture was stirred at room temperature for 30 min. Filtered the mixture and collected the solid. The solid was dried in drying oven to afford crude Compound 100 (1.28 kg).

The crude Compound 100 (1.28 kg) was dissolved in water (2.2 kg) at 0-10° C. The resulting solution was added dropwise into stirring EtOH (36.2 kg) at room temperature. The mixture was stirred at room temperature for 30 min. Filtered the solid material to give wet solid. Dried the solid in drying oven to afford 1.19 kg of Compound 100 as a solid with 98.3% purity and 93.7% yield. HPLC Method 2 was used for the purity analysis of Compound 100. Representative high-resolution mass (M+H) found 1073.3690, M+H calculated 1073.3699. The compound 100 was fully characterized by NMR, LC-MS and elemental analysis.

### Example 3. Synthesis of Compound 110

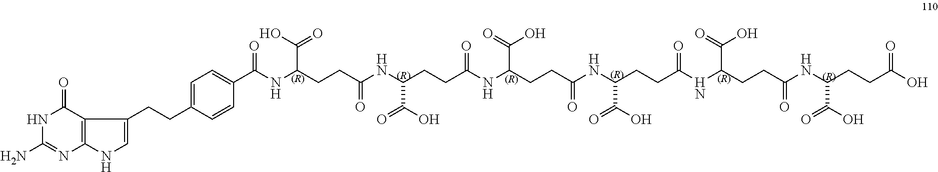

The compound 110 is the enantiomer of compound L shown in Example 2. It was synthesized similarly as shown in Examples 1 and 2, except the D-glutamate starting material/intermediates were used instead. Representative high-resolution Mass Spectral data: Calculated (M+H): 1073.3699, Found (M+H): 1073.3687.

The compound 110 can also be converted into its salts such as hepta-sodium salt similar to compound 100 as shown in Example 2.

### Example 4. Synthesis of Compound 200

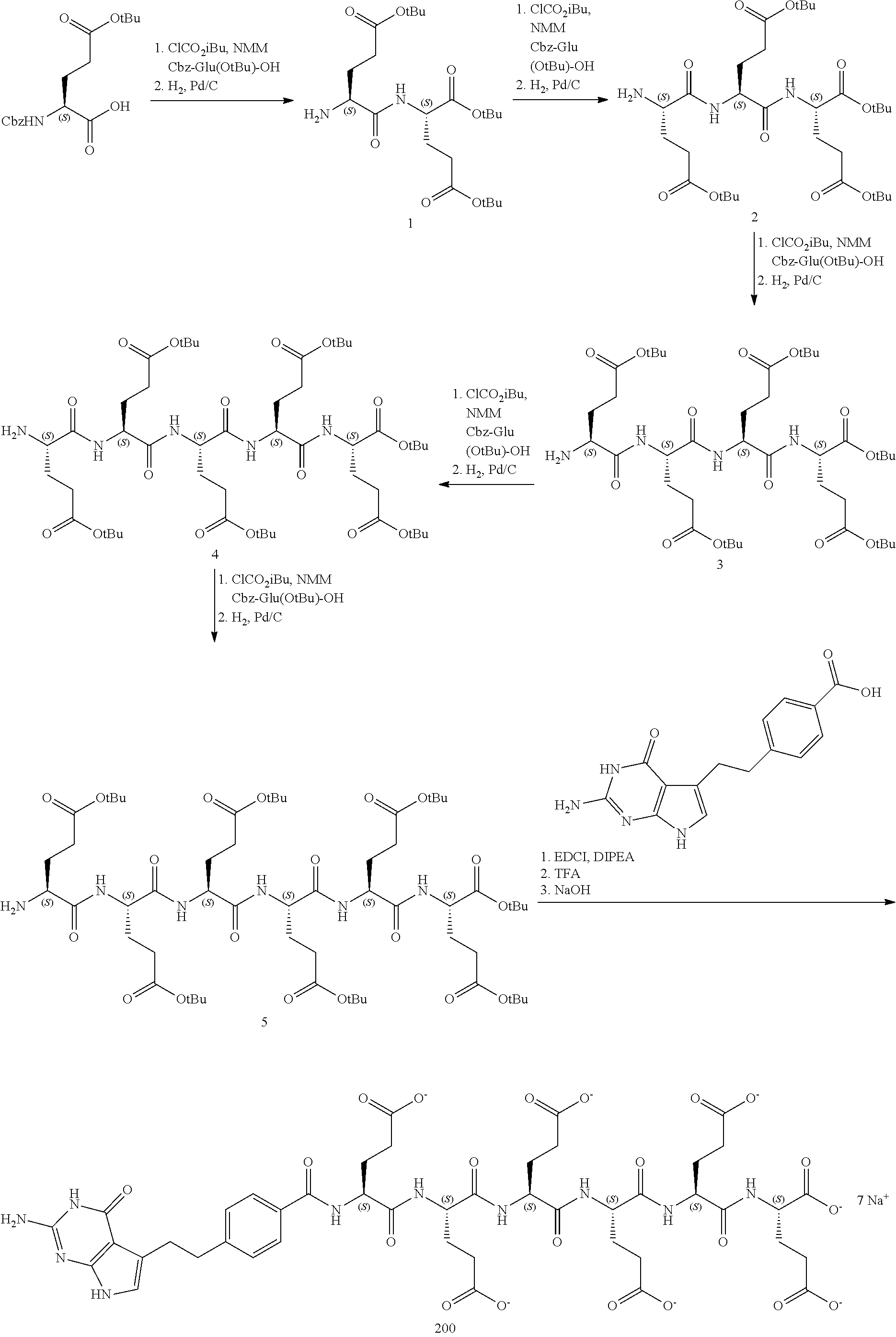

Compound 200 was prepared following similar procedures as described in Examples 1 and 2, except that for the preparation of the hexaglutamate, the amide coupling was reacted with ClCO₂i-Bu in the presence of an organic amine base, NMM (N-methylmorphine). Representative high-resolution Mass Spectral data: Calculated (M+H): 1073.3699, Found (M+H): 1073.3691.

### Example 5. Synthesis of Compound 210

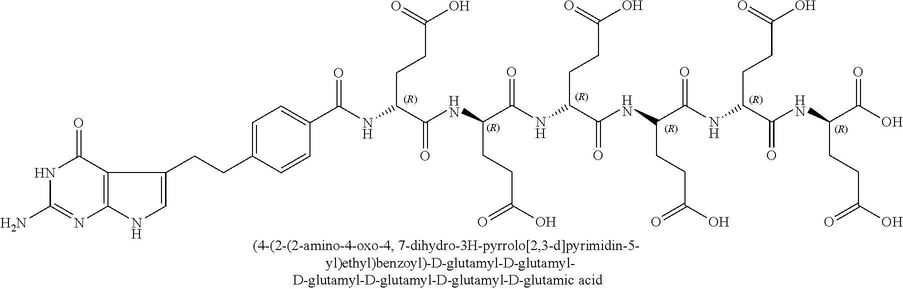

The compound 210 is the enantiomer of the free acid form of compound 200 shown in Example 4. It was synthesized similarly, except the D-glutamate starting material/intermediates were used instead. Representative high-resolution Mass Spectral data: Calculated (M+H): 1073.3699, Found (M+H): 1073.3691.

The compound 210 can also be converted into its salts such as hepta-sodium salt similar to compound 200 as shown in Example 4.

### Example 6. Solid Phase Synthesis of Compound 100 and 110

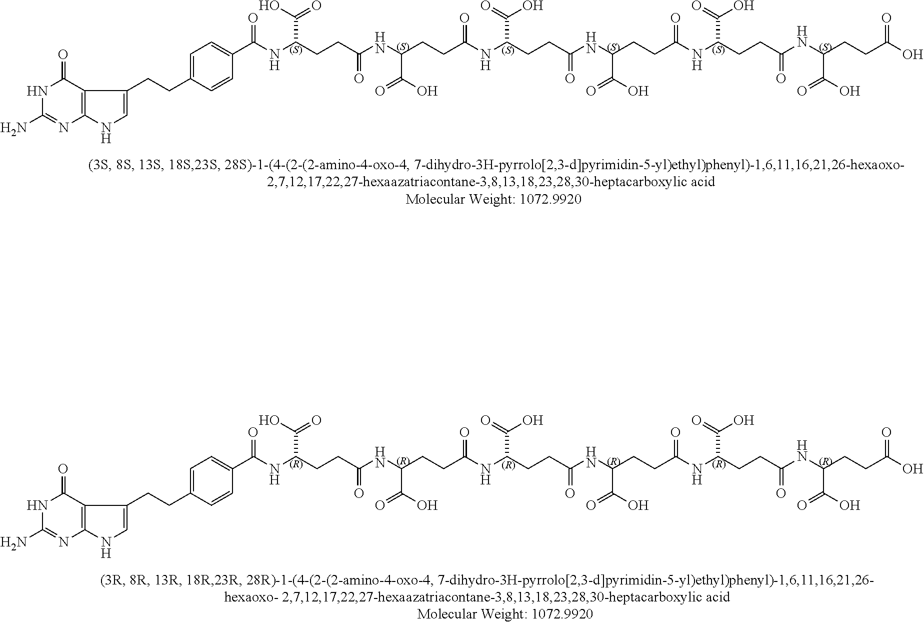

Compounds 100 and 110 can also be prepared by solid phase synthesis. A typical synthesis using a Symphony synthesizer (Gyros Protein Technologies Inc., USA) using CTC-resin (loading: 0.64 mmol/g, Lot-Nr.: 57423817744) per reaction vessel and 0.2 M amino acid solutions in DMF, 0.2 M HATU solution in DMF and 0.4 M NMM solution in DMF. Fmoc was removed with 20% piperidine in DMF for 3 minutes and a second time for 15 minutes. The washing steps are performed with NMP. The peptides were cleaved from the solid support using TFA (140 mL/mmol resin, containing 5% (v) milliQ-H₂O for 2.5 hours. The peptide containing TFA solution was collected and the resin was washed with TFA. The TFA solution was concentrated under reduced pressure and precipitated using 40 mL ice cold dietheyl ether/n-pentane (1:1, v/v) per 7 mL TFA mixture. After centrifugation the peptide is washed on additional time with ice cold dietheyl ether/n-pentane (1:1, v/v) and centrifuged. Afterwards, the crude peptide was dissolved in 5 mL ACN/water 1:1 and lyophilized. This step was repeated three times to give the desired product. Peptides were analyzed by UPLC/ESI-MS (gradient: 5-55% B in 2 min, flow: 1 mL/min, eluent A: 100% H₂O+0.05% TFA; eluent B: 100% ACN+0.05% TFA) and positive ion current for MS analysis. Then the peptide was coupled to the PEM-Acid in a standard amide coupling. The compounds were purified by Reverse-Phase preparative HPLC.

Or more generally, an initial glutamyl residue can be bonded to a Wang resin (or other suitable resins or solid supports) and additional glutamyl residues are added serially via solid phase peptide synthesis using F-moc chemistry. After the final glutamyl residue is added the Antifolate precursor (e.g., pemetrexed precursor) is coupled to the peptide and the molecule is cleaved from the resin. Or more generally, the synthesis of pemetrexed polyglutamates can be conducted as following: Synthesis of the polyglutamate peptides was performed using standard solid phase peptide synthesis using Fmoc/tBu-based chemistry on a 2-chlorotrityl resin. Linkage of the PEM moiety (Z) was performed on the solid support by coupling the unprotected des-glutamyl pemetrexed building block via its benzoic acid to the free N-terminus of the peptide using a suitable coupling reagent to form the amide bond. Cleavage of the PEM peptide from the resin and concomitant removal of the various sidechain protecting groups was performed using a cocktail of trifluoroacetic acid (TFA) with several scavengers. Subsequent workup involved precipitation from the cleavage cocktail followed by chromatographic purification using an appropriate buffer system tailored to the individual construct, yielding the PEM peptides with the requested purity and quantity. Lyophilization of the purified fractions provided the desired Pemetrexed polyglutamate peptides.

Two pemetrexed gamma polyglutamate (in L form or D form) were synthesized on solid phase resin and compounds were fully characterized by LC-MS, NMR with a HPLC purity of 96% and 98%, exact mass: 1072.3621; Calculated (M+H): 1073.3699, Found (M+H): 1073.3687.

Optical rotation in water at pH 10.8 for compound 100 [a]_{D} 10.5; Optical rotation in water at pH 10.8 for compound 110 [a]_{D}−9.2

Similarly, compounds 200 and 210 can also be prepared by solid phase synthesis using a Symphony synthesizer as compounds 100 and compound 110.

Two pemetrexed alpha polyglutamate (in L form or D form) were synthesized on solid phase resin and compounds were fully characterized by LC-MS, NMR with a HPLC purity of 96% to 98%, exact mass: 1072.3621; Calculated (M+H): 1073.3699, Found (M+H): 1073.3691.

Optical rotation in water for compound 200 [a]_{D}−33.9; Optical rotation in water at for compound 210 [a]_{D}+36.3.

### Example 7. Preparation of Hydrochloride Salts Compound 220 and 230

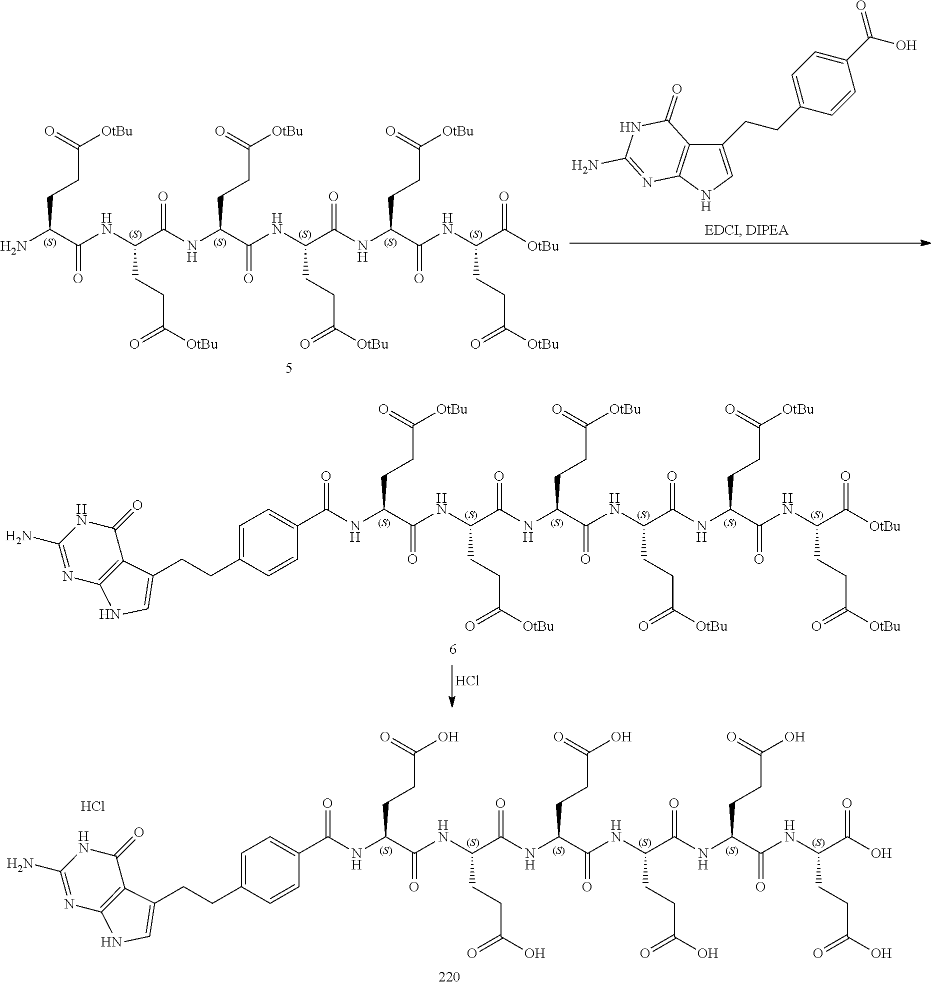

Alternatively, the alpha-hexapolyglutamated pemetrexed was prepared as an HCl salt. As shown in the above scheme, compound 6, which was obtained from compound 5 as shown in Example 4, was directly deprotected using hydrochloric acid to provide compound 220 as an HCl salt. The enantiomer of compound 220, referred to herein as compound 230, can be prepared similarly.

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A method of preparing a polyglutamated antifolate, or a pharmaceutically acceptable salt thereof, the method comprising:
reacting a protected polyglutamate of Formula I, or a salt thereof, with an antifolate having a formula of Z—COOH, or an activated form thereof, under an amide forming condition to form a compound of Formula II, or a salt thereof, 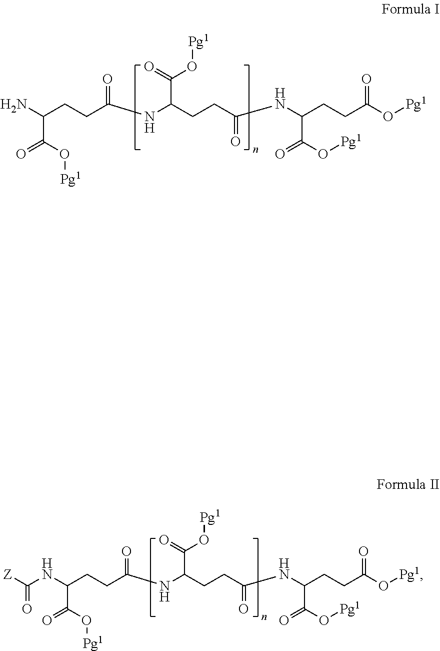
wherein:
each glutamate unit is independently in an L-form or D-form (e.g., all glutamate units are in L-form or all glutamate units are in D-form);
Pg¹ at each occurrence is independently a carboxylic acid protecting group,
n is an integer of 0-20 (e.g., 3, 4, or 5),
and Z is a residue of an antifolate selected from methotrexate (MTX), pemetrexed (PMX), lometrexol (LTX), AG2034, raltitrexed (RTX), pralatrexate, GW1843, aminopterin, LY309887 and LY222306.

2. The method of claim 1, wherein the Z is a residue of pemetrexed having the following formula:

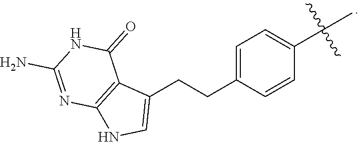 [SMILES: *c1ccc(CCc2c[nH]c3nc(N)[nH]c(=O)c23)cc1]

3. The method of claim 1 or 2, wherein n is 2-6 (e.g., 3 or 4).

4. The method of any one of claims 1-3, wherein the compound of Formula I, or salt thereof, is substantially pure.

5. The method of any one of claims 1-4, wherein Pg¹ at each occurrence is tert-butyl.

6. The method of any one of claims 1-5, wherein the reacting comprises reacting the compound of Formula I with the antifolate in the presence of an amide coupling reagent selected from chloroisobutyrate, DCC, DIC, PyBOP, PyAOP, EDCI, HATU, HBTU, TBTU, and T3P.

7. The method of any one of claims 1-6, further comprising deprotecting the compound of Formula II or a salt thereof to provide a compound of Formula III, or a salt thereof:

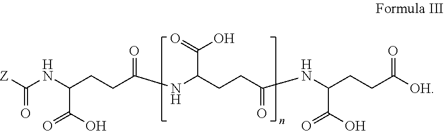

8. The method of claim 7, further comprising converting the compound of Formula III, or a salt thereof, into an alkali salt of Formula IV: 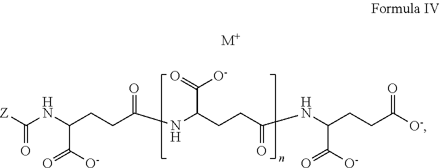
wherein M⁺ is an alkali counterion.

9. The method of any one of claims 1-8, wherein the protected polyglutamate of Formula I, or a salt thereof, is produced by a process comprising:
a) reacting an acid of Formula S-1, or an activated form thereof, with a protected polyglutamate of Formula S-2, or a salt thereof, under an amide forming condition to form a compound of Formula S-3, or a salt thereof 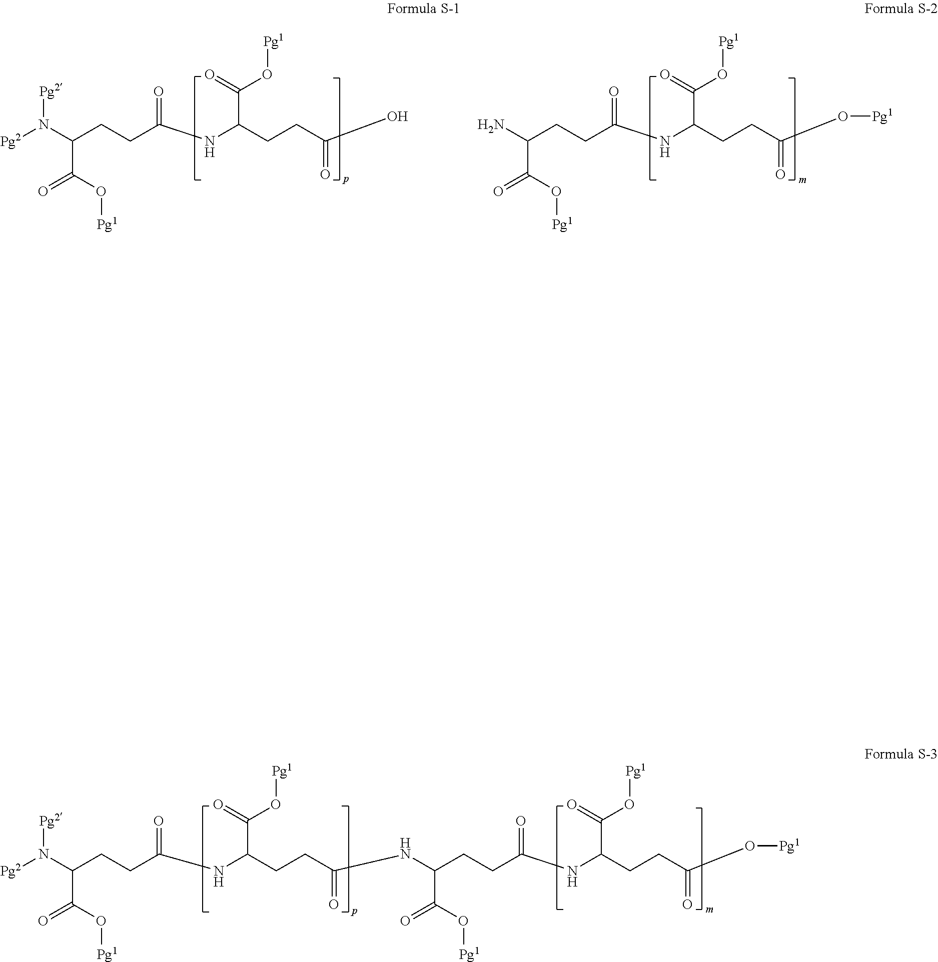
wherein:
each glutamate unit is independently in an L-form or D-form (e.g., all glutamate units are in L-form or all glutamate units are in D-form);
Pg¹ is defined above,
Pg² and Pg^{2′} are independently hydrogen or a nitrogen protecting group, provided that at least one of Pg² and Pg^{2′} is a nitrogen protecting group; or Pg² and Pg^{2′} together with the nitrogen atom they are attached to form cyclic protected amino group;
wherein m is an integer of 0-19; p is an integer of 0-19; provided that m+p=n; and
b) removing one or both of Pg² and Pg^{2′} to provide the protected polyglutamate of Formula I, or a salt thereof.

10. The method of claim 9, wherein p is 0.

11. The method of claim 9 or 10, wherein m is 2-6 (e.g., 3 or 4).

12. The method of any one of claims 9-11, wherein one of Pg² and Pg^{2′} is hydrogen, and the other of Pg² and Pg^{2′} is a nitrogen protecting group capable of being deprotected via hydrogenation, e.g., benzyloxycarbonyl.

13. A substantially pure compound of Formula III, or a pharmaceutically acceptable salt thereof: 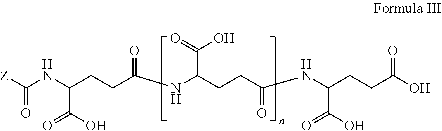
wherein:
each glutamate unit is independently in an L-form or D-form (e.g., all glutamate units are in L-form or all glutamate units are in D-form);
n is an integer of 0-20; and
Z is a residue of an antifolate selected from methotrexate (MTX), pemetrexed (PMX), lometrexol (LTX), AG2034, raltitrexed (RTX), pralatrexate, GW1843, aminopterin, LY309887 and LY222306.
wherein the substantially pure compound has a purity of at least 90% by HPLC and/or by weight.

14. The substantially pure compound of claim 13, wherein Z in Formula III is a residue of pemetrexed having the following formula:

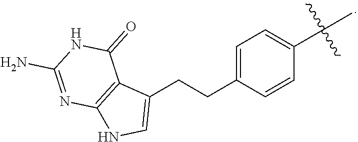 [SMILES: *c1ccc(CCc2c[nH]c3nc(N)[nH]c(=O)c23)cc1]

15. The substantially pure compound of claim 13 or 14, wherein n in Formula III is 2-6 (e.g., 3 or 4).

16. The substantially pure compound of any one of claims 13-15, wherein the compound of Formula III is in the form of a sodium salt.

17. The substantially pure compound of any one of claims 13-16, wherein the compound of Formula III is in the form of an acid addition salt.

18. A substantially pure compound of Formula III-1-L, or a pharmaceutically acceptable salt thereof: 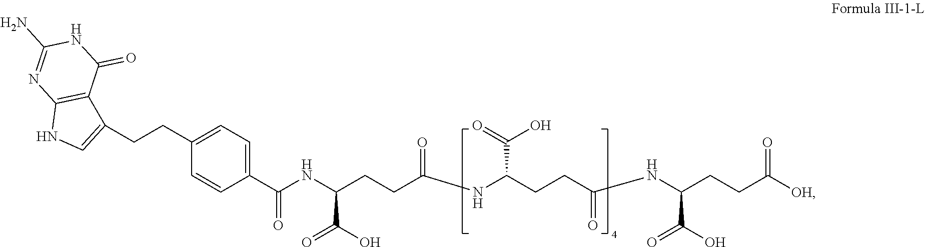
which is substantially free of a compound of Formula III-2, or a pharmaceutically acceptable salt thereof: 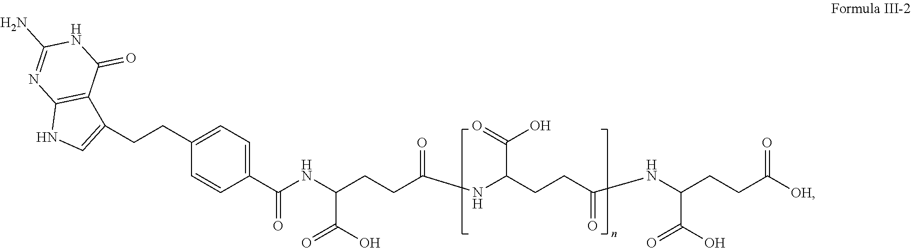
wherein n in Formula III-2 is an integer that is not 4, or n is 4 and at least one of the glutamate units is not in an L-form.

19. The substantially pure compound of claim 18, which is in the form of a sodium salt.

20. An alkali salt of Formula IV: 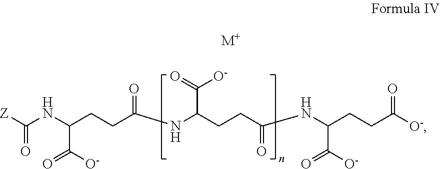
wherein:
each glutamate unit is independently in an L-form or D-form (e.g., all glutamate units are in L-form or all glutamate units are in D-form);
n is an integer of 0-20; and
Z is a residue of an antifolate selected from methotrexate (MTX), pemetrexed (PMX), lometrexol (LTX), AG2034, raltitrexed (RTX), pralatrexate, GW1843, aminopterin, LY309887 and LY222306,
wherein M⁺ is an alkali counterion.

21. The alkali salt of claim 20, wherein M⁺ is Na⁺ (e.g., n is 4, and the alkali salt of Formula IV is a hepta-sodium salt).

22. The alkali salt of claim 20 or 21, wherein Z is a residue of pemetrexed having the following formula:

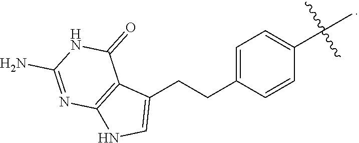 [SMILES: *c1ccc(CCc2c[nH]c3nc(N)[nH]c(=O)c23)cc1]

23. The alkali salt of any one of claims 20-22, wherein n is 2-6 (e.g., 3, 4, or 5).

24. The alkali salt of any one of claims 20-23, which is in a solid form, e.g., crystalline form, amorphous form, or a mixture thereof.

25. The alkali salt of any one of claims 20-24, which is in the form of an anhydrous form, hydrate or solvate.

26. The alkali salt of any one of claims 20-25, which has a purity by HPLC of at least 90% and/or by weight of at least 90%.

27. A pharmaceutical composition comprising the substantially pure compound of any one of claims 13-19 or the alkali salt of any one of claims 20-26.

28. The pharmaceutical composition of claim 27, formulated as an aqueous solution or suspension.

29. The pharmaceutical composition of claim 27, formulated as a liposomal composition, wherein the liposome is optionally pegylated.

30. The pharmaceutical composition of claim 29, wherein the liposomal composition has a drug load of at least 10%.

31. The pharmaceutical composition of claim 29 or 30, wherein the liposomal composition comprises a targeting moiety attached to one or both of a PEG and the exterior of the liposome, and wherein the targeting moiety has a specific affinity for a surface antigen on a target cell of interest.

32. A method for treating cancer that comprises administering an effective amount of the pharmaceutical composition of any of claims 27-31 to a subject having or at risk of having cancer.

33. The method of claim 32, wherein the cancer is selected from the group consisting of: lung cancer, pancreatic, breast cancer, ovarian cancer, lung cancer, prostate cancer, head and neck cancer, gastric cancer, gastrointestinal cancer, colon cancer, esophageal cancer, cervical cancer, kidney cancer, biliary duct cancer, gallbladder cancer, and a hematologic malignancy.

34. A method for treating cancer that comprises administering an effective amount of the composition of any of claims 27-31 to a subject having or at risk of having a cancer cell that expresses on its surface the folate receptor bound by the targeting moiety.

35. A maintenance therapy for subjects that are undergoing or have undergone cancer therapy comprising administering an effective amount of the composition of any of claims 27-31 to a subject that is undergoing or has undergone cancer therapy.

36. A method for treating a disorder of the immune system comprising administering an effective amount of the composition of any of claims 27-31 to a subject having or at risk of having a disorder of the immune system.

37. A method for treating an infectious disease comprising administering an effective amount of the composition of any of claims 27-31 to a subject having or at risk of having an infectious disease.

38. A method of delivering polyglutamated antifolate to a tumor expressing a folate receptor on its surface, the method comprising administering the composition of any of claims 27-31 to a subject having the tumor in an amount to deliver a therapeutically effective dose of the polyglutamated antifolate to the tumor.

39. A method of preparing a liposomal polyglutamated antifolate composition, the method comprising: forming a mixture comprising liposomal components and a polyglutamated antifolate in solution; homogenizing the mixture to form liposomes in the solution; and processing the mixture to form liposomes containing the polyglutamated antifolate, wherein the polyglutamated antifolate is the substantially pure compound of any of claims 13-19, or a pharmaceutically acceptable salt thereof, or the alkali salt of any of claims 20-26.

40. A method of preparing a liposomal polyglutamated antifolate composition, the method comprising: forming a mixture comprising: liposomal components and polyglutamated antifolate in a solution; homogenizing the mixture to form liposomes in the solution; processing the mixture to form liposomes entrapping and/or encapsulating polyglutamated antifolate; and providing the targeting moiety on a surface of the liposomes, the targeting moiety having the specific affinity for at least one of folate receptor alpha (FR-α), folate receptor beta (FR-β) and folate receptor delta (FR-δ), wherein the polyglutamated antifolate is the substantially pure compound of any of claims 13-19, or a pharmaceutically acceptable salt thereof, or the alkali salt of any of claims 20-26.

41. The method of claim 39 or 40, wherein the processing step includes one or more of: thin film hydration, extrusion, in-line mixing, ethanol injection technique, freezing-and-thawing technique, reverse-phase evaporation, dynamic high pressure microfluidization, microfluidic mixing, double emulsion, freeze-dried double emulsion, 3D printing, membrane contactor method, and stirring.

42. A method of preparing a polyglutamated antifolate, or a pharmaceutically acceptable salt thereof, the method comprising:
reacting a protected polyglutamate of Formula I-Alpha, or a salt thereof, with an antifolate having a formula of Z—COOH, or an activated form thereof, under an amide forming condition to form a compound of Formula II-Alpha, or a salt thereof, 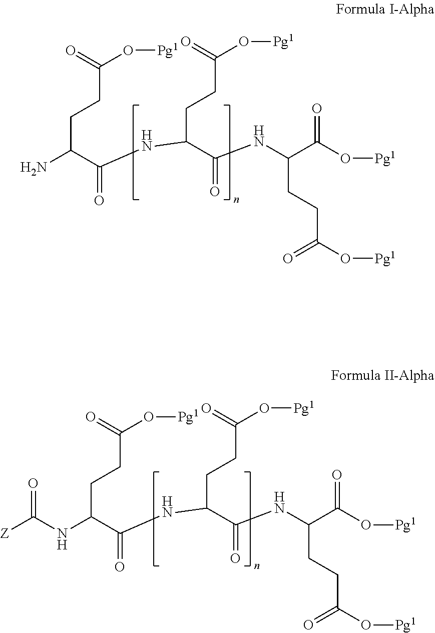
wherein:
each glutamate unit is independently in an L-form or D-form (e.g., all glutamate units are in L-form or all glutamate units are in D-form);
Pg¹ at each occurrence is independently a carboxylic acid protecting group,
n is an integer of 0-20 (e.g., 3, 4, or 5),
and Z is a residue of an antifolate selected from methotrexate (MTX), pemetrexed (PMX), lometrexol (LTX), AG2034, raltitrexed (RTX), pralatrexate, GW1843, aminopterin, LY309887 and LY222306.

43. The method of claim 42, wherein the Z is a residue of pemetrexed having the following formula:

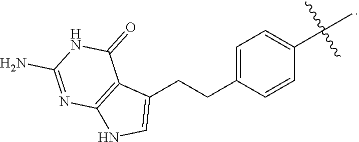 [SMILES: *c1ccc(CCc2c[nH]c3nc(N)[nH]c(=O)c23)cc1]

44. The method of claim 42 or 43, wherein n is 2-6 (e.g., 3 or 4).

45. The method of any one of claims 42-44, wherein the compound of Formula I-Alpha, or salt thereof, is substantially pure.

46. The method of any one of claims 42-45, wherein Pg¹ at each occurrence is tert-butyl.

47. The method of any one of claims 42-46, wherein the reacting comprises reacting the compound of Formula I-Alpha with the antifolate in the presence of an amide coupling reagent selected from chloroisobutyrate, DCC, DIC, PyBOP, PyAOP, EDCI, HATU, HBTU, TBTU, and T3P.

48. The method of any one of claims 42-47, further comprising deprotecting the compound of Formula II-Alpha or a salt thereof to provide a compound of Formula III-Alpha, or a salt thereof:

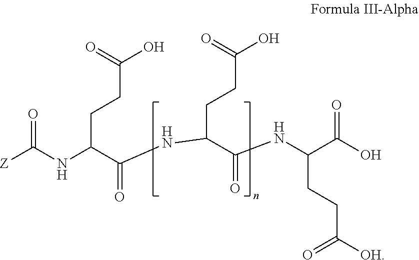

49. The method of claim 48, further comprising converting the compound of Formula III-Alpha, or a salt thereof, into an alkali salt of Formula IV-Alpha: 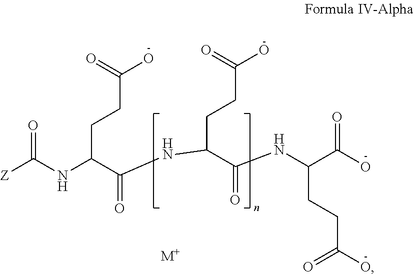
wherein M⁺ is an alkali counterion.

50. The method of any one of claims 42-49, wherein the protected polyglutamate of Formula I-Alpha, or a salt thereof, is produced by a process comprising:
a) reacting an acid of Formula S-1-Alpha, or an activated form thereof, with a protected polyglutamate of Formula S-2-Alpha, or a salt thereof, under an amide forming condition to form a compound of Formula S-3-Alpha, or a salt thereof 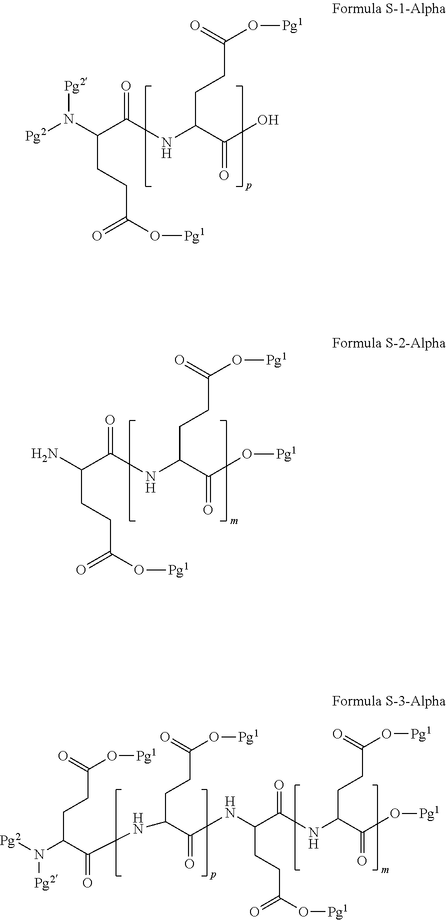
wherein:
each glutamate unit is independently in an L-form or D-form (e.g., all glutamate units are in L-form or all glutamate units are in D-form);
Pg¹ is defined above,
Pg² and Pg^{2′} are independently hydrogen or a nitrogen protecting group, provided that at least one of Pg² and Pg^{2′} is a nitrogen protecting group; or Pg² and Pg^{2′} together with the nitrogen atom they are attached to form cyclic protected amino group;
wherein m is an integer of 0-19; p is an integer of 0-19; provided that m+p=n; and
b) removing one or both of Pg² and Pg^{2′} to provide the protected polyglutamate of Formula I-Alpha, or a salt thereof.

51. The method of claim 50, wherein p is 0.

52. The method of claim 50 or 51, wherein m is 2-6 (e.g., 3 or 4).

53. The method of any one of claims 50-52, wherein one of Pg² and Pg^{2′} is hydrogen, and the other of Pg² and Pg^{2′} is a nitrogen protecting group capable of being deprotected via hydrogenation, e.g., benzyloxycarbonyl.

54. A substantially pure compound of Formula III-Alpha, or a pharmaceutically acceptable salt thereof: 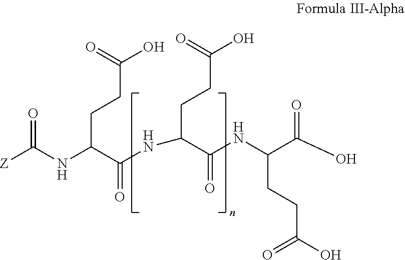
wherein:
each glutamate unit is independently in an L-form or D-form (e.g., all glutamate units are in L-form or all glutamate units are in D-form);
n is an integer of 0-20; and
Z is a residue of an antifolate selected from methotrexate (MTX), pemetrexed (PMX), lometrexol (LTX), AG2034, raltitrexed (RTX), pralatrexate, GW1843, aminopterin, LY309887 and LY222306,
wherein the substantially pure compound has a purity of at least 90% by HPLC and/or by weight.

55. The substantially pure compound of claim 54, wherein Z in Formula III-Alpha is a residue of pemetrexed having the following formula:

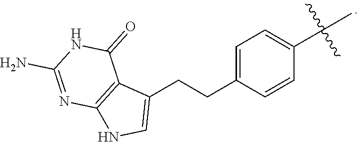 [SMILES: *c1ccc(CCc2c[nH]c3nc(N)[nH]c(=O)c23)cc1]

56. The substantially pure compound of claim 54 or 55, wherein n in Formula III-Alpha is 2-6 (e.g., 3 or 4).

57. The substantially pure compound of any one of claims 54-56, wherein the compound of Formula III-Alpha is in the form of a sodium salt.

58. The substantially pure compound of any one of claims 54-57, wherein the compound of Formula III-Alpha is in the form of an acid addition salt.

59. A substantially pure compound of Formula III-1-L-Alpha, or a pharmaceutically acceptable salt thereof: 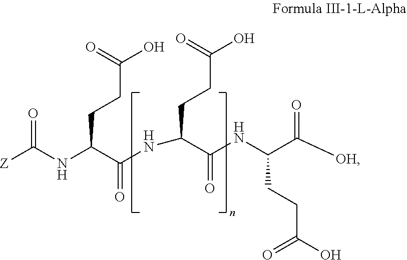
which is substantially free of a compound of Formula III-2-Alpha, or a pharmaceutically acceptable salt thereof: 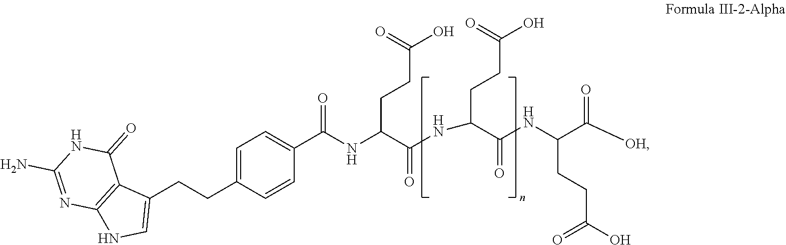
wherein n in Formula III-2-Alpha is an integer that is not 4, or n is 4 and at least one of the glutamate units is not in an L-form.

60. The substantially pure compound of claim 59, which is in the form of a sodium salt.

61. An alkali salt of Formula IV-Alpha: 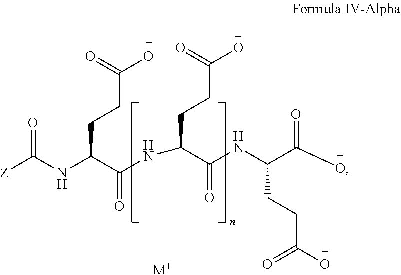
wherein:
each glutamate unit is independently in an L-form or D-form (e.g., all glutamate units are in L-form or all glutamate units are in D-form);
n is an integer of 0-20; and
Z is a residue of an antifolate selected from methotrexate (MTX), pemetrexed (PMX), lometrexol (LTX), AG2034, raltitrexed (RTX), pralatrexate, GW1843, aminopterin, LY309887 and LY222306,
wherein M⁺ is an alkali counterion.

62. The alkali salt of claim 61, wherein M⁺ is Na⁺ (e.g., n is 4, and the alkali salt of Formula IV is a hepta-sodium salt).

63. The alkali salt of claim 61 or 62, wherein Z is a residue of pemetrexed having the following formula:

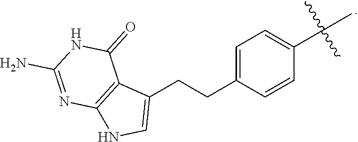 [SMILES: *c1ccc(CCc2c[nH]c3nc(N)[nH]c(=O)c23)cc1]

64. The alkali salt of any one of claims 61-63, wherein n is 2-6 (e.g., 3, 4, or 5).

65. The alkali salt of any one of claims 61-64, which is in a solid form, e.g., crystalline form, amorphous form, or a mixture thereof.

66. The alkali salt of any one of claims 61-65, which is in the form of an anhydrous form, hydrate or solvate.

67. The alkali salt of any one of claims 61-66, which has a purity by HPLC of at least 90% and/or by weight of at least 90%.

68. A pharmaceutical composition comprising the substantially pure compound of any one of claims 54-60 or the alkali salt of any one of claims 61-67.

69. The pharmaceutical composition of claim 68, formulated as an aqueous solution or suspension.

70. The pharmaceutical composition of claim 68, formulated as a liposomal composition, wherein the liposome is optionally pegylated.

71. The pharmaceutical composition of claim 70, wherein the liposomal composition has a drug load of at least 10%.

72. The pharmaceutical composition of claim 70 or 71, wherein the liposomal composition comprises a targeting moiety attached to one or both of a PEG and the exterior of the liposome, and wherein the targeting moiety has a specific affinity for a surface antigen on a target cell of interest.

73. A method for treating cancer that comprises administering an effective amount of the pharmaceutical composition of any of claims 68-72 to a subject having or at risk of having cancer.

74. The method of claim 73, wherein the cancer is selected from the group consisting of: lung cancer, pancreatic, breast cancer, ovarian cancer, lung cancer, prostate cancer, head and neck cancer, gastric cancer, gastrointestinal cancer, colon cancer, esophageal cancer, cervical cancer, kidney cancer, biliary duct cancer, gallbladder cancer, and a hematologic malignancy.

75. A method for treating cancer that comprises administering an effective amount of the composition of any of claims 68-72 to a subject having or at risk of having a cancer cell that expresses on its surface the folate receptor bound by the targeting moiety.

76. A maintenance therapy for subjects that are undergoing or have undergone cancer therapy comprising administering an effective amount of the composition of any of claims 68-72 to a subject that is undergoing or has undergone cancer therapy.

77. A method for treating a disorder of the immune system comprising administering an effective amount of the composition of any of claims 68-72 to a subject having or at risk of having a disorder of the immune system.

78. A method for treating an infectious disease comprising administering an effective amount of the composition of any of claims 68-72 to a subject having or at risk of having an infectious disease.

79. A method of delivering polyglutamated antifolate to a tumor expressing a folate receptor on its surface, the method comprising administering the composition of any of claims 68-72 to a subject having the tumor in an amount to deliver a therapeutically effective dose of the polyglutamated antifolate to the tumor.

80. A method of preparing a liposomal polyglutamated antifolate composition, the method comprising: forming a mixture comprising liposomal components and a polyglutamated antifolate in solution; homogenizing the mixture to form liposomes in the solution; and processing the mixture to form liposomes containing the polyglutamated antifolate, wherein the polyglutamated antifolate is the substantially pure compound of any of claims claims 54-60, or a pharmaceutically acceptable salt thereof, or the alkali salt of any one of claims 61-67.

81. A method of preparing a liposomal polyglutamated antifolate composition, the method comprising: forming a mixture comprising: liposomal components and polyglutamated antifolate in a solution; homogenizing the mixture to form liposomes in the solution; processing the mixture to form liposomes entrapping and/or encapsulating polyglutamated antifolate; and providing the targeting moiety on a surface of the liposomes, the targeting moiety having the specific affinity for at least one of folate receptor alpha (FR-a), folate receptor beta (FR-β) and folate receptor delta (FR-δ), wherein the polyglutamated antifolate is the substantially pure compound of any of claims 54-60, or a pharmaceutically acceptable salt thereof, or the alkali salt of any of claims 61-67.

82. The method of claim 80 or 81, wherein the processing step includes one or more of: thin film hydration, extrusion, in-line mixing, ethanol injection technique, freezing-and-thawing technique, reverse-phase evaporation, dynamic high pressure microfluidization, microfluidic mixing, double emulsion, freeze-dried double emulsion, 3D printing, membrane contactor method, and stirring.


SEQUENCE LISTING (44 sequences)

SEQ ID NO: 1 - protein, 25 aa, Artificial Sequence
  synthetic construct
RQIKIWFQNRRMKWKKRKKRRQRRR

SEQ ID NO: 2 - protein, 11 aa, Artificial Sequence
  synthetic construct
  MISC_FEATURE (6)..(6): Xaa can be any naturally occurring amino acid
RKKRRXRRRGC

SEQ ID NO: 3 - DNA, 13 bp, Artificial Sequence
  synthetic construct
ccgccaagaagcg

SEQ ID NO: 4 - DNA, 100 bp, Artificial Sequence
  synthetic construct
gcgtgcacacgcgcgtagacttcccccgcaagtcactcgttagcccgccaagaagcgacccctccggggcgagctgagcggcgtggcgcgggggcgtcat

SEQ ID NO: 5 - DNA, 101 bp, Artificial Sequence
  synthetic construct
acgtgcatacgcacgtagacattccccgcttcccactccaaagtccgccaagaagcgtatcccgctgagcggcgtggcgcgggggcgtcatccgtcagctc

SEQ ID NO: 6 - DNA, 58 bp, Artificial Sequence
  synthetic construct
acttcccccgcaagtcactcgttagcccgccaagaagcgacccctccggggcgagctg

SEQ ID NO: 7 - protein, 9 aa, Artificial Sequence
  synthetic construct
RKKRRQRRR

SEQ ID NO: 8 - protein, 13 aa, Artificial Sequence
  synthetic construct
GRKKRRQRRRTPQ

SEQ ID NO: 9 - protein, 11 aa, Artificial Sequence
  synthetic construct
YGRKKRRQRRR

SEQ ID NO: 10 - protein, 15 aa, Artificial Sequence
  synthetic construct
AAVALLPAVLLALLA

SEQ ID NO: 11 - protein, 15 aa, Artificial Sequence
  synthetic construct
MGLGLHLLVLAAALQ

SEQ ID NO: 12 - protein, 15 aa, Artificial Sequence
  synthetic construct
GALFLGFLGAAGSTM

SEQ ID NO: 13 - protein, 21 aa, Artificial Sequence
  synthetic construct
AGYLLGKINLKALAALAKKIL

SEQ ID NO: 14 - protein, 16 aa, Artificial Sequence
  synthetic construct
RVIRVWFQNKRCKDKK

SEQ ID NO: 15 - protein, 16 aa, Artificial Sequence
  synthetic construct
RQIKIWFQNRRMKWKK

SEQ ID NO: 16 - protein, 20 aa, Artificial Sequence
  synthetic construct
GLFEAIAGFIENGWEGMIDG

SEQ ID NO: 17 - protein, 15 aa, Artificial Sequence
  synthetic construct
GWTLNSAGYLLGKIN

SEQ ID NO: 18 - protein, 15 aa, Artificial Sequence
  synthetic construct
RSQSRSRYYRQRQRS

SEQ ID NO: 19 - protein, 8 aa, Artificial Sequence
  synthetic construct
LAIPEQEY

SEQ ID NO: 20 - protein, 8 aa, Artificial Sequence
  synthetic construct
LGIAEQEY

SEQ ID NO: 21 - protein, 8 aa, Artificial Sequence
  synthetic construct
LGIPAQEY

SEQ ID NO: 22 - protein, 8 aa, Artificial Sequence
  synthetic construct
LGIPEAEY

SEQ ID NO: 23 - protein, 8 aa, Artificial Sequence
  synthetic construct
LGIPEQAY

SEQ ID NO: 24 - protein, 8 aa, Artificial Sequence
  synthetic construct
LGIAEAEY

SEQ ID NO: 25 - protein, 8 aa, Artificial Sequence
  synthetic construct
LGIPEAAY

SEQ ID NO: 26 - protein, 8 aa, Artificial Sequence
  synthetic construct
LGIAEQAY

SEQ ID NO: 27 - protein, 8 aa, Artificial Sequence
  synthetic construct
LGIAEAAY

SEQ ID NO: 28 - protein, 18 aa, Artificial Sequence
  synthetic construct
LLIILRRRIRKQAHAHSK

SEQ ID NO: 29 - protein, 12 aa, Artificial Sequence
  synthetic construct
LKALAALAKKIL

SEQ ID NO: 30 - protein, 18 aa, Artificial Sequence
  synthetic construct
KLALKLALKALKAALKLA

SEQ ID NO: 31 - protein, 21 aa, Artificial Sequence
  synthetic construct
KETWWETWWTEWSQPKKKRKV

SEQ ID NO: 32 - protein, 8 aa, Artificial Sequence
  synthetic construct
DHQLNPAF

SEQ ID NO: 33 - protein, 8 aa, Artificial Sequence
  synthetic construct
DPKGDPKG

SEQ ID NO: 34 - protein, 18 aa, Artificial Sequence
  synthetic construct
VTVTVTVTVTGKGDPKPD

SEQ ID NO: 35 - protein, 16 aa, Artificial Sequence
  synthetic construct
RQIKIWFQNRRMKWKK

SEQ ID NO: 36 - protein, 13 aa, Artificial Sequence
  synthetic construct
GRKKRRQRRRPPQ

SEQ ID NO: 37 - protein, 27 aa, Artificial Sequence
  synthetic construct
GWTLNSAGYLLGKINLKALAALAKKIL

SEQ ID NO: 38 - protein, 10 aa, Artificial Sequence
  synthetic construct
GRKKRRQRRR

SEQ ID NO: 39 - protein, 7 aa, Artificial Sequence
  synthetic construct
RRRRRRR

SEQ ID NO: 40 - protein, 8 aa, Artificial Sequence
  synthetic construct
RRRRRRRR

SEQ ID NO: 41 - protein, 9 aa, Artificial Sequence
  synthetic construct
RRRRRRRRR

SEQ ID NO: 42 - protein, 10 aa, Artificial Sequence
  synthetic construct
RRRRRRRRRR

SEQ ID NO: 43 - protein, 10 aa, Artificial Sequence
  synthetic construct
RRRRRRRRRR

SEQ ID NO: 44 - protein, 33 aa, Artificial Sequence
  synthetic construct
  MISC_FEATURE (33)..(33): Xaa can be 2 to 15 Arg independently in the L and/or D form
YTIWMPENPRPGTPCDIFTNSRGKRASNGGGGX